(19) United States
(12) Patent Application Publication
(10) Pub. No.: US 2023/0227802 A1
(43) Pub. Date: Jul. 20, 2023

(54) COMPOSITIONS AND METHODS FOR THE TREATMENT OF NEUROLOGICAL DISORDERS RELATED TO GLUCOSYLCERAMIDASE BETA DEFICIENCY

(71) Applicant: VOYAGER THERAPEUTICS, INC., Cambridge, MA (US)

(72) Inventors: Giridhar MURLIDHARAN, Malden, MA (US); Jeffrey BROWN, Medway, MA (US); Elisabeth KNOLL, Melrose, MA (US); Yanqun SHU, Winchester, MA (US); Kelly BALES, Lincoln, MA (US); Jinzhao HOU, Lexington, MA (US); Adewale ADELUYI, Boston, MA (US); Brett HOFFMAN, Braintree, MA (US); Smita JAGTAP, Melrose, MA (US)

(21) Appl. No.: 18/018,017

(22) PCT Filed: Jul. 26, 2021
(86) PCT No.: PCT/US2021/043216

§ 371 Date: Jan. 25, 2023

Related U.S. Application Data
(60) Provisional application No. 63/057,265, filed on Jul. 27, 2020.

(51) Int. Cl. C12N 9/24; C12N 15/86; A61P 25/28

(52) U.S. Cl. CPC C12N 9/2402; C12N 15/86; A61P 25/28; C12N 2750/14143; C12Y 302/01045

(57) ABSTRACT

The disclosure relates to compositions and methods for altering, e.g., enhancing, the expression of GCase proteins, whether in vitro and/or in vivo. Such compositions include delivery of an adeno-associated viral (AAV) particle. The compositions and methods of the present disclosure are useful in the treatment of subjects diagnosed with, or suspected of having Parkinson Disease or related condition resulting from a deficiency in the quantity and/or function of GBA gene product or associated with decreased expression or protein levels of GCase protein.

### RELATED APPLICATIONS

This application claims priority to U.S. provisional application No. 63/057,265, filed on Jul. 27, 2020. The entire contents of the foregoing application are hereby incorporated herein by reference.

### SEQUENCE LISTING

The present application is being filed along with a Sequence Listing in electronic format. The Sequence Listing file, entitled 135333-00120_SL.txt, was created on Jul. 23, 2021, and is 6,773,307 bytes in size. The information in electronic format of the Sequence Listing is incorporated herein by reference in its entirety.

### FIELD OF THE DISCLOSURE

Described herein are compositions and methods relating to polynucleotides, e.g. polynucleotides encoding glucosylceramidase beta (GBA) proteins and peptides for use in the treatment of Parkinson Disease (PD) and related disorders, including Gaucher Disease, and Dementia with Lewy Bodies (collectively, “GBA-related disorders”). In some embodiments, compositions may be delivered in an adeno-associated viral (AAV) vector. In other embodiments, compositions described herein, may be used to treat a subject in need thereof, such as a human subject diagnosed with GBA-related disorders or other condition resulting from a deficiency in the quantity and/or function of GBA protein, or as a research tool in the study of diseases or conditions in cells or animal models of such disease or condition.

### BACKGROUND

Lysosomal acid glucosylceramidase, commonly called glucosylcerebrosidase or GCase, a D-glucosyl-N-acylsphingosine glucohydrolase, is a lysosomal membrane protein important in glycolipid metabolism. The enzyme is encoded by glucosylceramidase beta (GBA) gene (Ensembl Gene ID No. ENSG00000177628). This enzyme, together with Saposin A and Saposin C, catalyzes the hydrolysis of glucosylceramide to ceramide and glucose. See Vaccaro, Anna Maria, et al. Journal of Biological Chemistry 272.27 (1997): 16862-16867, the contents of which are incorporated herein by reference in their entirety.

Mutations in GBA are known to cause disease in human subjects. Homozygous or compound heterozygous GBA mutations lead to Gaucher disease (“GD”). See Sardi, S. Pablo, Jesse M. Cedarbaum, and Patrik Brundin. Movement Disorders 33.5 (2018): 684-696, the contents of which are herein incorporated by reference in their entirety. Gaucher disease is one of the most prevalent lysosomal storage disorders, with an estimated standardized birth incidence in the general population of between 0.4 to 5.8 individuals per 100,000. Heterozygous GBA mutations can lead to PD. Indeed, GBA mutations occur in 7-10% of total PD patients, making GBA mutations the most important genetic risk factor of PD. PD-GBA patients have reduced levels of lysosomal enzyme beta-glucocerebrosidase (GCase), which results in increased accumulations of glycosphingolipid glucosylceramide (GluCer), which in turn is correlated with exacerbated α-Synuclein aggregation and concomitant neurological symptoms. Gaucher disease and PD, as well as other lysosomal storage disorders including Lewy body diseases such as Dementia with Lewy Bodies, and related diseases, in some cases, share common etiology in the GBA gene. See Sidransky, E. and Lopez, G. Lancet Neurol. 2012 November; 11(11): 986-998, the contents of which are incorporated by reference in their entirety. Limited treatment options exist for such diseases.

Consequently, there remains a long felt-need to develop pharmaceutical compositions and methods for the treatment of PD and other GBA-related disorders and to ameliorate deficiencies of GCase protein in patients afflicted with GBA-related disorders.

### SUMMARY

The present disclosure addresses these challenges by providing AAV-based compositions and methods for treating GCase deficiency in patients. Disclosed herein are compositions and methods directed to AAV-based gene delivery of GCase to ameliorate loss-of-function and to improve intracellular lipid trafficking. The compositions and methods are useful to improve lysosomal glycolipid metabolism, and to slow, halt, or reverse neurodegenerative and other symptoms of PD and GBA-related disorders (e.g., dementia with Lewy Bodies (DLB), Gaucher disease (GD)) in a subject (e.g., a subject having a mutation in a GBA gene). A β-glucocerebrosidase (GBA) protein is also sometimes referred to as a GCase protein herein.

Accordingly, in one aspect, the present disclosure provides an isolated, e.g., recombinant, nucleic acid comprising a transgene encoding a GBA protein, wherein the nucleotide sequence encoding the GBA protein comprises a nucleotide sequence, e.g., a codon optimized nucleotide sequence, at least 88% (e.g., at least 89, 90, 92, 95, 96, 97, 98, or 99%) identical to the nucleotide sequence of SEQ ID NO: 1773. In some embodiments, the nucleic acid further encodes an enhancement element, e.g., an enhancement element described herein.

In another aspect, the disclosure provides an isolated, e.g., recombinant, nucleic acid comprising a transgene encoding a GBA protein and an enhancement element, wherein the encoded enhancement element comprises: a Saposin C polypeptide or functional fragment or variant thereof, optionally comprising the amino acid sequence of SEQ ID NO: 1789 or 1758, or an amino acid sequence at least 85% (e.g., at least 90%, 92%, 95%, 97%, 98%, or 99%) identical thereto; a cell penetrating peptide, optionally comprising the amino acid sequence of any of SEQ ID NOs: 1794, 1796, or 1798, or an amino acid sequence having at least one, two, or three but no more than four modifications, e.g., substitutions (e.g., conservative substitutions), relative to SEQ ID NOs: 1794, 1796, or 1798; and/or a lysosomal targeting sequence, optionally comprising the amino acid sequence of any of SEQ ID NOs: 1800, 1802, 1804, 1806, or 1808, or an amino acid sequence having at least one, two, or three but no more than four modifications, e.g., substitutions (e.g., conservative substitutions), relative to SEQ ID NOs: 1800, 1802, 1804, 1806, or 1808.

In another aspect, the present disclosure provides, an isolated, e.g., recombinant viral genome comprising a nucleic acid comprising a transgene encoding a GBA protein, and further comprising a nucleotide sequence encoding a miR binding site that modulates, e.g., reduces, expression of the encoded GBA protein in a cell or tissue of the DRG, liver, hematopoietic lineage, or a combination thereof. In some embodiments, the encoded miR binding site comprises a miR183 binding site. In some embodiments, the viral genome further encodes an enhancement element, e.g., an enhancement element described herein.

In yet another aspect, the present disclosure provides an isolated, e.g., recombinant viral genome comprising a promoter operably linked to a nucleic acid comprising a transgene encoding a GBA protein described herein. In some embodiments, the viral genome comprises an internal terminal repeat (ITR) sequence (e.g., an ITR region described herein), an enhancer (e.g., an enhancer described herein), an intron region (e.g., an intron region described herein), a Kozak sequence (e.g., a Kozak sequence described herein), an exon region (e.g., an exon region described herein), a nucleotide sequence encoding a miR binding site (e.g., a miR binding site described herein) and/or a poly A signal region (e.g., a poly A signal sequence described herein). In some embodiments, the viral genome comprises the nucleotide sequence of SEQ ID NO: 1812 or 1826, or a nucleotide sequence at least 95% identical thereto. In some embodiments, the viral genome comprises the nucleotide sequence of any one of SEQ ID NOs: 1759-1771, 1809-1811, or 1813-1827, or a nucleotide sequence at least 95% identical thereto.

In yet another aspect, the present disclosure provides an isolated, e.g., recombinant, AAV particle comprising a capsid protein and a viral genome comprising a promoter (e.g., a promoter described herein) operably linked transgene encoding a GBA protein described herein. In some embodiments, the capsid protein comprises an AAV capsid protein. In some embodiments, the capsid protein comprises a VOY101 capsid protein, an AAV9 capsid protein, or a functional variant thereof.

In yet another aspect, the present disclosure provides a method of making a viral genome described herein The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral from the backbone region, e.g., by cleaving the nucleic acid molecule at upstream and downstream of the viral genome.

In yet another aspect, the present disclosure provides a method of making an isolated, e.g., recombinant AAV particle. The method comprising providing a host cell comprising a viral genome described herein and incubating the host cell under conditions suitable to enclose the viral genome in the AAV particle, e.g., a VOY101 capsid protein, thereby making the isolated AAV particle.

In yet another aspect, the present disclosure provides method of delivering an exogenous GBA protein, to a subject. The method comprises administering an effective amount of an AAV particle or a plurality of AAV particles, described herein, said AAV particle comprising a viral genome described herein, e.g., a viral genome comprising a nucleic acid comprising a transgene encoding a GBA protein described herein.

In yet another aspect, the present disclosure provides method of treating a subject having or diagnosed with having a disease associated with GBA expression, a neurological disorder, or a neuromuscular disorder. The method comprises administering an effective amount of an AAV particle or a plurality of AAV particles, described herein, said AAV particle comprising a viral genome described herein, e.g., a viral genome comprising a nucleic acid comprising a transgene encoding a GBA protein described herein. In some embodiments, the disease associated with expression of GBA or the neurodegenerative or neuromuscular disorder comprises Parkinson's Disease (PD) (e.g., a PD associated with a mutation in a GBA gene), dementia with Lewy Bodies (DLB), Gaucher disease (GD), Spinal muscular atrophy (SMA), Multiple System Atrophy (MSA), or Multiple sclerosis (MS).

In some aspects, the present disclosure provides AAV viral genomes comprising at least one inverted terminal repeat (ITR) and a payload region, wherein the payload region encodes one or more GCase proteins including GCase peptides. In some embodiments, the AAV viral genome comprises a 5′ ITR, a promoter, a payload region comprising a nucleotide sequence encoding a GCase protein, and a 3′ ITR. The encoded protein may be a human (Homo sapiens) GCase, a cynomolgus monkey (Macaca fascicularis) GCase, or a rhesus monkey (Macaca mulatta) GCase, a synthetic (non-naturally occurring) GCase, or a derivative thereof, e.g., a variant that retains one or more function of a wild-type GCase protein. In some embodiments, the GCase may be at least partially humanized.

The GCase of the present disclosure can be co-expressed with a saposin protein. In some embodiments, the transgene encoding the GCase includes a nucleotide sequence encoding the saposin protein. In some embodiments, the saposin protein is saposin A (SapA). In some embodiments, the saposin protein in saposin C (SapC).

Viral genomes may be incorporated into an AAV particle, wherein the AAV particle comprises a viral genome and a capsid. In some embodiments, the capsid comprises a sequence as shown in Table 1.

In some embodiments, the AAV particles described herein may be used in pharmaceutical compositions. The pharmaceutical compositions may be used to treat a disorder or condition associated with decreased GCase expression, activity, or protein levels. In some embodiments, the disorder or condition is a lysosomal lipid storage disorder. In some embodiments, the disorder or condition associated with decreased GCase protein levels is PD (e.g., a PD associated with a mutation in a GBA gene), Gaucher disease (e.g., Type 1 GD (e.g., non-neuronopathic GD), Type 2 (e.g., acute neuronopathic GD), or Type 3 GD), or other GBA-related disorder (e.g., dementia with Lewy Bodies (DLB). In some embodiments, administration of AAV particles may result in enhanced GCase expression in a target cell.

In some aspects, the present disclosure provides methods of increasing GCase enzyme activity in patients using AAV mediated gene transfer of an optimized GBA transgene cassette. The AAV mediated gene transfer can be optimized to achieve widespread CNS distribution, and thereby decrease substrate glycosphingolipid glucosylceramide/GluCer levels and α-synuclein pathology, slowing or reversing disease pathogenesis in patients with GB A-related disorders, including GBA patients with Parkinson disease (GBA-PD), Gaucher disease (e.g., Type 2 or 3 GD), and Dementia with Lewy body disease. In some embodiments, the methods involve intrastriatal (ISTR) or intracisternal (ICM) administration of AAV vectors packaging optimized GBA gene replacement transgene cassettes as described herein to achieve widespread, cell-autonomous transduction and cross-correction of therapeutic GCase enzyme.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. An isolated, e.g., recombinant, nucleic acid comprising a transgene encoding a β-glucocerebrosidase (GBA) protein, wherein the nucleotide sequence encoding the GBA protein comprises a nucleotide sequence, e.g., a codon optimized nucleotide sequence, at least 88% (e.g., at least 89, 90, 92, 95, 96, 97, 98, or 99%) identical to the nucleotide sequence of SEQ ID NO: 1773.

2. The isolated nucleic acid of embodiment 1, wherein the nucleotide sequence encoding the GBA protein comprises a nucleotide sequence at least 90% identical to SEQ ID NO: 1773.

3. The isolated nucleic acid of embodiment 1 or 2, wherein the nucleotide sequence encoding the GBA protein comprises a nucleotide sequence at least 95% identical to SEQ ID NO: 1773.

4. The isolated nucleic acid of any one of embodiments 1-3, wherein the nucleotide sequence encoding the GBA protein comprises the nucleotide sequence of SEQ ID NO: 1773.

5. The isolated nucleic acid of any one of embodiments 1-4, further comprising an enhancement element.

6. An isolated, e.g., recombinant, nucleic acid comprising a transgene encoding a β-glucocerebrosidase (GBA) protein and an enhancement element, wherein the encoded enhancement element comprises:

(a) a Saposin C polypeptide or functional fragment or variant thereof, optionally comprising the amino acid sequence of SEQ ID NO: 1789 or 1758, or an amino acid sequence at least 85% (e.g., at least 90%, 92%, 95%, 97%, 98%, or 99%) identical thereto;

(b) a cell penetrating peptide, optionally comprising the amino acid sequence of any of SEQ ID NOs: 1794, 1796, or 1798, or an amino acid sequence having at least one, two, or three but no more than four modifications, e.g., substitutions (e.g., conservative substitutions), relative to SEQ ID NOs: 1794, 1796, or 1798; and/or

(c) a lysosomal targeting sequence, optionally comprising the amino acid sequence of any of SEQ ID NOs: 1800, 1802, 1804, 1806, or 1808, or an amino acid sequence having at least one, two, or three but no more than four modifications, e.g., substitutions (e.g., conservative substitutions), relative to SEQ ID NOs: 1800, 1802, 1804, 1806, or 1808.

7. An isolated, e.g., recombinant viral genome comprising a nucleic acid comprising a transgene encoding a β-glucocerebrosidase (GBA) protein, and further comprising a nucleotide sequence encoding a miR binding site that modulates, e.g., reduces, expression of the encoded GBA protein in a cell or tissue of the DRG, liver, hematopoietic lineage, or a combination thereof.

8. The viral genome of embodiment 7, wherein the nucleic acid further encodes an enhancement element.

9. The isolated nucleic acid of embodiment 5 or 6, or the viral genome of embodiment 8, wherein the encoded enhancement element comprises a Saposin C polypeptide or functional fragment or variant thereof.

10. The isolated nucleic acid of embodiment 5-6 or 9, or the viral genome of embodiment 8 or 9, wherein:

(i) the encoded Saposin C polypeptide or functional fragment or variant thereof comprises the amino acid sequence of SEQ ID NO: 1789 or 1758, or an amino acid sequence at least sequence at least 85% (e.g., at least 90%, 92%, 95%, 97%, 98%, or 99%) identical thereto; and/or

(ii) the nucleotide sequence encoding the encoded Saposin C polypeptide or functional fragment or variant thereof comprises the nucleotide sequence of SEQ ID NO: 1787 or 1791, or a nucleotide sequence at least 85% (e.g., at least 90%, 92%, 95%, 97%, 98%, or 99%) identical thereto.

11. The isolated nucleic acid of embodiment 5, or the viral genome of embodiment 8, wherein:

(i) the encoded enhancement element comprises the amino acid sequence of any of SEQ ID NOs: 1750, 1752, 1754, 1756-1758, 1784, or 1785, an amino acid sequence having at least one, two, or three but no more than four modifications, e.g., substitutions (e.g., conservative substitutions), relative to SEQ ID NO: 1750, 1752, 1754, 1756-1758, 1784, or 1785, or an amino acid sequence at least 85% (e.g., at least 90%, 92%, 95%, 97%, 98%, or 99%) identical thereto; and/or

(ii) the nucleotide sequence encoding the enhancement element comprises the nucleotide sequence of any one of SEQ ID NOs: 1751, 1753, 1755, 1858, or 1859, or a nucleotide sequence at least 85% (e.g., at least 90%, 92%, 95%, 97%, 98%, or 99%) identical thereto.

12. The isolated nucleic acid of any one of embodiments 5-6 or 9-11, or the viral genome of embodiment 8-11, wherein the encoded enhancement element comprises a cell penetrating peptide.

13. The isolated nucleic acid of embodiment 6 or 12, or the viral genome of embodiment 12, wherein:

(i) the cell penetrating peptide comprises the amino acid sequence of any of SEQ ID NOs: 1794, 1796, or 1798, or an amino acid sequence having at least one, two, or three but no more than four modifications, e.g., substitutions (e.g., conservative substitutions), relative to SEQ ID NOs: 1794, 1796, or 1798;

(ii) the nucleotide sequence encoding the cell penetrating peptide comprises the nucleotide sequence of any of SEQ ID NOs: 1793, 1795, or 1797, or a nucleotide sequence at least 80% (e.g., 85%, 90%, 92%, 95%, 96%, 97%, 98%, or 99%) identical thereto.

14. The isolated nucleic acid of any one of embodiments 5-6 or 9-13, or the viral genome of any one of embodiments 8-13, wherein the encoded enhancement element comprises a lysosomal targeting sequence.

15. The isolated nucleic of embodiment 6 or 14, or the viral genome of any one of embodiment 14, wherein:

(i) the encoded lysosomal targeting sequence comprises the amino acid sequence of any of SEQ ID NOs: 1800, 1802, 1804, 1806, or 1808, or an amino acid sequence having at least one, two, or three but no more than four modifications, e.g., substitutions (e.g., conservative substitutions), relative to SEQ ID NOs: 1800, 1802, 1804, 1806, or 1808;

(ii) the nucleotide sequence encoding the lysosomal targeting sequence comprises the nucleotide sequence of any of SEQ ID NO: 1799, 1801, 1803, 1805, or 1807, or a nucleotide sequence having at least one, two, or three but no more than four modifications, e.g., substitutions (e.g., conservative substitutions), relative to SEQ ID NOs: 1799, 1801, 1803, 1805, or 1807.

16. The isolated nucleic acid of any one of embodiments 5-6 or 9-15, or the viral genome of any one of embodiments 8-15, wherein the nucleic acid encodes at least 2, 3, 4 or more enhancement elements.

17. The isolated nucleic acid of any one of embodiments 5-6 or 9-16, or the viral genome of any one of embodiments 8-16, wherein the nucleic acid encodes two enhancement elements, wherein:

(i) the first enhancement element comprises a lysosomal targeting sequence, optionally wherein the lysosomal targeting sequence comprises the amino acid sequence of SEQ ID NO: 1802, or an amino acid sequence having at least one, two, or three but no more than four modifications, e.g., substitutions (e.g., conservative substitutions), relative to SEQ ID NO: 1802; and

(ii) the second enhancement element comprises Saposin C polypeptide or functional fragment or variant thereof, optionally wherein the Saposin C polypeptide or functional fragment or variant thereof comprises the amino acid sequence of SEQ ID NO: 1789, or an amino acid sequence having at least one, two, or three but no more than four modifications, e.g., substitutions (e.g., conservative substitutions), relative to SEQ ID NO: 1789.

18. The isolated nucleic acid or viral genome of embodiment 17, wherein the nucleic acid encoding the first enhancement element and the second enhancement element, comprises the nucleotide sequences of 1801 and 1787, a nucleotide sequence at least 85% (e.g., at least 90%, 92%, 95%, 97%, 98%, or 99%) identical to SEQ ID NOs: 1801 and 1787, or a nucleotide sequence having at least one, two, or three but no more than four modifications, e.g., substitutions (e.g., conservative substitutions), relative to SEQ ID NOs: 1801 and 1787.

19. The isolated nucleic acid of any one of embodiments 5-6 or 9-17, or the viral genome of any one of embodiments 8-18, wherein the nucleic acid encodes a first enhancement element and a second enhancement element, wherein:

(i) the first enhancement element a cell penetrating peptide, optionally wherein the cell penetrating peptide comprises the amino acid sequence of SEQ ID NO: 1798, or an amino acid sequence having at least one, two, or three but no more than four modifications, e.g., substitutions (e.g., conservative substitutions), relative to SEQ ID NO: 1798; and

(ii) the second enhancement element comprises a lysosomal targeting sequence, optionally wherein the lysosomal targeting sequence comprises the amino acid sequence of SEQ ID NO: 1802, or an amino acid sequence having at least one, two, or three but no more than four modifications, e.g., substitutions (e.g., conservative substitutions), relative to SEQ ID NO: 1802.

20. The isolated nucleic acid or viral genome of embodiment 19, wherein the nucleic acid encoding the first enhancement element and the second enhancement element, comprises the nucleotide sequences of 1797 and 1801, a nucleotide sequence at least 85% (e.g., at least 90%, 92%, 95%, 97%, 98%, or 99%) identical to SEQ ID NOs: 1797 and 1801, or a nucleotide sequence having at least one, two, or three but no more than four modifications, e.g., substitutions (e.g., conservative substitutions), relative to SEQ ID NOs: 1797 and 1801.

21. The isolated nucleic acid of any one of embodiments 5-6 or 9-20, or the viral genome of any one of embodiments 8-20, wherein the nucleic acid encodes a first enhancement element, a second enhancement element and a third enhancement element, wherein:

(i) the first enhancement element comprises a lysosomal targeting sequence, optionally wherein the lysosomal targeting sequence comprises the amino acid sequence of SEQ ID NO: 1802, or an amino acid sequence having at least one, two, or three but no more than four modifications, e.g., substitutions (e.g., conservative substitutions), relative to SEQ ID NO: 1802;

(ii) the second enhancement element comprises a cell penetrating peptide, optionally wherein the cell penetrating peptide comprises the amino acid sequence of SEQ ID NO: 1798, or an amino acid sequence having at least one, two, or three but no more than four modifications, e.g., substitutions (e.g., conservative substitutions), relative to SEQ ID NO: 1798; and

(iii) the third enhancement element comprises Saposin C polypeptide or functional fragment or variant thereof, optionally wherein the Saposin C polypeptide or functional fragment or variant thereof comprises amino acid sequence of SEQ ID NO: 1789, or an amino acid sequence having at least one, two, or three but no more than four modifications, e.g., substitutions (e.g., conservative substitutions), relative to SEQ ID NO: 1789.

22. The isolated nucleic acid or viral genome of embodiment 21, wherein the nucleic acid encoding the first enhancement element, the second enhancement element, and the third enhancement element, comprises the nucleotide sequences of 1801, 1797, and 1787, a nucleotide sequence at least 85% (e.g., at least 90%, 92%, 95%, 97%, 98%, or 99%) identical to SEQ ID NOs: 1801, 1797, and 1787, or a nucleotide sequence having at least one, two, or three but no more than four modifications, e.g., substitutions (e.g., conservative substitutions), relative to SEQ ID NOs: 1801, 1797, and 1787.

23. The isolated nucleic acid of any one of embodiments 1-6 or 9-22, or the viral genome of any one of embodiments 7-22, wherein the nucleic acid further encodes a linker.

24. The isolated nucleic acid of any one of embodiments 5-6 or 9-22, or the viral genome of any one of embodiments 8-22, wherein the encoded enhancement element and the encoded GBA protein are connected directly, e.g., without a linker.

25. The isolated nucleic acid of any one of embodiments 5-6 or 9-23, or the viral genome of any one of embodiments 8-23, wherein the encoded enhancement element and the encoded GBA protein are connected via the encoded linker.

26. The isolated nucleic acid or viral genome of embodiment 23 or 25, wherein:

(i) the encoded linker comprises the amino acid sequence of any of SEQ ID NOs: 1854, 1855, 1843, or 1845, or an amino acid sequence having at least one, two, or three but no more than four modifications, e.g., substitutions (e.g., conservative substitutions), relative to SEQ ID NOs: 1854, 1855, 1843, or 1845;

(ii) the nucleotide sequence encoding the linker comprises any of the nucleotide sequences of Table 2, or a nucleotide sequence having at least one, two, or three but no more than four modifications, e.g., substitutions (e.g., conservative substitutions), relative to the sequences of Table 2;

(iii) the nucleotide sequence encoding the linker comprises the nucleotide sequence of any one of SEQ ID NOs: 1724, 1726, 1729, or 1730, or a nucleotide sequence having at least one, two, or three but no more than four modifications, e.g., substitutions (e.g., conservative substitutions), relative to SEQ ID NOs: 1724, 1726, 1729, or 1730;

(iv) the encoded linker comprises a furin cleavage site;

(v) the encoded linker comprises a T2A polypeptide;

(vi) the encoded linker comprises a (Gly4Ser)n linker (SEQ ID NO: 1871), wherein n is 1-10, e.g., n is 3, 4, or 5; and/or

(vii) the encoded linker comprises a (Gly4Ser)3 linker (SEQ ID NO: 1845).

27. The isolated nucleic acid or the viral genome of any one of embodiments 23 or 25-26, wherein:

(i) the encoded linker comprises the amino acid sequence of SEQ ID NO: 1854 and/or the amino acid sequence of SEQ ID NO: 1855, or an amino acid sequence having at least one, two, or three but no more than four modifications, e.g., substitutions, relative to SEQ ID NO: 1854 and/or 1855; and/or

(ii) the nucleotide sequence encoding the linker comprises the nucleotide sequence of SEQ ID NO: 1724 and/or the nucleotide sequence of SEQ ID NO: 1726, or a nucleotide sequence having at least one, two, or three but no more than four modifications, e.g., substitutions, relative to SEQ ID NO: 1724 and/or 1726.

28. The isolated nucleic acid of any one of embodiments 23 or 25-27, or the viral genome of any one of embodiments 23 or 25-26, wherein:

(i) the encoded linker comprises the amino acid sequence of SEQ ID NO: 1845, or an amino acid sequence having at least one, two, or three but no more than four modifications, e.g., substitutions, relative to SEQ ID NO: 1845;

(ii) the nucleotide sequence encoding the linker comprises the nucleotide sequence of SEQ ID NO: 1730, or a nucleotide sequence having at least one, two, or three but no more than four modifications, e.g., substitutions, relative to SEQ ID NO: 1730.

29. The isolated nucleic acid of any one of embodiments 5-6 or 9-28, or the viral genome of any one of embodiments 8-28, wherein the encoded GBA protein and the encoded enhancement element are expressed as a single polypeptide.

30. The isolated nucleic acid of any one of embodiments 5-6 or 9-28, or the viral genome of any one of embodiments 8-28, wherein the single polypeptide comprises a cleavage site present between the encoded GBA protein and the encoded enhancement element, optionally wherein the cleavage site is an T2A and/or a furin cleavage site.

31. The isolated nucleic acid of any one of embodiments 5-6 or 9-30, or the viral genome of any one of embodiments 8-30, wherein:

(i) the nucleotide sequence encoding the enhancement element is located 5′ relative to the nucleotide sequence encoding the GBA protein; and/or

(ii) the nucleotide sequence encoding the enhancement element is located 3′ relative to the nucleotide sequence encoding the GBA protein.

32. The isolated nucleic acid of any one of embodiments 1-6 or 9-31, or the viral genome of any one of embodiments 7-31, wherein the encoded GBA protein comprises the amino acid sequence of SEQ ID NO: 1775, or an amino acid sequence at least 70% (e.g., at least 75%, 80%, 85%, 90%, 92%, 95%, 97%, 98%, or 99%) identical thereto.

33. The isolated nucleic acid of any one of embodiments 6 or 9-32, or the viral genome of any one of embodiments 7-32, wherein the nucleotide sequence encoding the GBA protein comprises the nucleotide sequence of any one of SEQ ID NOs: 1773, 1777, or 1781, or a nucleotide sequence at least 70% (e.g., at least 75%, 80%, 85%, 90%, 92%, 95%, 97%, 98%, or 99%) identical thereto.

34. The isolated nucleic acid of any one of embodiments 1-6 or 9-33, or the viral genome of any one of embodiments 7-33, wherein the nucleotide sequence encoding the GBA protein comprises the nucleotide sequence of SEQ ID NO: 1773.

35. The isolated nucleic acid of any one of embodiments 6 or 9-33, or the viral genome of any one of embodiments 7-33, wherein the nucleotide sequence encoding the GBA protein comprises the nucleotide sequence of SEQ ID NO: 1777.

36. The isolated nucleic acid of any one of embodiments 6 or 9-33, or the viral genome of any one of embodiments 7-33, wherein the nucleotide sequence encoding the GBA protein comprises the nucleotide sequence of SEQ ID NO: 1781.

37. The isolated nucleic acid of any one of embodiments 1-6 or 9-36, or the viral genome of any one of embodiments 7-36, wherein the nucleotide sequence encoding the GBA protein is codon optimized.

38. The isolated nucleic acid of any one of embodiments 1-6 or 9-37, or the viral genome of any one of embodiments 7-37, further encoding a signal sequence.

39. The isolated nucleic acid or the viral genome of embodiment 38, wherein the encoded signal sequence comprises the amino acid sequence of SEQ ID NO: 1853, or an amino acid sequence at least 85% (e.g., at least 90%, 92%, 95%, 97%, 98%, or 99%) identical thereto.

40. The isolated nucleic acid or the viral genome of embodiment 38 or 39, wherein the encoded signal sequence comprises the amino acid sequence of SEQ ID NO: 1857, or an amino acid sequence at least 85% (e.g., at least 90%, 92%, 95%, 97%, 98%, or 99%) identical thereto.

41. The isolated nucleic acid or the viral genome of any one of embodiments 38-40, wherein the nucleotide sequence encoding the signal sequence comprises the nucleotide sequence of any of SEQ ID NOs: 1850-1852 or 1856, or a nucleotide sequence at least 85% (e.g., at least 90%, 92%, 95%, 97%, 98%, or 99%) identical thereto.

42. The isolated nucleic acid or the viral genome of any one of embodiments 38-41, wherein the nucleotide sequence encoding the signal sequence is located:

(i) 5′ relative to the nucleotide sequence encoding the GBA protein; and/or

(ii) 5′ relative to the encoded enhancement element.

43. The isolated nucleic acid or the viral genome of any one of embodiments 38-42, wherein:

(i) the nucleotide sequence encoding the signal sequence comprises the nucleotide sequence of 1850 or a nucleotide sequence at least 85% (e.g., at least 90%, 92%, 95%, 97%, 98%, or 99%) identical thereto, and the nucleotide sequence encoding the GBA protein comprises the nucleotide sequence of SEQ ID NO: 1773, or a nucleotide sequence at least 70% (e.g., at least 75%, 80%, 85%, 90%, 92%, 95%, 97%, 98%, or 99%) identical thereto;

(ii) the nucleotide sequence encoding the signal sequence comprises the nucleotide sequence of 1851 or a nucleotide sequence at least 85% (e.g., at least 90%, 92%, 95%, 97%, 98%, or 99%) identical thereto, and the nucleotide sequence encoding the GBA protein comprises the nucleotide sequence of SEQ ID NO: 1777, or a nucleotide sequence at least 70% (e.g., at least 75%, 80%, 85%, 90%, 92%, 95%, 97%, 98%, or 99%) identical thereto;

(iii) the nucleotide sequence encoding the signal sequence comprises the nucleotide sequence of 1852 or a nucleotide sequence at least 85% (e.g., at least 90%, 92%, 95%, 97%, 98%, or 99%) identical thereto, and the nucleotide sequence encoding the GBA protein comprises the nucleotide sequence of SEQ ID NO: 1781, or a nucleotide sequence at least 70% (e.g., at least 75%, 80%, 85%, 90%, 92%, 95%, 97%, 98%, or 99%) identical thereto;

and optionally wherein the nucleotide sequence encoding the signal sequence is located 5′ relative to the nucleotide sequence encoding the GBA protein.

44. The isolated nucleic acid or the viral genome of any one of embodiments 38-43, wherein the encoded signal sequence comprises the amino acid sequence of SEQ ID NO: 1853 or an amino acid sequence at least 85% (e.g., at least 90%, 92%, 95%, 97%, 98%, or 99%) identical thereto; and the encoded GBA protein comprises the amino acid sequence of SEQ ID NO: 1775, or an amino acid sequence at least 70% (e.g., at least 75%, 80%, 85%, 90%, 92%, 95%, 97%, 98%, or 99%) identical thereto;

and optionally wherein the encoded signal sequence is located N-terminal relative to the encoded GBA protein.

45. The isolated nucleic acid or the viral genome of any one of embodiments 38-44, wherein:

(i) the nucleotide sequence encoding the signal sequence comprises the nucleotide sequence of any of SEQ ID NO: 1850-1852, or a nucleotide sequence at least 85% (e.g., at least 90%, 92%, 95%, 97%, 98%, or 99%) identical thereto; and the nucleotide sequence encoding the enhancement element comprises the nucleotide sequence of SEQ ID NO: 1801, or a nucleotide sequence at least 85% (e.g., at least 90%, 92%, 95%, 97%, 98%, or 99%) identical thereto; and optionally wherein the nucleotide sequence encoding the signal sequence is located 5′ relative to the nucleotide sequence encoding the enhancement element;

(ii) the encoded signal sequence comprises the amino acid sequence of SEQ ID NO: 1853, or an amino acid sequence at least 85% (e.g., at least 90%, 92%, 95%, 97%, 98%, or 99%) identical thereto; and the encoded enhancement element comprises the amino acid sequence of SEQ ID NO: 1802, or an amino acid sequence having at least one, two, or three but no more than four modifications, e.g., substitutions, relative to SEQ ID NO: 1802; and optionally wherein the encoded signal sequence is located N-terminal relative to the encoded enhancement element;

(iii) the nucleotide sequence encoding the signal sequence comprises the nucleotide sequence of SEQ ID NO: 1856, or a nucleotide sequence at least 85% (e.g., at least 90%, 92%, 95%, 97%, 98%, or 99%) identical thereto; and the nucleotide sequence encoding the enhancement element comprises the nucleotide sequence of SEQ ID NO: 1859, or a nucleotide sequence at least 85% (e.g., at least 90%, 92%, 95%, 97%, 98%, or 99%) identical thereto; and the nucleotide sequence encoding the enhancement element comprises the nucleotide sequence of SEQ ID NO: 1859; and optionally wherein the nucleotide sequence encoding the signal sequence is located 5′ relative to the nucleotide sequence encoding the enhancement element;

(iv) the encoded signal sequence comprises the amino acid sequence of SEQ ID NO: 1857, or an amino acid sequence at least 85% (e.g., at least 90%, 92%, 95%, 97%, 98%, or 99%) identical thereto; and the encoded enhancement element comprises the amino acid sequence of SEQ ID NO: 1785, or an amino acid sequence at least 85% (e.g., at least 90%, 92%, 95%, 97%, 98%, or 99%) identical thereto; and optionally wherein the encoded signal sequence is located N-terminal relative to the encoded enhancement element;

(v) the nucleotide sequence encoding the signal sequence comprises the nucleotide sequence of SEQ ID NO: 1856, or a nucleotide sequence at least 85% (e.g., at least 90%, 92%, 95%, 97%, 98%, or 99%) identical thereto; and the nucleotide sequence encoding the enhancement element comprises the nucleotide sequence of SEQ ID NO: 1787, or a nucleotide sequence at least 85% (e.g., at least 90%, 92%, 95%, 97%, 98%, or 99%) identical thereto; and the nucleotide sequence encoding the enhancement element comprises the nucleotide sequence of SEQ ID NO: 1787; and optionally wherein the nucleotide sequence encoding the signal sequence is located 5′ relative to the nucleotide sequence encoding the enhancement element;

(vi) the encoded signal sequence comprises the amino acid sequence of SEQ ID NO: 1857, or an amino acid sequence at least 85% (e.g., at least 90%, 92%, 95%, 97%, 98%, or 99%) identical thereto; and the encoded enhancement element comprises the amino acid sequence of SEQ ID NO: 1789, or an amino acid sequence having at least one, two, or three but no more than four modifications, e.g., substitutions, relative to SEQ ID NO: 1789; and optionally wherein the encoded signal sequence is located N-terminal relative to the encoded enhancement element;

(vii) the nucleotide sequence encoding the signal sequence comprises the nucleotide sequence of SEQ ID NO: 1856, or a nucleotide sequence at least 85% (e.g., at least 90%, 92%, 95%, 97%, 98%, or 99%) identical thereto; and the nucleotide sequence encoding the enhancement element comprises the nucleotide sequence of SEQ ID NO: 1791, or a nucleotide sequence at least 85% (e.g., at least 90%, 92%, 95%, 97%, 98%, or 99%) identical thereto; and the nucleotide sequence encoding the enhancement element comprises the nucleotide sequence of SEQ ID NO: 1791 and optionally wherein the nucleotide sequence encoding the signal sequence is located 5′ relative to the nucleotide sequence encoding the enhancement element;

(viii) the encoded signal sequence comprises the amino acid sequence of SEQ ID NO: 1857, or an amino acid sequence at least 85% (e.g., at least 90%, 92%, 95%, 97%, 98%, or 99%) identical thereto; and the encoded enhancement element comprises the amino acid sequence of SEQ ID NO: 1758, or an amino acid sequence having at least one, two, or three but no more than four modifications, e.g., substitutions, relative to SEQ ID NO: 1758; and optionally wherein the encoded signal sequence is located N-terminal relative to the encoded enhancement element;

(ix) the nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; and the nucleotide sequence encoding an enhancement element comprising the nucleotide sequence of SEQ ID NO: 1793, or a nucleotide sequence at least 85% (e.g., at least 90%, 92%, 95%, 96%, 97%, 98%, or 99%) identical thereto; and optionally wherein the nucleotide sequence encoding the signal sequence is located 5′ relative to the nucleotide sequence encoding the enhancement element; and/or

(x) the encoded signal sequence comprises the amino acid sequence of SEQ ID NO: 1853, or an amino acid sequence at least 85% (e.g., at least 90%, 92%, 95%, 96%, 97%, 98%, or 99%) identical thereto; and the encoded enhancement element comprises the amino acid sequence of SEQ ID NO: 1794, or an amino acid sequence having at least one, two, or three but no more than four modifications, e.g., substitutions, relative to SEQ ID NO: 1794; and optionally wherein the encoded signal sequence is located N-terminal relative to the encoded enhancement element.

46. The isolated nucleic acid of any one of embodiments 1-6 or 9-45, or the viral genome of any one of embodiments 7-45, wherein the nucleic acid comprises in 5′ to 3′ order: a nucleotide sequence encoding a signal sequence comprising the nucleotide sequence of SEQ ID NO: 1850, or a nucleotide sequence at least 85% (e.g., at least 90%, 92%, 95%, 96%, 97%, 98%, or 99%) identical thereto; and a nucleotide sequence encoding a GBA protein comprising the nucleotide sequence of SEQ ID NO: 1773, or a nucleotide sequence at least 85% (e.g., at least 90%, 92%, 95%, 96%, 97%, 98%, or 99%) identical thereto.

47. The isolated nucleic acid of any one of embodiments 1-6 or 9-46, or the viral genome of any one of embodiments 7-46, wherein the nucleic acid comprises in 5′ to 3′ order:

(i) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; and a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto;

(ii) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1799, or a nucleotide sequence at least 85% (e.g., at least 90%, 92%, 95%, 96%, 97%, 98%, or 99%) identical thereto;

(iii) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding an enhancement element comprising the nucleotide sequence of SEQ ID NO: 1801, or a nucleotide sequence at least 85% (e.g., at least 90%, 92%, 95%, 96%, 97%, 98%, or 99%) identical thereto; and a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto;

(iv) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1803, or a nucleotide sequence at least 85% (e.g., at least 90%, 92%, 95%, 96%, 97%, 98%, or 99%) identical thereto;

(v) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1805, or a nucleotide sequence at least 85% (e.g., at least 90%, 92%, 95%, 96%, 97%, 98%, or 99%) identical thereto;

(vi) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1797, or a nucleotide sequence at least 85% (e.g., at least 90%, 92%, 95%, 96%, 97%, 98%, or 99%) identical thereto;

(vii) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1793, or a nucleotide sequence at least 85% (e.g., at least 90%, 92%, 95%, 96%, 97%, 98%, or 99%) identical thereto;

(viii) a nucleotide sequence encoding a first signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1859, or a nucleotide sequence at least 85% (e.g., at least 90%, 92%, 95%, 96%, 97%, 98%, or 99%) identical thereto;

(ix) a nucleotide sequence encoding a first signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1787, or a nucleotide sequence at least 85% (e.g., at least 90%, 92%, 95%, 96%, 97%, 98%, or 99%) identical thereto;

(x) a nucleotide sequence encoding a first signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1791, or a nucleotide sequence at least 85% (e.g., at least 90%, 92%, 95%, 96%, 97%, 98%, or 99%) identical thereto;

(xi) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1795, or a nucleotide sequence at least 85% (e.g., at least 90%, 92%, 95%, 96%, 97%, 98%, or 99%) identical thereto;

(xii) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding an enhancement element comprising the nucleotide sequence of SEQ ID NO: 1793, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto;

(xiii) a nucleotide sequence encoding a signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1807, or a nucleotide sequence at least 85% (e.g., at least 90%, 92%, 95%, 96%, 97%, 98%, or 99%) identical thereto;

(xiv) a nucleotide sequence encoding a first signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a first enhancement element comprising the nucleotide sequence of SEQ ID NO: 1801, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding a second enhancement element comprising the nucleotide sequence of SEQ ID NO: 1787, or a nucleotide sequence at least 85% (e.g., at least 90%, 92%, 95%, 96%, 97%, 98%, or 99%) identical thereto;

(xv) a nucleotide sequence encoding a first signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; a nucleotide sequence encoding a first enhancement element comprising the nucleotide sequence of SEQ ID NO: 1797, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding a second enhancement element comprising the nucleotide sequence of SEQ ID NO: 1787, or a nucleotide sequence at least 85% (e.g., at least 90%, 92%, 95%, 96%, 97%, 98%, or 99%) identical thereto;

(xvi) a nucleotide sequence encoding a first signal sequence comprising the nucleotide sequence of SEQ ID NO: 1852, or a nucleotide sequence at least 85% (e.g., at least 90%, 92%, 95%, 96%, 97%, 98%, or 99%) identical thereto; a nucleotide sequence encoding a first enhancement element comprising the nucleotide sequence of SEQ ID NO: 1801, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1781, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; a nucleotide sequence encoding a first enhancement element comprising the nucleotide sequence of SEQ ID NO: 1797, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding a second enhancement element comprising the nucleotide sequence of SEQ ID NO: 1787, or a nucleotide sequence at least 85% (e.g., at least 90%, 92%, 95%, 96%, 97%, 98%, or 99%) identical thereto;

(xvii) a nucleotide sequence encoding a signal sequence comprising the nucleotide sequence of SEQ ID NO: 1851, or a nucleotide sequence at least 85% (e.g., at least 90%, 92%, 95%, 96%, 97%, 98%, or 99%) identical thereto; and a nucleotide sequence encoding a GBA protein comprising the nucleotide sequence of SEQ ID NO: 1777, or a nucleotide sequence at least 85% (e.g., at least 90%, 92%, 95%, 96%, 97%, 98%, or 99%) identical thereto;

(xviii) a nucleotide sequence encoding a first signal sequence comprising the nucleotide sequence of SEQ ID NO: 1851, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1777, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1787, or a nucleotide sequence at least 85% (e.g., at least 90%, 92%, 95%, 96%, 97%, 98%, or 99%) identical thereto;

(xix) a nucleotide sequence encoding a signal sequence comprising the nucleotide sequence of SEQ ID NO: 1851, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1777, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1797, or a nucleotide sequence at least 85% (e.g., at least 90%, 92%, 95%, 96%, 97%, 98%, or 99%) identical thereto;

(xx) a nucleotide sequence encoding a signal sequence comprising the nucleotide sequence of SEQ ID NO: 1851, or a nucleotide sequence at least 85% (e.g., at least 90%, 92%, 95%, 96%, 97%, 98%, or 99%) identical thereto; a nucleotide sequence encoding an enhancement element comprising the nucleotide sequence of SEQ ID NO: 1801, or a nucleotide sequence at least 85% (e.g., at least 90%, 92%, 95%, 96%, 97%, 98%, or 99%) identical thereto; and a nucleotide sequence encoding a GBA protein comprising the nucleotide sequence of SEQ ID NO: 1777, or a nucleotide sequence at least 85% (e.g., at least 90%, 92%, 95%, 96%, 97%, 98%, or 99%) identical thereto;

(xxi) a nucleotide sequence encoding a signal sequence comprising the nucleotide sequence of SEQ ID NO: 1851, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1777, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1805, or a nucleotide sequence at least 85% (e.g., at least 90%, 92%, 95%, 96%, 97%, 98%, or 99%) identical thereto;

(xxii) a nucleotide sequence encoding a first signal sequence comprising the nucleotide sequence of SEQ ID NO: 1850, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1773, or a nucleotide sequence at least 85% (e.g., at least 90%, 92%, 95%, 96%, 97%, 98%, or 99%) identical thereto; a nucleotide sequence encoding a furin cleavage site comprising the nucleotide sequence of SEQ ID NO: 1724, or a nucleotide sequence at least 85% (e.g., at least 90%, 92%, 95%, 96%, 97%, 98%, or 99%) identical thereto; a nucleotide sequence encoding a T2A polypeptide comprising the nucleotide sequence of SEQ ID NO: 1726, or a nucleotide sequence at least 85% (e.g., at least 90%, 92%, 95%, 96%, 97%, 98%, or 99%) identical thereto; a nucleotide sequence encoding a second signal sequence comprising the nucleotide sequence of SEQ ID NO: 1856, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1787, or a nucleotide sequence at least 85% (e.g., at least 90%, 92%, (xxiii) a nucleotide sequence encoding a signal sequence comprising the nucleotide sequence of SEQ ID NO: 1850, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1773, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1797, or a nucleotide sequence at least 85% (e.g., at least 90%, 92%, 95%, 96%, 97%, 98%, or 99%) identical thereto;

(xxiv) a nucleotide sequence encoding a signal sequence comprising the nucleotide sequence of SEQ ID NO: 1850, or a nucleotide sequence at least 85% (e.g., at least 90%, 92%, 95%, 96%, 97%, 98%, or 99%) identical thereto; a nucleotide sequence encoding an enhancement element comprising the nucleotide sequence of SEQ ID NO: 1801, or a nucleotide sequence at least 85% (e.g., at least 90%, 92%, 95%, 96%, 97%, 98%, or 99%) identical thereto; and a nucleotide sequence encoding a GBA protein comprising the nucleotide sequence of SEQ ID NO: 1773, or a nucleotide sequence at least 85% (e.g., at least 90%, 92%, 95%, 96%, 97%, 98%, or 99%) identical thereto;

(xxv) a nucleotide sequence encoding a signal sequence comprising the nucleotide sequence of SEQ ID NO: 1850, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1773, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1805, or a nucleotide sequence at least 85% (e.g., at least 90%, 92%, 95%, 96%, 97%, 98%, or 99%) identical thereto;

(xxvi) a nucleotide sequence encoding a signal sequence comprising the nucleotide sequence of SEQ ID NO: 1851, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1777, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1793, or a nucleotide sequence at least 85% (e.g., at least 90%, 92%, 95%, 96%, 97%, 98%, or 99%) identical thereto; or

(xxvii) a nucleotide sequence encoding a signal sequence comprising the nucleotide sequence of SEQ ID NO: 1850, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1773, or a nucleotide sequence at least 85% (e.g., at least 90%, 92%, 95%, 96%, 97%, 98%, or 99%) identical thereto; a nucleotide sequence encoding a linker comprising the nucleotide sequence of SEQ ID NO: 1730, or a nucleotide sequence at least 85% (e.g., at least 90%, 92%, 95%, 96%, 97%, 98%, or 99%) identical thereto; and a nucleotide sequence encoding an enhancement element comprising the nucleotide sequence of SEQ ID NO: 1793, or a nucleotide sequence at least 85% (e.g., at least 90%, 92%, 95%, 96%, 97%, 98%, or 99%) identical thereto.

48. The isolated nucleic acid of any one of embodiments 1-6 or 9-47, or the viral genome of any one of embodiments 7-47, wherein the nucleic acid encodes in 5′ to 3′ order: a signal sequence comprising the amino acid sequence of SEQ ID NO: 1853, or an amino acid sequence at least 85% (e.g., at least 90%, 92%, 95%, 96%, 97%, 98%, or 99%) identical thereto; and a GBA protein comprising the amino acid sequence of SEQ ID NO: 1775, or an amino acid sequence at least 85% (e.g., at least 90%, 92%, 95%, 96%, 97%, 98%, or 99%) identical thereto.

49. The isolated nucleic acid of any one of embodiments 1-6 or 9-48, or the viral genome of any one of embodiments 7-48, wherein the nucleic acid encodes in 5′ to 3′ order:

(i) a signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; and an enhancement element comprising the amino acid sequence of SEQ ID NO: 1800, or an amino acid sequence having at least one, two, or three but no more than four modifications, e.g., substitutions, relative to SEQ ID NO: 1800;

(ii) a signal sequence comprising the amino acid sequence of SEQ ID NO: 1853, or an amino acid sequence at least 85% (e.g., at least 90%, 92%, 95%, 96%, 97%, 98%, or 99%) identical thereto; an enhancement element comprising the amino acid sequence of SEQ ID NO: 1802, or an amino acid sequence at least 85% (e.g., at least 90%, 92%, 95%, 96%, 97%, 98%, or 99%) identical thereto; and a GBA protein comprising the amino acid sequence of SEQ ID NO: 1775, or an amino acid sequence at least 85% (e.g., at least 90%, 92%, 95%, 96%, 97%, 98%, or 99%) identical thereto;

(iii) a signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; and an enhancement element comprising the amino acid sequence of SEQ ID NO: 1804, or an amino acid sequence having at least one, two, or three but no more than four modifications, e.g., substitutions, relative to SEQ ID NO: 1804;

(iv) a signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; and an enhancement element comprising the amino acid sequence of SEQ ID NO: 1806, or an amino acid sequence having at least one, two, or three but no more than four modifications, e.g., substitutions, relative to SEQ ID NO: 1806;

(v) a signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a linker comprising the amino acid sequence of SEQ ID NO: 1845, or an amino acid sequence having at least one, two, or three but no more than four modifications, e.g., substitutions, relative to SEQ ID NO: 1845; and an enhancement element comprising the amino acid sequence of SEQ ID NO: 1798, or an amino acid sequence having at least one, two, or three but no more than four modifications, e.g., substitutions, relative to SEQ ID NO: 1798;

(vi) a signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a linker comprising the amino acid sequence of SEQ ID NO: 1845, or an amino acid sequence having at least one, two, or three but no more than four modifications, e.g., substitutions, relative to SEQ ID NO: 1845; and an enhancement element comprising the amino acid sequence of SEQ ID NO: 1794, or an amino acid sequence having at least one, two, or three but no more than four modifications, e.g., substitutions, relative to SEQ ID NO: 1794;

(vii) a first signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1785, or an amino acid sequence at least 85% (e.g., at least 90%, 92%, 95%, 96%, 97%, 98%, or 99%) identical thereto;

(viii) a first signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1789, or an amino acid sequence at least 85% (e.g., at least 90%, 92%, 95%, 96%, 97%, 98%, or 99%) identical thereto;

(ix) a first signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1758, or an amino acid sequence at least 85% (e.g., at least 90%, 92%, 95%, 96%, 97%, 98%, or 99%) identical thereto;

(x) a signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a linker comprising the amino acid sequence of SEQ ID NO: 1845, or an amino acid sequence having at least one, two, or three but no more than four modifications, e.g., substitutions, relative to SEQ ID NO: 1845; and an enhancement element comprising the amino acid sequence of SEQ ID NO: 1796, or an amino acid sequence at least 85% (e.g., at least 90%, 92%, 95%, 96%, 97%, 98%, or 99%) identical thereto;

(xi) a signal sequence comprising the amino acid sequence of SEQ ID NO: 1853, or an amino acid sequence at least 85% (e.g., at least 90%, 92%, 95%, 96%, 97%, 98%, or 99%) identical thereto; an enhancement element comprising the amino acid sequence of SEQ ID NO: 1794, or an amino acid sequence having at least one, two, or three but no more than four modifications, e.g., substitutions, relative to SEQ ID NO: 1794; a linker comprising the amino acid sequence of SEQ ID NO: 1845, or an amino acid sequence having at least one, two, or three but no more than four modifications, e.g., substitutions, relative to SEQ ID NO: 1845; and a GBA protein comprising the amino acid sequence of SEQ ID NO: 1775, or an amino acid sequence at least 85% (e.g., at least 90%, 92%, 95%, 96%, 97%, 98%, or 99%) identical thereto;

(xii) a signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; and an enhancement element comprising the amino acid sequence of SEQ ID NO: 1808, or an amino acid sequence having at least one, two, or three but no more than four modifications, e.g., substitutions, relative to SEQ ID NO: 1808;

(xiii) a first signal sequence comprising the amino acid sequence of SEQ ID NO: 1853, or an amino acid sequence at least 85% (e.g., at least 90%, 92%, 95%, 96%, 97%, 98%, or 99%) identical thereto; a first enhancement element comprising the amino acid sequence of SEQ ID NO: 1802, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and a second enhancement element comprising the amino acid sequence of SEQ ID NO: 1789, or an amino acid sequence at least 85% (e.g., at least 90%, 92%, 95%, 96%, 97%, 98%, or 99%) identical thereto;

(xiv) a first signal sequence comprising the amino acid sequence of SEQ ID NO: 1853, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a linker comprising the amino acid sequence of SEQ ID NO: 1845, or an amino acid sequence having at least one, two, or three but no more than four modifications, e.g., substitutions, relative to SEQ ID NO: 1845; a first enhancement element comprising the amino acid sequence of SEQ ID NO: 1798, or an amino acid sequence having at least one, two, or three but no more than four modifications, e.g., substitutions, relative to SEQ ID NO: 1798; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1789, or an amino sequence at least 85% (e.g., at least 90%, 92%, 95%, 96%, 97%, 98%, or 99%) identical thereto; or

(xv) a first signal sequence comprising the amino acid sequence of SEQ ID NO: 1853, or an amino acid sequence at least 85% (e.g., at least 90%, 92%, 95%, 96%, 97%, 98%, or 99%) identical thereto; a first enhancement element comprising the amino acid sequence of SEQ ID NO: 1802, or an amino acid sequence at least 85% (e.g., at least 90%, 92%, 95%, 96%, 97%, 98%, or 99%) identical thereto; a GBA protein comprising the amino acid sequence of SEQ ID NO: 1775, or an amino acid sequence at least 85% (e.g., at least 90%, 92%, 95%, 96%, 97%, 98%, or 99%) identical thereto; a linker comprising the amino acid sequence of SEQ ID NO: 1845, or an amino acid sequence having at least one, two, or three but no more than four modifications, e.g., substitutions, relative to SEQ ID NO: 1845; a first enhancement element comprising the amino acid sequence of SEQ ID NO: 1798, or an amino acid sequence having at least one, two, or three but no more than four modifications, e.g., substitutions, relative to SEQ ID NO: 1798; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1789, or an amino acid sequence at least 85% (e.g., at least 90%, 92%, 95%, 96%, 97%, 98%, or 99%) identical thereto.

50. An isolated, e.g., recombinant viral genome comprising a promoter operably linked to the nucleic acid of any one of embodiments 1-6 or 9-49.

51. The viral genome of any one of embodiments 7-49, further comprising a promoter operably linked to the nucleic acid comprising the transgene encoding the GBA protein.

52. The viral genome of any one of embodiments 7-50, which further comprises an enhancer.

53. The viral genome of embodiment 52, wherein the enhancer comprises a CMVie enhancer.

54. The viral genome of embodiment 52 or 53, wherein the enhancer comprises the nucleotide sequence of SEQ ID NO: 1831, or a nucleotide sequence at least 95% identical thereto.

55. The viral genome of any one of embodiments 50-54, wherein the promoter comprises a tissue specific promoter or a ubiquitous promoter.

56. The viral genome of any one of embodiments 50-55, wherein the promoter comprises:

(i) an EF-1a promoter, a chicken β-actin (CBA) promoter and/or its derivative CAG, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof; and/or

(ii) the nucleotide sequence of any of SEQ ID NOs: 1832, 1833, 1834, 1835, 1836, 1839, 1840, or a nucleotide sequence at least 95% identical thereto.

57. The viral genome of any one of embodiments 50-56, wherein the promoter comprises a CB promoter or functional variant thereof.

58. The viral genome of embodiment 57, wherein the CB promoter or functional variant thereof comprises the nucleotide sequence of SEQ ID NO: 1834, or a nucleotide sequence at least 95% identical thereto.

59. The viral genome of any one of embodiments 50-58, wherein the promoter comprises a CMVie enhancer and a CB promoter.

60. The viral genome of embodiment 59, wherein the CMVie enhancer comprises the nucleotide sequence of SEQ ID NO: 1831, or a nucleotide sequence at least 95% identical thereto, and the CB promoter comprises the nucleotide sequence of SEQ ID NO: 1834, or a nucleotide sequence at least 95% identical thereto.

61. The viral genome of any one of embodiments 50-61, wherein the promoter comprises an EF-1α promoter or functional variant thereof.

62. The viral genome of embodiment 61, wherein the EF-1α promoter or functional variant thereof comprises the nucleotide sequence of SEQ ID NO: 1839 or 1840, or a nucleotide sequence at least 95% identical thereto.

63. The viral genome of embodiment 61 or 62, wherein the EF-1α promoter or functional variant thereof comprises an intron, e.g., an intron comprising the nucleotide sequence of positions 242-1,180 of SEQ ID NO: 1839 or an intron comprising the nucleotide sequence of SEQ ID NO: 1841, or a nucleotide sequence at least 95% identical thereto.

64. The viral genome of any one of embodiments 61-63, wherein the EF-1α promoter or functional variant thereof does not comprise an intron, e.g., an intron comprising the nucleotide sequence of positions 242-1,180 of SEQ ID NO: 1839 or an intron comprising the nucleotide sequence of SEQ ID NO: 1841, or a nucleotide sequence at least 95% identical thereto.

65. The viral genome of any one of embodiments 50-64, wherein the promoter comprises a CBA promoter or functional variant thereof.

66. The viral genome of embodiment 65, wherein the CBA promoter functional variant thereof comprises the nucleotide sequence of SEQ ID NO: 1836, or a nucleotide sequence at least 95% identical thereto.

67. The viral genome of any one of embodiments 50-66, wherein the promoter comprises a CMVie enhancer, a CBA promoter or functional variant thereof, and an intron.

68. The viral genome of embodiment 67, wherein:

(i) the CMVie enhancer comprises the nucleotide sequence of SEQ ID NO: 1831, or a nucleotide sequence at least 95% identical thereto;

(ii) the CBA promoter or functional variant thereof comprises the nucleotide sequence of SEQ ID NO: 1836, or a nucleotide sequence at least 95% identical thereto; and

(iii) the intron comprises the nucleotide sequence of SEQ ID NO: 1837, or a nucleotide sequence at least 95% identical thereto.

69. The viral genome of any one of embodiments 50-68, wherein the promoter comprises a CAG promoter region.

70. The viral genome of any one of embodiments 50-69, wherein the promoter comprises a CAG promoter region comprises:

(i) a CMVie enhancer, a CBA promoter or functional variant thereof, and an intron; and/or

(ii) the nucleotide sequence of SEQ ID NO: 1835, or a nucleotide sequence at least 95% identical thereto.

71. The viral genome of any one of embodiments 50-70, wherein the promoter comprises a CMV promoter or functional variant thereof.

72. The viral genome of embodiment 71, wherein the CMV promoter or functional variant thereof comprises the nucleotide sequence of SEQ ID NO: 1832, or a nucleotide sequence at least 95% identical thereto.

73. The viral genome of any one of embodiments 50-72, wherein the promoter comprises a CMVie enhancer and a CMV promoter or functional variant thereof, optionally wherein the CMVie enhancer comprises the nucleotide sequence of SEQ ID NO: 1831, or a nucleotide sequence at least 95% identical thereto, and the CMV promoter or functional variant thereof comprises the nucleotide sequence of SEQ ID NO: 1832, or a nucleotide sequence at least 95% identical thereto.

74. The viral genome of any one of embodiments 50-73, wherein the promoter comprises a CMV promoter region.

75. The viral genome of embodiment 74, wherein the CMV promoter region comprises:

(i) a CMVie enhancer and a CMV promoter or functional variant thereof;

(ii) the nucleotide sequence of SEQ ID NO: 1833, or a nucleotide sequence at least 95% identical thereto.

76. The viral genome of any one of embodiments 7-76, which further comprises an inverted terminal repeat (ITR) sequence.

77. The viral genome of embodiment 76, wherein the ITR sequence is positioned 5′ relative to the nucleic acid comprising the transgene encoding the GBA protein.

78. The viral genome of embodiment 75 or 76, wherein the ITR sequence is positioned 3′ relative to the nucleic acid comprising the transgene encoding the GBA protein.

79. The viral genome of any one of embodiments 7-78, which comprises an ITR positioned 5′ relative to the nucleic acid comprising the transgene encoding the GBA protein and an ITR positioned 3′ relative to the nucleic acid comprising the transgene encoding the GBA protein.

80. The viral genome of any one of embodiments 76-79, wherein the ITR comprises a nucleic acid sequence of SEQ ID NO: 1829, 1830, or 1862, or a nucleotide sequence at least 95% identical thereto.

81. The viral genome of any one of embodiments 76-80, wherein the ITR comprises the nucleotide sequence of SEQ ID NO: 1860 and/or 1861, or a nucleotide sequence having at least one, two, or three modifications, but no more than four modifications of SEQ ID NO: 1860 and/or 1861.

82. The viral genome of any one of embodiments 76-81, wherein the ITR is positioned 5′ relative to the nucleic acid comprising the transgene encoding the GBA protein and comprises the nucleotide sequence of SEQ ID NO: 1860 and/or 1861, or a nucleotide sequence having at least one, two, or three modifications, but no more than four modifications of SEQ ID NO: 1860 or 1861.

83. The viral genome of any one of embodiments 76-81, wherein the ITR is positioned 3′ relative to the nucleic acid comprising the transgene encoding the GBA protein and comprises the nucleotide sequence of SEQ ID NO: 1860 or 1861, or a nucleotide sequence having at least one, two, or three modifications, but no more than four modifications of SEQ ID NO: 1860 and/or 1861.

84. The viral genome of any one of embodiments 76-83, wherein:

(i) the ITR positioned 5′ relative to the nucleic acid comprising the transgene encoding the GBA protein comprises the nucleotide sequence of SEQ ID NO: 1829, or a nucleotide sequence at least 95% identical thereto; and/or

(ii) the ITR positioned 3′ relative to the nucleic acid comprising the transgene encoding the GBA protein comprises the nucleotide sequence of SEQ ID NO: 1830, or a nucleotide sequence at least 95% identical thereto.

85. The viral genome of any one of embodiments 7-84, which further comprises a polyadenylation (polyA) signal region.

86. The viral genome of embodiment 85, wherein the polyA signal region comprises the nucleotide sequence of SEQ ID NO: 1846, or a nucleotide sequence at least 95% identical thereto.

87. The viral genome of any one of embodiments 7-86, which further comprises an intron region.

88. The viral genome of embodiment 87, wherein the intron comprises a beta-globin intron.

89. The viral genome of embodiment 87 or 88, wherein the intron comprises the nucleotide sequence of SEQ ID NO: 1842, or a nucleotide sequence at least 95% identical thereto.

90. The viral genome of any one of embodiments 7-89, which further comprises an exon region, e.g., at least one, two, or three exon regions.

91. The viral genome of any one of embodiments 7-90, which further comprises a Kozak sequence.

92. The viral genome of any one of embodiments 50-91, which further comprises a nucleotide sequence encoding a miR binding site, e.g., a miR binding site that modulates, e.g., reduces, expression of the GBA protein encoded by the viral genome in a cell or tissue where the corresponding miRNA is expressed.

93. The viral genome of embodiment 7-92, wherein the encoded miRNA binding site is complementary, e.g., fully complementary or partially complementary, to a miRNA expressed in a cell or tissue of the DRG, liver, hematopoietic, or a combination thereof.

94. The viral genome of any one of embodiments 50-93, wherein the encoded miR binding site modulates, e.g., reduces, expression of the encoded GBA protein in a cell or tissue of the DRG, liver, hematopoietic lineage, or a combination thereof.

95. The viral genome of any one of embodiments 7-94, which comprises at least 1-5 copies of the encoded miR binding site, e.g., at least 1, 2, 3, 4, or 5 copies.

96. The viral genome of any one of embodiments 7-95, which comprises at least 4 copies of an encoded miR binding sites, optionally wherein all four copies comprise the same miR binding site, or at least one, two, three, or all of the copies comprise a different miR binding site.

97. The viral genome of embodiment 96, wherein the 4 copies of the encoded miR binding sites are continuous (e.g., not separated by a spacer), or are separated by a spacer.

98. The viral genome of embodiment 97, wherein the spacer comprises the nucleotide sequence of SEQ ID NO: 1848, or a nucleotide sequence having at least one, two, or three modifications, but no more than four modifications of SEQ ID NO: 1848.

99. The viral genome of any one of embodiments 7-98, wherein the encoded miR binding site comprises a miR183 binding site, a miR122 binding site, a miR-142-3p, or a combination thereof, optionally wherein:

(i) the encoded miR183 binding site comprises the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1847;

(ii) the encoded miR122 binding site comprises the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1865; and/or

(iii) the encoded miR-142-3p binding site comprises the nucleotide sequence of SEQ ID NO: 1869, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1869.

100. The viral genome of any one of embodiments 7-99, wherein the viral genome comprises an encoded miR183 binding site.

101. The viral genome of any one of embodiments 7-100, wherein the viral genome comprises at least 1-5 copies, e.g., 4 copies of a miR183 binding site, optionally wherein each copy is continuous (e.g., not separated by a spacer), or each copy is separated by a spacer.

102. The viral genome of embodiment 100 or 101, wherein the encoded miR183 binding site comprises the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1847.

103. The viral genome of any one of embodiments 7-102, wherein the viral genome comprises:

(i) a first encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1847;

(ii) a first spacer sequence comprising the nucleotide sequence of SEQ ID NO: 1848, or a nucleotide sequence having at least one, two, or three modifications, but no more than four modifications of SEQ ID NO: 1848;

(iii) a second encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1847;

(iv) a second spacer sequence comprising the nucleotide sequence of SEQ ID NO: 1848, or a nucleotide sequence having at least one, two, or three modifications, but no more than four modifications of SEQ ID NO: 1848;

(v) a third encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1847;

(vi) a third spacer sequence comprising the nucleotide sequence of SEQ ID NO: 1848, or a nucleotide sequence having at least one, two, or three modifications, but no more than four modifications of SEQ ID NO: 1848; and

(vii) a fourth encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1847.

104. The viral genome of any one of embodiments 7-103, which comprises a miR183 binding site series, which comprises four copies of a miR183 binding site, wherein each copy of the miR binding site in the series is separated by a spacer.

105. The viral genome of embodiment 104, wherein the encoded miR183 binding site series comprises the nucleotide sequence of SEQ ID NO: 1849, or a nucleotide sequence at least 95% identical thereto.

106. The viral genome of any one of embodiments 7-105, which is self-complementary.

107. The viral genome of any one of embodiments 7-106, which is single-stranded.

108. An isolated, e.g., recombinant, viral genome comprising in 5′ to 3′ order:

(i) a 5′ adeno-associated (AAV) ITR, optionally wherein the 5′ AAV ITR comprises the nucleotide sequence of SEQ ID NO: 1829, or a nucleotide sequence at least 95% identical thereto;

(ii) a CMVie enhancer, optionally wherein the CMVie enhancer comprises the nucleotide sequence of SEQ ID NO: 1831, or a nucleotide sequence at least 95% identical thereto;

(iii) a CB promoter or functional variant thereof, optionally wherein the CB promoter or functional variant thereof comprises the nucleotide sequence of SEQ ID NO: 1834, or a nucleotide sequence at least 95% identical thereto;

(iv) an intron, optionally wherein the intron comprises the nucleotide sequence of SEQ ID NO: 1842, or a nucleotide sequence at least 95% identical thereto;

(v) a nucleotide sequence encoding a signal sequence, optionally wherein the nucleotide sequence encoding the signal sequence comprises the nucleotide sequence of SEQ ID NO: 1850, or a nucleotide sequence at least 95% identical thereto;

(vi) a transgene encoding a GBA protein, wherein the nucleotide sequence encoding the GBA protein comprises the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773;

(vii) a polyA signal region, optionally wherein the polyA signal region comprises the nucleotide sequence of SEQ ID NO: 1846, or a nucleotide sequence at least 95% identical thereto; and

(viii) a 3′ AAV ITR, optionally wherein the 3′ AAV ITR comprises the nucleotide sequence of SEQ ID NO: 1830, or a nucleotide sequence at least 95% identical thereto.

109. An isolated, e.g., recombinant, viral genome comprising in 5′ to 3′ order:

(i) a 5′ adeno-associated (AAV) ITR, optionally wherein the 5′ AAV ITR comprises the nucleotide sequence of SEQ ID NO: 1829, or a nucleotide sequence at least 95% identical thereto;

(ii) a CMVie enhancer, optionally wherein the CMVie enhancer comprises the nucleotide sequence of SEQ ID NO: 1831, or a nucleotide sequence at least 95% identical thereto;

(iii) a CB promoter or functional variant thereof, optionally wherein the CB promoter or functional variant thereof comprises the nucleotide sequence of SEQ ID NO: 1834, or a nucleotide sequence at least 95% identical thereto;

(iv) an intron, optionally wherein the intron comprises the nucleotide sequence of SEQ ID NO: 1842, or a nucleotide sequence at least 95% identical thereto;

(v) a nucleotide sequence encoding a signal sequence, optionally wherein the nucleotide sequence encoding the signal sequence comprises the nucleotide sequence of SEQ ID NO: 1850, or a nucleotide sequence at least 95% identical thereto;

(vi) a transgene encoding a GBA protein, optionally wherein the nucleotide sequence encoding the GBA protein comprises the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773;

(vii) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;

(viii) a spacer sequence, optionally wherein the spacer comprises the nucleotide sequence of SEQ ID NO: 1848, or a nucleotide sequence having at least one, two, or three modifications, but no more than four modifications of SEQ ID NO: 1848;

(ix) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;

(x) a spacer sequence, optionally wherein the spacer comprises the nucleotide sequence of SEQ ID NO: 1848, or a nucleotide sequence having at least one, two, or three modifications, but no more than four modifications of SEQ ID NO: 1848;

(xi) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;

(xii) a spacer sequence, optionally wherein the spacer comprises the nucleotide sequence of SEQ ID NO: 1848, or a nucleotide sequence having at least one, two, or three modifications, but no more than four modifications of SEQ ID NO: 1848;

(xiii) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;

(xiv) a polyA signal region, optionally wherein the polyA signal region comprises the nucleotide sequence of SEQ ID NO: 1846, or a nucleotide sequence at least 95% identical thereto; and

(xv) a 3′ AAV ITR, optionally wherein the 3′ AAV ITR comprises the nucleotide sequence of SEQ ID NO: 1830, or a nucleotide sequence at least 95% identical thereto.

110. The viral genome of any one of embodiments 50-109, which comprises the nucleotide sequence of SEQ ID NO: 1812, or a nucleotide sequence at least 95% identical thereto.

111. The viral genome of any one of embodiments 50-110, which comprises the nucleotide sequence of SEQ ID NO: 1826, or a nucleotide sequence at least 95% identical thereto.

112. An isolated, e.g., recombinant, viral genome comprising in 5′ to 3′ order:

(i) a 5′ adeno-associated (AAV) ITR, optionally wherein the 5′ AAV ITR comprises the nucleotide sequence of SEQ ID NO: 1829, or a nucleotide sequence at least 95% identical thereto;

(ii) a CMVie enhancer, optionally wherein the CMVie enhancer comprises the nucleotide sequence of SEQ ID NO: 1831, or a nucleotide sequence at least 95% identical thereto;

(iii) a CB promoter or functional variant thereof, optionally wherein the CB promoter or functional variant thereof comprises the nucleotide sequence of SEQ ID NO: 1834, or a nucleotide sequence at least 95% identical thereto;

(iv) an intron, optionally wherein the intron comprises the nucleotide sequence of SEQ ID NO: 1842, or a nucleotide sequence at least 95% identical thereto;

(v) a nucleic acid comprising a transgene encoding a β-glucocerebrosidase (GBA) protein of any one of embodiments 1-6 or 9-49;

(vi) a polyA signal region, optionally wherein the polyA signal region comprises the nucleotide sequence of SEQ ID NO: 1846, or a nucleotide sequence at least 95% identical thereto; and

(vii) a 3′ AAV ITR, optionally wherein the 3′ AAV ITR comprises the nucleotide sequence of SEQ ID NO: 1830, or a nucleotide sequence at least 95% identical thereto.

113. An isolated, e.g., recombinant, viral genome comprising in 5′ to 3′ order:

(i) a 5′ adeno-associated (AAV) ITR, optionally wherein the 5′ AAV ITR comprises the nucleotide sequence of SEQ ID NO: 1829, or a nucleotide sequence at least 95% identical thereto;

(ii) an EF-1α promoter or functional variant thereof, optionally wherein the EF-1α promoter or functional variant thereof comprises the nucleotide sequence of SEQ ID NO: 1839 or 1840, or a nucleotide sequence at least 95% identical thereto;

(iii) a nucleic acid comprising a transgene encoding a β-glucocerebrosidase (GBA) protein of any one of embodiments 1-6 or 9-49;

(iv) a polyA signal region, optionally wherein the polyA signal region comprises the nucleotide sequence of SEQ ID NO: 1846, or a nucleotide sequence at least 95% identical thereto; and

(v) a 3′ AAV ITR, optionally wherein the 3′ AAV ITR comprises the nucleotide sequence of SEQ ID NO: 1830, or a nucleotide sequence at least 95% identical thereto.

114. An isolated, e.g., recombinant, viral genome comprising in 5′ to 3′ order:

(i) a 5′ adeno-associated (AAV) ITR, optionally wherein the 5′ AAV ITR comprises the nucleotide sequence of SEQ ID NO: 1829, or a nucleotide sequence at least 95% identical thereto;

(ii) a CMVie enhancer, optionally wherein the CMVie enhancer comprises the nucleotide sequence of SEQ ID NO: 1831, or a nucleotide sequence at least 95% identical thereto;

(iii) a CMV promoter or functional variant thereof, optionally wherein the CMV promoter or functional variant thereof comprises the nucleotide sequence of SEQ ID NO: 1832, or a nucleotide sequence at least 95% identical thereto;

(iv) an intron, optionally wherein the intron comprises the nucleotide sequence of SEQ ID NO: 1842, or a nucleotide sequence at least 95% identical thereto;

(v) a nucleic acid comprising a transgene encoding a β-glucocerebrosidase (GBA) protein of any one of embodiments 1-6 or 9-49;

(vi) a polyA signal region, optionally wherein the polyA signal region comprises the nucleotide sequence of SEQ ID NO: 1846, or a nucleotide sequence at least 95% identical thereto; and

(vii) a 3′ AAV ITR, optionally wherein the 3′ AAV ITR comprises the nucleotide sequence of SEQ ID NO: 1830, or a nucleotide sequence at least 95% identical thereto.

115. An isolated, e.g., recombinant, viral genome comprising in 5′ to 3′ order:

(i) a 5′ adeno-associated (AAV) ITR, optionally wherein the 5′ AAV ITR comprises the nucleotide sequence of SEQ ID NO: 1829, or a nucleotide sequence at least 95% identical thereto;

(ii) an CAG promoter or functional variant thereof, optionally wherein the CAG promoter or functional variant thereof comprises the nucleotide sequence of SEQ ID NO: 1835, or a nucleotide sequence at least 95% identical thereto;

(iii) a nucleic acid comprising a transgene encoding a β-glucocerebrosidase (GBA) protein of any one of embodiments 1-6 or 9-49;

(iv) a polyA signal region, optionally wherein the polyA signal region comprises the nucleotide sequence of SEQ ID NO: 1846, or a nucleotide sequence at least 95% identical thereto; and

(v) a 3′ AAV ITR, optionally wherein the 3′ AAV ITR comprises the nucleotide sequence of SEQ ID NO: 1830, or a nucleotide sequence at least 95% identical thereto.

116. The viral genome of any one of embodiments 7-107 or 112-115, which comprises the nucleotide sequence of any one of SEQ ID NOs: 1759-1771, 1809-1811, 1813-1827, or 1870, or a nucleotide sequence at least 95% identical thereto.

117. The viral genome of any one of embodiments 7-116, which further comprises a nucleic acid encoding a capsid protein, e.g., a structural protein, wherein the capsid protein comprises a VP1 polypeptide, a VP2 polypeptide, and/or a VP3 polypeptide.

118. The viral genome of embodiment 117, wherein the VP1 polypeptide, the VP2 polypeptide, and/or the VP3 polypeptide are encoded by at least one Cap gene.

119. The viral genome of any one of embodiments 7-118, which further comprises a nucleic acid encoding a Rep protein, e.g., a non-structural protein, wherein the Rep protein comprises a Rep78 protein, a Rep68, Rep52 protein, and/or a Rep40 protein.

120. The viral genome of embodiment 119, wherein the Rep78 protein, the Rep68 protein, the Rep52 protein, and/or the Rep40 protein are encoded by at least one Rep gene.

121. An isolated, e.g., recombinant GBA protein encoded by the isolated nucleic acid of any one of embodiments 1-6 or 9-49, or the viral genome of any one of embodiments 7-120.

122. An isolated, e.g., recombinant, AAV particle comprising:

(i) a capsid protein; and

(ii) the viral genome of any one of embodiments 7-120.

123. The AAV particle of embodiment 122, wherein:

(i) the capsid protein comprises the amino acid sequence of SEQ ID NO: 138, or an amino acid sequence with at least 80% (e.g., at least about 85, 90, 95, 96, 97, 98, or 99%) sequence identity thereto;

(ii) the capsid protein comprises an amino acid sequence having at least one, two or three modifications but not more than 30, 20 or 10 modifications of the amino acid sequence of SEQ ID NO: 138;

(iii) the capsid protein comprises the amino acid sequence of SEQ ID NO: 11, or an amino acid sequence with at least 80% (e.g., at least about 85, 90, 95, 96, 97, 98, or 99%) sequence identity thereto;

(iv) the capsid protein comprises an amino acid sequence having at least one, two or three modifications but not more than 30, 20 or 10 modifications of the amino acid sequence of SEQ ID NO: 11;

(v) the capsid protein comprises an amino acid sequence encoded by the nucleotide sequence of SEQ ID NO: 137, or a sequence with at least 80% (e.g., at least about 85, 90, 95, 96, 97, 98, or 99%) sequence identity thereto; and/or

(vi) the nucleotide sequence encoding the capsid protein comprises the nucleotide sequence of SEQ ID NO: 137, or a sequence with at least 80% (e.g., at least about 85, 90, 95, 96, 97, 98, or 99%) sequence identity thereto.

124. The AAV particle of embodiment 122 or 123, wherein the capsid protein comprises:

(i) an amino acid substitution at position K449, e.g., a K449R substitution, numbered according to SEQ ID NO:138;

(ii) an insert comprising the amino acid sequence of TLAVPFK (SEQ ID NO: 1262), optionally wherein the insert is present immediately subsequent to position 588, relative to a reference sequence numbered according to SEQ ID NO:138;

(iii) an amino acid other than “A” at position 587 and/or an amino acid other than “Q” at position 588, numbered according to SEQ ID NO: 138;

(iv) the amino acid substitution of A587D and/or Q588G, numbered according to SEQ ID NO:138.

125. The AAV particle of any one of embodiments 122-124, wherein the capsid protein comprises (i) the amino acid substitution of K449R numbered according to SEQ ID NO:138; and (ii) an insert comprising the amino acid sequence of TLAVPFK (SEQ ID NO: 1262), optionally wherein the insert is present immediately subsequent to position 588 of SEQ ID NO:138.

126. The AAV particle of any one of embodiments 122-124, wherein the capsid protein comprises (i) the amino acid substitution of K449R numbered according to SEQ ID NO:138; (ii) an insert comprising the amino acid sequence of TLAVPFK (SEQ ID NO: 1262), optionally wherein the insert is present immediately subsequent to position 588, relative to a reference sequence numbered according to SEQ ID NO:138; and (iii) the amino acid substitutions of A587D and Q588G, numbered according to SEQ ID NO:138.

127. The AAV particle of any one of embodiments 122-124, wherein the capsid protein comprises (i) an insert comprising the amino acid sequence of TLAVPFK (SEQ ID NO: 1262), optionally wherein the insert is present immediately subsequent to position 588, relative to a reference sequence numbered according to SEQ ID NO:138; and (ii) the amino acid substitutions of A587D and Q588G, numbered according to SEQ ID NO:138.

128. The AAV particle of any one of embodiments 122-127, wherein the capsid protein comprises any of the capsid proteins listed in Table 1 or a functional variant thereof.

129. The AAV particle of any one of embodiments 122-128, wherein the capsid protein comprises a VOY101, VOY201, AAVPHP.N (PHP.N), AAVPHP.B (PHP.B), AAVPHP.A (PHP.A), PHP.B2, PHP.B3, G2B4, G2B5, AAV9, AAVrh10, or a functional variant thereof.

130. The AAV particle of any one of embodiments 122-129, wherein:

(i) the capsid protein comprises the amino acid sequence of SEQ ID NO: 1, or an amino acid sequence substantially identical (e.g., having at least 70%, 75%, 80%, 85%, 90%, 92%, 95%, 97%, 98%, or 99% sequence identity) thereto;

(ii) the capsid protein comprises an amino acid sequence comprising at least one, two, or three modifications but no more than 30, 20, or 10 modifications, e.g., substitutions, relative to the amino acid sequence of SEQ ID NO: 1;

(iii) the capsid protein comprises an amino acid sequence encoded by the nucleotide sequence of SEQ ID NO: 2 or a nucleotide sequence substantially identical (e.g., having at least 70%, 75%, 80%, 85%, 90%, 92%, 95%, 97%, 98%, or 99% sequence identity) thereto; and/or

(iv) the nucleotide sequence encoding the capsid protein comprises the nucleotide sequence of SEQ ID NO: 2, or a nucleotide sequence substantially identical (e.g., having at least 70%, 75%, 80%, 85%, 90%, 92%, 95%, 97%, 98%, or 99% sequence identity) thereto.

131. The AAV particle of any one of embodiments 122-130, wherein the capsid protein comprises:

(i) a VP1 polypeptide, VP2 polypeptide, VP3 polypeptide, or a combination thereof;

(ii) the amino acid sequence corresponding to positions 138-743, e.g., a VP2, of SEQ ID NO: 1, or a sequence with at least 80% (e.g., at least about 85, 90, 92, 95, 96, 97, 98, or 99%) sequence identity thereto;

(iii) the amino acid sequence corresponding to positions 203-743, e.g., a VP3, of SEQ ID NO: 1, or a sequence with at least 80% (e.g., at least about 85, 90, 92, 95, 96, 97, 98, or 99%) sequence identity thereto; and/or

(iv) the amino acid sequence corresponding to positions 1-743, e.g., a VP1, of SEQ ID NO: 1, or a sequence with at least 80% (e.g., at least about 85, 90, 92, 95, 96, 97, 98, or 99%) sequence identity thereto.

132. The AAV particle of any one of embodiments 122-131, wherein the nucleotide sequence encoding the capsid protein comprises:

(i) a CTG initiation codon; and/or

(ii) the nucleotide sequence of SEQ ID NO: 137 which comprises 3-20 mutations, e.g., substitutions, e.g., 3-15 mutations, 3-10 mutations, 3-5 mutations, 5-20 mutations, 5-15 mutations, 5-10 mutations, 10-20 mutations, 10-15 mutations, 15-20 mutations, 3 mutations, 5 mutations, 10 mutations, 12 mutations, 15 mutations, 18 mutations, or 20 mutations.

133. A vector comprising the isolated nucleic acid of any one of embodiments 1-6 or 9-49, or the viral genome of any one of embodiments 7-120.

134. A cell comprising the viral genome of any one of embodiments 7-120, the viral particle of any one of embodiments 122-132, or the vector of embodiment 133.

135. The cell of embodiment 134, which a mammalian cell, e.g., an HEK293 cell, an insect cell, e.g., an Sf9 cell, or a bacterial cell.

136. A nucleic acid comprising the viral genome of any one of embodiments 7-120, and a backbone region suitable for replication of the viral genome in a cell, e.g., a bacterial cell (e.g., wherein the backbone region comprises one or both of a bacterial origin of replication and a selectable marker).

137. The nucleic acid of embodiment 136, wherein the viral genome comprises a nucleotide sequence of any one of SEQ ID NOs: 1799-1082, 1752-1759, 1803-1821, or 1824-1830.

138. A method of making a viral genome, the method comprising:

(i) providing the nucleic acid molecule comprising the viral genome embodiment 136 or 137, or a nucleic acid encoding the viral genome of any one of embodiments 7-120; and

(ii) excising the viral genome from the backbone region, e.g., by cleaving the nucleic acid molecule at upstream and downstream of the viral genome.

139. A method of making an isolated, e.g., recombinant, AAV particle, the method comprising

(i) providing a host cell comprising the viral genome of any one of embodiments 7-120 or the nucleic acid encoding the viral genome of embodiment 136 or 137; and

(ii) incubating the host cell under conditions suitable to enclose the viral genome in a capsid protein, e.g., a VOY101 capsid protein;

thereby making the isolated AAV particle.

140. The method of embodiment 139, further comprising, prior to step (i), introducing a first nucleic acid molecule comprising the viral genome into the host cell.

141. The method of embodiment 139 or 140, wherein the host cell comprises a second nucleic acid encoding a capsid protein, e.g., a VOY101 capsid protein.

142. The method of embodiment 141, further comprising introducing the second nucleic acid into the cell.

143. The method of embodiment 141 or 142, wherein the second nucleic acid molecule is introduced into the host cell prior to, concurrently with, or after the first nucleic acid molecule.

144. The method of any one of embodiments 139-143, wherein the host cell comprises a mammalian cell, e.g., an HEK293 cell, an insect cell, e.g., an Sf9 cell, or a bacterial cell.

145. A pharmaceutical composition comprising the AAV particle of any one of embodiments 122-132, or an AAV particle comprising the viral genome of any one of embodiments 7-120, and a pharmaceutically acceptable excipient.

146. A method of delivering an exogenous GBA protein to a subject, comprising administering an effective amount of the pharmaceutical composition of embodiment 145, the AAV particle of any one of embodiments 122-132, an AAV particle comprising the viral genome of any one of embodiments 7-120, or an AAV particle comprising a viral genome comprising the nucleic acid of any one of embodiments 1-6 or 9-49, thereby delivering the exogenous GBA to the subject.

147. The method of embodiment 146, wherein the subject has, has been diagnosed with having, or is at risk of having a disease associated with expression of GBA, e.g., aberrant or reduced GBA expression, e.g., expression of an GBA gene, GBA mRNA, and/or GBA protein.

148. The method of embodiment 146 or 147, wherein the subject has, has been diagnosed with having, or is at risk of having a neurodegenerative or neuromuscular disorder.

149. A method of treating a subject having or diagnosed with having a disease associated with GBA expression comprising administering an effective amount of the pharmaceutical composition of embodiment 145, the AAV particle of any one of embodiments 122-132, an AAV particle comprising the viral genome of any one of embodiments 7-120, or an AAV particle comprising a viral genome comprising the nucleic acid of any one of embodiments 1-6 or 9-49, thereby treating the disease associated with GBA expression in the subject.

150. A method of treating a subject having or diagnosed with having a neurodegenerative or neuromuscular disorder, comprising administering an effective amount of the pharmaceutical composition of embodiment 145, the AAV particle of any one of embodiments 122-132, an AAV particle comprising the viral genome of any one of embodiments 7-120, or an AAV particle comprising a viral genome comprising the nucleic acid of any one of embodiments 1-6 or 9-49, thereby treating the neurodegenerative or neuromuscular disorder in the subject.

151. The method of any one of embodiments 147-150, wherein the disease associated with expression of GBA or the neurodegenerative or neuromuscular disorder comprises Parkinson's Disease (PD), dementia with Lewy Bodies (DLB), Gaucher disease (GD), Spinal muscular atrophy (SMA), Multiple System Atrophy (MSA), or Multiple sclerosis (MS).

152. A method of treating a subject having or diagnosed with having Parkinson's Disease (PD) (e.g., PD associated with a mutation in a GBA gene) comprising administering an effective amount of the pharmaceutical composition of embodiment 145, the AAV particle of any one of embodiments 122-132, an AAV particle comprising the viral genome of any one of embodiments 7-120, or an AAV particle comprising a viral genome comprising the nucleic acid of any one of embodiments 1-6 or 9-49, thereby treating PD in the subject.

153. The method of embodiment 151 or 152, wherein the PD is associated with a mutation in a GBA gene.

154. The method of any one of embodiments 151-153, wherein the PD is an early onset PD (e.g., before 50 years of age) or a juvenile PD (e.g., before 20 years of age).

155. The method of embodiment 151-154, wherein the PD is a tremor dominant, postural instability gait difficulty PD (PIGD) or a sporadic PD (e.g., a PD not associated with a mutation).

156. A method of treating a subject having or diagnosed with having Gaucher Disease (GD) comprising administering an effective amount of the pharmaceutical composition of embodiment 145, the AAV particle of any one of embodiments 122-132, an AAV particle comprising the viral genome of any one of embodiments 7-120, or an AAV particle comprising a viral genome comprising the nucleic acid of any one of embodiments 1-6 or 9-49, thereby treating GD in the subject.

157. The method of embodiment 151 or 156, wherein the GD is neuronopathic GD (e.g., affect a cell or tissue of the CNS, e.g., a cell or tissue of the brain and/or spinal cord), non-neuronopathic GD (e.g., does not affect a cell or tissue of the CNS), or combination thereof.

158. The method of any one of embodiments 151 or 156-157, wherein the GD is Type I GD (GD1), Type 2 GD (GD2), or Type 3 GD (GD3).

159. The method of embodiment 158, wherein the GD1 is non-neuronopathic GD.

160. The method of embodiment 158, wherein the GD2 is a neuronopathic GD.

161. The method of any one of embodiments 146-160, wherein the subject has a reduced level of GCase activity as compared to a reference level, when measured by an assay, e.g., an assay as described in Example 7.

162. The method of embodiment 161, wherein the reference level comprises the level of GCase activity in a subject that does not have a disease associated with GBA expression, a neuromuscular and/or a neurodegenerative disorder.

163. The method of any one of embodiments 149-162, wherein treating comprises prevention of progression of the disease in the subject.

164. The method of any one of embodiments 149-163, wherein treating results in amelioration of at least one symptom of the disease associated with GBA expression, the neurodegenerative disorder, and/or the neuromuscular disorder in the subject.

165. The method of embodiment 164, wherein the symptom of the disease associated with GBA expression, the neurodegenerative disorder, and/or the neuromuscular disorder comprises reduced GCase activity, accumulation of glucocerebroside and other glycolipids, e.g., within immune cells (e.g., macrophages), build-up of synuclein aggregates (e.g., Lewy bodies), developmental delay, progressive encephalopathy, progressive dementia, ataxia, myoclonus, oculomotor dysfunction, bulbar palsy, generalized weakness, trembling of a limb, depression, visual hallucinations, cognitive decline, or a combination thereof.

166. The method of any one of embodiments 146-165, wherein the subject is a human.

167. The method of any one of embodiments 146-166, wherein the subject is a juvenile, e.g., between 6 years of age to 20 years of age.

168. The method of any one of embodiments 146-167, wherein the subject is an adult, e.g., above 20 years of age.

169. The method of any one of embodiments 146-168, wherein the subject has a mutation in a GBA gene, GBA mRNA, and/or GBA protein.

170. The method of any one of embodiments 146-169, wherein the AAV particle is administered to the subject intravenously, intracerebrally, via intrathalamic (ITH) administration, intramuscularly, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, or via intra-cisterna magna injection (ICM).

171. The method of any one of embodiments 146-170, wherein the AAV particle is administered via dual ITH and ICM administration.

172. The method of any one of embodiments 146-170, wherein the AAV particle is administered via intravenous injection, optionally wherein the intravenous injection is via focused ultrasound (FUS), e.g., coupled with the intravenous administration of microbubbles (FUS-MB), or MRI-guided FUS coupled with intravenous administration.

173. The method of any one of embodiments 146-172, wherein the AAV particle is administered to a cell, tissue, or region of the CNS, e.g., a region of the brain or spinal cord, e.g., the parenchyma, the cortex, substantia nigra, caudate cerebellum, striatum, corpus callosum, cerebellum, brain stem caudate-putamen, thalamus, superior colliculus, the spinal cord, or a combination thereof.

174. The method of any one of embodiments 146-173, wherein the AAV particle is administered to a cell, tissue, or region of the periphery, e.g., a lung cell or tissue, a heart cell or tissue, a spleen cell or tissue, a liver cell or tissue, or a combination thereof.

175. The method of any one of embodiments 146-174, wherein the AAV particle is administered to the cerebral spinal fluid, the serum, or a combination thereof.

176. The method of any one of embodiments 146-175, wherein the AAV particle is administered to at least two tissues, or regions of the CNS, e.g., bilateral administration.

177. The method of any one of embodiments 146-176, further comprising performing a blood test, performing an imaging test, collecting a CNS biopsy sample, collecting a tissue biopsy, (e.g., a biopsy of the lung, liver, or spleen), collecting a blood or serum sample, or collecting an aqueous cerebral spinal fluid biopsy.

178. The method of any one of embodiments 146-177, which further comprises evaluating, e.g., measuring, the level of GBA expression, e.g., GBA gene, GBA mRNA, and/or GBA protein expression, in the subject, e.g., in a cell, tissue, or fluid, of the subject, optionally wherein the level of GBA protein is measured by an assay described herein, e.g., an ELISA, a Western blot, or an immunohistochemistry assay.

179. The method of embodiment 178, wherein measuring the level of GBA expression is performed prior to, during, or subsequent to treatment with the AAV particle.

180. The method of embodiment 178 or 179, wherein the cell or tissue is a cell or tissue of the central nervous system (e.g., parenchyma) or a peripheral cell or tissue (e.g., the liver, heart, and/or spleen).

181. The method of any one of embodiments 146-180, wherein the administration results in increased level of GBA protein expression in a cell or tissue of the subject, relative to reference level, e.g., a subject that has not received treatment, e.g., has not been administered the AAV particle.

182. The method of any one of embodiments 146-181, which further comprises evaluating, e.g., measuring, the level of GCase activity in the subject, e.g., in a cell or tissue of the subject, optionally wherein the level of GCase activity is measured by an assay described herein, e.g., assay as described in Example 7.

183. The method of any one of embodiments 146-182, wherein the administration results in an increase in at least one, two, or all of:

(i) the level of GCase activity in a cell, tissue, (e.g., a cell or tissue of the CNS, e.g., the cortex, striatum, thalamus, cerebellum, and/or brainstem), and/or fluid (e.g., CSF and/or serum), of the subject, optionally wherein the level of GCase activity is increased by at least 3, 4, 4.5, 4.6, 4.7, 4.8, 4.9, 5, 5.1, 5.2, 5.3, 5.4, or 5.5 fold, as compared to a reference level, e.g., a subject that has not received treatment, e.g., has not been administered the AAV particle;

(ii) the level of viral genomes (VG) per cell in a CNS tissue (e.g., the cortex, striatum, thalamus, cerebellum, brainstem, and/or spinal cord) of the subject, optionally wherein the VG level is increased by greater than 50 VGs per cell, as compared to a peripheral tissue, wherein the level of VGs per cell is at least 4-10 fold lower than the levels in the CNS tissue, e.g., as measured by an assay as described herein; and/or

(iii) the level of GBA mRNA expression in a cell or tissue (e.g. a cell or tissue of the CNS, e.g., the cortex, thalamus, and/or brainstem), optionally wherein the level of GBA mRNA is increased by at least 100-1300 fold, e.g., 100 fold, 200 fold, 500 fold, 600 fold, 850 fold, 900 fold, 950 fold, 1000 fold, 1050 fold, 1100 fold, 1150 fold, 1200 fold, 1250 fold, or 1300 fold as compared to a reference level, e.g., a subject that has not received treatment (e.g., has not been administered the AAV particle), or endogenous GBA mRNA levels, e.g., as measured by an assay as described herein.

184. The method of any one of embodiments 146-183, wherein further comprising administration of an additional therapeutic agent and/or therapy suitable for treatment or prevention of the disease associated GBA expression, the neurodegenerative disorder, and/or the neuromuscular disorder.

185. The method of embodiment 184, wherein the additional therapeutic agent comprises enzyme replacement therapy (ERT) (e.g., imiglucerase, velaglucerase alfa, or taliglucerase alfa); substrate reduction therapy (SRT) (e.g., eliglustat or miglustat), blood transfusion, levodopa, carbidopa, Safinamide, dopamine agonists (e.g., pramipexole, rotigotine, or ropinirole), anticholinergics (e.g., benztropine or trihexyphenidyl), cholinesterase inhibitors (e.g., rivastigmine, donepezil, or galantamine), an N-methyl-d-aspartate (NMDA) receptor antagonist (e.g., memantine), or a combination thereof.

186. The isolated nucleic acid of any one of embodiments 1-6 or 9-49, the viral genome of any one of embodiments 7-120, the AAV particle of any one of embodiments 122-132, or the pharmaceutical composition of embodiment 145 for use in the manufacture of a medicament.

187. The isolated nucleic acid of any one of embodiments 1-6 or 9-49, the viral genome of any one of embodiments 7-120, the AAV particle of any one of embodiments 122-132, or the pharmaceutical composition of embodiment 145 for use in the treatment of a disease associated with GBA expression, a neuromuscular and/or a neurodegenerative disorder.

188. Use of an effective amount of an AAV particle comprising the genome of any one of embodiments 7-120, an AAV particle comprising a genome comprising the nucleic acid of any one of embodiments 1-6 or 9-49, the AAV particle of any one of embodiments 122-132, or the pharmaceutical composition of embodiment 145, in the manufacture of a medicament for the treatment of a disease associated with GBA expression, a neuromuscular and/or a neurodegenerative disorder.

189. An adeno-associated viral (AAV) vector genome comprising a sequence selected from any of SEQ ID NO: 1759-1771 190. An AAV particle comprising the AAV vector genome of claim 189 and a capsid selected from a group consisting of those listed in Table 1.

191. The AAV particle of claim 190, wherein the capsid comprises an AAV2 serotype. 192. A pharmaceutical composition comprising the AAV particle of claim 190 or claim 191.

193. A method of treating a neurological or neuromuscular disorder, said method comprising administering to a subject the pharmaceutical composition of claim 192.

194. The method of claim 193, wherein the neurological or neuromuscular disorder is Parkinson's Disease, Gaucher disease, or Dementia with Lewy Bodies, or a related disorder.

195. The method of claim 194, wherein the neurological or neuromuscular disorder is a disorder associated with decreased GCase protein levels.

The details of various aspects or embodiments of the present disclosure are set forth below. Other features, objects, and advantages of the disclosure will be apparent from the description and the claims. In the description, the singular forms also include the plural unless the context clearly dictates otherwise. Unless defined otherwise, all technical and scientific terms used herein have the same meaning as commonly understood by one of ordinary skill in the art in the field of this disclosure. In the case of conflict, the present description will control.

### BRIEF DESCRIPTION OF THE DRAWINGS

FIGS. 1A-1B depict LC-MS/MS results quantifying levels of GBA substrate glucosylsphingosine (GlcSph) in cell lysates of Gaucher disease patient derived fibroblasts (GD1 patient GM04394, GD1 Patient GM00852, and GD2 patient GM00877) and healthy control fibroblasts (CLT GM05758, CTL GM02937 and CTL GM08402). Data are shown as GlcSph normalized to actin (FIG. 1A) or normalized to lysosomal protein Lamp1 (FIG. 1B).

FIG. 1C depicts GBA protein levels detected in lysates of Gaucher patient-derived fibroblasts (GD1 and GD2) compared to healthy control fibroblast (HC) by LC-MS/MS. Data are shown as concentration of GBA protein (ng) relative to total protein (mg).

FIGS. 2A-2B depict GCase activity (RFU/mL normalized to mg of protein) in GD-II GM00877 fibroblast cell pellets (FIG. 2A) or conditioned media (FIG. 2B) at Day 7 after transduction with AAV2 viral particles comprising the viral genome construct on the X-axis from left to right: GBA_VG1 (SEQ ID NO: 1759), GBA_VG9 (SEQ ID NO: 1767), GBA_VG10 (SEQ ID NO: 1768), GBA_VG11 (SEQ ID NO: 1769), GBA_VG6 (SEQ ID NO: 1764), GBA_VG7 (SEQ ID NO: 1765), GBA_VG12 (SEQ ID NO: 1770), GBA_VG3 (SEQ ID NO: 1761), GBA_VG4 (SEQ ID NO: 1762), GBA_VG5 (SEQ ID NO: 1763), and GBA_VG13 (SEQ ID NO: 1771), at MOI of 10^{3.5}. The dotted line indicates the baseline level (vehicle treatment).

FIG. 3 depicts levels of GBA substrate glucosylsphingosine (GlcSph) in the cell lysates (ng/mg Lamp1) collected from GD-II patient fibroblasts (GM00877) at Day 7 after transduction with transduction of a no AAV control or AAV2 vectors comprising the viral genome indicated on the X-axis (from left to right: GBA_VG1 (SEQ ID NO: 1759), GBA_VG9 (SEQ ID NO: 1767), GBA_VG6 (SEQ ID NO: 1764), GBA_VG7 (SEQ ID NO: 1765), GBA_VG3 (SEQ ID NO: 1761), GBA_VG4 (SEQ ID NO: and GBA_VG5(SEQ ID NO: 1763)).

FIG. 4A depicts GCase activity measured as RFU per mL normalized to mg of protein in GD-II patient fibroblasts (GD-II GM00877) on day 7 post-transduction with AAV2 vectors comprising the viral genome indicated on the X-axis (from left to right: GBA_VG1 (SEQ ID NO: 1759), GBA_VG14 (SEQ ID NO: 1809), GBA_VG15 (SEQ ID NO: 1810), GBA_VG16 (SEQ ID NO: 1811), GBA_VG17 (SEQ ID NO: 1812), GBA_VG18 (SEQ ID NO: 1813), GBA_VG19 (SEQ ID NO: 1814), and GBA_VG20 (SEQ ID NO: 1815)) at an MOI of 10^{2.5} (first bar), 10³ (second bar), 10^{3.5} and 10⁴ (third bar). FIG. 4B depicts the level of the GBA substrate glucosylsphingosine (GlcSph, ng/mg Lamp1) in the cell lysate from GD-II patient-derived fibroblasts at day 7 after transduction with AAV2 vectors comprising the viral genome indicated on the X-axis (from left to right: GBA_VG1 (SEQ ID NO: 1759), GBA_VG14 (SEQ ID NO: 1809), GBA_VG15 (SEQ ID NO: 1810), GBA_VG16 (SEQ ID NO: 1811), GBA_VG17 (SEQ ID NO: 1812), GBA_VG18 (SEQ ID NO: 1813), GBA_VG19 (SEQ ID NO: 1814), and GBA_VG20 (SEQ ID NO: 1815)) at an MOI of 10^{2.5} (first bar), 10³ (second bar), 10^{3.5} and 10⁴ (third bar).

FIG. 5 depicts the GC content and distribution of a first codon-optimized nucleotide sequence encoding a GBA protein of SEQ ID NO: 1773, a second codon-optimized nucleotide sequence encoding a GBA protein of SEQ ID NO: 1781, and a wild-type nucleotide sequence encoding a GBA protein of SEQ ID NO: 1777.

FIGS. 6A-6B compare activity of a GBA protein expressed by AAV2 vectorized viral genome constructs: GBA_VG1 (SEQ ID NO: 1759), GBA_VG17 (SEQ ID NO: 1812), and GBA_VG21 (SEQ ID NO: 1816). FIG. 6A depicts the GCase activity (RFU/mL) normalized to mg of protein in GD-II patient fibroblasts treated with AAV2 viral particles at an MOI of 10^{4.5} comprising the viral genome constructs indicated on the X-axis (GBA_VG1 (SEQ ID NO: 1759), GBA_VG17 (SEQ ID NO: 1812), and GBA_VG21 (SEQ ID NO: 1816)) compared to a no AAV control. FIG. 6B depicts glucosylsphingosine (GlcSph) (ng/mL Lamp1) in the cell lysate from GD-II patient fibroblasts treated with AAV2 viral particles comprising the viral genome constructs indicated on the X-axis (from left to right GBA_VG1 (SEQ ID NO: 1759), GBA_VG17 (SEQ ID NO: 1812), and GBA_VG21 (SEQ ID NO: 1816)) at an MOI of 10⁶, or a no AAV treatment control.

FIG. 7 depicts the GCase activity (RFU/mL) per mg of protein in rat embryonic dorsal root ganglion (DRG) neurons transduced an AAV2 vector comprising GBA_VG33 (SEQ ID NO: 1828) or an AAV2 vector comprising GBA_VG17 (SEQ ID NO: 1812) at an MOI of 10^{3.5} or 10^{4.5}, compared to a no AAV control.

FIG. 8 depicts the biodistribution (VG/cell) versus GCase activity (RFU/mL, fold over endogenous GCase activity, normalized to mg of protein) in the cortex, striatum, thalamus, brainstem, cerebellum, and liver in wild-type mice at one-month post-IV injection of VOY101.GBA_VG17 (SEQ ID NO: 1812) at 2e13 vg/kg.

### DETAILED DESCRIPTION

### Overview

Described herein, inter alia, are compositions comprising isolated, e.g., recombinant, viral particles, e.g., AAV particles, for delivery, e.g., vectorized delivery, of a protein, e.g., a GBA protein, and methods of making and using the same. Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload. The genome of the virus may be modified to contain a minimum of components for the assembly of a functional recombinant virus, or viral particle, which is loaded with or engineered to express or deliver a desired nucleic acid construct or payload, e.g., a transgene, polypeptide-encoding polynucleotide, e.g., a GBA protein, e.g., a GCase, GCase and PSAP, GCase and SapA, or GCase and SapC, GCase and a cell penetration peptide (e.g., an ApoEII peptide, a TAT peptide, or an ApoB peptide), or GCase and a lysosomal targeting sequence (LTS), which may be delivered to a target cell, tissue, or organism. In some embodiments, the target cell is a CNS cell. In some embodiments, the target tissue is a CNS tissue. The target CNS tissue may be brain tissue. In some embodiments, the brain target comprises caudate, putamen, thalamus, superior colliculus, cortex, and corpus collosum.

Gene therapy presents an alternative approach for PD and related diseases sharing single-gene etiology, such as Gaucher disease and Dementia with Lewy Bodies and related disorders. AAVs are commonly used in gene therapy approaches as a result of a number of advantageous features. Without wishing to be bound by theory, it is believed in some embodiments, that expression vectors, e.g., an adeno-associated viral vector (AAVs) or AAV particle, e.g., an AAV particle described herein, can be used to administer and/or deliver a GBA protein (e.g., GCase and related proteins), in order to achieve sustained, high concentrations, allowing for longer lasting efficacy, fewer dose treatments, broad biodistribution, and/or more consistent levels of the GBA protein, relative to a non-AAV therapy.

As demonstrated in the Examples herein below, the compositions and methods described herein provides improved features compared to prior enzyme replacement approaches, including (i) increased GCase activity in a cell, tissue, (e.g., a cell or tissue of the CNS, e.g., the cortex, striatum, thalamus, cerebellum, and/or brainstem), and/or fluid (e.g., CSF and/or serum), of the subject; (ii) increased biodistribution throughout the CNS (e.g., the cortex, striatum, thalamus, cerebellum, brainstem, and/or spinal cord), and the periphery (e.g., the liver), and/or (iii) elevated payload expression, e.g., GBA mRNA expression, in multiple brain regions (e.g., cortex, thalamus, and brain stem) and the periphery (e.g., the liver). In some embodiments, an AAV viral genome encoding a GBA protein described herein which comprise an optimized nucleotide sequence encoding the GBA protein (e.g., SEQ ID NO: 1773) result in high biodistribution in the CNS; increased GCase activity in the CNS, peripheral tissues, and/or fluid; and successful transgene transcription and expression. The compositions and methods described herein can be used in the treatment of disorders associated with a lack of a GBA protein and/or GCase activity, such as neuronopathic (affects the CNS) and non-neuronopathic (affects non-CNS) Gaucher's disease (e.g., Type 1 GD, Type 2 GD, or Type 3 GD), a PD associated with a mutation in a GBA gene, and a dementia with Lewy Bodies (DLB).

### I. Compositions

Adeno-associated viral (AAV) vectors

AAV have a genome of about 5,000 nucleotides in length which contains two open reading frames encoding the proteins responsible for replication (Rep) and the structural protein of the capsid (Cap). The open reading frames are flanked by two Inverted Terminal Repeat (ITR) sequences, which serve as the origin of replication of the viral genome. The wild-type AAV viral genome comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. As another non-limiting example, VP1 refers to amino acids 1-743 numbered according to SEQ ID NO: 1, VP2 refers to amino acids 138-743 numbered according to SEQ ID NO: 1, and VP3 refers to amino acids 203-743 numbered according to SEQ ID NO: 1. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

The AAV vector typically requires a co-helper (e.g., adenovirus) to undergo productive infection in cells. In the absence of such helper functions, the AAV virions essentially enter host cells but do not integrate into the cells' genome.

AAV vectors have been investigated for delivery of gene therapeutics because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector, and (v) the non-integrative nature in a host chromosome thereby reducing potential for long-term genetic alterations. Moreover, infection with AAV vectors has minimal influence on changing the pattern of cellular gene expression (Stilwell and Samulski et al., Biotechniques, 2003, 34, 148, the contents of which are herein incorporated by reference in their entirety).

Typically, AAV vectors for GCase protein delivery may be recombinant viral vectors which are replication defective as they lack sequences encoding functional Rep and Cap proteins within the viral genome. In some cases, the defective AAV vectors may lack most or all coding sequences and essentially only contain one or two AAV ITR sequences and a payload sequence. In certain embodiments, the viral genome encodes GCase protein. In some embodiments, the viral genome encodes GCase protein and SapA protein. In some embodiments, the viral genome encodes GCase protein and SapC protein. For example, the viral genome can encode human GCase, human GCase+SapA, or human GCase+SapC protein(s).

In some embodiments, the viral genome may comprise one or more lysosomal targeting sequences (LTS).

In some embodiments, the viral genome may comprise one or more cell penetrating peptide sequences (CPP).

In some embodiments, a viral genome may comprise one or more lysosomal targeting sequences and one or more cell penetrating sequences.

In some embodiments, the AAV particles of the present disclosure may be introduced into mammalian cells.

AAV vectors may be modified to enhance the efficiency of delivery. Such modified AAV vectors of the present disclosure can be packaged efficiently and can be used to successfully infect the target cells at high frequency and with minimal toxicity.

In other embodiments, AAV particles of the present disclosure may be used to deliver GCase protein to the central nervous system (see, e.g., U.S. Pat. No. 6,180,613; the contents of which are herein incorporated by reference in their entirety) or to specific tissues of the CNS.

As used herein, the term “AAV vector” or “AAV particle” comprises a capsid and a viral genome comprising a payload. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, e.g., GCase protein.

It is understood that the compositions described herein may have additional conservative or non-essential amino acid substitutions, which do not have a substantial effect on their functions.

### AAV Serotypes

AAV particles of the present disclosure may comprise or be derived from any natural or recombinant AAV serotype. According to the present disclosure, the AAV particles may utilize or be based on a serotype or include a peptide selected from any of the following VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the AAV serotype may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAVS-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607), AAV-LKO2 (SEQ ID NO:3 of US20150376607), AAV-LKO3 (SEQ ID NO:4 of US20150376607), AAV-LKO4 (SEQ ID NO:5 of US20150376607), AAV-LKO5 (SEQ ID NO:6 of US20150376607), AAV-LKO6 (SEQ ID NO:7 of US20150376607), AAV-LKO7 (SEQ ID NO:8 of US20150376607), AAV-LKO8 (SEQ ID NO:9 of US20150376607), AAV-LKO9 (SEQ ID NO:10 of US20150376607), AAV-LK10 (SEQ ID NO:11 of US20150376607), AAV-LK11 (SEQ ID NO:12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV serotype may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV serotype may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAV capsid serotype selection or use may be from a variety of species. In some embodiments, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in their entirety.

In some embodiments, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO: 2 and 20 of WO2016049230), AAVF2/HSC2 (SEQ ID NO: 3 and 21 of WO2016049230), AAVF3/HSC3 (SEQ ID NO: 5 and 22 of WO2016049230), AAVF4/HSC4 (SEQ ID NO: 6 and 23 of WO2016049230), AAVF5/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In some embodiments, the AAV serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV CLv-D1 (SEQ ID NO: 22 and 96 of U.S. Pat. No. 8,734,809), AAV CLv-D2 (SEQ ID NO: 23 and 97 of U.S. Pat. No. 8,734,809), AAV CLv-D3 (SEQ ID NO: 24 and 98 of U.S. Pat. No. 8,734,809), AAV CLv-D4 (SEQ ID NO: 25 and 99 of U.S. Pat. No. 8,734,809), AAV CLv-D5 (SEQ ID NO: 26 and 100 of U.S. Pat. No. 8,734,809), AAV CLv-D6 (SEQ ID NO: 27 and 101 of U.S. Pat. No. 8,734,809), AAV CLv-D7 (SEQ ID NO: 28 and 102 of U.S. Pat. No. 8,734,809), AAV CLv-D8 (SEQ ID NO: 29 and 103 of U.S. Pat. No. 8,734,809), AAV CLv-E1 (SEQ ID NO: 13 and 87 of U.S. Pat. No. 8,734,809), AAV CLv-R1 (SEQ ID NO: 30 and 104 of U.S. Pat. No. 8,734,809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: X and X of U.S. Pat. No. 8,734,809), AAV CLv-R9 (SEQ ID NO: X and X of U.S. Pat. No. 8,734,809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of U.S. Pat. No. 8,734,809), AAV CLg-F4 (SEQ ID NO: 42 and 116 of U.S. Pat. No. 8,734,809), AAV CLg-F5 (SEQ ID NO: 43 and 117 of U.S. Pat. No. 8,734,809), AAV CLg-F6 (SEQ ID NO: 43 and 117 of U.S. Pat. No. 8,734,809), AAV CLg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of U.S. Pat. No. 8,734,809), AAV CSp-10 (SEQ ID NO: 46 and 120 of U.S. Pat. No. 8,734,809), AAV CSp-11 (SEQ ID NO: 47 and 121 of U.S. Pat. No. 8,734,809), AAV CSp-2 (SEQ ID NO: 48 and 122 of U.S. Pat. No. 8,734,809), AAV CSp-3 (SEQ ID NO: 49 and 123 of U.S. Pat. No. 8,734,809), AAV CSp-4 (SEQ ID NO: 50 and 124 of U.S. Pat. No. 8,734,809), AAV CSp-6 (SEQ ID NO: 51 and 125 of U.S. Pat. No. 8,734,809), AAV CSp-7 (SEQ ID NO: 52 and 126 of U.S. Pat. No. 8,734,809), AAV CSp-8 (SEQ ID NO: 53 and 127 of U.S. Pat. No. 8,734,809), AAV CSp-9 (SEQ ID NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of U.S. Pat. No. 8,734,809), AAV CKd-3 (SEQ ID NO: 60 and 134 of U.S. Pat. No. 8,734,809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of U.S. Pat. No. 8,734,809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809), AAV CLv-3 (SEQ ID NO: 69 and 143 of U.S. Pat. No. 8,734,809), AAV CLv-4 (SEQ ID NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ ID NO: 77 and 151 of U.S. Pat. No. 8,734,809), AAV CKd-B6 (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809), AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of U.S. Pat. No. 8,734,809), AAV CKd-H5 (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of U.S. Pat. No. 8,734,809), AAV CLv1-1 (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U.S. Pat. No. 8,734,809), AAV.VR-355 (SEQ ID NO: 181 of U.S. Pat. No. 8,734,809), AAV.hu.48R3 (SEQ ID NO: 183 of U.S. Pat. No. 8,734,809), or variants or derivatives thereof.

In some embodiments, the AAV serotype may be, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7.10 (SEQ ID NO: 11 and 61 of WO2016065001), AAV CKd-N3 (SEQ ID NO: 12 and 62 of WO2016065001), AAV CKd-N4 (SEQ ID NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In some embodiments, the AAV particle may have, or may be a serotype selected from any of those found in Table 1.

In some embodiments, the AAV capsid may comprise a sequence, fragment or variant thereof, of any of the sequences in Table 1.

In some embodiments, the AAV capsid may be encoded by a sequence, fragment or variant as described in Table 1.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

TABLE 1
AAV Serotypes

| Serotype | SEQ ID NO: | Reference Information |
|---|---|---|
| VOY101 | 1 | — |
| VOY101 | 2 | — |
| VOY201 | 3 | — |
| PHP.N/PHP.B-DGT | 4 | WO2017100671 SEQ ID NO: 46 |
| AAVPHP.B or G2B-26 | 5 | WO2015038958 SEQ ID NO: 8 and 13 |
| AAVPHP.B | 6 | WO2015038958 SEQ ID NO: 9 |
| AAVG2B-13 | 7 | WO2015038958 SEQ ID NO: 12 |
| AAVTH1.1-32 | 8 | WO2015038958 SEQ ID NO: 14 |
| AAVTH1.1-35 | 9 | WO2015038958 SEQ ID NO: 15 |
| PHP.S/G2A12 | 10 | WO2017100671 SEQ ID NO: 47 |
| AAV9/hu.14 K449R | 11 | WO2017100671 SEQ ID NO: 45 |
| AAV1 | 12 | US20150159173 SEQ ID NO: 11, |
| | | US20150315612 SEQ ID NO: 202 |
| AAV1 | 13 | US20160017295 SEQ ID NO: 1, |
| | | US20030138772 SEQ ID NO: 64, |
| | | US20150159173 SEQ ID NO: 27, |
| | | US20150315612 SEQ ID NO: 219, |
| | | U.S. Pat. No. 7,198,951 SEQ ID NO: 5 |
| AAV1 | 14 | US20030138772 SEQ ID NO: 6 |
| AAV1.3 | 15 | US20030138772 SEQ ID NO: 14 |
| AAV10 | 16 | US20030138772 SEQ ID NO: 117 |
| AAV10 | 17 | WO2015121501 SEQ ID NO: 9 |
| AAV10 | 18 | WO2015121501 SEQ ID NO: 8 |
| AAV11 | 19 | US20030138772 SEQ ID NO: 118 |
| AAV12 | 20 | US20030138772 SEQ ID NO: 119 |
| AAV2 | 21 | US20150159173 SEQ ID NO: 7, |
| | | US20150315612 SEQ ID NO: 211 |
| AAV2 | 22 | US20030138772 SEQ ID NO: 70, |
| | | US20150159173 SEQ ID NO: 23, |
| | | US20150315612 SEQ ID NO: 221, US20160017295 |
| | | SEQ ID NO: 2, U.S. Pat. No. 6,156,303 |
| | | SEQ ID NO: 4, U.S. Pat. No. 7,198,951 |
| | | SEQ ID NO: 4, WO2015121501 SEQ ID NO: 1 |
| AAV2 | 23 | U.S. Pat. No. 6,156,303 SEQ ID NO: 8 |
| AAV2 | 24 | US20030138772 SEQ ID NO: 7 |
| AAV2 | 25 | U.S. Pat. No. 6,156,303 SEQ ID NO: 3 |
| AAV2.5T | 26 | U.S. Pat. No. 9,233,131 SEQ ID NO: 42 |
| AAV223.10 | 27 | US20030138772 SEQ ID NO: 75 |
| AAV223.2 | 28 | US20030138772 SEQ ID NO: 49 |
| AAV223.2 | 29 | US20030138772 SEQ ID NO: 76 |
| AAV223.4 | 30 | US20030138772 SEQ ID NO: 50 |
| AAV223.4 | 31 | US20030138772 SEQ ID NO: 73 |
| AAV223.5 | 32 | US20030138772 SEQ ID NO: 51 |
| AAV223.5 | 33 | US20030138772 SEQ ID NO: 74 |
| AAV223.6 | 34 | US20030138772 SEQ ID NO: 52 |
| AAV223.6 | 35 | US20030138772 SEQ ID NO: 78 |
| AAV223.7 | 36 | US20030138772 SEQ ID NO: 53 |
| AAV223.7 | 37 | US20030138772 SEQ ID NO: 77 |
| AAV29.3 | 38 | US20030138772 SEQ ID NO: 82 |
| AAV29.4 | 39 | US20030138772 SEQ ID NO: 12 |
| AAV29.5 | 40 | US20030138772 SEQ ID NO: 83 |
| AAV29.5 (AAVbb.2) | 41 | US20030138772 SEQ ID NO: 13 |
| AAV3 | 42 | US20150159173 SEQ ID NO: 12 |
| AAV3 | 43 | US20030138772 SEQ ID NO: 71, |
| | | US20150159173 SEQ ID NO: 28, |
| | | US20160017295 SEQ ID NO: 3, |
| | | U.S. Pat. No. 7,198,951 SEQ ID NO. 6 |
| AAV3 | 44 | US20030138772 SEQ ID NO: 8 |
| AAV3.3b | 45 | US20030138772 SEQ ID NO: 72 |
| AAV3-3 | 46 | US20150315612 SEQ ID NO: 200 |
| AAV3-3 | 47 | US20150315612 SEQ ID NO: 217 |
| AAV3a | 48 | U.S. Pat. No. 6,156,303 SEQ ID NO: 5 |
| AAV3a | 49 | U.S. Pat. No. 6,156,303 SEQ ID NO: 9 |
| AAV3b | 50 | U.S. Pat. No. 6,156,303 SEQ ID NO: 6 |
| AAV3b | 51 | U.S. Pat. No. 6,156,303 SEQ ID NO: 10 |
| AAV3b | 52 | U.S. Pat. No. 6,156,303 SEQ ID NO: 1 |
| AAV4 | 53 | US20140348794 SEQ ID NO: 17 |
| AAV4 | 54 | US20140348794 SEQ ID NO: 5 |
| AAV4 | 55 | US20140348794 SEQ ID NO: 3 |
| AAV4 | 56 | US20140348794 SEQ ID NO: 14 |
| AAV4 | 57 | US20140348794 SEQ ID NO: 15 |
| AAV4 | 58 | US20140348794 SEQ ID NO: 19 |
| AAV4 | 59 | US20140348794 SEQ ID NO: 12 |
| AAV4 | 60 | US20140348794 SEQ ID NO: 13 |
| AAV4 | 61 | US20140348794 SEQ ID NO: 7 |
| AAV4 | 62 | US20140348794 SEQ ID NO: 8 |
| AAV4 | 63 | US20140348794 SEQ ID NO: 9 |
| AAV4 | 64 | US20140348794 SEQ ID NO: 2 |
| AAV4 | 65 | US20140348794 SEQ ID NO: 10 |
| AAV4 | 66 | US20140348794 SEQ ID NO. 11 |
| AAV4 | 67 | US20140348794 SEQ ID NO: 18 |
| AAV4 | 68 | US20030138772 SEQ ID NO: 63, |
| | | US20160017295 SEQ ID NO: 4, |
| | | US20140348794 SEQ ID NO: 4 |
| AAV4 | 69 | US20140348794 SEQ ID NO: 16 |
| AAV4 | 70 | US20140348794 SEQ ID NO: 20 |
| AAV4 | 71 | US20140348794 SEQ ID NO: 6 |
| AAV4 | 72 | US20140348794 SEQ ID NO: 1 |
| AAV42.2 | 73 | US20030138772 SEQ ID NO: 9 |
| AAV42.2 | 74 | US20030138772 SEQ ID NO: 102 |
| AAV42.3b | 75 | US20030138772 SEQ ID NO: 36 |
| AAV42.3B | 76 | US20030138772 SEQ ID NO: 107 |
| AAV42.4 | 77 | US20030138772 SEQ ID NO: 33 |
| AAV42.4 | 78 | US20030138772 SEQ ID NO: 88 |
| AAV42.8 | 79 | US20030138772 SEQ ID NO: 27 |
| AAV42.8 | 80 | US20030138772 SEQ ID NO: 85 |
| AAV43.1 | 81 | US20030138772 SEQ ID NO: 39 |
| AAV43.1 | 82 | US20030138772 SEQ ID NO: 92 |
| AAV43.12 | 83 | US20030138772 SEQ ID NO: 41 |
| AAV43.12 | 84 | US20030138772 SEQ ID NO: 93 |
| AAV43.20 | 85 | US20030138772 SEQ ID NO: 42 |
| AAV43.20 | 86 | US20030138772 SEQ ID NO. 99 |
| AAV43.21 | 87 | US20030138772 SEQ ID NO: 43 |
| AAV43.21 | 88 | US20030138772 SEQ ID NO: 96 |
| AAV43.23 | 89 | US20030138772 SEQ ID NO: 44 |
| AAV43.23 | 90 | US20030138772 SEQ ID NO: 98 |
| AAV43.25 | 91 | US20030138772 SEQ ID NO: 45 |
| AAV43.25 | 92 | US20030138772 SEQ ID NO: 97 |
| AAV43.5 | 93 | US20030138772 SEQ ID NO: 40 |
| AAV43.5 | 94 | US20030138772 SEQ ID NO: 94 |
| AAV4-4 | 95 | US20150315612 SEQ ID NO: 201 |
| AAV4-4 | 96 | US20150315612 SEQ ID NO: 218 |
| AAV44.1 | 97 | US20030138772 SEQ ID NO: 46 |
| AAV44.1 | 98 | US20030138772 SEQ ID NO: 79 |
| AAV44.5 | 99 | US20030138772 SEQ ID NO: 47 |
| AAV44.5 | 100 | US20030138772 SEQ ID NO: 80 |
| AAV4407 | 101 | US20150315612 SEQ ID NO: 90 |
| AAV5 | 102 | U.S. Pat. No. 7,427,396 SEQ ID NO: 1 |
| AAV5 | 103 | US20030138772 SEQ ID NO: 114 |
| AAV5 | 104 | US20160017295 SEQ ID NO: 5, |
| | | U.S. Pat. No. 7,427,396 SEQ ID NO: 2, |
| | | US20150315612 SEQ ID NO: 216 |
| AAV5 | 105 | US20150315612 SEQ ID NO: 199 |
| AAV6 | 106 | US20150159173 SEQ ID NO: 13 |
| AAV6 | 107 | US20030138772 SEQ ID NO: 65, |
| | | US20150159173 SEQ ID NO: 29, |
| | | US20160017295 SEQ ID NO: 6, |
| | | U.S. Pat. No. 6,156,303 SEQ ID NO: 7 |
| AAV6 | 108 | U.S. Pat. No. 6,156,303 SEQ ID NO: 11 |
| AAV6 | 109 | U.S. Pat. No. 6,156,303 SEQ ID NO: 2 |
| AAV6 | 110 | US20150315612 SEQ ID NO: 203 |
| AAV6 | 111 | US20150315612 SEQ ID NO: 220 |
| AAV6.1 | 112 | US20150159173 |
| AAV6.12 | 113 | US20150159173 |
| AAV6.2 | 114 | US20150159173 |
| AAV7 | 115 | US20150159173 SEQ ID NO: 14 |
| AAV7 | 116 | US20150315612 SEQ ID NO: 183 |
| AAV7 | 117 | US20030138772 SEQ ID NO: 2, |
| | | US20150159I73 SEQ ID NO: 30, |
| | | US20150315612 SEQ ID NO: 181, |
| | | US20160017295 SEQ ID NO: 7 |
| AAV7 | 118 | US20030138772 SEQ ID NO: 3 |
| AAV7 | 119 | US20030138772 SEQ ID NO: 1, |
| | | US20150315612 SEQ ID NO: 180 |
| AAV7 | 120 | US20150315612 SEQ ID NO: 213 |
| AAV7 | 121 | US20150315612 SEQ ID NO: 222 |
| AAV8 | 122 | US20150159173 SEQ ID NO: 15 |
| AAV8 | 123 | US20150376240 SEQ ID NO: 7 |
| AAV8 | 124 | US20030138772 SEQ ID NO: 4, |
| | | US20150315612 SEQ ID NO: 182 |
| AAV8 | 125 | US20030138772 SEQ ID NO: 95, |
| | | US20140359799 SEQ ID NO: 1, |
| | | US20150159173 SEQ ID NO: 31, |
| | | US20160017295 SEQ ID NO: 8, |
| | | U.S. Pat. No. 7,198,951 SEQ ID NO: 7, |
| | | US20150315612 SEQ ID NO: 223 |
| AAV8 | 126 | US20150376240 SEQ ID NO: 8 |
| AAV8 | 127 | US20150315612 SEQ ID NO: 214 |
| AAV-8b | 128 | US20150376240 SEQ ID NO: 5 |
| AAV-8b | 129 | US20150376240 SEQ ID NO: 3 |
| AAV-8h | 130 | US20150376240 SEQ ID NO: 6 |
| AAV-8h | 131 | US20150376240 SEQ ID NO: 4 |
| AAV9 | 132 | US20030138772 SEQ ID NO: 5 |
| AAV9 | 133 | U.S. Pat. No. 7,198,951 SEQ ID NO: 1 |
| AAV9 | 134 | US20160017295 SEQ ID NO: 9 |
| AAV9 | 135 | US20030138772 SEQ ID NO: 100, |
| | | U.S. Pat. No. 7,198,951 SEQ ID NO: 2 |
| AAV9 | 136 | U.S. Pat. No. 7,198,951 SEQ ID NO: 3 |
| AAV9 (AAVhu.14) | 137 | U.S. Pat. No. 7,906,111 SEQ ID NO: 3; |
| | | WO2015038958 SEQ ID NO: 11 |
| AAV9 (AAVhu.l4) | 138 | U.S. Pat. No. 7,906,111 SEQ ID NO: 123; |
| | | WO2015038958 SEQ ID NO: 2 |
| AAVA3.1 | 139 | US20030138772 SEQ ID NO: 120 |
| AAVA3.3 | 140 | US20030138772 SEQ ID NO: 57 |
| AAVA3.3 | 141 | US20030138772 SEQ ID NO: 66 |
| AAVA3.4 | 142 | US20030138772 SEQ ID NO: 54 |
| AAVA3.4 | 143 | US20030138772 SEQ ID NO: 68 |
| AAVA3.5 | 144 | US20030138772 SEQ ID NO: 55 |
| AAVA3.5 | 145 | US20030138772 SEQ ID NO: 69 |
| AAVA3.7 | 146 | US20030138772 SEQ ID NO: 56 |
| AAVA3.7 | 147 | US20030138772 SEQ ID NO: 67 |
| AAV29.3 (AAVbb.1) | 148 | US20030138772 SEQ ID NO: 11 |
| AAVC2 | 149 | US20030138772 SEQ ID NO: 61 |
| AAVCh.5 | 150 | US20150159173 SEQ ID NO: 46, |
| | | US20150315612 SEQ ID NO: 234 |
| AAVcy.2 (AAV13.3) | 151 | US20030138772 SEQ ID NO: 15 |
| AAV24.1 | 152 | US20030138772 SEQ ID NO: 101 |
| AAVcy.3 (AAV24.1) | 153 | US20030138772 SEQ ID NO: 16 |
| AAV27.3 | 154 | US20030138772 SEQ ID NO: 104 |
| AAVcy.4 (AAV27.3) | 155 | US20030138772 SEQ ID NO: 17 |
| AAVcy.5 | 156 | US20150315612 SEQ ID NO: 227 |
| AAV7.2 | 157 | US20030138772 SEQ ID NO: 103 |
| AAVcy.5 (AAV7.2) | 158 | US20030138772 SEQ ID NO: 18 |
| AAV16.3 | 159 | US20030138772 SEQ ID NO: 105 |
| AAVcy.6 (AAV16.3) | 160 | US20030138772 SEQ ID NO: 10 |
| AAVcy.5 | 161 | US20150159173 SEQ ID NO: 8 |
| AAVcy.5 | 162 | US20150159173 SEQ ID NO: 24 |
| AAVCy.5R1 | 163 | US20150159173 |
| AAVCy.5R2 | 164 | US20150159173 |
| AAVCy.5R3 | 165 | US20150159173 |
| AAVCy.5R4 | 166 | US20150159173 |
| AAVDJ | 167 | US20140359799 SEQ ID NO: 3, |
| | | U.S. Pat. No. 7,588,772 SEQ ID NO: 2 |
| AAVDJ | 168 | US20140359799 SEQ ID NO: 2, |
| | | U.S. Pat. No. 7,588,772 SEQ ID NO: 1 |
| AAVDJ-8 | 169 | U.S. Pat. No. 7,588,772; Grimm et al 2008 |
| AAVDJ-8 | 170 | U.S. Pat. No. 7,588,772; Grimm et al 2008 |
| AAVF5 | 171 | US20030138772 SEQ ID NO: 110 |
| AAVH2 | 172 | US20030138772 SEQ ID NO: 26 |
| AAVH6 | 173 | US20030138772 SEQ ID NO: 25 |
| AAVhE1.1 | 174 | U.S. Pat. No. 9,233,131 SEQ ID NO: 44 |
| AAVhEr1.14 | 175 | U.S. Pat. No. 9,233,131 SEQ ID NO: 46 |
| AAVhEr1.16 | 176 | U.S. Pat. No. 9,233,131 SEQ ID NO: 48 |
| AAVhEr1.18 | 177 | U.S. Pat. No. 9,233,131 SEQ ID NO: 49 |
| AAVhEr1.23 (AAVhEr2.29) | 178 | U.S. Pat. No. 9,233,131 SEQ ID NO: 53 |
| AAVhEr1.35 | 179 | U.S. Pat. No. 9,233,131 SEQ ID NO: 50 |
| AAVhEr1.36 | 180 | U.S. Pat. No. 9,233,131 SEQ ID NO: 52 |
| AAVhEr1.5 | 181 | U.S. Pat. No. 9,233,131 SEQ ID NO: 45 |
| AAVhEr1.7 | 182 | U.S. Pat. No. 9,233,131 SEQ ID NO: 51 |
| AAVhEr1.8 | 183 | U.S. Pat. No. 9,233,131 SEQ ID NO: 47 |
| AAVhEr2.16 | 184 | U.S. Pat. No. 9,233,131 SEQ ID NO: 55 |
| AAVhEr2.30 | 185 | U.S. Pat. No. 9,233,131 SEQ ID NO: 56 |
| AAVhEr2.31 | 186 | U.S. Pat. No. 9,233,131 SEQ ID NO: 58 |
| AAVhEr2.36 | 187 | U.S. Pat. No. 9,233,131 SEQ ID NO: 57 |
| AAVhEr2.4 | 188 | U.S. Pat. No. 9,233,131 SEQ ID NO: 54 |
| AAVhEr3.1 | 189 | U.S. Pat. No. 9,233,131 SEQ ID NO: 59 |
| AAVhu.1 | 190 | US20150315612 SEQ ID NO: 46 |
| AAVhu.1 | 191 | US20150315612 SEQ ID NO: 144 |
| AAVhu.10 (AAV16.8) | 192 | US20150315612 SEQ ID NO: 56 |
| AAVhu.10 (AAV16.8) | 193 | US20150315612 SEQ ID NO: 156 |
| AAVhu.11 (AAV16.12) | 194 | US20150315612 SEQ ID NO: 57 |
| AAVhu.11 (AAV16.12) | 195 | US20150315612 SEQ ID NO: 153 |
| AAVhu.12 | 196 | US20150315612 SEQ ID NO: 59 |
| AAVhu.12 | 197 | US20150315612 SEQ ID NO: 154 |
| AAVhu.13 | 198 | US20150159173 SEQ ID NO: 16, |
| | | US20150315612 SEQ ID NO: 71 |
| AAVhu.13 | 199 | US20150159173 SEQ ID NO: 32, |
| | | US20150315612 SEQ ID NO: 129 |
| AAVhu.136.1 | 200 | US20150315612 SEQ ID NO: 165 |
| AAVhu.140.1 | 201 | US20150315612 SEQ ID NO: 166 |
| AAVhu.140.2 | 202 | US20150315612 SEQ ID NO: 167 |
| AAVhu.145.6 | 203 | US20150315612 SEQ ID No: 178 |
| AAVhu.15 | 204 | US20150315612 SEQ ID NO: 147 |
| AAVhu.15 (AAV33.4) | 205 | US20150315612 SEQ ID NO: 50 |
| AAVhu.156.1 | 206 | US20150315612 SEQ ID No: 179 |
| AAVhu.16 | 207 | US20150315612 SEQ ID NO: 148 |
| AAVhu.16 (AAV33.8) | 208 | US20150315612 SEQ ID NO: 51 |
| AAVhu.17 | 209 | US20150315612 SEQ ID NO: 83 |
| AAVhu.17 (AAV33.12) | 210 | US20150315612 SEQ ID NO: 4 |
| AAVhu.172.1 | 211 | US20150315612 SEQ ID NO: 171 |
| AAVhu.172.2 | 212 | US20150315612 SEQ ID NO: 172 |
| AAVhu.173.4 | 213 | US20150315612 SEQ ID NO: 173 |
| AAVhu.173.8 | 214 | US20150315612 SEQ ID NO: 175 |
| AAVhu.18 | 215 | US20150315612 SEQ ID NO: 52 |
| AAVhu.18 | 216 | US20150315612 SEQ ID NO: 149 |
| AAVhu.19 | 217 | US20150315612 SEQ ID NO: 62 |
| AAVhu.19 | 218 | US20150315612 SEQ ID NO: 133 |
| AAVhu.2 | 219 | US20150315612 SEQ ID NO: 48 |
| AAVhu.2 | 220 | US20150315612 SEQ ID NO: 143 |
| AAVhu.20 | 221 | US20150315612 SEQ ID NO: 63 |
| AAVhu.20 | 222 | US20150315612 SEQ ID NO: 134 |
| AAVhu.21 | 223 | US20150315612 SEQ ID NO: 65 |
| AAVhu.21 | 224 | US20150315612 SEQ ID NO: 135 |
| AAVhu.22 | 225 | US20150315612 SEQ ID NO: 67 |
| AAVhu.22 | 226 | US20150315612 SEQ ID NO: 138 |
| AAVhu.23 | 227 | US20150315612 SEQ ID NO: 60 |
| AAVhu.23.2 | 228 | US20150315612 SEQ ID NO: 137 |
| AAVhu.24 | 229 | US20150315612 SEQ ID NO: 66 |
| AAVhu.24 | 230 | US20150315612 SEQ ID NO: 136 |
| AAVhu.25 | 231 | US20150315612 SEQ ID NO: 49 |
| AAVhu.25 | 232 | US20150315612 SEQ ID NO: 146 |
| AAVhu.26 | 233 | US20150159173 SEQ ID NO: 17, |
| | | US20150315612 SEQ ID NO: 61 |
| AAVhu.26 | 234 | US20150159173 SEQ ID NO: 33, |
| | | US20150315612 SEQ ID NO: 139 |
| AAVhu.27 | 235 | US20150315612 SEQ ID NO: 64 |
| AAVhu.27 | 236 | US20150315612 SEQ ID NO: 140 |
| AAVhu.28 | 237 | US20150315612 SEQ ID NO: 68 |
| AAVhu.28 | 238 | US20150315612 SEQ ID NO: 130 |
| AAVhu.29 | 239 | US20150315612 SEQ ID NO: 69 |
| AAVhu.29 | 240 | US20150159173 SEQ ID NO: 42, |
| | | US20150315612 SEQ ID NO: 132 |
| AAVhu.29 | 241 | US20150315612 SEQ ID NO: 225 |
| AAVhu.29R | 242 | US20150159173 |
| AAVhu.3 | 243 | US20150315612 SEQ ID NO: 44 |
| AAVhu.3 | 244 | US20150315612 SEQ ID NO: 145 |
| AAVhu.30 | 245 | US20150315612 SEQ ID NO: 70 |
| AAVhu.30 | 246 | US20150315612 SEQ ID NO: 131 |
| AAVhu.31 | 247 | US20150315612 SEQ ID NO: 1 |
| AAVhu.31 | 248 | US20150315612 SEQ ID NO: 121 |
| AAVhu.32 | 249 | US20150315612 SEQ ID NO: 2 |
| AAVhu.32 | 250 | US20150315612 SEQ ID NO: 122 |
| AAVhu.33 | 251 | US20150315612 SEQ ID NO: 75 |
| AAVhu.33 | 252 | US20150315612 SEQ ID NO: 124 |
| AAVhu.34 | 253 | US20150315612 SEQ ID NO: 72 |
| AAVhu.34 | 254 | US20150315612 SEQ ID NO: 125 |
| AAVhu.35 | 255 | US20150315612 SEQ ID NO: 73 |
| AAVhu.35 | 256 | US20150315612 SEQ ID NO: 164 |
| AAVhu.36 | 257 | US20150315612 SEQ ID NO: 74 |
| AAVhu.36 | 258 | US20150315612 SEQ ID NO: 126 |
| AAVhu.37 | 259 | US20150159173 SEQ ID NO: 34, |
| | | US20150315612 SEQ ID NO: 88 |
| AAVhu.37 (AAV106.1) | 260 | US20150315612 SEQ ID NO: 10, |
| | | US20150159173 SEQ ID NO: 18 |
| AAVhu.38 | 261 | US20150315612 SEQ ID NO: 161 |
| AAVhu.39 | 262 | US20150315612 SEQ ID NO: 102 |
| AAVhu.39 (AAVLG-9) | 263 | US20150315612 SEQ ID NO: 24 |
| AAVhu.4 | 264 | US20150315612 SEQ ID NO: 47 |
| AAVhu.4 | 265 | US20150315612 SEQ ID NO: 141 |
| AAVhu.40 | 266 | US20150315612 SEQ ID NO: 87 |
| AAVhu.40 (AAV114.3) | 267 | US20150315612 SEQ ID No: 11 |
| AAVhu.41 | 268 | US20150315612 SEQ ID NO: 91 |
| AAVhu.41 (AAV127.2) | 269 | US20150315612 SEQ ID NO: 6 |
| AAVhu.42 | 270 | US20150315612 SEQ ID NO: 85 |
| AAVhu.42 (AAV127.5) | 271 | US20150315612 SEQ ID NO: 8 |
| AAVhu.43 | 272 | US20150315612 SEQ ID NO: 160 |
| AAVhu.43 | 273 | US20150315612 SEQ ID NO: 236 |
| AAVhu.43 (AAV128.1) | 274 | US20150315612 SEQ ID NO: 80 |
| AAVhu.44 | 275 | US20150159173 SEQ ID NO: 45, |
| | | US20150315612 SEQ ID NO: 158 |
| AAVhu.44 (AAV128.3) | 276 | US20150315612 SEQ ID NO: 81 |
| AAVhu.44R1 | 277 | US20150159173 |
| AAVhu.44R2 | 278 | US20150159173 |
| AAVhu.44R3 | 279 | US20150159173 |
| AAVhu.45 | 280 | US20150315612 SEQ ID NO: 76 |
| AAVhu.45 | 281 | US20150315612 SEQ ID NO: 127 |
| AAVhu.46 | 282 | US20150315612 SEQ ID NO: 82 |
| AAVhu.46 | 283 | US20150315612 SEQ ID NO: 159 |
| AAVhu.46 | 284 | US20150315612 SEQ ID NO: 224 |
| AAVhu.47 | 285 | US20150315612 SEQ ID NO: 77 |
| AAVhu.47 | 286 | US20150315612 SEQ ID NO: 128 |
| AAVhu.48 | 287 | US20150159173 SEQ ID NO: 38 |
| AAVhu.48 | 288 | US20150315612 SEQ ID NO: 157 |
| AAVhu.48 (AAV130.4) | 289 | US20150315612 SEQ ID NO: 78 |
| AAVhu.48R1 | 290 | US20150159173 |
| AAVhu.48R2 | 291 | US20150159173 |
| AAVhu.48R3 | 292 | US20150159173 |
| AAVhu.49 | 293 | US20150315612 SEQ ID NO: 209 |
| AAVhu.49 | 294 | US20150315612 SEQ ID NO: 189 |
| AAVhu.5 | 295 | US20150315612 SEQ ID NO: 45 |
| AAVhu.5 | 296 | US20150315612 SEQ ID NO: 142 |
| AAVhu.51 | 297 | US20150315612 SEQ ID NO: 208 |
| AAVhu.51 | 298 | US20150315612 SEQ ID NO: 190 |
| AAVhu.52 | 299 | US20150315612 SEQ ID NO: 210 |
| AAVhu.52 | 300 | US20150315612 SEQ ID NO: 191 |
| AAVhu.53 | 301 | US20150159173 SEQ ID NO: 19 |
| AAVhu.53 | 302 | US20150159173 SEQ ID NO: 35 |
| AAVhu.53 (AAV145.1) | 303 | US20150315612 SEQ ID NO: 176 |
| AAVhu.54 | 304 | US20150315612 SEQ ID NO: 188 |
| AAVhu.54 (AAV145.5) | 305 | US20150315612 SEQ ID No: 177 |
| AAVhu.55 | 306 | US20150315612 SEQ ID NO: 187 |
| AAVhu.56 | 307 | US20150315612 SEQ ID NO: 205 |
| AAVhu.56 (AAV145.6) | 308 | US20150315612 SEQ ID NO: 168 |
| AAVhu.56 (AAV145.6) | 309 | US20150315612 SEQ ID NO: 192 |
| AAVhu.57 | 310 | US20150315612 SEQ ID NO: 206 |
| AAVhu.57 | 311 | US20150315612 SEQ ID NO: 169 |
| AAVhu.57 | 312 | US20150315612 SEQ ID NO: 193 |
| AAVhu.58 | 313 | US20150315612 SEQ ID NO: 207 |
| AAVhu.58 | 314 | US201503I5612 SEQ ID NO: 194 |
| AAVhu.6 (AAV3.1) | 315 | US20150315612 SEQ ID NO: 5 |
| AAVhu.6 (AAV3.1) | 316 | US20150315612 SEQ ID NO: 84 |
| AAVhu.60 | 317 | US20150315612 SEQ ID NO: 184 |
| AAVhu.60 (AAV161.10) | 318 | US20150315612 SEQ ID NO: 170 |
| AAVhu.61 | 319 | US20150315612 SEQ ID NO: 185 |
| AAVhu.61 (AAV161.6) | 320 | US20150315612 SEQ ID NO: 174 |
| AAVhu.63 | 321 | US20150315612 SEQ ID NO: 204 |
| AAVhu.63 | 322 | US20150315612 SEQ ID NO: 195 |
| AAVhu.64 | 323 | US20150315612 SEQ ID NO: 212 |
| AAVhu.64 | 324 | US20150315612 SEQ ID NO: 196 |
| AAVhu.66 | 325 | US20150315612 SEQ ID NO: 197 |
| AAVhu.67 | 326 | US20150315612 SEQ ID NO: 215 |
| AAVhu.67 | 327 | US20150315612 SEQ ID NO: 198 |
| AAVhu.7 | 328 | US20150315612 SEQ ID NO: 226 |
| AAVhu.7 | 329 | US20150315612 SEQ ID NO: 150 |
| AAVhu.7 (AAV7.3) | 330 | US20150315612 SEQ ID NO: 55 |
| AAVhu.71 | 331 | US20150315612 SEQ ID NO: 79 |
| AAVhu.8 | 332 | US20150315612 SEQ ID NO: 53 |
| AAVhu.8 | 333 | US20150315612 SEQ ID NO: 12 |
| AAVhu.8 | 334 | US20150315612 SEQ ID NO: 151 |
| AAVhu.9 (AAV3.1) | 335 | US20150315612 SEQ ID NO: 58 |
| AAVhu.9 (AAV3.1) | 336 | US20150315612 SEQ ID NO: 155 |
| AAV-LK01 | 337 | US20150376607 SEQ ID NO: 2 |
| AAV-LK01 | 338 | US20150376607 SEQ ID NO: 29 |
| AAV-LK02 | 339 | US20150376607 SEQ ID NO: 3 |
| AAV-LK02 | 340 | US20150376607 SEQ ID NO: 30 |
| AAV-LK03 | 341 | US20150376607 SEQ ID NO: 4 |
| AAV-LK03 | 342 | WO2015121501 SEQ ID NO: 12, |
| | | US20150376607 SEQ ID NO: 31 |
| AAV-LK04 | 343 | US20150376607 SEQ ID NO: 5 |
| AAV-LK04 | 344 | US20150376607 SEQ ID NO: 32 |
| AAV-LK05 | 345 | US20150376607 SEQ ID NO: 6 |
| AAV-LK05 | 346 | US20150376607 SEQ ID NO: 33 |
| AAV-LK06 | 347 | US20150376607 SEQ ID NO: 7 |
| AAV-LK06 | 348 | US20150376607 SEQ ID NO: 34 |
| AAV-LK07 | 349 | US20150376607 SEQ ID NO: 8 |
| AAV-LK07 | 350 | US20150376607 SEQ ID NO: 35 |
| AAV-LK08 | 351 | US20150376607 SEQ ID NO: 9 |
| AAV-LK08 | 352 | US20150376607 SEQ ID NO: 36 |
| AAV-LK09 | 353 | US20150376607 SEQ ID NO: 10 |
| AAV-LK09 | 354 | US20150376607 SEQ ID NO: 37 |
| AAV-LK10 | 355 | US20150376607 SEQ ID NO: 11 |
| AAV-LK10 | 356 | US20150376607 SEQ ID NO: 38 |
| AAV-LK11 | 357 | US20150376607 SEQ ID NO: 12 |
| AAV-LK11 | 358 | US20150376607 SEQ ID NO: 39 |
| AAV-LK12 | 359 | US20150376607 SEQ ID NO: 13 |
| AAV-LK12 | 360 | US20150376607 SEQ ID NO: 40 |
| AAV-LK13 | 361 | US20150376607 SEQ ID NO: 14 |
| AAV-LK13 | 362 | US20150376607 SEQ ID NO: 41 |
| AAV-LK14 | 363 | US20150376607 SEQ ID NO: 15 |
| AAV-LK14 | 364 | US20150376607 SEQ ID NO: 42 |
| AAV-LK15 | 365 | US20150376607 SEQ ID NO: 16 |
| AAV-LK15 | 366 | US20150376607 SEQ ID NO: 43 |
| AAV-LK16 | 367 | US20150376607 SEQ ID NO: 17 |
| AAV-LK16 | 368 | US20150376607 SEQ ID NO: 44 |
| AAV-LK17 | 369 | US20150376607 SEQ ID NO: 18 |
| AAV-LK17 | 370 | US20150376607 SEQ ID NO: 45 |
| AAV-LK18 | 371 | US20150376607 SEQ ID NO: 19 |
| AAV-LK18 | 372 | US20150376607 SEQ ID NO: 46 |
| AAV-LK19 | 373 | US20150376607 SEQ ID NO: 20 |
| AAV-LK19 | 374 | US20150376607 SEQ ID NO: 47 |
| AAV-PAEC | 375 | US20150376607 SEQ ID NO: 1 |
| AAV-PAEC | 376 | US20150376607 SEQ ID NO: 48 |
| AAV-PAEC11 | 377 | US20150376607 SEQ ID NO: 26 |
| AAV-PAEC11 | 378 | US20150376607 SEQ ID NO: 54 |
| AAV-PAEC12 | 379 | US20150376607 SEQ ID NO: 27 |
| AAV-PAEC12 | 380 | US20150376607 SEQ ID NO: 51 |
| AAV-PAEC13 | 381 | US20150376607 SEQ ID NO: 28 |
| AAV-PAEC13 | 382 | US20150376607 SEQ ID NO: 49 |
| AAV-PAEC2 | 383 | US20150376607 SEQ ID NO: 21 |
| AAV-PAEC2 | 384 | US20150376607 SEQ ID NO: 56 |
| AAV-PAEC4 | 385 | US20150376607 SEQ ID NO: 22 |
| AAV-PAEC4 | 386 | US20150376607 SEQ ID NO: 55 |
| AAV-PAEC6 | 387 | US20150376607 SEQ ID NO: 23 |
| AAV-PAEC6 | 388 | US20150376607 SEQ ID NO: 52 |
| AAV-PAEC7 | 389 | US20150376607 SEQ ID NO: 24 |
| AAV-PAEC7 | 390 | US20150376607 SEQ ID NO: 53 |
| AAV-PAEC8 | 391 | US20150376607 SEQ ID NO: 25 |
| AAV-PAEC8 | 392 | US20150376607 SEQ ID NO: 50 |
| AAVpi.1 | 393 | US20150315612 SEQ ID NO: 28 |
| AAVpi.1 | 394 | US20150315612 SEQ ID NO: 93 |
| AAVpi.2 | 395 | US20150315612 SEQ ID NO: 30 |
| AAVpi.2 | 396 | US20150315612 SEQ ID NO: 95 |
| AAVpi.3 | 397 | US20150315612 SEQ ID NO: 29 |
| AAVpi.3 | 398 | US20150315612 SEQ ID NO: 94 |
| AAVrh.10 | 399 | US20150159173 SEQ ID NO: 9 |
| AAVrh.10 | 400 | US20150159173 SEQ ID NO: 25 |
| AAV44.2 | 401 | US20030138772 SEQ ID NO: 59 |
| AAVrh.10 (AAV44.2) | 402 | US20030138772 SEQ ID NO: 81 |
| AAV42.1B | 403 | US20030138772 SEQ ID NO: 90 |
| AAVrh.12 (AAV42.1b) | 404 | US20030138772 SEQ ID NO: 30 |
| AAVrh.13 | 405 | US20150159173 SEQ ID NO: 10 |
| AAVrh.13 | 406 | US20150159173 SEQ ID NO: 26 |
| AAVrh.13 | 407 | US20150315612 SEQ ID NO: 228 |
| AAVrh.13R | 408 | US20150159173 |
| AAV42.3A | 409 | US20030138772 SEQ ID NO: 87 |
| AAVrh.14 (AAV42.3a) | 410 | US20030138772 SEQ ID NO: 32 |
| AAV42.5A | 411 | US20030138772 SEQ ID NO: 89 |
| AAVrh.17 (AAV42.5a) | 412 | US20030138772 SEQ ID NO: 34 |
| AAV42.5B | 413 | US20030138772 SEQ ID NO: 91 |
| AAVrh.18 (AAV42.5b) | 414 | US20030138772 SEQ ID NO: 29 |
| AAV42.6B | 415 | US20030138772 SEQ ID NO: 112 |
| AAVrh.19 (AAV42.6b) | 416 | US20030138772 SEQ ID NO: 38 |
| AAVrh.2 | 417 | US20150159173 SEQ ID NO: 39 |
| AAVrh.2 | 418 | US20150315612 SEQ ID NO: 231 |
| AAVrh.20 | 419 | US20150159173 SEQ ID NO: 1 |
| AAV42.10 | 420 | US20030138772 SEQ ID NO: 106 |
| AAVrh.21 (AAV42.10) | 421 | US20030138772 SEQ ID NO: 35 |
| AAV42.11 | 422 | US20030138772 SEQ ID NO: 108 |
| AAVrh.22 (AAV42.11) | 423 | US20030138772 SEQ ID NO: 37 |
| AAV42.12 | 424 | US20030138772 SEQ ID NO: 113 |
| AAVrh.23 (AAV42.12) | 425 | US20030138772 SEQ ID NO: 58 |
| AAV42.13 | 426 | US20030138772 SEQ ID NO: 86 |
| AAVrh.24 (AAV42.13) | 427 | US20030138772 SEQ ID NO: 31 |
| AAV42.15 | 428 | US20030138772 SEQ ID NO: 84 |
| AAVrh.25 (AAV42.15) | 429 | US20030138772 SEQ ID NO: 28 |
| AAVrh.2R | 430 | US20150159173 |
| AAVrh.31 (AAV223.1) | 431 | US20030138772 SEQ ID NO: 48 |
| AAVC1 | 432 | US20030138772 SEQ ID NO: 60 |
| AAVrh.32 (AAVC1) | 433 | US20030138772 SEQ ID NO: 19 |
| AAVrh.32/33 | 434 | US20150159173 SEQ ID NO: 2 |
| AAVrh.33 (AAVC3) | 435 | US20030138772 SEQ ID NO: 20 |
| AAVC5 | 436 | US20030138772 SEQ ID NO: 62 |
| AAVrh.34 (AAVC5) | 437 | US20030138772 SEQ ID NO: 21 |
| AAVF1 | 438 | US20030138772 SEQ ID NO: 109 |
| AAVrh.35 (AAVF1) | 439 | US20030138772 SEQ ID NO: 22 |
| AAVF3 | 440 | US20030138772 SEQ ID NO: 111 |
| AAVrh.36 (AAVF3) | 441 | US20030138772 SEQ ID NO: 23 |
| AAVrh.37 | 442 | US20030I38772 SEQ ID NO: 24 |
| AAVrh.37 | 443 | US20150159173 SEQ ID NO: 40 |
| AAVrh.37 | 444 | US20150315612 SEQ ID NO: 229 |
| AAVrh.37R2 | 445 | US20150159173 |
| AAVrh.38 (AAVLG-4) | 446 | US20150315612 SEQ ID NO: 7 |
| AAVrh.38 (AAVLG-4) | 447 | US20150315612 SEQ ID NO: 86 |
| AAVrh.39 | 448 | US20150159173 SEQ ID NO: 20, |
| | | US20150315612 SEQ ID NO: 13 |
| AAVrh.39 | 449 | US20150159173 SEQ ID NO: 3, |
| | | US20150159173 SEQ ID NO: 36, |
| | | US20150315612 SEQ ID NO: 89 |
| AAVrh.40 | 450 | US20150315612 SEQ ID NO: 92 |
| AAVrh.40 (AAVLG-10) | 451 | US20150315612 SEQ ID NO: 14 |
| AAVrh.43 (AAVN721-8) | 452 | US20150315612 SEQ ID NO: 43, |
| | | US20150159173 SEQ ID NO: 21 |
| AAVrh.43 (AAVN721-8) | 453 | US20150315612 SEQ ID NO: 163, |
| | | US20150159173 SEQ ID NO: 37 |
| AAVrh.44 | 454 | US20150315612 SEQ ID NO: 34 |
| AAVrh.44 | 455 | US20150315612 SEQ ID NO: 111 |
| AAVrh.45 | 456 | US20150315612 SEQ ID NO: 41 |
| AAVrh.45 | 457 | US20150315612 SEQ ID NO: 109 |
| AAVrh.46 | 458 | US20150159173 SEQ ID NO: 22, |
| | | US20150315612 SEQ ID NO: 19 |
| AAVrh.46 | 459 | US20150159173 SEQ ID NO: 4, |
| | | US20150315612 SEQ ID NO: 101 |
| AAVrh.47 | 460 | US20150315612 SEQ ID NO: 38 |
| AAVrh.47 | 461 | US201503156I2 SEQ ID NO: 118 |
| AAVrh.48 | 462 | US20150159173 SEQ ID NO: 44, |
| | | US20150315612 SEQ ID NO: 115 |
| AAVrh.48.1 | 463 | US20150159173 |
| AAVrh.48.1.2 | 464 | US20150159173 |
| AAVrh.48.2 | 465 | US20150159173 |
| AAVrh.48 (AAV1-7) | 466 | US20150315612 SEQ ID NO: 32 |
| AAVrh.49 (AAV1-8) | 467 | US20150315612 SEQ ID NO: 25 |
| AAVrh.49 (AAV1-8) | 468 | US20150315612 SEQ ID NO: 103 |
| AAVrh.50 (AAV2-4) | 469 | US20150315612 SEQ ID NO: 23 |
| AAVrh.50 (AAV2-4) | 470 | US20150315612 SEQ ID NO: 108 |
| AAVrh.51 (AAV2-5) | 471 | US20150315612 SEQ ID No: 22 |
| AAVrh.51 (AAV2-5) | 472 | US20150315612 SEQ ID NO: 104 |
| AAVrh.52 (AAV3-9) | 473 | US20150315612 SEQ ID NO: 18 |
| AAVrh.52 (AAV3-9) | 474 | US20150315612 SEQ ID NO: 96 |
| AAVrh.53 | 475 | US20150315612 SEQ ID NO: 97 |
| AAVrh.53 (AAV3-11) | 476 | US20150315612 SEQ ID NO: 17 |
| AAVrh.53 (AAV3-11) | 477 | US20150315612 SEQ ID NO: 186 |
| AAVrh.54 | 478 | US20150315612 SEQ ID NO: 40 |
| AAVrh.54 | 479 | US20150159173 SEQ ID NO: 49, |
| | | US20150315612 SEQ ID NO: 116 |
| AAVrh.55 | 480 | US20150315612 SEQ ID NO: 37 |
| AAVrh.55 (AAV4-19) | 481 | US20150315612 SEQ ID NO: 117 |
| AAVrh.56 | 482 | US20150315612 SEQ ID NO: 54 |
| AAVrh.56 | 483 | US20150315612 SEQ ID NO: 152 |
| AAVrh.57 | 484 | US20150315612 SEQ ID NO: 26 |
| AAVrh.57 | 485 | US2015031.5612 SEQ ID NO: 105 |
| AAVrh.58 | 486 | US20150315612 SEQ ID NO: 27 |
| AAVrh.58 | 487 | US20150159173 SEQ ID NO: 48, |
| | | US20150315612 SEQ ID NO: 106 |
| AAVrh.58 | 488 | US20150315612 SEQ ID NO: 232 |
| AAVrh.59 | 489 | US20150315612 SEQ ID NO: 42 |
| AAVrh.59 | 490 | US20150315612 SEQ ID NO: 110 |
| AAVrh.60 | 491 | US20150315612 SEQ ID NO: 31 |
| AAVrh.60 | 492 | US20150315612 SEQ ID NO: 120 |
| AAVrh.61 | 493 | US20150315612 SEQ ID NO: 107 |
| AAVrh.61 (AAV2-3) | 494 | US20150315612 SEQ ID NO: 21 |
| AAVrh.62 (AAV2-15) | 495 | US20150315612 SEQ ID No: 33 |
| AAVrh.62 (AAV2-15) | 496 | US20150315612 SEQ ID NO: 114 |
| AAVrh.64 | 497 | US20150315612 SEQ ID No: 15 |
| AAVrh.64 | 498 | US20150159173 SEQ ID NO: 43, |
| | | US20150315612 SEQ ID NO: 99 |
| AAVrh.64 | 499 | US20150315612 SEQ ID NO: 233 |
| AAVRh.64R1 | 500 | US20150159173 |
| AAVRh.64R2 | 501 | US20150159173 |
| AAVrh.65 | 502 | US20150315612 SEQ ID NO: 35 |
| AAVrh.65 | 503 | US20150315612 SEQ ID NO: 112 |
| AAVrh.67 | 504 | US20150315612 SEQ ID NO: 36 |
| AAVrh.67 | 505 | US20150315612 SEQ ID NO: 230 |
| AAVrh.67 | 506 | US20150159173 SEQ ID NO: 47, |
| | | US20150315612 SEQ ID NO: 113 |
| AAVrh.68 | 507 | US20150315612 SEQ ID NO: 16 |
| AAVrh.68 | 508 | US20150315612 SEQ ID NO: 100 |
| AAVrh.69 | 509 | US20150315612 SEQ ID NO: 39 |
| AAVrh.69 | 510 | US20150315612 SEQ ID NO: 119 |
| AAVrh.70 | 511 | US20150315612 SEQ ID NO: 20 |
| AAVrh.70 | 512 | US20150315612 SEQ ID NO: 98 |
| AAVrh.71 | 513 | US20150315612 SEQ ID NO: 162 |
| AAVrh.72 | 514 | US20150315612 SEQ ID NO: 9 |
| AAVrh.73 | 515 | US20150159I73 SEQ ID NO: 5 |
| AAVrh.74 | 516 | US20150159173 SEQ ID NO: 6 |
| AAVrh.8 | 517 | US20150159173 SEQ ID NO: 41 |
| AAVrh.8 | 518 | US20150315612 SEQ ID NO: 235 |
| AAVrh.8R | 519 | US20150159173, WO2015168666 SEQ ID NO: 9 |
| AAVrh.8R A586R mutant | 520 | WO2015168666 SEQ ID NO: 10 |
| AAVrh.8R R533A mutant | 521 | WO2015168666 SEQ ID NO: 11 |
| BAAV (bovine AAV) | 522 | U.S. Pat. No. 9,193,769 SEQ ID NO: 8 |
| BAAV (bovine AAV) | 523 | U.S. Pat. No. 9,193,769 SEQ ID NO: 10 |
| BAAV (bovine AAV) | 524 | U.S. Pat. No. 9,193,769 SEQ ID NO: 4 |
| BAAV (bovine AAV) | 525 | U.S. Pat. No. 9,193,769 SEQ ID NO: 2 |
| BAAV (bovine AAV) | 526 | U.S. Pat. No. 9,193,769 SEQ ID NO: 6 |
| BAAV (bovine AAV) | 527 | U.S. Pat. No. 9,193,769 SEQ ID NO: 1 |
| BAAV (bovine AAV) | 528 | U.S. Pat. No. 9,193,769 SEQ ID NO: 5 |
| BAAV (bovine AAV) | 529 | U.S. Pat. No. 9,193,769 SEQ ID NO: 3 |
| BAAV (bovine AAV) | 530 | U.S. Pat. No. 9,193,769 SEQ ID NO: 11 |
| BAAV (bovine AAV) | 531 | U.S. Pat. No. 7,427,396 SEQ ID NO: 5 |
| BAAV (bovine AAV) | 532 | U.S. Pat. No. 7,427,396 SEQ ID NO: 6 |
| BAAV (bovine AAV) | 533 | U.S. Pat. No. 9,193,769 SEQ ID NO: 7 |
| BAAV (bovine AAV) | 534 | U.S. Pat. No. 9,193,769 SEQ ID NO: 9 |
| BNP61 AAV | 535 | US20150238550 SEQ ID NO: 1 |
| BNP61 AAV | 536 | US20150238550 SEQ ID NO: 2 |
| BNP62 AAV | 537 | US20150238550 SEQ ID NO: 3 |
| BNP63 AAV | 538 | US20150238550 SEQ ID NO: 4 |
| caprine AAV | 539 | U.S. Pat. No. 7,427,396 SEQ ID NO: 3 |
| caprine AAV | 540 | U.S. Pat. No. 7,427,396 SEQ ID NO: 4 |
| true type AAV (ttAAV) | 541 | WO2015121501 SEQ ID NO: 2 |
| AAAV (Avian AAV) | 542 | U.S. Pat. No. 9,238,800 SEQ ID NO: 12 |
| AAAV (Avian AAV) | 543 | U.S. Pat. No. 9,238,800 SEQ ID NO: 2 |
| AAAV (Avian AAV) | 544 | U.S. Pat. No. 9,238,800 SEQ ID NO: 6 |
| AAAV (Avian AAV) | 545 | U.S. Pat. No. 9,238,800 SEQ ID NO: 4 |
| AAAV (Avian AAV) | 546 | U.S. Pat. No. 9,238,800 SEQ ID NO: 8 |
| AAAV (Avian AAV) | 547 | U.S. Pat. No. 9,238,800 SEQ ID NO: 14 |
| AAAV (Avian AAV) | 548 | U.S. Pat. No. 9,238,800 SEQ ID NO: 10 |
| AAAV (Avian AAV) | 549 | U.S. Pat. No. 9,238,800 SEQ ID NO: 15 |
| AAAV (Avian AAV) | 550 | U.S. Pat. No. 9,238,800 SEQ ID NO: 5 |
| AAAV (Avian AAV) | 551 | U.S. Pat. No. 9,238,800 SEQ ID NO: 9 |
| AAAV (Avian AAV) | 552 | U.S. Pat. No. 9,238,800 SEQ ID NO: 3 |
| AAAV (Avian AAV) | 553 | U.S. Pat. No. 9,238,800 SEQ ID NO: 7 |
| AAAV (Avian AAV) | 554 | U.S. Pat. No. 9,238,800 SEQ ID NO: 11 |
| AAAV (Avian AAV) | 555 | U.S. Pat. No. 9,238,800 SEQ ID NO: 13 |
| AAAV (Avian AAV) | 556 | U.S. Pat. No. 9,238,800 SEQ ID NO: 1 |
| AAV Shuffle 100-1 | 557 | US20160017295 SEQ ID NO: 23 |
| AAV Shuffle 100-1 | 558 | US20160017295 SEQ ID NO: 11 |
| AAV Shuffle 100-2 | 559 | US20160017295 SEQ ID NO: 37 |
| AAV Shuffle 100-2 | 560 | US20160017295 SEQ ID NO: 29 |
| AAV Shuffle 100-3 | 561 | US20160017295 SEQ ID NO: 24 |
| AAV Shuffle 100-3 | 562 | US20160017295 SEQ ID NO: 12 |
| AAV Shuffle 100-7 | 563 | US20160017295 SEQ ID NO: 25 |
| AAV Shuffle 100-7 | 564 | US20160017295 SEQ ID NO: 13 |
| AAV Shuffle 10-2 | 565 | US20160017295 SEQ ID NO: 34 |
| AAV Shuffle 10-2 | 566 | US20160017295 SEQ ID NO: 26 |
| AAV Shuffle 10-6 | 567 | US20160017295 SEQ ID NO: 35 |
| AAV Shuffle 10-6 | 568 | US20160017295 SEQ ID NO: 27 |
| AAV Shuffle 10-8 | 569 | US20160017295 SEQ ID NO: 36 |
| AAV Shuffle 10-8 | 570 | US20160017295 SEQ ID NO: 28 |
| AAV SM 100-10 | 571 | US20160017295 SEQ ID NO: 41 |
| AAV SM 100-10 | 572 | US20160017295 SEQ ID NO: 33 |
| AAV SM 100-3 | 573 | US20160017295 SEQ ID NO: 40 |
| AAV SM 100-3 | 574 | US20160017295 SEQ ID NO: 32 |
| AAV SM 10-1 | 575 | US20160017295 SEQ ID NO: 38 |
| AAV SM 10-1 | 576 | US20160017295 SEQ ID NO: 30 |
| AAV SM 10-2 | 577 | US20160017295 SEQ ID NO: 10 |
| AAV SM 10-2 | 578 | US20160017295 SEQ ID NO: 22 |
| AAV SM 10-8 | 579 | US20160017295 SEQ ID NO: 39 |
| AAV SM 10-8 | 580 | US20160017295 SEQ ID NO: 31 |
| AAVF1/HSC1 | 581 | WO2016049230 SEQ ID NO: 20 |
| AAVF2/HSC2 | 582 | WO2016049230 SEQ ID NO: 21 |
| AAVF3/HSC3 | 583 | WO2016049230 SEQ ID NO: 22 |
| AAVF4/HSC4 | 584 | WO2016049230 SEQ ID NO: 23 |
| AAVF5/HSC5 | 585 | WO2016049230 SEQ ID NO: 25 |
| AAVF6/HSC6 | 586 | WO2016049230 SEQ ID NO: 24 |
| AAVF7/HSC7 | 587 | WO2016049230 SEQ ID NO: 27 |
| AAVF8/HSC8 | 588 | WO2016049230 SEQ ID NO: 28 |
| AAVF9/HSC9 | 589 | WO2016049230 SEQ ID NO: 29 |
| AAVF11/HSC11 | 590 | WO2016049230 SEQ ID NO: 26 |
| AAVF12/HSC12 | 591 | WO2016049230 SEQ ID NO: 30 |
| AAVF13/HSC13 | 592 | WO2016049230 SEQ ID NO: 31 |
| AAVF14/HSC14 | 593 | WO2016049230 SEQ ID NO: 32 |
| AAVF15/HSC15 | 594 | WO2016049230 SEQ ID NO: 33 |
| AAVF16/HSC16 | 595 | WO2016049230 SEQ ID NO: 34 |
| AAVF17/HSC17 | 596 | WO2016049230 SEQ ID NO: 35 |
| AAVF1/HSC1 | 597 | WO2016049230 SEQ ID NO: 2 |
| AAVF2/HSC2 | 598 | WO2016049230 SEQ ID NO: 3 |
| AAVF3/HSC3 | 599 | WO2016049230 SEQ ID NO: 5 |
| AAVF4/HSC4 | 600 | WO2016049230 SEQ ID NO: 6 |
| AAVF5/HSC5 | 601 | WO2016049230 SEQ ID NO: 11 |
| AAVF6/HSC6 | 602 | WO2016049230 SEQ ID NO: 7 |
| AAVF7/HSC7 | 603 | WO2016049230 SEQ ID NO: 8 |
| AAVF8/HSC8 | 604 | WO2016049230 SEQ ID NO: 9 |
| AAVF9/HSC9 | 605 | WO2016049230 SEQ ID NO: 10 |
| AAVF11/HSC11 | 606 | WO2016049230 SEQ ID NO: 4 |
| AAVF12/HSC12 | 607 | WO2016049230 SEQ ID NO: 12 |
| AAVF13/HSC13 | 608 | WO2016049230 SEQ ID NO: 14 |
| AAVF14/HSC14 | 609 | WO2016049230 SEQ ID NO: 15 |
| AAVF15/HSC15 | 610 | WO2016049230 SEQ ID NO: 16 |
| AAVF16/HSC16 | 611 | WO2016049230 SEQ ID NO: 17 |
| AAVF17/HSC17 | 612 | WO2016049230 SEQ ID NO: 13 |
| AAV CBr-E1 | 613 | U.S. Pat. No. 8,734,809 SEQ ID NO: 13 |
| AAV CBr-E2 | 614 | U.S. Pat. No. 8,734,809 SEQ ID NO: 14 |
| AAV CBr-E3 | 615 | U.S. Pat. No. 8,734,809 SEQ ID NO: 15 |
| AAV CBr-E4 | 616 | U.S. Pat. No. 8,734,809 SEQ ID NO: 16 |
| AAV CBr-E5 | 617 | U.S. Pat. No. 8,734,809 SEQ ID NO: 17 |
| AAV CBr-e5 | 618 | U.S. Pat. No. 8,734,809 SEQ ID NO: 18 |
| AAV CBr-E6 | 619 | U.S. Pat. No. 8,734,809 SEQ ID NO: 19 |
| AAV CBr-E7 | 620 | U.S. Pat. No. 8,734,809 SEQ ID NO: 20 |
| AAV CBr-E8 | 621 | U.S. Pat. No. 8,734,809 SEQ ID NO: 21 |
| AAV CLv-D1 | 622 | U.S. Pat. No. 8,734,809 SEQ ID NO: 22 |
| AAV CLv-D2 | 623 | U.S. Pat. No. 8,734,809 SEQ ID NO: 23 |
| AAV CLv-D3 | 624 | U.S. Pat. No. 8,734,809 SEQ ID NO: 24 |
| AAV CLv-D4 | 625 | U.S. Pat. No. 8,734,809 SEQ ID NO: 25 |
| AAV CLv-D5 | 626 | U.S. Pat. No. 8,734,809 SEQ ID NO: 26 |
| AAV CLv-D6 | 627 | U.S. Pat. No. 8,734,809 SEQ ID NO: 27 |
| AAV CLv-D7 | 628 | U.S. Pat. No. 8,734,809 SEQ ID NO: 28 |
| AAV CLv-D8 | 629 | U.S. Pat. No. 8,734,809 SEQ ID NO: 29 |
| AAV CLv-E1 | 630 | U.S. Pat. No. 8,734,809 SEQ ID NO: 13 |
| AAV CLv-R1 | 631 | U.S. Pat. No. 8,734,809 SEQ ID NO: 30 |
| AAV CLv-R2 | 632 | U.S. Pat. No. 8,734,809 SEQ ID NO: 31 |
| AAV CLv-R3 | 633 | U.S. Pat. No. 8,734,809 SEQ ID NO: 32 |
| AAV CLv-R4 | 634 | U.S. Pat. No. 8,734,809 SEQ ID NO: 33 |
| AAV CLv-R5 | 635 | U.S. Pat. No. 8,734,809 SEQ ID NO: 34 |
| AAV CLv-R6 | 636 | U.S. Pat. No. 8,734,809 SEQ ID NO: 35 |
| AAV CLv-R7 | 637 | U.S. Pat. No. 8,734,809 SEQ ID NO: 36 |
| AAV CLv-R8 | 638 | U.S. Pat. No. 8,734,809 SEQ ID NO: 37 |
| AAV CLv-R9 | 639 | U.S. Pat. No. 8,734,809 SEQ ID NO: 38 |
| AAV CLg-F1 | 640 | U.S. Pat. No. 8,734,809 SEQ ID NO: 39 |
| AAV CLg-F2 | 641 | U.S. Pat. No. 8,734,809 SEQ ID NO: 40 |
| AAV CLg-F3 | 642 | U.S. Pat. No. 8,734,809 SEQ ID NO: 41 |
| AAV CLg-F4 | 643 | U.S. Pat. No. 8,734,809 SEQ ID NO: 42 |
| AAV CLg-F5 | 644 | U.S. Pat. No. 8,734,809 SEQ ID NO: 43 |
| AAV CLg-F6 | 645 | U.S. Pat. No. 8,734,809 SEQ ID NO: 43 |
| AAV CLg-F7 | 646 | U.S. Pat. No. 8,734,809 SEQ ID NO: 44 |
| AAV CLg-F8 | 647 | U.S. Pat. No. 8,734,809 SEQ ID NO: 43 |
| AAV CSp-1 | 648 | U.S. Pat. No. 8,734,809 SEQ ID NO: 45 |
| AAV CSp-10 | 649 | U.S. Pat. No. 8,734,809 SEQ ID NO: 46 |
| AAV CSp-11 | 650 | U.S. Pat. No. 8,734,809 SEQ ID NO: 47 |
| AAV CSp-2 | 651 | U.S. Pat. No. 8,734,809 SEQ ID NO: 48 |
| AAV CSp-3 | 652 | U.S. Pat. No. 8,734,809 SEQ ID NO: 49 |
| AAV CSp-4 | 653 | U.S. Pat. No. 8,734,809 SEQ ID NO: 50 |
| AAV CSp-6 | 654 | U.S. Pat. No. 8,734,809 SEQ ID NO: 51 |
| AAV CSp-7 | 655 | U.S. Pat. No. 8,734,809 SEQ ID NO: 52 |
| AAV CSp-8 | 656 | U.S. Pat. No. 8,734,809 SEQ ID NO: 53 |
| AAV CSp-9 | 657 | U.S. Pat. No. 8,734,809 SEQ ID NO: 54 |
| AAV CHt-2 | 658 | U.S. Pat. No. 8,734,809 SEQ ID NO: 55 |
| AAV CHt-3 | 659 | U.S. Pat. No. 8,734,809 SEQ ID NO: 56 |
| AAV CKd-1 | 660 | U.S. Pat. No. 8,734,809 SEQ ID NO: 57 |
| AAV CKd-10 | 661 | U.S. Pat. No. 8,734,809 SEQ ID NO: 58 |
| AAV CKd-2 | 662 | U.S. Pat. No. 8,734,809 SEQ ID NO: 59 |
| AAV CKd-3 | 663 | U.S. Pat. No. 8,734,809 SEQ ID NO: 60 |
| AAV CKd-4 | 664 | U.S. Pat. No. 8,734,809 SEQ ID NO: 61 |
| AAV CKd-6 | 665 | U.S. Pat. No. 8,734,809 SEQ ID NO: 62 |
| AAVC Kd-7 | 666 | U.S. Pat. No. 8,734,809 SEQ ID NO: 63 |
| AAVC Kd-8 | 667 | U.S. Pat. No. 8,734,809 SEQ ID NO: 64 |
| AAV CLv-1 | 668 | U.S. Pat. No. 8,734,809 SEQ ID NO: 65 |
| AAV CLv-12 | 669 | U.S. Pat. No. 8,734,809 SEQ ID NO: 66 |
| AAV CLv-13 | 670 | U.S. Pat. No. 8,734,809 SEQ ID NO: 67 |
| AAVC Lv-2 | 671 | U.S. Pat. No. 8,734,809 SEQ ID NO: 68 |
| AAVC Lv-3 | 672 | U.S. Pat. No. 8,734,809 SEQ ID NO: 69 |
| AAV CLv-4 | 673 | U.S. Pat. No. 8,734,809 SEQ ID NO: 70 |
| AAVC Lv-6 | 674 | U.S. Pat. No. 8,734,809 SEQ ID NO: 71 |
| AAVC Lv-8 | 675 | U.S. Pat. No. 8,734,809 SEQ ID NO: 72 |
| AAVC Kd-B1 | 676 | U.S. Pat. No. 8,734,809 SEQ ID NO: 73 |
| AAV CKd-B2 | 677 | U.S. Pat. No. 8,734,809 SEQ ID NO: 74 |
| AAV CKd-B3 | 678 | U.S. Pat. No. 8,734,809 SEQ ID NO: 75 |
| AAV CKd-B4 | 679 | U.S. Pat. No. 8,734,809 SEQ ID NO: 76 |
| AAVC Kd-B5 | 680 | U.S. Pat. No. 8,734,809 SEQ ID NO: 77 |
| AAV CKd-B6 | 681 | U.S. Pat. No. 8,734,809 SEQ ID NO: 78 |
| AAV CKd-B7 | 682 | U.S. Pat. No. 8,734,809 SEQ ID NO: 79 |
| AAV CKd-B8 | 683 | U.S. Pat. No. 8,734,809 SEQ ID NO: 80 |
| AAV CKd-H1 | 684 | U.S. Pat. No. 8,734,809 SEQ ID NO: 81 |
| AAV CKd-H2 | 685 | U.S. Pat. No. 8,734,809 SEQ ID NO: 82 |
| AAV CKd-H3 | 686 | U.S. Pat. No. 8,734,809 SEQ ID NO: 83 |
| AAV CKd-H4 | 687 | U.S. Pat. No. 8,734,809 SEQ ID NO: 84 |
| AAV CKd-H5 | 688 | U.S. Pat. No. 8,734,809 SEQ ID NO: 85 |
| AAV CKd-H6 | 689 | U.S. Pat. No. 8,734,809 SEQ ID NO: 77 |
| AAV CHt-1 | 690 | U.S. Pat. No. 8,734,809 SEQ ID NO: 86 |
| AAV CLv1-1 | 691 | U.S. Pat. No. 8,734,809 SEQ ID NO: 171 |
| AAV CLv1-2 | 692 | U.S. Pat. No. 8,734,809 SEQ ID NO: 172 |
| AAV CLv1-3 | 693 | U.S. Pat. No. 8,734,809 SEQ ID NO: 173 |
| AAV CLv1-4 | 694 | U.S. Pat. No. 8,734,809 SEQ ID NO: 174 |
| AAV CLv1-7 | 695 | U.S. Pat. No. 8,734,809 SEQ ID NO: 175 |
| AAV Clv1-8 | 696 | U.S. Pat. No. 8,734,809 SEQ ID NO: 176 |
| AAV Clv1-9 | 697 | U.S. Pat. No. 8,734,809 SEQ ID NO: 177 |
| AAV Clv1-10 | 698 | U.S. Pat. No. 8,734,809 SEQ ID NO: 178 |
| AAV.VR-355 | 699 | U.S. Pat. No. 8,734,809 SEQ ID NO: 181 |
| AAV.hu.48R3 | 700 | U.S. Pat. No. 8,734,809 SEQ ID NO: 183 |
| AAV CBr-E1 | 701 | U.S. Pat. No. 8,734,809 SEQ ID NO: 87 |
| AAV CBr-E2 | 702 | U.S. Pat. No. 8,734,809 SEQ ID NO: 88 |
| AAV CBr-E3 | 703 | U.S. Pat. No. 8,734,809 SEQ ID NO: 89 |
| AAV CBr-E4 | 704 | U.S. Pat. No. 8,734,809 SEQ ID NO: 90 |
| AAV CBr-E5 | 705 | U.S. Pat. No. 8,734,809 SEQ ID NO: 91 |
| AAV CBr-e5 | 706 | U.S. Pat. No. 8,734,809 SEQ ID NO: 92 |
| AAVC Br-E6 | 707 | U.S. Pat. No. 8,734,809 SEQ ID NO: 93 |
| AAVC Br-E7 | 708 | U.S. Pat. No. 8,734,809 SEQ ID NO: 94 |
| AAV CBr-E8 | 709 | U.S. Pat. No. 8,734,809 SEQ ID NO: 95 |
| AAV CLv-D1 | 710 | U.S. Pat. No. 8,734,809 SEQ ID NO: 96 |
| AAV CLv-D2 | 711 | U.S. Pat. No. 8,734,809 SEQ ID NO: 97 |
| AAV CLv-D3 | 712 | U.S. Pat. No. 8,734,809 SEQ ID NO: 98 |
| AAV CLv-D4 | 713 | U.S. Pat. No. 8,734,809 SEQ ID NO: 99 |
| AAV CLv-D5 | 714 | U.S. Pat. No. 8,734,809 SEQ ID NO: 100 |
| AAV CLv-D6 | 715 | U.S. Pat. No. 8,734,809 SEQ ID NO: 101 |
| AAV CLv-D7 | 716 | U.S. Pat. No. 8,734,809 SEQ ID NO: 102 |
| AAV CLv-D8 | 717 | U.S. Pat. No. 8,734,809 SEQ ID NO: 103 |
| AAV CLv-E1 | 718 | U.S. Pat. No. 8,734,809 SEQ ID NO: 87 |
| AAV CLv-R1 | 719 | U.S. Pat. No. 8,734,809 SEQ ID NO: 104 |
| AAV CLv-R2 | 720 | U.S. Pat. No. 8,734,809 SEQ ID NO: 105 |
| AAV CLv-R3 | 721 | U.S. Pat. No. 8,734,809 SEQ ID NO: 106 |
| AAV CLv-R4 | 722 | U.S. Pat. No. 8,734,809 SEQ ID NO: 107 |
| AAV CLv-R5 | 723 | U.S. Pat. No. 8,734,809 SEQ ID NO: 108 |
| AAV CLv-R6 | 724 | U.S. Pat. No. 8,734,809 SEQ ID NO: 109 |
| AAV CLv-R7 | 725 | U.S. Pat. No. 8,734,809 SEQ ID NO: 110 |
| AAV CLv-R8 | 726 | U.S. Pat. No. 8,734,809 SEQ ID NO: 111 |
| AAV CLv-R9 | 727 | U.S. Pat. No. 8,734,809 SEQ ID NO: 112 |
| AAV CLg-F1 | 728 | U.S. Pat. No. 8,734,809 SEQ ID NO: 113 |
| AAV CLg-F2 | 729 | U.S. Pat. No. 8,734,809 SEQ ID NO: 114 |
| AAV CLg-F3 | 730 | U.S. Pat. No. 8,734,809 SEQ ID NO: 115 |
| AAV CLg-F4 | 731 | U.S. Pat. No. 8,734,809 SEQ ID NO: 116 |
| AAV CLg-F5 | 732 | U.S. Pat. No. 8,734,809 SEQ ID NO: 117 |
| AAV CLg-F6 | 733 | U.S. Pat. No. 8,734,809 SEQ ID NO: 117 |
| AAV CLg-F7 | 734 | U.S. Pat. No. 8,734,809 SEQ ID NO: 118 |
| AAV CLg-F8 | 735 | U.S. Pat. No. 8,734,809 SEQ ID NO: 117 |
| AAV CSp-1 | 736 | U.S. Pat. No. 8,734,809 SEQ ID NO: 119 |
| AAV CSp-10 | 737 | U.S. Pat. No. 8,734,809 SEQ ID NO: 120 |
| AAV CSp-11 | 738 | U.S. Pat. No. 8,734,809 SEQ ID NO: 121 |
| AAV CSp-2 | 739 | U.S. Pat. No. 8,734,809 SEQ ID NO: 122 |
| AAV CSp-3 | 740 | U.S. Pat. No. 8,734,809 SEQ ID NO: 123 |
| AAV CSp-4 | 741 | U.S. Pat. No. 8,734,809 SEQ ID NO: 124 |
| AAV CSp-6 | 742 | U.S. Pat. No. 8,734,809 SEQ ID NO: 125 |
| AAV CSp-7 | 743 | U.S. Pat. No. 8,734,809 SEQ ID NO: 126 |
| AAV CSp-8 | 744 | U.S. Pat. No. 8,734,809 SEQ ID NO: 127 |
| AAV CSp-9 | 745 | U.S. Pat. No. 8,734,809 SEQ ID NO: 128 |
| AAV CHt-2 | 746 | U.S. Pat. No. 8,734,809 SEQ ID NO: 129 |
| AAV CHt-3 | 747 | U.S. Pat. No. 8,734,809 SEQ ID NO: 130 |
| AAV CKd-1 | 748 | U.S. Pat. No. 8,734,809 SEQ ID NO: 131 |
| AAV CKd-10 | 749 | U.S. Pat. No. 8,734,809 SEQ ID NO: 132 |
| AAV CKd-2 | 750 | U.S. Pat. No. 8,734,809 SEQ ID NO: 133 |
| AAV CKd-3 | 751 | U.S. Pat. No. 8,734,809 SEQ ID NO: 134 |
| AAV CKd-4 | 752 | U.S. Pat. No. 8,734,809 SEQ ID NO: 135 |
| AAV CKd-6 | 753 | U.S. Pat. No. 8,734,809 SEQ ID NO: 136 |
| AAV CKd-7 | 754 | U.S. Pat. No. 8,734,809 SEQ ID NO: 137 |
| AAV CKd-8 | 755 | U.S. Pat. No. 8,734,809 SEQ ID NO: 138 |
| AAV CLv-1 | 756 | U.S. Pat. No. 8,734,809 SEQ ID NO: 139 |
| AAV CLv-12 | 757 | U.S. Pat. No. 8,734,809 SEQ ID NO: 140 |
| AAV CLv-13 | 758 | U.S. Pat. No. 8,734,809 SEQ ID NO: 141 |
| AAV CLv-2 | 759 | U.S. Pat. No. 8,734,809 SEQ ID NO: 142 |
| AAV CLv-3 | 760 | U.S. Pat. No. 8,734,809 SEQ ID NO: 143 |
| AAV CLv-4 | 761 | U.S. Pat. No. 8,734,809 SEQ ID NO: 144 |
| AAV CLv-6 | 762 | U.S. Pat. No. 8,734,809 SEQ ID NO: 145 |
| AAV CLv-8 | 763 | U.S. Pat. No. 8,734,809 SEQ ID NO: 146 |
| AAV CKd-B1 | 764 | U.S. Pat. No. 8,734,809 SEQ ID NO: 147 |
| AAV CKd-B2 | 765 | U.S. Pat. No. 8,734,809 SEQ ID NO: 148 |
| AAV CKd-B3 | 766 | U.S. Pat. No. 8,734,809 SEQ ID NO: 149 |
| AAV CKd-B4 | 767 | U.S. Pat. No. 8,734,809 SEQ ID NO: 150 |
| AAV CKd-B5 | 768 | U.S. Pat. No. 8,734,809 SEQ ID NO: 151 |
| AAV CKd-B6 | 769 | U.S. Pat. No. 8,734,809 SEQ ID NO: 152 |
| AAV CKd-B7 | 770 | U.S. Pat. No. 8,734,809 SEQ ID NO: 153 |
| AAV CKd-B8 | 771 | U.S. Pat. No. 8,734,809 SEQ ID NO: 154 |
| AAV CKd-H1 | 772 | U.S. Pat. No. 8,734,809 SEQ ID NO: 155 |
| AAV CKd-H2 | 773 | U.S. Pat. No. 8,734,809 SEQ ID NO: 156 |
| AAV CKd-H3 | 774 | U.S. Pat. No. 8,734,809 SEQ ID NO: 157 |
| AAV CKd-H4 | 775 | U.S. Pat. No. 8,734,809 SEQ ID NO: 158 |
| AAV CKd-H5 | 776 | U.S. Pat. No. 8,734,809 SEQ ID NO: 159 |
| AAV CKd-H6 | 777 | U.S. Pat. No. 8,734,809 SEQ ID NO: 151 |
| AAV CHt-1 | 778 | U.S. Pat. No. 8,734,809 SEQ ID NO: 160 |
| AAV CHt-P2 | 779 | WO2016065001 SEQ ID NO: 1 |
| AAV CHt-P5 | 780 | WO2016065001 SEQ ID NO: 2 |
| AAV CHt-P9 | 781 | WO2016065001 SEQ ID NO: 3 |
| AAV CBr-7.1 | 782 | WO2016065001 SEQ ID NO: 4 |
| AAV CBr-7.2 | 783 | WO2016065001 SEQ ID NO: 5 |
| AAV CBr-7.3 | 784 | WO2016065001 SEQ ID NO: 6 |
| AAV CBr-7.4 | 785 | WO2016065001 SEQ ID NO: 7 |
| AAV CBr-7.5 | 786 | WO2016065001 SEQ ID NO: 8 |
| AAV CBr-7.7 | 787 | WO2016065001 SEQ ID NO: 9 |
| AAV CBr-7.8 | 788 | WO2016065001 SEQ ID NO: 10 |
| AAV CBr-7.10 | 789 | WO2016065001 SEQ ID NO: 11 |
| AAV CKd-N3 | 790 | WO2016065001 SEQ ID NO: 12 |
| AAV CKd-N4 | 791 | WO2016065001 SEQ ID NO: 13 |
| AAV CKd-N9 | 792 | WO2016065001 SEQ ID NO: 14 |
| AAV CLv-L4 | 793 | WO2016065001 SEQ ID NO: 15 |
| AAV CLv-L5 | 794 | WO2016065001 SEQ ID NO: 16 |
| AAV CLv-L6 | 795 | WO2016065001 SEQ ID NO: 17 |
| AAV CLv-K1 | 796 | WO2016065001 SEQ ID NO: 18 |
| AAV CLv-K3 | 797 | WO2016065001 SEQ ID NO: 19 |
| AAV CLv-K6 | 798 | WO2016065001 SEQ ID NO: 20 |
| AAV CLv-M1 | 799 | WO2016065001 SEQ ID NO: 21 |
| AAV CLv-M11 | 800 | WO2016065001 SEQ ID NO: 22 |
| AAV CLv-M2 | 801 | WO2016065001 SEQ ID NO: 23 |
| AAV CLv-M5 | 802 | WO2016065001 SEQ ID NO: 24 |
| AAV CLv-M6 | 803 | WO2016065001 SEQ ID NO: 25 |
| AAV CLv-M7 | 804 | WO2016065001 SEQ ID NO: 26 |
| AAV CLv-M8 | 805 | WO2016065001 SEQ ID NO: 27 |
| AAV CLv-M9 | 806 | WO2016065001 SEQ ID NO: 28 |
| AAV CHt-P1 | 807 | WO2016065001 SEQ ID NO: 29 |
| AAV CHt-P6 | 808 | WO2016065001 SEQ ID NO: 30 |
| AAV CHt-P8 | 809 | WO2016065001 SEQ ID NO: 31 |
| AAV CHt-6.1 | 810 | WO2016065001 SEQ ID NO: 32 |
| AAV CHt-6.10 | 811 | WO2016065001 SEQ ID NO: 33 |
| AAV CHt-6.5 | 812 | WO2016065001 SEQ ID NO: 34 |
| AAV CHt-6.6 | 813 | WO2016065001 SEQ ID NO: 35 |
| AAV CHt-6.7 | 814 | WO2016065001 SEQ ID NO: 36 |
| AAV CHt-6.8 | 815 | WO2016065001 SEQ ID NO: 37 |
| AAV CSp-8.10 | 816 | WO2016065001 SEQ ID NO: 38 |
| AAV CSp-8.2 | 817 | WO2016065001 SEQ ID NO: 39 |
| AAV CSp-8.4 | 818 | WO2016065001 SEQ ID NO: 40 |
| AAV CSp-8.5 | 819 | WO2016065001 SEQ ID NO: 41 |
| AAV CSp-8.6 | 820 | WO2016065001 SEQ ID NO: 42 |
| AAV CSp-8.7 | 821 | WO2016065001 SEQ ID NO: 43 |
| AAV CSp-8.8 | 822 | WO2016065001 SEQ ID NO: 44 |
| AAV CSp-8.9 | 823 | WO2016065001 SEQ ID NO: 45 |
| AAV CBr-B7.3 | 824 | WO2016065001 SEQ ID NO: 46 |
| AAV CBr-B7.4 | 825 | WO2016065001 SEQ ID NO: 47 |
| AAV3B | 826 | WO2016065001 SEQ ID NO: 48 |
| AAV4 | 827 | WO2016065001 SEQ ID NO: 49 |
| AAV5 | 828 | WO2016065001 SEQ ID NO: 50 |
| AAV CHt-P2 | 829 | WO2016065001 SEQ ID NO: 51 |
| AAV CHt-P5 | 830 | WO2016065001 SEQ ID NO: 52 |
| AAV CHt-P9 | 831 | WO2016065001 SEQ ID NO: 53 |
| AAV CBr-7.1 | 832 | WO2016065001 SEQ ID NO: 54 |
| AAV CBr-7.2 | 833 | WO2016065001 SEQ ID NO: 55 |
| AAV CBr-7.3 | 834 | WO2016065001 SEQ ID NO: 56 |
| AAV CBr-7.4 | 835 | WO2016065001 SEQ ID NO: 57 |
| AAV CBr-7.5 | 836 | WO2016065001 SEQ ID NO: 58 |
| AAV CBr-7.7 | 837 | WO2016065001 SEQ ID NO: 59 |
| AAV CBr-7.8 | 838 | WO2016065001 SEQ ID NO: 60 |
| AAV CBr-7.10 | 839 | WO2016065001 SEQ ID NO: 61 |
| AAV CKd-N3 | 840 | WO2016065001 SEQ ID NO: 62 |
| AAV CK4-N4 | 841 | WO2016065001 SEQ ID NO: 63 |
| AAV CKd-N9 | 842 | WO2016065001 SEQ ID NO: 64 |
| AAV CLv-L4 | 843 | WO2016065001 SEQ ID NO: 65 |
| AAV CLv-L5 | 844 | WO2016065001 SEQ ID NO: 66 |
| AAV CLv-L6 | 845 | WO2016065001 SEQ ID NO: 67 |
| AAV CLv-K1 | 846 | WO2016065001 SEQ ID NO: 68 |
| AAV CLv-K3 | 847 | WO2016065001 SEQ ID NO: 69 |
| AAV CLv-K6 | 848 | WO2016065001 SEQ ID NO: 70 |
| AAV CLv-M1 | 849 | WO2016065001 SEQ ID NO: 71 |
| AAV CLv-M11 | 850 | WO2016065001 SEQ ID NO: 72 |
| AAV CLv-M2 | 851 | WO2016065001 SEQ ID NO: 73 |
| AAV CLv-M5 | 852 | WO2016065001 SEQ ID NO: 74 |
| AAV CLv-M6 | 853 | WO2016065001 SEQ ID NO: 75 |
| AAV CLv-M7 | 854 | WO2016065001 SEQ ID NO: 76 |
| AAV CLv-M8 | 855 | WO2016065001 SEQ ID NO: 77 |
| AAV CLv-M9 | 856 | WO2016065001 SEQ ID NO: 78 |
| AAV CHt-P1 | 857 | WO2016065001 SEQ ID NO: 79 |
| AAV CHt-P6 | 858 | WO2016065001 SEQ ID NO: 80 |
| AAV CHt-P8 | 859 | WO2016065001 SEQ ID NO: 81 |
| AAV CHt-6.1 | 860 | WO2016065001 SEQ ID NO: 82 |
| AAV CHt-6.10 | 861 | WO2016065001 SEQ ID NO: 83 |
| AAV CHt-6.5 | 862 | WO2016065001 SEQ ID NO: 84 |
| AAV CHt-6.6 | 863 | WO2016065001 SEQ ID NO: 85 |
| AAV CHt-6.7 | 864 | WO2016065001 SEQ ID NO: 86 |
| AAV CHt-6.8 | 865 | WO2016065001 SEQ ID NO: 87 |
| AAV CSp-8.10 | 866 | WO2016065001 SEQ ID NO: 88 |
| AAV CSp-8.2 | 867 | WO2016065001 SEQ ID NO: 89 |
| AAV CSp-8.4 | 868 | WO2016065001 SEQ ID NO: 90 |
| AAV CSp-8.5 | 869 | WO2016065001 SEQ ID NO: 91 |
| AAV CSp-8.6 | 870 | WO2016065001 SEQ ID NO: 92 |
| AAV CSp-8.7 | 871 | WO2016065001 SEQ ID NO: 93 |
| AAV CSp-8.8 | 872 | WO2016065001 SEQ ID NO: 94 |
| AAV CSp-8.9 | 873 | WO2016065001 SEQ ID NO: 95 |
| AAV CBr-B7.3 | 874 | WO2016065001 SEQ ID NO: 96 |
| AAV CBr-B7.4 | 875 | WO2016065001 SEQ ID NO: 97 |
| AAV3B | 876 | WO2016065001 SEQ ID NO: 98 |
| AAV4 | 877 | WO2016065001 SEQ ID NO: 99 |
| AAV5 | 878 | WO2016065001 SEQ ID NO: 100 |
| GPV | 879 | U.S. Pat. No. 9,624,274B2 SEQ ID NO: 192 |
| B19 | 880 | U.S. Pat. No. 9,624,274B2 SEQ ID NO: 193 |
| MVM | 881 | U.S. Pat. No. 9,624,274B2 SEQ ID NO: 194 |
| FPV | 882 | U.S. Pat. No. 9,624,274B2 SEQ ID NO: 195 |
| CPV | 883 | U.S. Pat. No. 9,624,274B2 SEQ ID NO: 196 |
| AAV6 | 884 | U.S. Pat. No. 9,546,112B2 SEQ ID NO: 5 |
| AAV6 | 885 | U.S. Pat. No. 9,457,103B2 SEQ ID NO: 1 |
| AAV2 | 886 | U.S. Pat. No. 9,457,103B2 SEQ ID NO: 2 |
| ShH10 | 887 | U.S. Pat. No. 9,457,103B2 SEQ ID NO: 3 |
| ShH13 | 888 | U.S. Pat. No. 9,457,103B2 SEQ ID NO: 4 |
| ShH10 | 889 | U.S. Pat. No. 9,457,103B2 SEQ ID NO: 5 |
| ShH10 | 890 | U.S. Pat. No. 9,457,103B2 SEQ ID NO: 6 |
| ShH10 | 891 | U.S. Pat. No. 9,457,103B2 SEQ ID NO: 7 |
| ShH10 | 892 | U.S. Pat. No. 9,457,103B2 SEQ ID NO: 8 |
| ShH10 | 893 | U.S. Pat. No. 9,457,103B2 SEQ ID NO: 9 |
| rh74 | 894 | U.S. Pat. No. 9,434,928B2 SEQ ID NO: 1, |
| | | US2015023924A1 SEQ ID NO: 2 |
| rh74 | 895 | U.S. Pat. No. 9,434,928B2 SEQ ID NO: 2, |
| | | US2015023924A1 SEQ ID NO: 1 |
| AAV8 | 896 | U.S. Pat. No. 9,434,928B2 SEQ ID NO: 4 |
| rh74 | 897 | U.S. Pat. No. 9,434,928B2 SEQ ID NO: 5 |
| rh74 (RHM4-1) | 898 | US2015023924A1 SEQ ID NO: 5, US20160375110A1 |
| | | SEQ ID NO: 4 |
| rh74 (RHM15-1) | 899 | US2015023924A1 SEQ ID NO: 6, US20160375110A1 |
| | | SEQ ID NO: 5 |
| rh74 (RHM15-2) | 900 | US2015023924A1 SEQ ID NO: 7, US20160375110A1 |
| | | SEQ ID NO: 6 |
| rh74 (RHM15-3/RHM15-5) | 901 | US2015023924A1 SEQ ID NO: 8, US20160375110A1 |
| | | SEQ ID NO: 7 |
| rh74 (RHM15-4) | 902 | US2015023924A1 SEQ ID NO: 9, US20160375110A1 |
| | | SEQ ID NO: 8 |
| rh74 (RHM15-6) | 903 | US2015023924A1 SEQ ID NO: 10, US20160375110A1 |
| | | SEQ ID NO: 9 |
| rh74 (RHM4-1) | 904 | US2015023924A1 SEQ ID NO: 11 |
| rh74 (RHM15-1) | 905 | US2015023924A1 SEQ ID NO: 12 |
| rh74 (RHM15-2) | 906 | US2015023924A1 SEQ ID NO: 13 |
| rh74 (RHM15-3/RHM15-5) | 907 | US2015023924A1 SEQ ID NO: 14 |
| rh74 (RHM15-4) | 908 | US2015023924A1 SEQ ID NO: 15 |
| rh74 (RHM15-6) | 909 | US2015023924A1 SEQ ID NO: 16 |
| AAV2 (comprising lung | 910 | US20160175389A1 SEQ ID NO: 9 |
| specific polypeptide) | | |
| AAV2 (comprising lung | 911 | US20160175389A1 SEQ ID NO: 10 |
| specific polypeptide) | | |
| Anc80 | 912 | US20170051257A1 SEQ ID NO: 1 |
| Anc80 | 913 | US20170051257A1 SEQ ID NO: 2 |
| Anc81 | 914 | US20170051257A1 SEQ ID NO: 3 |
| Anc80 | 915 | US20170051257A1 SEQ ID NO: 4 |
| Anc82 | 916 | US20170051257A1 SEQ ID NO: 5 |
| Anc82 | 917 | US20170051257A1 SEQ ID NO: 6 |
| Anc83 | 918 | US20170051257A1 SEQ ID NO: 7 |
| Anc83 | 919 | US20170051257A1 SEQ ID NO: 8 |
| Anc84 | 920 | US20170051257A1 SEQ ID NO: 9 |
| Anc84 | 921 | US20170051257A1 SEQ ID NO: 10 |
| Anc94 | 922 | US20170051257A1 SEQ ID NO: 11 |
| Anc94 | 923 | US20170051257A1 SEQ ID NO: 12 |
| Anc113 | 924 | US20170051257A1 SEQ ID NO: 13 |
| Anc113 | 925 | US20170051257A1 SEQ ID NO: 14 |
| Anc126 | 926 | US20170051257A1 SEQ ID NO: 15 |
| Anc126 | 927 | US20170051257A1 SEQ ID NO: 16 |
| Anc127 | 928 | US20170051257A1 SEQ ID NO: 17 |
| Anc127 | 929 | US20170051257A1 SEQ ID NO: 18 |
| Anc80L27 | 930 | US20170051257A1 SEQ ID NO: 19 |
| Anc80L59 | 931 | US20170051257A1 SEQ ID NO: 20 |
| Anc80L60 | 932 | US20170051257A1 SEQ ID NO: 21 |
| Anc80L62 | 933 | US20170051257A1 SEQ ID NO: 22 |
| Anc80L65 | 934 | US20170051257A1 SEQ ID NO: 23 |
| Anc80L33 | 935 | US20170051257A1 SEQ ID NO: 24 |
| Anc80L36 | 936 | US20170051257A1 SEQ ID NO: 25 |
| Anc80L44 | 937 | US20170051257A1 SEQ ID NO: 26 |
| Anc80L1 | 938 | US2017005I257A1 SEQ ID NO: 35 |
| Anc80L1 | 939 | US20170051257A1 SEQ ID NO: 36 |
| AAV-X1 | 940 | U.S. Pat. No. 8,283,151B2 SEQ ID NO: 11 |
| AAV-X1b | 941 | U.S. Pat. No. 8,283,151B2 SEQ ID NO: 12 |
| AAV-X5 | 942 | U.S. Pat. No. 8,283,151B2 SEQ ID NO: 13 |
| AAV-X19 | 943 | U.S. Pat. No. 8,283,151B2 SEQ ID NO: 14 |
| AAV-X21 | 944 | U.S. Pat. No. 8,283,151B2 SEQ ID NO: 15 |
| AAV-X22 | 945 | U.S. Pat. No. 8,283,151B2 SEQ ID NO: 16 |
| AAV-X23 | 946 | U.S. Pat. No. 8,283,151B2 SEQ ID NO: 17 |
| AAV-X24 | 947 | U.S. Pat. No. 8,283,151B2 SEQ ID NO: 18 |
| AAV-X25 | 948 | U.S. Pat. No. 8,283,151B2 SEQ ID NO: 19 |
| AAV-X26 | 949 | U.S. Pat. No. 8,283,151B2 SEQ ID NO: 20 |
| AAV-X1 | 950 | U.S. Pat. No. 8,283,151B2 SEQ ID NO: 21 |
| AAV-X1b | 951 | U.S. Pat. No. 8,283,151B2 SEQ ID NO: 22 |
| AAV-X5 | 952 | U.S. Pat. No. 8,283,151B2 SEQ ID NO: 23 |
| AAV-X19 | 953 | U.S. Pat. No. 8,283,151B2 SEQ ID NO: 24 |
| AAV-X21 | 954 | U.S. Pat. No. 8,283,151B2 SEQ ID NO: 25 |
| AAV-X22 | 955 | U.S. Pat. No. 8,283,151B2 SEQ ID NO: 26 |
| AAV-X23 | 956 | U.S. Pat. No. 8,283,151B2 SEQ ID NO: 27 |
| AAV-X24 | 957 | U.S. Pat. No. 8,283,151B2 SEQ ID NO: 28 |
| AAV-X25 | 958 | U.S. Pat. No. 8,283,151B2 SEQ ID NO: 29 |
| AAV-X26 | 959 | U.S. Pat. No. 8,283,151B2 SEQ ID NO: 30 |
| AAVrh8 | 960 | WO2016054554A1 SEQ ID NO: 8 |
| AAVrh8VP2FC5 | 961 | WO2016054554A1 SEQ ID NO: 9 |
| AAVrh8VP2FC44 | 962 | WO2016054554A1 SEQ ID NO: 10 |
| AAVrh8VP2ApoB100 | 963 | WO2016054554A1 SEQ ID NO: 11 |
| AAVrh8VP2RVG | 964 | WO2016054554A1 SEQ ID NO: 12 |
| AAVrh8VP2Angiopep-2 | 965 | WO2016054554A1 SEQ ID NO: 13 |
| VP2 | | |
| AAV9.47VP1.3 | 966 | WO2016054554A1 SEQ ID NO: 14 |
| AAV9.47VP2ICAMg3 | 967 | WO2016054554A1 SEQ ID NO: 15 |
| AAV9.47VP2RVG | 968 | WO2016054554A1 SEQ ID NO: 16 |
| AAV9.47VP2Angiopep-2 | 969 | WO2016054554A1 SEQ ID NO: 17 |
| AAV9.47VP2A-string | 970 | WO2016054554A1 SEQ ID NO: 18 |
| AAVrh8VP2FC5 VP2 | 971 | WO2016054554A1 SEQ ID NO: 19 |
| AAVrb8VP2FC44 VP2 | 972 | WO2016054554A1 SEQ ID NO: 20 |
| AAVrh8VP2ApoB100 VP2 | 973 | WO2016054554A1 SEQ ID NO: 21 |
| AAVrh8VP2RVG VP2 | 974 | WO2016054554A1 SEQ ID NO: 22 |
| AAVrh8VP2Angiopep-2 | 975 | WO2016054554A1 SEQ ID NO: 23 |
| VP2 | | |
| AAV9.47VP2ICAMg3 VP2 | 976 | WO2016054554A1 SEQ ID NO: 24 |
| AAV9.47VP2RVG VP2 | 977 | WO2016054554A1 SEQ ID NO: 25 |
| AAV9.47VP2Angiopep-2 | 978 | WO2016054554A1 SEQ ID NO: 26 |
| VP2 | | |
| AAV9.47VP2A-string VP2 | 979 | WO2016054554A1 SEQ ID NO: 27 |
| rAAV-B1 | 980 | WO2016054557A1 SEQ ID NO: 1 |
| rAAV-B2 | 981 | WO2016054557A1 SEQ ID NO: 2 |
| rAAV-B3 | 982 | WO2016054557A1 SEQ ID NO: 3 |
| rAAV-B4 | 983 | WO2016054557A1 SEQ ID NO: 4 |
| rAAV-B1 | 984 | WO2016054557A1 SEQ ID NO: 5 |
| rAAV-B2 | 985 | WO2016054557A1 SEQ ID NO: 6 |
| rAAV-B3 | 986 | WO2016054557A1 SEQ ID NO: 7 |
| rAAV-B4 | 987 | WO2016054557A1 SEQ ID NO: 8 |
| rAAV-L1 | 988 | WO2016054557A1 SEQ ID NO: 9 |
| rAAV-L2 | 989 | WO2016054557A1 SEQ ID NO: 10 |
| rAAV-L3 | 990 | WO2016054557A1 SEQ ID NO: 11 |
| rAAV-L4 | 991 | WO2016054557A1 SEQ ID NO: 12 |
| rAAV-L1 | 992 | WO2016054557A1 SEQ ID NO: 13 |
| rAAV-L2 | 993 | WO2016054557A1 SEQ ID NO: 14 |
| rAAV-L3 | 994 | WO2016054557A1 SEQ ID NO: 15 |
| rAAV-L4 | 995 | WO2016054557A1 SEQ ID NO: 16 |
| AAV9 | 996 | WO2016073739A1 SEQ ID NO: 3 |
| rAAV | 997 | WO2016081811A1 SEQ ID NO: 1 |
| rAAV | 998 | WO2016081811A1 SEQ ID NO: 2 |
| rAAV | 999 | WO2016081811A1 SEQ ID NO: 3 |
| rAAV | 1000 | WO2016081811A1 SEQ ID NO: 4 |
| rAAV | 1001 | WO2016081811A1 SEQ ID NO: 5 |
| rAAV | 1002 | WO2016081811A1 SEQ ID NO: 6 |
| rAAV | 1003 | WO2016081811A1 SEQ ID NO: 7 |
| rAAV | 1004 | WO2016081811A1 SEQ ID NO: 8 |
| rAAV | 1005 | WO2016081811A1 SEQ ID NO: 9 |
| rAAV | 1006 | WO2016081811A1 SEQ ID NO: 10 |
| rAAV | 1007 | WO2016081811A1 SEQ ID NO: 11 |
| rAAV | 1008 | WO2016081811A1 SEQ ID NO: 12 |
| rAAV | 1009 | WO2016081811A1 SEQ ID NO: 13 |
| rAAV | 1010 | WO2016081811A1 SEQ ID NO: 14 |
| rAAV | 1011 | WO2016081811A1 SEQ ID NO: 15 |
| rAAV | 1012 | WO2016081811A1 SEQ ID NO: 16 |
| rAAV | 1013 | WO2016081811A1 SEQ ID NO: 17 |
| rAAV | 1014 | WO2016081811A1 SEQ ID NO: 18 |
| rAAV | 1015 | WO2016081811A1 SEQ ID NO: 19 |
| rAAV | 1016 | WO2016081811A1 SEQ ID NO: 20 |
| rAAV | 1017 | WO2016081811A1 SEQ ID NO: 21 |
| rAAV | 1018 | WO2016081811A1 SEQ ID NO: 22 |
| rAAV | 1019 | WO2016081811A1 SEQ ID NO: 23 |
| rAAV | 1020 | WO2016081811A1 SEQ ID NO: 24 |
| rAAV | 1021 | WO2016081811A1 SEQ ID NO: 25 |
| rAAV | 1022 | WO2016081811A1 SEQ ID NO: 26 |
| rAAV | 1023 | WO2016081811A1 SEQ ID NO: 27 |
| rAAV | 1024 | WO2016081811A1 SEQ ID NO: 28 |
| rAAV | 1025 | WO2016081811A1 SEQ ID NO: 29 |
| rAAV | 1026 | WO2016081811A1 SEQ ID NO: 30 |
| rAAV | 1027 | WO2016081811A1 SEQ ID NO: 31 |
| rAAV | 1028 | WO2016081811A1 SEQ ID NO: 32 |
| rAAV | 1029 | WO2016081811A1 SEQ ID NO: 33 |
| rAAV | 1030 | WO2016081811A1 SEQ ID NO: 34 |
| rAAV | 1031 | WO2016081811A1 SEQ ID NO: 35 |
| rAAV | 1032 | WO2016081811A1 SEQ ID NO: 36 |
| rAAV | 1033 | WO2016081811A1 SEQ ID NO: 37 |
| rAAV | 1034 | WO2016081811A1 SEQ ID NO: 38 |
| rAAV | 1035 | WO2016081811A1 SEQ ID NO: 39 |
| rAAV | 1036 | WO2016081811A1 SEQ ID NO: 40 |
| rAAV | 1037 | WO2016081811A1 SEQ ID NO: 41 |
| rAAV | 1038 | WO2016081811A1 SEQ ID NO: 42 |
| rAAV | 1039 | WO2016081811A1 SEQ ID NO: 43 |
| rAAV | 1040 | WO2016081811A1 SEQ ID NO: 44 |
| rAAV | 1041 | WO201608I811A1 SEQ ID NO: 45 |
| rAAV | 1042 | WO2016081811A1 SEQ ID NO: 46 |
| rAAV | 1043 | WO2016081811A1 SEQ ID NO: 47 |
| rAAV | 1044 | WO2016081811A1 SEQ ID NO: 48 |
| rAAV | 1045 | WO2016081811A1 SEQ ID NO: 49 |
| rAAV | 1046 | WO2016081811A1 SEQ ID NO: 50 |
| rAAV | 1047 | WO2016081811A1 SEQ ID NO: 51 |
| rAAV | 1048 | WO2016081811A1 SEQ ID NO: 52 |
| rAAV | 1049 | WO2016081811A1 SEQ ID NO: 53 |
| rAAV | 1050 | WO2016081811A1 SEQ ID NO: 54 |
| rAAV | 1051 | WO2016081811A1 SEQ ID NO: 55 |
| rAAV | 1052 | WO2016081811A1 SEQ ID NO: 56 |
| rAAV | 1053 | WO2016081811A1 SEQ ID NO: 57 |
| rAAV | 1054 | WO2016081811A1 SEQ ID NO: 58 |
| rAAV | 1055 | WO2016081811A1 SEQ ID NO: 59 |
| rAAV | 1056 | WO2016081811A1 SEQ ID NO: 60 |
| rAAV | 1057 | WO2016081811A1 SEQ ID NO: 61 |
| rAAV | 1058 | WO2016081811A1 SEQ ID NO: 62 |
| rAAV | 1059 | WO2016081811A1 SEQ ID NO: 63 |
| rAAV | 1060 | WO2016081811A1 SEQ ID NO: 64 |
| rAAV | 1061 | WO2016081811A1 SEQ ID NO: 65 |
| rAAV | 1062 | WO2016081811A1 SEQ ID NO: 66 |
| rAAV | 1063 | WO2016081811A1 SEQ ID NO: 67 |
| rAAV | 1064 | WO2016081811A1 SEQ ID NO: 68 |
| rAAV | 1065 | WO2016081811A1 SEQ ID NO: 69 |
| rAAV | 1066 | WO2016081811A1 SEQ ID NO: 70 |
| rAAV | 1067 | WO2016081811A1 SEQ ID NO: 71 |
| rAAV | 1068 | WO2016081811A1 SEQ ID NO: 72 |
| rAAV | 1069 | WO2016081811A1 SEQ ID NO: 73 |
| rAAV | 1070 | WO2016081811A1 SEQ ID NO: 74 |
| rAAV | 1071 | WO2016081811A1 SEQ ID NO: 75 |
| rAAV | 1072 | WO2016081811A1 SEQ ID NO: 76 |
| rAAV | 1073 | WO2016081811A1 SEQ ID NO: 77 |
| rAAV | 1074 | WO2016081811A1 SEQ ID NO: 78 |
| rAAV | 1075 | WO2016081811A1 SEQ ID NO: 79 |
| rAAV | 1076 | WO2016081811A1 SEQ ID NO: 80 |
| rAAV | 1077 | WO2016081811A1 SEQ ID NO: 81 |
| rAAV | 1078 | WO2016081811A1 SEQ ID NO: 82 |
| rAAV | 1079 | WO2016081811A1 SEQ ID NO: 83 |
| rAAV | 1080 | WO2016081811A1 SEQ ID NO: 84 |
| rAAV | 1081 | WO2016081811A1 SEQ ID NO: 85 |
| rAAV | 1082 | WO2016081811A1 SEQ ID NO: 86 |
| rAAV | 1083 | WO2016081811A1 SEQ ID NO: 87 |
| rAAV | 1084 | WO2016081811A1 SEQ ID NO: 88 |
| rAAV | 1085 | WO2016081811A1 SEQ ID NO: 89 |
| rAAV | 1086 | WO2016081811A1 SEQ ID NO: 90 |
| rAAV | 1087 | WO2016081811A1 SEQ ID NO: 91 |
| rAAV | 1088 | WO2016081811A1 SEQ ID NO: 92 |
| rAAV | 1089 | WO2016081811A1 SEQ ID NO: 93 |
| rAAV | 1090 | WO2016081811A1 SEQ ID NO: 94 |
| rAAV | 1091 | WO2016081811A1 SEQ ID NO: 95 |
| rAAV | 1092 | WO2016081811A1 SEQ ID NO: 96 |
| rAAV | 1093 | WO2016081811A1 SEQ ID NO: 97 |
| rAAV | 1094 | WO2016081811A1 SEQ ID NO: 98 |
| rAAV | 1095 | WO2016081811A1 SEQ ID NO: 99 |
| rAAV | 1096 | WO2016081811A1 SEQ ID NO: 100 |
| rAAV | 1097 | WO2016081811A1 SEQ ID NO: 101 |
| rAAV | 1098 | WO2016081811A1 SEQ ID NO: 102 |
| rAAV | 1099 | WO2016081811A1 SEQ ID NO: 103 |
| rAAV | 1100 | WO2016081811A1 SEQ ID NO: 104 |
| rAAV | 1101 | WO2016081811A1 SEQ ID NO: 105 |
| rAAV | 1102 | WO2016081811A1 SEQ ID NO: 106 |
| rAAV | 1103 | WO2016081811A1 SEQ ID NO: 107 |
| rAAV | 1104 | WO2016081811A1 SEQ ID NO: 108 |
| rAAV | 1105 | WO2016081811A1 SEQ ID NO: 109 |
| rAAV | 1106 | WO2016081811A1 SEQ ID NO: 110 |
| rAAV | 1107 | WO2016081811A1 SEQ ID NO: 111 |
| rAAV | 1108 | WO2016081811A1 SEQ ID NO: 112 |
| rAAV | 1109 | WO2016081811A1 SEQ ID NO: 113 |
| rAAV | 1110 | WO2016081811A1 SEQ ID NO: 114 |
| rAAV | 1111 | WO2016081811A1 SEQ ID NO: 115 |
| rAAV | 1112 | WO2016081811A1 SEQ ID NO: 116 |
| rAAV | 1113 | WO2016081811A1 SEQ ID NO: 117 |
| rAAV | 1114 | WO2016081811A1 SEQ ID NO: 118 |
| rAAV | 1115 | WO2016081811A1 SEQ ID NO: 119 |
| rAAV | 1116 | WO2016081811A1 SEQ ID NO: 120 |
| rAAV | 1117 | WO2016081811A1 SEQ ID NO: 121 |
| rAAV | 1118 | WO2016081811A1 SEQ ID NO: 122 |
| rAAV | 1119 | WO2016081811A1 SEQ ID NO: 123 |
| rAAV | 1120 | WO2016081811A1 SEQ ID NO: 124 |
| rAAV | 1121 | WO2016081811A1 SEQ ID NO: 125 |
| rAAV | 1122 | WO2016081811A1 SEQ ID NO: 126 |
| rAAV | 1123 | WO2016081811A1 SEQ ID NO: 127 |
| rAAV | 1124 | WO2016081811A1 SEQ ID NO: 128 |
| AAV8 E532K | 1125 | WO2016081811A1 SEQ ID NO: 133 |
| AAV8 E532K | 1126 | WO2016081811A1 SEQ ID NO: 134 |
| rAAV4 | 1127 | WO2016115382A1 SEQ ID NO: 2 |
| rAAV4 | 1128 | WO2016115382A1 SEQ ID NO: 3 |
| rAAV4 | 1129 | WO2016115382A1 SEQ ID NO: 4 |
| rAAV4 | 1130 | WO2016115382A1 SEQ ID NO: 5 |
| rAAV4 | 1131 | WO2016115382A1 SEQ ID NO: 6 |
| rAAV4 | 1132 | WO2016115382A1 SEQ ID NO: 7 |
| rAAV4 | 1133 | WO2016115382A1 SEQ ID NO: 8 |
| rAAV4 | 1134 | WO2016115382A1 SEQ ID NO: 9 |
| rAAV4 | 1135 | WO2016115382A1 SEQ ID NO: 10 |
| rAAV4 | 1136 | WO2016115382A1 SEQ ID NO: 11 |
| rAAV4 | 1137 | WO2016115382A1 SEQ ID NO: 12 |
| rAAV4 | 1138 | WO2016115382A1 SEQ ID NO: 13 |
| rAAV4 | 1139 | WO2016115382A1 SEQ ID NO: 14 |
| rAAV4 | 1140 | WO2016115382A1 SEQ ID NO: 15 |
| rAAV4 | 1141 | WO2016115382A1 SEQ ID NO: 16 |
| rAAV4 | 1142 | WO2016115382A1 SEQ ID NO: 17 |
| rAAV4 | 1143 | WO2016115382A1 SEQ ID NO: 18 |
| rAAV4 | 1144 | WO2016115382A1 SEQ ID NO: 19 |
| rAAV4 | 1145 | WO2016115382A1 SEQ ID NO: 20 |
| rAAV4 | 1146 | WO2016115382A1 SEQ ID NO: 21 |
| AAV11 | 1147 | WO2016115382A1 SEQ ID NO: 22 |
| AAV12 | 1148 | WO2016115382A1 SEQ ID NO: 23 |
| rh32 | 1149 | WO2016115382A1 SEQ ID NO: 25 |
| rh33 | 1150 | WO2016115382A1 SEQ ID NO: 26 |
| rh34 | 1151 | WO2016115382A1 SEQ ID NO: 27 |
| rAAV4 | 1152 | WO2016115382A1 SEQ ID NO: 28 |
| rAAV4 | 1153 | WO2016115382A1 SEQ ID NO: 29 |
| rAAV4 | 1154 | WO2016115382A1 SEQ ID NO: 30 |
| rAAV4 | 1155 | WO2016115382A1 SEQ ID NO: 31 |
| rAAV4 | 1156 | WO2016115382A1 SEQ ID NO: 32 |
| rAAV4 | 1157 | WO2016115382A1 SEQ ID NO: 33 |
| AAV2/8 | 1158 | WO2016131981A1 SEQ ID NO: 47 |
| AAV2/8 | 1159 | WO2016131981A1 SEQ ID NO: 48 |
| ancestral AAV | 1160 | WO2016154344A1 SEQ ID NO: 7 |
| ancestral AAV variant C4 | 1161 | WO2016154344A1 SEQ ID NO: 13 |
| ancestral AAV variant C7 | 1162 | WO2016154344A1 SEQ ID NO: 14 |
| ancestral AAV variant G4 | 1163 | WO2016154344A1 SEQ ID NO: 15 |
| consensus amino acid | 1164 | WO2016154344A1 SEQ ID NO: 16 |
| sequence of ancestral AAV | | |
| variants, C4, C7 and G4 | | |
| consensus amino acid | 1165 | WO2016154344A1 SEQ ID NO: 17 |
| sequence of ancestral AAV | | |
| variants, C4 and C7 | | |
| AAV8 (with a AAV2 | 1166 | WO2016150403A1 SEQ ID NO: 13 |
| phospholipase domain) | | |
| AAV VR-942n | 1167 | US20160289275A1 SEQ ID NO: 10 |
| AAV5-A (M569V) | 1168 | US20160289275A1 SEQ ID NO: 13 |
| AAV5-A (M569V) | 1169 | US20160289275A1 SEQ ID NO: 14 |
| AAV5-A (Y585V) | 1170 | US20160289275A1 SEQ ID NO: 16 |
| AAV5-A (Y585V) | 1171 | US20160289275A1 SEQ ID NO: 17 |
| AAV5-A (L587T) | 1172 | US20160289275A1 SEQ ID NO: 19 |
| AAV5-A (L587T) | 1173 | US20160289275A1 SEQ ID NO: 20 |
| AAV5-A (Y585V/L587T) | 1174 | US20160289275A1 SEQ ID NO: 22 |
| AAV5-A (Y585V/L587T) | 1175 | US20160289275A1 SEQ ID NO: 23 |
| AAV5-B (D652A) | 1176 | US20160289275A1 SEQ ID NO: 25 |
| AAV5-B (D652A) | 1177 | US20160289275A1 SEQ ID NO: 26 |
| AAV5-B (T362M) | 1178 | US20160289275A1 SEQ ID NO: 28 |
| AAV5-B (T362M) | 1179 | US20160289275A1 SEQ ID NO: 29 |
| AAV5-B (Q359D) | 1180 | US20160289275A1 SEQ ID NO: 31 |
| AAV5-B (Q359D) | 1181 | US20160289275A1 SEQ ID NO: 32 |
| AAV5-B (E350Q) | 1182 | US20160289275A1 SEQ ID NO: 34 |
| AAV5-B (E350Q) | 1183 | US20160289275A1 SEQ ID NO: 35 |
| AAV5-B (P533S) | 1184 | US20160289275A1 SEQ ID NO: 37 |
| AAV5-B (P533S) | 1185 | US20160289275A1 SEQ ID NO: 38 |
| AAV5-B (P533G) | 1186 | US20160289275A1 SEQ ID NO: 40 |
| AAV5-B (P533G) | 1187 | US20160289275A1 SEQ ID NO: 41 |
| AAV5-mutation in loop VII | 1188 | US20160289275A1 SEQ ID NO: 43 |
| AAV5-mutation in loop VII | 1189 | US20160289275A1 SEQ ID NO: 44 |
| AAV8 | 1190 | US20160289275A1 SEQ ID NO: 47 |
| Mut A (LK03/AAV8) | 1191 | WO2016181123A1 SEQ ID NO: 1 |
| Mut B (LK03/AAV5) | 1192 | WO2016181123A1 SEQ ID NO: 2 |
| Mut C (AAV8/AAV3B) | 1193 | WO2016181123A1 SEQ ID NO: 3 |
| Mut D (AAV5/AAV3B) | 1194 | WO2016181123A1 SEQ ID NO: 4 |
| Mut E (AAV8/AAV3B) | 1195 | WO2016181123A1 SEQ ID NO: 5 |
| Mut F (AAV3B/AAV8) | 1196 | WO2016181123A1 SEQ ID NO: 6 |
| AAV44.9 | 1197 | WO2016183297A1 SEQ ID NO: 4 |
| AAV44.9 | 1198 | WO2016183297A1 SEQ ID NO: 5 |
| AAVrh8 | 1199 | WO2016183297A1 SEQ ID NO: 6 |
| AAV44.9 (S470N) | 1200 | WO2016183297A1 SEQ ID NO: 9 |
| rh74 VP1 | 1201 | US20160375110A1 SEQ ID NO: 1 |
| AAV-LK03 (L125I) | 1202 | WO2017015102A1 SEQ ID NO: 5 |
| AAV3B (S663V + T492V) | 1203 | WO2017015102A1 SEQ ID NO: 6 |
| Anc80 | 1204 | WO2017019994A2 SEQ ID NO: 1 |
| Anc80 | 1205 | WO2017019994A2 SEQ ID NO: 2 |
| Anc81 | 1206 | WO2017019994A2 SEQ ID NO: 3 |
| Anc81 | 1207 | WO2017019994A2 SEQ ID NO: 4 |
| Anc82 | 1208 | WO2017019994A2 SEQ ID NO: 5 |
| Anc82 | 1209 | WO2017019994A2 SEQ ID NO: 6 |
| Anc83 | 1210 | WO2017019994A2 SEQ ID NO: 7 |
| Anc83 | 1211 | WO2017019994A2 SEQ ID NO: 8 |
| Anc84 | 1212 | WO2017019994A2 SEQ ID NO: 9 |
| Anc84 | 1213 | WO2017019994A2 SEQ ID NO: 10 |
| Anc94 | 1214 | WO2017019994A2 SEQ ID NO: 11 |
| Anc94 | 1215 | WO2017019994A2 SEQ ID NO: 12 |
| Anc113 | 1216 | WO2017019994A2 SEQ ID NO: 13 |
| Anc113 | 1217 | WO2017019994A2 SEQ ID NO: 14 |
| Anc126 | 1218 | WO2017019994A2 SEQ ID NO: 15 |
| Anc126 | 1219 | WO2017019994A2 SEQ ID NO: 16 |
| Anc127 | 1220 | WO2017019994A2 SEQ ID NO: 17 |
| Anc127 | 1221 | WO2017019994A2 SEQ ID NO: 18 |
| Anc80L27 | 1222 | WO2017019994A2 SEQ ID NO: 19 |
| Anc80L59 | 1223 | WO2017019994A2 SEQ ID NO: 20 |
| Anc80L60 | 1224 | WO2017019994A2 SEQ ID NO: 21 |
| Anc80L62 | 1225 | WO2017019994A2 SEQ ID NO: 22 |
| Anc80L65 | 1226 | WO2017019994A2 SEQ ID NO: 23 |
| Anc80L33 | 1227 | WO2017019994A2 SEQ ID NO: 24 |
| Anc80L36 | 1228 | WO2017019994A2 SEQ ID NO: 25 |
| Anc80L44 | 1229 | WO2017019994A2 SEQ ID NO: 26 |
| Anc80L1 | 1230 | WO2017019994A2 SEQ ID NO: 35 |
| Anc80L1 | 1231 | WO2017019994A2 SEQ ID NO: 36 |
| AAVrh10 | 1232 | WO2017019994A2 SEQ ID NO: 41 |
| Anc110 | 1233 | WO2017019994A2 SEQ ID NO: 42 |
| Anc110 | 1234 | WO2017019994A2 SEQ ID NO: 43 |
| AAVrh32.33 | 1235 | WO2017019994A2 SEQ ID NO: 45 |
| AAVrh74 | 1236 | WO2017049031A1 SEQ ID NO: 1 |
| AAV2 | 1237 | WO2017053629A2 SEQ ID NO: 49 |
| AAV2 | 1238 | WO2017053629A2 SEQ ID NO: 50 |
| AAV2 | 1239 | WO2017053629A2 SEQ ID NO: 82 |
| Parvo-like virus | 1240 | WO2017070476A2 SEQ ID NO: 1 |
| Parvo-like virus | 1241 | WO2017070476A2 SEQ ID NO: 2 |
| Parvo-like virus | 1242 | WO2017070476A2 SEQ ID NO: 3 |
| Parvo-like virus | 1243 | WO2017070476A2 SEQ ID NO: 4 |
| Parvo-like virus | 1244 | WO2017070476A2 SEQ ID NO: 5 |
| Parvo-like virus | 1245 | WO2017070476A2 SEQ ID NO: 6 |
| AAVrh.10 | 1246 | WO2017070516A1 SEQ ID NO: 7 |
| AAVrh.10 | 1247 | WO2017070516A1 SEQ ID NO: 14 |
| AAV2tYF | 1248 | WO2017070491A1 SEQ ID NO: 1 |
| AAV-SPK | 1249 | WO2017075619A1 SEQ ID NO: 28 |
| AAV2.5 | 1250 | US20170128528A1 SEQ ID NO: 13 |
| AAV1.1 | 1251 | US20170128528A1 SEQ ID NO: 15 |
| AAV6.1 | 1252 | US20170128528A1 SEQ ID NO: 17 |
| AAV6.3.1 | 1253 | US20170128528A1 SEQ ID NO: 18 |
| AAV2i8 | 1254 | US20170128528A1 SEQ ID NO: 28 |
| AAV2i8 | 1255 | US20170128528A1 SEQ ID NO: 29 |
| ttAAV | 1256 | US20170128528A1 SEQ ID NO: 30 |
| ttAAV-S312N | 1257 | US20170128528A1 SEQ ID NO: 32 |
| ttAAV-S312N | 1258 | US20170128528A1 SEQ ID NO: 33 |
| AAV6 (Y705, Y731, | 1259 | WO2016134337A1 SEQ ID NO: 24 |
| and T492) | | |
| AAV2 | 1260 | WO2016134375A1 SEQ ID NO: 9 |
| AAV2 | 1261 | WO2016134375A1 SEQ ID NO: 10 |

In some embodiments, the AAV serotype may be, or may have a sequence as described in International Patent Publication WO2015038958, the contents of which are herein incorporated by reference in their entirety, such as, but not limited to, AAV9 (SEQ ID NO: 2 and 11 of WO2015038958 or SEQ ID NO: 137 and 138 respectively herein), PHP.B (SEQ ID NO: 8 and 9 of WO2015038958, herein SEQ ID NO: 5 and 6), G2B-13 (SEQ ID NO: 12 of WO2015038958, herein SEQ ID NO: 7), G2B-26 (SEQ ID NO: 13 of WO2015038958, herein SEQ ID NO: 5), TH1.1-32 (SEQ ID NO: 14 of WO2015038958, herein SEQ ID NO: 8), TH1.1-35 (SEQ ID NO: 15 of WO2015038958, herein SEQ ID NO: 9) or variants thereof. Further, any of the targeting peptides or amino acid inserts described in WO2015038958, may be inserted into any parent AAV serotype, such as, but not limited to, AAV9 (SEQ ID NO: 137 for the DNA sequence and SEQ ID NO: 138 for the amino acid sequence). In another embodiment, the amino acid insert is inserted between amino acids 588-589 of the parent AAV sequence. The amino acid insert may be, but is not limited to, any of the following amino acid sequences, TLAVPFK (herein SEQ ID NO: 1262), KFPVALT (SEQ ID NO: 1263), LAVPFK (SEQ ID NO: 1264), AVPFK (SEQ ID NO: 1265), VPFK (SEQ ID NO: 1266), TLAVPF (SEQ ID NO: 1267), TLAVP (SEQ ID NO: 1268), TLAV (SEQ ID NO: 1269), SVSKPFL (SEQ ID NO: 1270), FTLTTPK (SEQ ID NO: 1271), MNATKNV (SEQ ID NO: 1272), QSSQTPR (SEQ ID NO: 1273), ILGTGTS (SEQ ID NO: 1274), TRTNPEA (SEQ ID NO: 1275), NGGTSSS (SEQ ID NO: 1276), or YTLSQGW (SEQ ID NO: 1277). Non-limiting examples of nucleotide sequences that may encode the amino acid inserts include the following, SEQ ID NO: 1278, SEQ ID NO: 1279, SEQ ID NO: 1280, SEQ ID NO: 1281, SEQ ID NO: 1282, SEQ ID NO: 1283, SEQ ID NO: 1284, SEQ ID NO: 1285, SEQ ID NO: 1286, or SEQ ID NO: 1287.

In some embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 11), PHP.N (SEQ ID NO: 46 of WO2017100671, herein SEQ ID NO: 4), PHP.S (SEQ ID NO: 47 of WO2017100671, herein SEQ ID NO: 10), or variants thereof. Further, any of the targeting peptides or amino acid inserts described in WO2017100671 may be inserted into any parent AAV serotype, such as, but not limited to, AAV9. In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, AQTLAVPFKAQ (SEQ ID NO: 1288), AQSVSKPFLAQ (SEQ ID NO: 1289), AQFTLTTPKAQ (SEQ ID NO: 1290), DGTLAVPFKAQ (SEQ ID NO: 1291), ESTLAVPFKAQ (SEQ ID NO: 1292), GGTLAVPFKAQ (SEQ ID NO: 1293), AQTLATPFKAQ (SEQ ID NO: 1294), ATTLATPFKAQ (SEQ ID NO: 1295), DGTLATPFKAQ (SEQ ID NO: 1296), GGTLATPFKAQ (SEQ ID NO: 1297), SGSLAVPFKAQ (SEQ ID NO: 1298), AQTLAQPFKAQ (SEQ ID NO: 1299), AQTLQQPFKAQ (SEQ ID NO: 1300), AQTLSNPFKAQ (SEQ ID NO: 1301), AQTLAVPFSNP (SEQ ID NO: 1302), QGTLAVPFKAQ (SEQ ID NO: 1303), NQTLAVPFKAQ (SEQ ID NO: 1304), EGSLAVPFKAQ (SEQ ID NO: 1305), SGNLAVPFKAQ (SEQ ID NO: 1306), EGTLAVPFKAQ (SEQ ID NO: 1307), DSTLAVPFKAQ (SEQ ID NO: 1308), AVTLAVPFKAQ (SEQ ID NO: 1309), AQTLSTPFKAQ (SEQ ID NO: 1310), AQTLPQPFKAQ (SEQ ID NO: 1311), AQTLSQPFKAQ (SEQ ID NO: 1312), AQTLQLPFKAQ (SEQ ID NO: 1313), AQTLTMPFKAQ (SEQ ID NO: 1314), AQTLTTPFKAQ (SEQ ID NO: 1315), AQYTLSQGWAQ (SEQ ID NO: 1316), AQMNATKNVAQ (SEQ ID NO: 1317), AQVSGGHHSAQ (SEQ ID NO: 1318), AQTLTAPFKAQ (SEQ ID NO: 1319), AQTLSKPFKAQ (SEQ ID NO: 1320), QAVRTSL (SEQ ID NO: 1321), YTLSQGW (SEQ ID NO: 1277), LAKERLS (SEQ ID NO: 1322), TLAVPFK (SEQ ID NO: 1262), SVSKPFL (SEQ ID NO: 1270), FTLTTPK (SEQ ID NO: 1271), MNSTKNV (SEQ ID NO: 1323), VSGGHHS (SEQ ID NO: 1324), SAQTLAVPFKAQAQ (SEQ ID NO: 1325), SXXXLAVPFKAQAQ (wherein X may be any amino acid; SEQ ID NO: 1326), SAQXXXVPFKAQAQ (wherein X may be any amino acid; SEQ ID NO: 1327), SAQTLXXXFKAQAQ (wherein X may be any amino acid; SEQ ID NO: 1328), SAQTLAVXXXAQAQ (wherein X may be any amino acid; SEQ ID NO: 1329), SAQTLAVPFXXXAQ (wherein X may be any amino acid; SEQ ID NO: 1330), TNHQSAQ (SEQ ID NO: 1331), AQAQTGW (SEQ ID NO: 1332), DGTLATPFK (SEQ ID NO: 1333), DGTLATPFKXX (wherein X may be any amino acid; SEQ ID NO: 1334), LAVPFKAQ (SEQ ID NO: 1335), VPFKAQ (SEQ ID NO: 1336), FKAQ (SEQ ID NO: 1337), AQTLAV (SEQ ID NO: 1338), AQTLAVPF (SEQ ID NO: 1339), QAVR (SEQ ID NO: 1340), AVRT (SEQ ID NO: 1341), VRTS (SEQ ID NO: 1342), RTSL (SEQ ID NO: 1343), QAVRT (SEQ ID NO: 1344), AVRTS (SEQ ID NO: 1345), VRTSL (SEQ ID NO: 1346), QAVRTS (SEQ ID NO: 1347), or AVRTSL (SEQ ID NO: 1348).Non-limiting examples of nucleotide sequences that may encode the amino acid inserts include the following, SEQ ID NO: 1349, SEQ ID NO: 1350, SEQ ID NO: 1351, SEQ ID NO: 1352, SEQ ID NO: 1353, SEQ ID NO: 1354, SEQ ID NO: 1355, SEQ ID NO: 1356, SEQ ID NO: 1357, SEQ ID NO: 1358 (wherein N may be A, C, T, or G), SEQ ID NO: 1359 (wherein N may be A, C, T, or G), SEQ ID NO: 1360 (wherein N may be A, C, T, or G), SEQ ID NO: 1361 (wherein N may be A, C, T, or G); herein SEQ ID NO: 1362 (wherein N may be A, C, T, or G), SEQ ID NO: 1279, SEQ ID NO: 1280, SEQ ID NO: 1281, SEQ ID NO: 1287, or SEQ ID NO: 1363.

In some embodiments, the AAV serotype may be, or may have a sequence as described in U.S. Pat. No. 9,624,274, the contents of which are herein incorporated by reference in their entirety, such as, but not limited to, AAV1 (SEQ ID NO: 181 of U.S. Pat. No. 9,624,274), AAV6 (SEQ ID NO: 182 of U.S. Pat. No. 9,624,274), AAV2 (SEQ ID NO: 183 of U.S. Pat. No. 9,624,274), AAV3b (SEQ ID NO: 184 of U.S. Pat. No. 9,624,274), AAV7 (SEQ ID NO: 185 of U.S. Pat. No. 9,624,274), AAV8 (SEQ ID NO: 186 of U.S. Pat. No. 9,624,274), AAV10 (SEQ ID NO: 187 of U.S. Pat. No. 9,624,274), AAV4 (SEQ ID NO: 188 of U.S. Pat. No. 9,624,274), AAV11 (SEQ ID NO: 189 of U.S. Pat. No. 9,624,274), bAAV (SEQ ID NO: 190 of U.S. Pat. No. 9,624,274), AAVS (SEQ ID NO: 191 of U.S. Pat. No. 9,624,274), GPV (SEQ ID NO: 192 of U.S. Pat. No. 9,624,274; herein SEQ ID NO: 879), B19 (SEQ ID NO: 193 of U.S. Pat. No. 9,624,274; herein SEQ ID NO: 880), MVM (SEQ ID NO: 194 of U.S. Pat. No. 9,624,274; herein SEQ ID NO: 881), FPV (SEQ ID NO: 195 of U.S. Pat. No. 9,624,274; herein SEQ ID NO: 882), CPV (SEQ ID NO: 196 of U.S. Pat. No. 9,624,274; herein SEQ ID NO: 883) or variants thereof. Further, any of the structural protein inserts described in U.S. Pat. No. 9,624,274, may be inserted into, but not limited to, 1-453 and 1-587 of any parent AAV serotype, such as, but not limited to, AAV2 (SEQ ID NO: 183 of U.S. Pat. No. 9,624,274). The amino acid insert may be, but is not limited to, any of the following amino acid sequences, VNLTWSRASG (SEQ ID NO: 1364), EFCINHRGYWVCGD (SEQ ID NO: 1365), EDGQVMDVDLS (SEQ ID NO: 1366), EKQRNGTLT (SEQ ID NO: 1367), TYQCRVTHPHLPRALMR (SEQ ID NO: 1368), RHSTTQPRKTKGSG (SEQ ID NO: 1369), DSNPRGVSAYLSR (SEQ ID NO: 1370), TITCLWDLAPSK (SEQ ID NO: 1371), KTKGSGFFVF (SEQ ID NO: 1372), THPHLPRALMRS (SEQ ID NO: 1373), GETYQCRVTHPHLPRALMRSTTK (SEQ ID NO: 1374), LPRALMRS (SEQ ID NO: 1375), INHRGYWV (SEQ ID NO: 1376), CDAGSVRTNAPD (SEQ ID NO: 1377), AKAVSNLTESRSESLQS (SEQ ID NO: 1378), SLTGDEFKKVLET (SEQ ID NO: 1379), REAVAYRFEED (SEQ ID NO: 1380), INPEIITLDG (SEQ ID NO: 1381), DISVTGAPVITATYL (SEQ ID NO: 1382), DISVTGAPVITA (SEQ ID NO: 1383), PKTVSNLTESSSESVQS (SEQ ID NO: 1384), SLMGDEFKAVLET (SEQ ID NO: 1385), QHSVAYTFEED (SEQ ID NO: 1386), INPEIITRDG (SEQ ID NO: 1387), DISLTGDPVITASYL (SEQ ID NO: 1388), DISLTGDPVITA (SEQ ID NO: 1389), DQSIDFEIDSA (SEQ ID NO: 1390), KNVSEDLPLPTFSPTLLGDS (SEQ ID NO: 1391), KNVSEDLPLPT (SEQ ID NO: 1392), CDSGRVRTDAPD (SEQ ID NO: 1393), FPEHLLVDFLQSLS (SEQ ID NO: 1394), DAEFRHDSG (SEQ ID NO: 1395), HYAAAQWDFGNTMCQL (SEQ ID NO: 1396), YAAQWDFGNTMCQ (SEQ ID NO: 1397), RSQKEGLHYT (SEQ ID NO: 1398), SSRTPSDKPVAHWANPQAE (SEQ ID NO: 1399), SRTPSDKPVAHWANP (SEQ ID NO: 1400), SSRTPSDKP (SEQ ID NO: 1401), NADGNVDYHMNSVP (SEQ ID NO: 1402), DGNVDYHMNSV (SEQ ID NO: 1403), RSFKEFLQSSLRALRQ (SEQ ID NO: 1404); FKEFLQSSLRA (SEQ ID NO: 1405), or QMWAPQWGPD (SEQ ID NO: 1406).

In some embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not be limited to, the amino acid sequence RGNRQA (SEQ ID NO: 1407), SSSTDP (SEQ ID NO: 1408), SSNTAP (SEQ ID NO: 1409), SNSNLP (herein SEQ ID NO: 1410), SSTTAP (SEQ ID NO: 1411), AANTAA (SEQ ID NO: 1412), QQNTAP (SEQ ID NO: 1413), SAQAQA (SEQ ID NO: 1414), QANTGP (SEQ ID NO: 1415), NATTAP (SEQ ID NO: 1416), SSTAGP (SEQ ID NO: 1417), QQNTAA (SEQ ID NO: 1418), PSTAGP (SEQ ID NO: 1419), NQNTAP (SEQ ID NO: 1420), QAANAP (SEQ ID NO: 1421), SIVGLP (SEQ ID NO: 1422), AASTAA (SEQ ID NO: 1423), SQNTTA (SEQ ID NO: 1424), QQDTAP (SEQ ID NO: 1425), QTNTGP (SEQ ID NO: 1426), QTNGAP (SEQ ID NO: 1427), QQNAAP (SEQ ID NO: 1428), or AANTQA (SEQ ID NO: 1429). In some embodiments, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences, NGRAHA (SEQ ID NO: 1430), QPEHSST (SEQ ID NO: 1431), VNTANST (SEQ ID NO: 1432), HGPMQKS (SEQ ID NO: 1433), PHKPPLA (SEQ ID NO: 1434), IKNNEMW (SEQ ID NO: 1435), RNLDTPM (SEQ ID NO: 1436), VDSHRQS (SEQ ID NO: 1437), YDSKTKT (SEQ ID NO: 1438), SQLPHQK (SEQ ID NO: 1439), STMQQNT (SEQ ID NO: 1440), TERYMTQ (SEQ ID NO: 1441), DASLSTS (SEQ ID NO: 1442), DLPNKKT (SEQ ID NO: 1443), DLTAARL (SEQ ID NO: 1444), EPHQFNY (SEQ ID NO: 1445), EPQSNHT (SEQ ID NO: 1446), MSSWPSQ (SEQ ID NO: 1447), NPKHNAT (SEQ ID NO: 1448), PDGMRTT (SEQ ID NO: 1449), PNNNKTT (SEQ ID NO: 1450), QSTTHDS (SEQ ID NO: 1451), TGSKQKQ (SEQ ID NO: 1452), SLKHQAL (SEQ ID NO: 1453), SPIDGEQ (SEQ ID NO: 1454), WIFPWIQL (SEQ ID NO: 1455), CDCRGDCFC (SEQ ID NO: 1456), CNGRC (SEQ ID NO: 1457), CPRECES (SEQ ID NO: 1458), CTTHWGFTLC (SEQ ID NO: 1459), CGRRAGGSC (SEQ ID NO: 1460), CKGGRAKDC (SEQ ID NO: 1461), CVPELGHEC (SEQ ID NO: 1462), CRRETAWAK (SEQ ID NO: 1463), VSWFSHRYSPFAVS (SEQ ID NO: 1464), GYRDGYAGPILYN (SEQ ID NO: 1465), XXXYXXX (SEQ ID NO: 1466), YXNW (SEQ ID NO: 1467), RPLPPLP (SEQ ID NO: 1468), APPLPPR (SEQ ID NO: 1469), DVFYPYPYASGS (SEQ ID NO: 1470), MYWYPY (SEQ ID NO: 1471), DITWDQLWDLMK (SEQ ID NO: 1472), CWDDXWLC (SEQ ID NO: 1473), EWCEYLGGYLRCYA (SEQ ID NO: 1474), YXCXXGPXTWXCXP (SEQ ID NO: 1475), IEGPTLRQWLAARA (SEQ ID NO: 1476), LWXXX (SEQ ID NO: 1477), XFXXYLW (SEQ ID NO: 1478), SSIISHFRWGLCD (SEQ ID NO: 1479), MSRPACPPNDKYE (SEQ ID NO: 1480), CLRSGRGC (SEQ ID NO: 1481), CHWMFSPWC (SEQ ID NO: 1482), WXXF (SEQ ID NO: 1483), CSSRLDAC (SEQ ID NO: 1484), CLPVASC (SEQ ID NO: 1485), CGFECVRQCPERC (SEQ ID NO: 1486), CVALCREACGEGC (SEQ ID NO: 1487), SWCEPGWCR (SEQ ID NO: 1488), YSGKWGW (SEQ ID NO: 1489), GLSGGRS (SEQ ID NO: 1490), LMLPRAD (SEQ ID NO: 1491), CSCFRDVCC (SEQ ID NO: 1492), CRDVVSVIC (SEQ ID NO: 1493), MARSGL (SEQ ID NO: 1494), MARAKE (SEQ ID NO: 1495), MSRTMS (SEQ ID NO: 1496, KCCYSL (SEQ ID NO: 1497), MYWGDSHWLQYWYE (SEQ ID NO: 1498), MQLPLAT (SEQ ID NO: 1499), EWLS (SEQ ID NO: 1500), SNEW (SEQ ID NO: 1501), TNYL (SEQ ID NO: 1502), WDLAWMFRLPVG (SEQ ID NO: 1503), CTVALPGGYVRVC (SEQ ID NO: 1504), CVAYCIEHHCWTC (SEQ ID NO: 1505), CVFAHNYDYLVC (SEQ ID NO: 1506), CVFTSNYAFC (SEQ ID NO: 1507), VHSPNKK (SEQ ID NO: 1508), CRGDGWC (SEQ ID NO: 1509), XRGCDX (SEQ ID NO: 1510), PXXX (SEQ ID NO: 1511), SGKGPRQITAL (SEQ ID NO: 1512), AAAAAAAAAXXXXX (SEQ ID NO: 1513), VYMSPF (SEQ ID NO: 1514), ATWLPPR (SEQ ID NO: 1515), HTMYYHHYQHHL (SEQ ID NO: 1516), SEVGCRAGPLQWLCEKYFG (SEQ ID NO: 1517), CGLLPVGRPDRNVWRWLC (SEQ ID NO: 1518), CKGQCDRFKGLPWEC (SEQ ID NO: 1519), SGRSA (SEQ ID NO: 1520), WGFP (SEQ ID NO: 1521), AEPMPHSLNFSQYLWYT (SEQ ID NO: 1522), WAYXSP (SEQ ID NO: 1523), IELLQAR (SEQ ID NO: 1524), AYTKCSRQWRTCMTTH (SEQ ID NO: 1525), PQNSKIPGPTFLDPH (SEQ ID NO: 1526), SMEPALPDWWWKMFK (SEQ ID NO: 1527), ANTPCGPYTHDCPVKR (SEQ ID NO: 1528), TACHQHVRMVRP (SEQ ID NO: 1529), VPWMEPAYQRFL (SEQ ID NO: 1530), DPRATPGS (SEQ ID NO: 1531), FRPNRAQDYNTN (SEQ ID NO: 1532), CTKNSYLMC (SEQ ID NO: 1533), CXXTXXXGXGC (SEQ ID NO: 1534), CPIEDRPMC (SEQ ID NO: 1535), HEWSYLAPYPWF (SEQ ID NO: 1536), MCPKHPLGC (SEQ ID NO: 1537), RMWPSSTVNLSAGRR (SEQ ID NO: 1538), SAKTAVSQRVWLPSHRGGEP (SEQ ID NO: 1539), KSREHVNNSACPSKRITAAL (SEQ ID NO: 1540), EGFR (SEQ ID NO: 1541), AGLGVR (SEQ ID NO: 1542), GTRQGHTMRLGVSDG (SEQ ID NO: 1543), IAGLATPGWSHWLAL (SEQ ID NO: 1544), SMSIARL (SEQ ID NO: 1545), HTFEPGV (SEQ ID NO: 1546), NTSLKRISNKRIRRK (SEQ ID NO: 1547), LRIKRKRRKRKKTRK (SEQ ID NO: 1548), GGG, GFS, LWS, EGG, LLV, LSP, LBS, AGG, GRR, GGH and GTV.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 1549) or variants thereof, wherein the specific site is at least one site selected from sites R447, G453, S578, N587, N587+1, S662 of VP1 or fragment thereof.

Further, any of the mutated sequences described in US 20160369298, may be or may have, but not limited to, any of the following sequences SDSGASN (SEQ ID NO: 1550), SPSGASN (SEQ ID NO: 1551), SHSGASN (SEQ ID NO: 1552), SRSGASN (SEQ ID NO: 1553), SKSGASN (SEQ ID NO: 1554), SNSGASN (SEQ ID NO: 1555), SGSGASN (SEQ ID NO: 1556), SASGASN (SEQ ID NO: 1557), SESGTSN (SEQ ID NO: 1558), STTGGSN (SEQ ID NO: 1559), SSAGSTN (SEQ ID NO: 1560), NNDSQA (SEQ ID NO: 1561), NNRNQA (SEQ ID NO: 1562), NNNKQA (SEQ ID NO: 1563), NAKRQA (SEQ ID NO: 1564), NDEHQA (SEQ ID NO: 1565), NTSQKA (SEQ ID NO: 1566), YYLSRTNTPSGTDTQSRLVFSQAGA (SEQ ID NO: 1567), YYLSRTNTDSGTETQSGLDFSQAGA (SEQ ID NO: 1568), YYLSRTNTESGTPTQSALEFSQAGA (SEQ ID NO: 1569), YYLSRTNTHSGTHTQSPLHFSQAGA (SEQ ID NO: 1570), YYLSRTNTSSGTITISHLIFSQAGA (SEQ ID NO: 1571), YYLSRTNTRSGIMTKSSLMFSQAGA (SEQ ID NO: 1572), YYLSRTNTKSGRKTLSNLSFSQAGA (SEQ ID NO: 1573), YYLSRTNDGSGPVTPSKLRFSQRGA (SEQ ID NO: 1574), YYLSRTNAASGHATHSDLKFSQPGA (SEQ ID NO: 1575), YYLSRTNGQAGSLTMSELGFSQVGA (SEQ ID NO: 1576), YYLSRTNSTGGNQTTSQLLFSQLSA (SEQ ID NO: 1577), YFLSRTNNNTGLNTNSTLNFSQGRA (SEQ ID NO: 1578), SKTGADNNNSEYSWTG (SEQ ID NO: 1579), SKTDADNNNSEYSWTG (SEQ ID NO: 1580), SKTEADNNNSEYSWTG (SEQ ID NO: 1581), SKTPADNNNSEYSWTG (SEQ ID NO: 1582), SKTHADNNNSEYSWTG (SEQ ID NO: 1583), SKTQADNNNSEYSWTG (SEQ ID NO: 1584), SKTIADNNNSEYSWTG (SEQ ID NO: 1585), SKTMADNNNSEYSWTG (SEQ ID NO: 1586), SKTRADNNNSEYSWTG (SEQ ID NO: 1587), SKTNADNNNSEYSWTG (SEQ ID NO: 1588), SKTVGRNNNSEYSWTG (SEQ ID NO: 1589), SKTADRNNNSEYSWTG (SEQ ID NO: 1590), SKKLSQNNNSKYSWQG (SEQ ID NO: 1591), SKPTTGNNNSDYSWPG (SEQ ID NO: 1592), STQKNENNNSNYSWPG (SEQ ID NO: 1593), HKDDEGKF (SEQ ID NO: 1594), HKDDNRKF (SEQ ID NO: 1595), HKDDTNKF (SEQ ID NO: 1596), HEDSDKNF (SEQ ID NO: 1597), HRDGADSF (SEQ ID NO: 1598), HGDNKSRF (SEQ ID NO: 1599), KQGSEKTNVDFEEV (SEQ ID NO: 1600), KQGSEKTNVDSEEV (SEQ ID NO: 1601), KQGSEKTNVDVEEV (SEQ ID NO: 1602), KQGSDKTNVDDAGV (SEQ ID NO: 1603), KQGSSKTNVDPREV (SEQ ID NO: 1604), KQGSRKTNVDHKQV (SEQ ID NO: 1605), KQGSKGGNVDTNRV (SEQ ID NO: 1606), KQGSGEANVDNGDV (SEQ ID NO: 1607), KQDAAADNIDYDHV (SEQ ID NO: 1608), KQSGTRSNAAASSV (SEQ ID NO: 1609), KENTNTNDTELTNV (SEQ ID NO: 1610), QRGNNVAATADVNT (SEQ ID NO: 1611), QRGNNEAATADVNT (SEQ ID NO: 1612), QRGNNPAATADVNT (SEQ ID NO: 1613), QRGNNHAATADVNT (SEQ ID NO: 1614), QEENNIAATPGVNT (SEQ ID NO: 1615), QPPNNMAATHEVNT (SEQ ID NO: 1616), QHHNNSAATTIVNT (SEQ ID NO: 1617), QTTNNRAAFNMVET (SEQ ID NO: 1618), QKKNNNAASKKVAT (SEQ ID NO: 1619), QGGNNKAADDAVKT (SEQ ID NO: 1620), QAAKGGAADDAVKT (SEQ ID NO: 1621), QDDRAAAANESVDT (SEQ ID NO: 1622), QQQHDDAAYQRVHT (SEQ ID NO: 1623), QSSSSLAAVSTVQT (SEQ ID NO: 1624), QNNQTTAAIRNVTT (SEQ ID NO: 1625), NYNKKSDNVDFT (SEQ ID NO: 1626), NYNKKSENVDFT (SEQ ID NO: 1627), NYNKKSLNVDFT (SEQ ID NO: 1628), NYNKKSPNVDFT (SEQ ID NO: 1629), NYSKKSHCVDFT (SEQ ID NO: 1630), NYRKTIYVDFT (SEQ ID NO: 1631), NYKEKKDVHFT (SEQ ID NO: 1632), NYGHRAIVQFT (SEQ ID NO: 1633), NYANHQFVVCT (SEQ ID NO: 1634), NYDDDPTGVLLT (SEQ ID NO: 1635), NYDDPTGVLLT (SEQ ID NO: 1636), NFEQQNSVEWT (SEQ ID NO: 1637), SQSGASN (SEQ ID NO: 1638), NNGSQA (SEQ ID NO: 1639), YYLSRTNTPSGTTTWSRLQFSQAGA (SEQ ID NO: 1640), SKTSADNNNSEYSWTG (SEQ ID NO: 1641), HKDDEEKF (SEQ ID NO: 1642), KQGSEKTNVDIEEV (SEQ ID NO: 1643), QRGNNQAATADVNT (SEQ ID NO: 1644), NYNKKSVNVDFT (SEQ ID NO: 1645), SQSGASNYNTPSGTTTQSRLQFSTSADNNNSEYSWTGATKYH (SEQ ID NO: 1646), SAS GASNFNSEGGSLTQSSLGFSTDGENNNSDFSWTGATKYH (SEQ ID NO: 1647), SQSGASNYNTPSGTTTQSRLQFSTDGENNNSDFSWTGATKYH (SEQ ID NO: 1648), SASGASNYNTPSGTTTQSRLQFSTSADNNNSEFSWPGATTYH (SEQ ID NO: 1649), SQSGASNFNSEGGSLTQSSLGFSTDGENNNSDFSWTGATKYH (SEQ ID NO: 1650), SASGASNYNTPSGSLTQSSLGFSTDGENNNSDFSWTGATKYH (SEQ ID NO: 1651), SQSGASNYNTPSGTTTQSRLQFSTSADNNNSDFSWTGATKYH (SEQ ID NO: 1652), SGAGASNFNSEGGSLTQSSLGFSTDGENNNSDFSWTGATKYH (SEQ ID NO: 1653), SGAGASN (SEQ ID NO: 1654), NSEGGSLTQSSLGFS (SEQ ID NO: 1655), TDGENNNSDFS (SEQ ID NO: 1656), SEFSWPGATT (SEQ ID NO: 1657), TSADNNNSDFSWT (SEQ ID NO: 1658), SQSGASNY (SEQ ID NO: 1659), NTPSGTTTQSRLQFS (SEQ ID NO: 1660), TSADNNNSEYSWTGATKYH (SEQ ID NO: 1661), SASGASNF (SEQ ID NO: 1662), TDGENNNSDFSWTGATKYH (SEQ ID NO: 1663), SASGASNY (SEQ ID NO: 1664), TSADNNNSEFSWPGATTYH (SEQ ID NO: 1665), NTPSGSLTQSSLGFS (SEQ ID NO: 1666), TSADNNNSDFSWTGATKYH (SEQ ID NO: 1667), SGAGASNF (SEQ ID NO: 1668), CTCCAGVVSVVSMRSRVCVNSGCAGCTDHCVVSRNSGTCVMSACACAA (SEQ ID NO: 1669), CTCCAGAGAGGCAACAGACAAGCAGCTACCGCAGATGTCAACACACAA (SEQ ID NO: 1670), SAAGASN (SEQ ID NO: 1671), YFLSRTNTESGSTTQSTLRFSQAG (SEQ ID NO: 1672), SKTSADNNNSDFS (SEQ ID NO: 1673), KQGSEKTDVDIDKV (SEQ ID NO: 1674), STAGASN (SEQ ID NO: 1675), YFLSRTNTTSGIETQSTLRFSQAG (SEQ ID NO: 1676), SKTDGENNNSDFS (SEQ ID NO: 1677), KQGAAADDVEIDGV (SEQ ID NO: 1678), SEAGASN (SEQ ID NO: 1679), YYLSRTNTPSGTTTQSRLQFSQAG (SEQ ID NO: 1680), SKTSADNNNSEYS (SEQ ID NO: 1681), KQGSEKTNVDIEKV (SEQ ID NO: 1682), YFLSRTNDASGSDTKSTLLFSQAG (SEQ ID NO: 1683), STTPSENNNSEYS (SEQ ID NO: 1684), SAAGATN (SEQ ID NO: 1685), YFLSRTNGEAGSATLSELRFSQAG (SEQ ID NO: 1686), HGDDADRF (SEQ ID NO: 1687), KQGAEKSDVEVDRV (SEQ ID NO: 1688), KQDSGGDNIDIDQV (SEQ ID NO: 1689), SDAGASN (SEQ ID NO: 1690), YFLSRTNTEGGHDTQSTLRFSQAG (SEQ ID NO: 1691), KEDGGGSDVAIDEV (SEQ ID NO: 1692), SNAGASN (SEQ ID NO: 1693), and YFLSRTNGEAGSATLSELRFSQPG (SEQ ID NO: 1694). Non-limiting examples of nucleotide sequences that may encode the amino acid mutated sites include the following, SEQ ID NO: 1695, SEQ ID NO: 1696, SEQ ID NO: 1697, SEQ ID NO: 1698, SEQ ID NO: 1699, SEQ ID NO: 1700, SEQ ID NO: 1701, SEQ ID NO: 1702, SEQ ID NO: 1703, SEQ ID NO: 1704, SEQ ID NO: 1705, SEQ ID NO: 1706, SEQ ID NO: 1707, SEQ ID NO: 1708, SEQ ID NO: 1709, SEQ ID NO: 1710, AGCAGGAGCTCCTTGGCCTCAGCGTGCGAG (SEQ ID NO: 264 of US20160369298; herein SEQ ID NO: 1711), SEQ ID NO: 1712, SEQ ID NO: 1713, SEQ ID NO: 1714, SEQ ID NO: 1715, SEQ ID NO: 1716, and SEQ ID NO: 1717.

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID NO:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID NO: 1718), or AAV9 (SEQ ID NO: 11 of WO2016134375; herein SEQ ID NO: 1719). In some embodiments, modifications, such as insertions are made in AAV2 proteins at P34-A35, T138-A139, A139-P140, G453-T454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of WO2016134375; herein SEQ ID NO: 1720), or GETRAPL (SEQ ID NO: 4 of WO2016134375; herein SEQ ID NO: 1721).

In some embodiments, the AAV serotype may be modified as described in the United States Publication US 20170145405 the contents of which are herein incorporated by reference in their entirety. AAV serotypes may include, modified AAV2 (e.g., modifications at Y444F, Y500F, Y730F and/or S662V), modified AAV3 (e.g., modifications at Y705F, Y731F and/or T492V), and modified AAV6 (e.g., modifications at S663V and/or T492V).

In some embodiments, the AAV serotype may be modified as described in the International Publication WO2017083722 the contents of which are herein incorporated by reference in their entirety. AAV serotypes may include, AAV1 (Y705+731F+T492V), AAV2 (Y444+500+730F+T491V), AAV3 (Y705+731F), AAV5, AAV 5(Y436+693+719F), AAV6 (VP3 variant Y705F/Y731F/T492V), AAV8 (Y733F), AAV9, AAV9 (VP3 variant Y731F), and AAV10 (Y733F).

In some embodiments, the AAV serotype may comprise, as described in International Patent Publication WO2017015102, the contents of which are herein incorporated by reference in their entirety, an engineered epitope comprising the amino acids SPAKFA (SEQ ID NO: 24 of WO2017015102; herein SEQ ID NO: 1722) or NKDKLN (SEQ ID NO:2 of WO2017015102; herein SEQ ID NO: 1723). The epitope may be inserted in the region of amino acids 665 to 670 based on the numbering of the VP1 capsid of AAV8 (SEQ ID NO: 3 of WO2017015102) and/or residues 664 to 668 of AAV3B (SEQ ID NO: 3).

In some embodiments, the AAV serotype may be, or may have a sequence as described in International Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, 716-722 of AAV1, in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV. The amino acid substitution may be, but is not limited to, any of the amino acid sequences described in WO2017058892. In some embodiments, the AAV may comprise an amino acid substitution at residues 256L, 258K, 259Q, 261S, 263A, 264S, 265T, 266G, 272H, 385S, 386Q, S472R, V473D, N500E 547S, 709A, 710N, 716D, 717N, 718N, 720L, A456T, Q457T, N458Q, K459S, T492S, K493A, S586R, S587G, S588N, T589R and/or 722T of AAV1 (SEQ ID NO: 1 of WO2017058892) in any combination, 244N, 246Q, 248R, 249E, 2501, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of AAV5 (SEQ ID NO:5 of WO2017058892) in any combination, 248R, 316V, 317Q, 318D, 319S, 443N, 530N, 531S, 532Q 533P, 534A, 535N, 540A, 541 T, 542Y, 543L, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 5891 of AAV6 (SEQ ID NO:6 of WO2017058892) in any combination, 457T, 459N, 496G, 499N, 500N, 589Q, 590N and/or 592A of AAV8 (SEQ ID NO: 8 of WO2017058892) in any combination, 451I, 452N, 453G, 454S, 455G, 456Q, 457N and/or 458Q of AAV9 (SEQ ID NO: 9 of WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764, the contents of which are herein incorporated by reference in their entirety. The sequences of amino acid may be, but not limited to, N—S—S, S—X—S, S—S—Y, N—X—S, N—S—Y, S—X—Y and N—X—Y, where N, X and Y are, but not limited to, independently non-serine, or non-threonine amino acids, wherein the AAV may be, but not limited to AAV1, AAV2, AAV3, AAV4, AAV5, AAV6, AAV7, AAV8, AAV9, AAV10, AAV11 and AAV12. In some embodiments, the AAV may include a deletion of at least one amino acid at positions 156, 157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

In some embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may include a peptide such as, but not limited to, PHP.B, PHP.B2, PHP.B3, PHP.A, PHP.S, G2A12, G2A15, G2A3, G2B4, and G2B5. In some embodiments, these AAV serotypes may be AAV9 (SEQ ID NO: 11 or 138) derivatives with a 7-amino acid insert between amino acids 588-589. Non-limiting examples of these 7-amino acid inserts include TLAVPFK (PHP.B; SEQ ID NO: 1262), SVSKPFL (PHP.B2; SEQ ID NO: 1270), FTLTTPK (PHP.B3; SEQ ID NO: 1271), YTLSQGW (PHP.A; SEQ ID NO: 1277), QAVRTSL (PHP.S; SEQ ID NO: 1321), LAKERLS (G2A3; SEQ ID NO: 1322), MNSTKNV (G2B4; SEQ ID NO: 1323), and/or VSGGHHS (G2B5; SEQ ID NO: 1324).

In some embodiments, the AAV serotype may be as described in Jackson et al (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (e.g., CBA or CMV).

In some embodiments, the AAV serotype is a serotype comprising the AAVPHP.N (PHP.N) peptide, or a variant thereof. In some embodiments the AAV serotype is a serotype comprising the AAVPHP.B (PHP.B) peptide, or a variant thereof. In some embodiments, the AAV serotype is a serotype comprising the AAVPHP.A (PHP.A) peptide, or a variant thereof. In some embodiments, the AAV serotype is a serotype comprising the PHP.S peptide, or a variant thereof. In some embodiments, the AAV serotype is a serotype comprising the PHP.B2 peptide, or a variant thereof. In some embodiments, the AAV serotype is a serotype comprising the PHP.B3 peptide, or a variant thereof. In some embodiments, the AAV serotype is a serotype comprising the G2B4 peptide, or a variant thereof. In some embodiments, the AAV serotype is a serotype comprising the G2B5 peptide, or a variant thereof. In some embodiments the AAV serotype is VOY101, or a variant thereof. In some embodiments, the AAV serotype is VOY201, or a variant thereof.

In some embodiments the AAV serotype of an AAV particle, e.g., an AAV particle for the vectorized delivery of a GBA protein described herein, is AAV9, or a variant thereof. In some embodiments, the AAV particle, e.g., a recombinant AAV particle described herein, comprises an AAV9 capsid protein. In some embodiments, the AAV9 capsid protein comprises the amino acid sequence of SEQ ID NO: 138. In some embodiments, the nucleic acid sequence encoding the AAV9 capsid protein comprises the nucleotide sequence of SEQ ID NO: 137. In some embodiments, the AAV9 capsid protein comprises an amino acid sequence at least 70% identical to SEQ ID NO: 138, such as, 70%, 75%, 80%, 85%, 90%, 91%, 92%, 93%, 94%, 95%, 96%, 97%, 98%, 99%, or greater than 99%. In some embodiments, the nucleic acid sequence encoding the AAV9 capsid protein comprises a nucleotide sequence at least 70% identical to SEQ ID NO: 137, such as, 70%, 75%, 80%, 85%, 90%, 91%, 92%, 93%, 94%, 95%, 96%, 97%, 98%, 99%, or greater than 99%.

In some embodiments, the capsid protein comprises the amino acid sequence of SEQ ID NO: 11 or an amino acid sequence substantially identical (e.g., having at least 70%, 75%, 80%, 85%, 90%, 92%, 95%, 97%, 98%, or 99% sequence identity) thereto. In some embodiments the capsid protein comprises an amino acid sequence comprising at least one, two, or three modifications but no more than 30, 20, or 10 modifications, e.g., substitutions, relative to the amino acid sequence of SEQ ID NO: 11, optionally provided that position 449 does not comprise K, e.g., is R.

In some embodiments, the capsid protein, comprises the amino acid sequence of SEQ ID NO: 1 or an amino acid sequence substantially identical (e.g., having at least 70%, 75%, 80%, 85%, 90%, 92%, 95%, 97%, 98%, or 99% sequence identity) thereto. In some embodiments the capsid protein comprises an amino acid sequence comprising at least one, two, or three modifications but no more than 30, 20, or 10 modifications, e.g., substitutions, relative to the amino acid sequence of SEQ ID NO: 1. In some embodiments, the capsid protein comprises an amino acid sequence encoded by the nucleotide sequence of SEQ ID NO: 2 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the capsid protein comprises the nucleotide sequence of SEQ ID NO: 2 or a sequence substantially identical (e.g., having at least 70%, 75%, 80%, 85%, 90%, 92%, 95%, 97%, 98%, or 99% sequence identity) thereto.

In some embodiments, the capsid protein, e.g., an AAV9 capsid protein, comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the capsid protein comprises an amino acid sequence comprising at least one, two, or three modifications but no more than 30, 20, or 10 modifications, e.g., substitutions, relative to the amino acid sequence of SEQ ID NO: 138. In some embodiments, the capsid protein comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the capsid protein comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the capsid protein comprises substitution at position K449, e.g., a K449R substitution, numbered according to SEQ ID NO: 138.

In some embodiments, the capsid protein comprises an insert comprising the amino acid sequence of TLAVPFK (SEQ ID NO: 1262). In some embodiments, the insert is present immediately subsequent to position 588, relative to a reference sequence numbered according to SEQ ID NO: 138. In some embodiments, the capsid protein comprises the amino acid substitutions of A587D and Q588G, numbered according to SEQ ID NO: 138.

In some embodiments, the capsid protein comprises the amino acid substitution of K449R, numbered according to SEQ ID NO: 138; and an insert comprising the amino acid sequence of TLAVPFK (SEQ ID NO: 1262), wherein the insert is present immediately subsequent to position 588, relative to a reference sequence numbered according to SEQ ID NO: 138.

In some embodiments, the capsid protein comprises the amino acid substitution of K449R, numbered according to SEQ ID NO: 138; an insert comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the capsid protein comprises an insert comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV serotype of the AAV particle, e.g., an AAV particle for the vectorized delivery of antibody molecule described herein (e.g., an anti-beta-amyloid antibody molecule), is an AAV9 K449R, or a variant thereof. In some embodiments, the AAV particle comprises an AAV9 K449 capsid protein. In some embodiments, the AAV9 K449R capsid protein comprises the amino acid sequence of SEQ ID NO: 11. In some embodiments, the AAV9 K449R capsid protein comprises an amino acid sequence at least 70% identical to SEQ ID NO: 11, such as, 70%, 75%, 80%, 85%, 90%, 91%, 92%, 93%, 94%, 95%, 96%, 97%, 98%, 99%, or greater than 99%.

In some embodiments, the AAV capsid of an AAV particle, e.g., an AAV particle for the vectorized delivery of a GBA protein described herein, allows for blood brain barrier penetration following intravenous administration. Non-limiting examples of such AAV capsids include AAV9, AAV9 K449R, VOY101, VOY201, or AAV capsids comprising a peptide insert such as, but not limited to, AAVPHP.N (PHP.N), AAVPHP.B (PHP.B), PHP.S, G2A3, G2B4, G2B5, G2A12, G2A15, PHP.B2, PHP.B3, AAV2.BR1, or AAVPHP.A (PHP.A).

In some embodiments, the AAV serotype is selected for use due to its tropism for cells of the central nervous system. In some embodiments, the cells of the central nervous system are neurons. In another embodiment, the cells of the central nervous system are astrocytes.

In some embodiments, the AAV serotype is selected for use due to its tropism for cells of the muscle(s).

In some embodiments, the initiation codon for translation of the AAV VP1 capsid protein may be CTG, TTG, or GTG as described in U.S. Pat. No. 8,163,543, the contents of which are herein incorporated by reference in their entirety. In some embodiments, the nucleotide sequence encoding the capsid protein, e.g., a VP1 capsid protein, comprises 3-20 mutations (e.g., substitutions), e.g., 3-15 mutations, 3-10 mutations, 3-5 mutations, 5-20 mutations, 5-15 mutations, 5-10 mutations, 10-20 mutations, 10-15 mutations, 15-20 mutations, 3 mutations, 5 mutations, 10 mutations, 12 mutations, 15 mutations, 18 mutations, or 20 mutations, relative to the nucleotide sequence of SEQ ID NO: 137.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific “SEQ ID NO:” (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met 1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1-) of the 736 amino acid AA1+sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1-), and combinations thereof (Met+/AA1+ and Met−/AA1-).

As a non-limiting example, an AAV capsid serotype can include VP1 (Met+/AA1+), VP1 (Met−/AA1-), or a combination of VP1 (Met+/AA1+) and VP1 (Met−/AA1-). An AAV capsid serotype can also include VP3 (Met+/AA1+), VP3 (Met−/AA1-), or a combination of VP3 (Met+/AA1+) and VP3 (Met−/AA1-); and can also include similar optional combinations of VP2 (Met+/AA1) and VP2 (Met−/AA1-).

### AAV Viral Genome

In some aspects, the AAV particle of the present disclosure serves as an expression vector comprising a viral genome which encodes a GCase protein. The viral genome can encode a GCase protein and an enhancement, e.g., prosaposin (PSAP) or sapsosin (Sap) polypeptide or functional variant thereof (e.g., a SapA protein or a SapC protein), a cell penetrating peptide (e.g., an ApoEII peptide, a TAT peptide, or an ApoB peptide), a lysosomal targeting sequence (LTS), or a combination thereof. In some embodiments, expression vectors are not limited to AAV and may be adenovirus, retrovirus, lentivirus, plasmid, vector, or any variant thereof.

In some embodiments, an AAV particle, e.g., an AAV particle for the vectorized delivery of anGBA protein described herein, comprises a viral genome, e.g., an AAV viral genome (e.g., a vector genome or AAV vector genome). In some embodiments, the viral genome, e.g., the AAV viral genome, further comprises an inverted terminal repeat (ITR) region, an enhancer, a promoter, an intron region, a Kozak sequence, an exon region, a nucleic acid encoding a transgene encoding a payload (e.g., a GBA protein described herein) with or without an enhancement element, a nucleotide sequence encoding a miR binding site (e.g., a miR183 binding site), a poly A signal region, or a combination thereof.

### Viral Genome Component: Inverted Terminal Repeats (ITRs)

In some embodiments, the viral genome may comprise at least one inverted terminal repeat (ITR) region. The AAV particles of the present disclosure comprise a viral genome with at least one ITR region and a payload region. In some embodiments, the viral genome has two ITRs. These two ITRs flank the payload region at the 5′ and 3′ ends. In some embodiments, the ITR functions as an origin of replication comprising a recognition site for replication. In some embodiments, the ITR comprises a sequence region which can be complementary and symmetrically arranged. In some embodiments, the ITR incorporated into a viral genome described herein may be comprised of a naturally occurring polynucleotide sequence or a recombinantly derived polynucleotide sequence.

The ITRs may be derived from the same serotype as the capsid, selected from any of the serotypes listed in Table 1, or a derivative thereof. The ITR may be of a different serotype than the capsid. In some embodiments, the AAV particle has more than one ITR. In a non-limiting example, the AAV particle has a viral genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the viral genome of the AAV particle are AAV2 ITRs.

Independently, each ITR may be about 100 to about 150 nucleotides in length. In some embodiments, the ITR comprises 100-180 nucleotides in length, e.g., about 100-115, about 100-120, about 100-130, about 100-140, about 100-150, about 100-160, about 100-170, about 100-180, about 110-120, about 110-130, about 110-140, about 110-150, about 110-160, about 110-170, about 110-180, about 120-130, about 120-140, about 120-150, about 120-160, about 120-170, about 120-180, about 130-140, about 130-150, about 130-160, about 130-170, about 130-180, about 140-150, about 140-160, about 140-170, about 140-180, about 150-160, about 150-170, about 150-180, about 160-170, about 160-180, or about 170-180 nucleotides in length. In some embodiments, the ITR comprises about 120-140 nucleotides in length, e.g., about 130 nucleotides in length. In some embodiments, the ITRs are 140-142 nucleotides in length, e.g., 141 nucleotides in length. In some embodiments, the ITR comprises 1205-135 nucleotides in length, e.g., 130 nucleotides in length. Non-limiting examples of ITR length are 102, 130, 140, 141, 142, 145 nucleotides in length, and those having at least 95% identity thereto.

In some embodiments, each ITR may be 141 nucleotides in length. In some embodiments, each ITR may be 130 nucleotides in length. In some embodiments, the AAV particles comprise two ITRs and one ITR is 141 nucleotides in length and the other ITR is 130 nucleotides in length.

In some embodiments, the ITR comprises the nucleotide sequence of any one of SEQ ID NOs: 1829, 1830, or 1862, or a nucleotide sequence substantially identical (e.g., at least 70%, 75%, 80%, 85%, 90%, 95%, 99% or 100% identical) to any of the aforesaid sequences. In some embodiments, the ITR comprises the nucleotide sequence of any of SEQ ID NOs: 1860, 1861, 1863, or 1864, or a nucleotide sequence having one, two, or three but no more than four modifications, e.g., substitutions, relative to SEQ ID NOs: 1860, 1861, 1863, or 1864.

### Viral Genome Component: Promoters and Expression Enhancers

In some embodiments, the payload region of the viral genome comprises at least one element to enhance the transgene target specificity and expression. See, e.g., Powell et al. Viral Expression Cassette Elements to Enhance Transgene Target Specificity and Expression in Gene Therapy, 2015; the contents of which are herein incorporated by reference in their entirety. Non-limiting examples of elements to enhance the transgene target specificity and expression include promoters, endogenous miRNAs, post-transcriptional regulatory elements (PREs), polyadenylation (PolyA) signal sequences, upstream enhancers (USEs), CMV enhancers, and introns.

In some embodiments, expression of the polypeptides in a target cell may be driven by a specific promoter, including but not limited to, a promoter that is species specific, inducible, tissue-specific, or cell cycle-specific (Parr et al., Nat. Med.3:1145-9 (1997); the contents of which are herein incorporated by reference in their entirety).

In some embodiments, the viral genome comprises a that is sufficient for expression, e.g., in a target cell, of a payload (e.g., a GBA protein) encoded by a transgene. In some embodiments, the promoter is deemed to be efficient when it drives expression of the polypeptide(s) encoded in the payload region of the viral genome of the AAV particle.

In some embodiments, the promoter is a promoter deemed to be efficient when it drives expression in the cell or tissue being targeted.

In some embodiments, the promoter drives expression of the GCase, GCase and SapA, or GCase and SapC protein(s) for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiments, the promoter drives expression of a polypeptide (e.g., a GCase polypeptide, a GCase polypeptide and a prosaposin (PSAP) polypeptide, a GCase polypeptide and a SapA polypeptide, a GCase polypeptide and a SapC polypeptide, a GCase polypeptide and a cell penetrating peptide (e.g., an ApoEII peptide, a TAT peptide, and/or a ApoB peptide), or a GCase polypeptide and a lysosomal targeting peptide) for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

In some embodiments, the viral genome comprises a promoter that results in expression in one or more, e.g., multiple, cells and/or tissues, e.g., a ubiquitous promoter. In some embodiments, a promoter which drives or promotes expression in most mammalian tissues includes, but is not limited to, human elongation factor 1α-subunit (EF1α), cytomegalovirus (CMV) immediate-early enhancer and/or promoter, chicken β-actin (CBA) and its derivative CAG, 0 glucuronidase (GUSB), and ubiquitin C (UBC). Tissue-specific expression elements can be used to restrict expression to certain cell types such as, but not limited to, CNS-specific promoters, B cell promoters, monocyte promoters, leukocyte promoters, macrophage promoters, pancreatic acinar cell promoters, endothelial cell promoters, lung tissue promoters, astrocyte promoters, or various specific nervous system cell- or tissue-type promoters which can be used to restrict expression to neurons, astrocytes, or oligodendrocytes, for example.

In some embodiments, the viral genome comprises a nervous system specific promoter, e.g., a promoter that results in expression of a payload in a neuron, an astrocyte, and/or an oligodendrocyte. Non-limiting examples of tissue-specific expression elements for neurons include neuron-specific enolase (NSE), platelet-derived growth factor (PDGF), platelet-derived growth factor B-chain (PDGF-β), synapsin (Syn), synapsin 1 (Synl), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter. Prion promoter represents an additional tissue specific promoter useful for driving protein expression in CNS tissue (see Loftus, Stacie K., et al. Human molecular genetics 11.24 (2002): 3107-3114, the disclosure of which is incorporated by reference in its entirety).

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800, or 700-800 nucleotides.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 nucleotides. In some embodiments, the promoter is a combination of a 382 nucleotide CMV-enhancer sequence and a 260 nucleotide CBA-promoter sequence.

In some embodiments, the viral genome comprises a ubiquitous promoter. Non-limiting examples of ubiquitous promoters include CMV, CBA (including derivatives CAG, CB6, CBh, etc.), EF-1a, PGK, UBC, GUSB (hGBp), and UCOE (promoter of HNRPA2B1-CBX3). In some embodiments, the viral genome comprises an EF-1α promoter or EF-1α promoter variant.

In some embodiments, the promoter is a ubiquitous promoter as described in Yu et al. (Molecular Pain 2011, 7:63), Soderblom et al. (E. Neuro 2015), Gill et al., (Gene Therapy 2001, Vol. 8, 1539-1546), and Husain et al. (Gene Therapy 2009), each of which are incorporated by reference in their entirety.

In some embodiments, the promoter is not cell specific.

In some embodiments, the promoter is a ubiquitin c (UBC) promoter. The UBC promoter may have a size of 300-350 nucleotides. As a non-limiting example, the UBC promoter is 332 nucleotides. In some embodiments, the promoter is a β-glucuronidase (GUSB) promoter. The GUSB promoter may have a size of 350-400 nucleotides. As a non-limiting example, the GUSB promoter is 378 nucleotides. In some embodiments, the promoter is a neurofilament light (NFL) promoter. The NFL promoter may have a size of 600-700 nucleotides. As a non-limiting example, the NFL promoter is 650 nucleotides. In some embodiments, the promoter is a neurofilament heavy (NFH) promoter. The NFH promoter may have a size of 900-950 nucleotides. As a non-limiting example, the NFH promoter is 920 nucleotides. In some embodiments, the promoter is a scn8a promoter. The scn8a promoter may have a size of 450-500 nucleotides. As a non-limiting example, the scn8a promoter is 470 nucleotides.

In some embodiments, the promoter is a phosphoglycerate kinase 1 (PGK) promoter.

In some embodiments, the promoter is a chicken β-actin (CBA) promoter, or a functional variant thereof.

In some embodiments, the promoter is a CB6 promoter, or a functional variant thereof.

In some embodiments, the promoter is a CB promoter, or a functional variant thereof. In some embodiments, the promoter is a minimal CB promoter, or a functional variant thereof.

In some embodiments, the promoter is a CBA promoter, or functional variant thereof. In some embodiments, the promoter is a minimal CBA promoter, or functional variant thereof.

In some embodiments, the promoter is a cytomegalovirus (CMV) promoter, or a functional variant thereof.

In some embodiments, the promoter is a CAG promoter, or a functional variant thereof.

In some embodiments, the promoter is an EF1α promoter or functional variant thereof.

In some embodiments, the promoter is a GFAP promoter (as described, for example, in Zhang, Min, et al. Journal of neuroscience research 86.13 (2008): 2848-2856, the disclosure of which is incorporated by reference in its entirety) to drive expression of a GCase polypeptide, or a GCase polypeptide and an enhancement element (e.g., GCase and SapA, or GCase and SapC protein expression) in astrocytes.

In some embodiments, the promoter is a synapsin promoter, or a functional variant thereof.

In some embodiments, the promoter is an RNA pol III promoter. As a non-limiting example, the RNA pol III promoter is U6. As a non-limiting example, the RNA pol III promoter is H1.

In some embodiments, the viral genome comprises two promoters. As a non-limiting example, the promoters are an EF1α promoter and a CMV promoter.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter; and (9) GFAP promoter.

In some embodiments, the viral genome comprises an enhancer. In some embodiments, the enhancer comprises a CMVie enhancer.

In some embodiments the viral genome comprises a CMVie enhancer and a CB promoter. In some embodiments, the viral genome comprises a CMVie enhancer and a CMV promoter (e.g., a CMV promoter region). In some embodiments, the viral genome comprises a CMVie enhancer, a CBA promoter or functional variant thereof, and an intron (e.g., a CAG promoter).

In some embodiments, the viral genome comprises an engineered promoter. In another embodiments, the viral genome comprises a promoter from a naturally expressed protein.

In some embodiments, a CBA promoter is used in a viral genomes of an AAV particle described herein, e.g., a viral genome encoding a GCase protein, or a GCase protein and an enhancement element (e.g., a GCase and SapA proteins, GCase and SapC proteins, or GCase protein and a cell penetrating peptide or variants thereof). In some embodiments, the CBA promoter is engineered for optimal expression of a GCase polypeptide or a GCase polypeptide and an enhancement element described herein (e.g., a prosaposin or saposin protein or variant thereof; a cell penetrating peptide or variant thereof; or a lysosomal targeting signal).

### Viral Genome Component: Introns

In some embodiments, the vector genome comprises at least one intron or a fragment or derivative thereof. In some embodiments, the at least one intron may enhance expression of a GCase protein and/or an enhancement element described herein (e.g., a prosaposin protein or a SapC protein or variant thereof; a cell penetrating peptide (e.g., a ApoEII peptide, a TAT peptide, or a ApoB peptide) or variant thereof; and/or a lysosomal targeting signal) (see e.g., Powell et al. Viral Expression Cassette Elements to Enhance Transgene Target Specificity and Expression in Gene Therapy, 2015; the contents of which are herein incorporated by reference in their entirety). Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps), and hybrid adenovirus splice donor/IgG splice acceptor (230 bps).

In some embodiments, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

In some embodiments, the AAV vector may comprise an SV40 intron or fragment or variant thereof. In some embodiments, the promoter may be a CMV promoter. In some embodiments, the promoter may be CBA. In some embodiments, the promoter may be H1.

In some embodiments, the AAV vector may comprise a beta-globin intron or a fragment or variant thereof. In some embodiments, the intron comprises one or more human beta-globin sequences (e.g., including fragments/variants thereof). In some embodiments the promoter may be a CB promoter. In some embodiments, the promoter comprises a CMV promoter. In some embodiments, the promoter comprises a minimal CBA promoter.

In some embodiments, the encoded protein(s) may be located downstream of an intron in an expression vector such as, but not limited to, SV40 intron or beta globin intron or others known in the art. Further, the encoded GBA protein may also be located upstream of the polyadenylation sequence in an expression vector. In some embodiments, the encoded proteins may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides downstream from the promoter comprising an intron (e.g., 3′ relative to the promoter comprising an intron) and/or upstream of the polyadenylation sequence (e.g., 5′ relative to the polyadenylation sequence) in an expression vector. In some embodiments, the encoded GBA protein may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30, or 25-30 nucleotides downstream from the intron (e.g., 3′ relative to the intron) and/or upstream of the polyadenylation sequence (e.g., 5′ relative to the polyadenylation sequence) in an expression vector. In some embodiments, the encoded proteins may be located within the first 1%, 2%, 3%, 4%, 5%, 6%, 7%, 8%, 9%, 10%, 15%, 20%, 25%, or more than 25% of the nucleotides downstream from the intron (e.g., 3′ relative to the intron) and/or upstream of the polyadenylation sequence (e.g., 5′ relative to the polyadenylation sequence) in an expression vector. In some embodiments, the encoded proteins may be located within the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% of the sequence downstream from the intron (e.g., 3′ relative to the intron) and/or upstream of the polyadenylation sequence (e.g., 5′ relative to the polyadenylation sequence) in an expression vector.

In certain embodiments, the intron sequence is not an enhancer sequence. In some embodiments, the intron sequence is not a sub-component of a promoter sequence. In some embodiments, the intron sequence is a sub-component of a promoter sequence.

### Viral Genome Component: Untranslated Regions (UTRs)

In some embodiments, a wild type untranslated region (UTR) of a gene is transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the disclosure to enhance expression in hepatic cell lines or liver.

In some embodiments, the viral genome encoding a transgene described herein (e.g., a transgene encoding a GBA protein) comprises a Kozak sequence. While not wishing to be bound by theory, wild-type 5′ untranslated regions (UTRs) include features that play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

While not wishing to be bound by theory, wild-type 3′ UTRs are known to have stretches of adenosines and uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in their entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

Any UTR from any gene known in the art may be incorporated into the viral genome of the AAV particle. These UTRs, or portions thereof, may be placed in the same orientation as in the gene from which they were selected or they may be altered in orientation or location. In some embodiments, the UTR used in the viral genome of the AAV particle may be inverted, shortened, lengthened, or made with one or more other 5′ UTRs or 3′ UTRs known in the art. As used herein, the term “altered,” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTR, which is not a variant of a wild type UTR.

In some embodiments, the viral genome of the AAV particle comprises UTRs which have been selected from a family of transcripts whose proteins share a common function, structure, feature, or property.

### Viral Genome Component: miR Binding Site

Tissue- or cell-specific expression of the AAV viral particles of the invention can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome of the invention, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed. In some embodiments, the miR binding site modulates, e.g., reduces, expression of the encoded GBA protein in a cell or tissue of the DRG, liver, hematopoietic lineage, or a combination thereof.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleic acid sequence encoding a payload), e.g., before the polyA sequence, 5′-UTR region of the viral genome (e.g., 5′ relative to the nucleic acid sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, the encoded miR binding site series comprises 4 copies of a miR binding site. In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer is about 8 nucleotides in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of SEQ ID NO: 1848, or a nucleotide sequence having at least one, two, or three modifications, but no more than four modifications of SEQ ID NO: 1848.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of SEQ ID NO: 1848, or a nucleotide sequence having at least one, two, or three modifications, but no more than four modifications of SEQ ID NO: 1848.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complementary), to the miR in the host cell. In some embodiments, the sequence complementary to the nucleotide sequence encoding the miR binding site comprises at least 1, 2, 3, 4, or 5 mismatches or no more than 6, 7, 8, 9, or 10 mismatches relative to the corresponding miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complementary to the miR in the host cell.

In some embodiments, the encoded miR binding site or the encoded miR binding site series is about 10 to about 125 nucleotides in length, e.g., about 10 to 50 nucleotides, 10 to 100 nucleotides, 50 to 100 nucleotides, 50 to 125 nucleotides, or 100 to 125 nucleotides in length. In some embodiments, an encoded miR binding site or the encoded miR binding site series is about 7 to about 28 nucleotides in length, e.g., about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., full complementary or partially complementary) to the seed sequence of a miRNA (e.g., a miR122, a miR142, a miR183).

In some embodiments, the encoded miR binding site is complementary (e.g., fully complementary or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 1865), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1865, e.g., wherein the modification can result in a mismatch between the encoded miR binding site and the corresponding miRNA. In some embodiments, the viral genome comprises at least 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGTCA CACTCCA (SEQ ID NO: 1866), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1866, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of SEQ ID NO: 1848, or a nucleotide sequence having at least one, two, or three modifications, but no more than four modifications of SEQ ID NO: 1848.

In some embodiments, the encoded miR binding site is complementary (e.g., fully complementary or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site is complementary (e.g., fully complementary or partially complementary) to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 1869), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1842, e.g., wherein the modification can result in a mismatch between the encoded miR binding site and the corresponding miRNA. In some embodiments, the viral genome comprises at least 3, 4, or 5 copies of an encoded miR-142-3p binding site, e.g., an encoded miR-142-3p binding site series. In some embodiments, the at least 3, 4, or 5 copies (e.g., 4 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of SEQ ID NO: 1848, or a nucleotide sequence having at least one, two, or three modifications, but no more than four modifications of SEQ ID NO: 1848.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary (e.g., fully complementary or partially complementary) to a miR expressed in expressed in a DRG neuron. In some embodiments, the encoded miR binding site comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 1847), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1847, e.g., wherein the modification can result in a mismatch between the encoded miR binding site and the corresponding miRNA. In some embodiments, the sequence complementary (e.g., fully complementary or partially complementary) to the seed sequence corresponds to the double underlined of the encoded miR-183 binding site sequence. In some embodiments, the viral genome comprises at least comprises at least 3, 4, or 5 copies (e.g., 4 copies) of the encoded miR183 binding site, e.g. an encoded miR183 binding site. In some embodiments, the viral genome comprises at least comprises 4 copies of the encoded miR183 binding site, e.g. an encoded miR183 binding site comprising 4 copies of a miR183 binding site. In some embodiments, the at least 3, 4, or 5 copies (e.g., 4 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of SEQ ID NO: 1848, or a nucleotide sequence having at least one, two, or three modifications, but no more than four modifications of SEQ ID NO: 1848. In some embodiments, the encoded miR183 binding site series comprises the nucleotide sequence of SEQ ID NO: 1849, or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1849.

In some embodiments, the encoded miR binding site or encoded miR binding site series comprises a miR182 binding site sequence. In some embodiments, the encoded miR182 binding site comprises, the nucleotide sequence of AGTGTGAGTTCTACCATTGCCAAA (SEQ ID NO: 1867), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1867, e.g., wherein the modification can result in a mismatch between the encoded miR binding site and the corresponding miRNA. In some embodiments, the viral genome comprises at least 3, 4, or 5 copies of the encoded miR182 binding site, e.g., an encoded miR182 binding site series. In some embodiments, the at least 3, 4, or 5 copies (e.g., 4 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of SEQ ID NO: 1848, or a nucleotide sequence having at least one, two, or three modifications, but no more than four modifications of SEQ ID NO: 1848.

In some embodiments, the encoded miR binding site or encoded miR binding site series comprises a miR96 binding site sequence. In some embodiments, the encoded miR96 binding site comprises the nucleotide sequence of AGCAAAAATGTGCTAGTGCCAAA (SEQ ID NO: 1868), a sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1868, e.g., wherein the modification can result in a mismatch between the encoded miR binding site and the corresponding miRNA. In some embodiments, the viral genome comprises at least 3, 4, or 5 copies of the encoded miR96 binding site, e.g., an encoded miR96 binding site series. In some embodiments, the at least 3, 4, or 5 copies (e.g., 4 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of SEQ ID NO: 1848, or a nucleotide sequence having at least one, two, or three modifications, but no more than four modifications of SEQ ID NO: 1848.

In some embodiments, the encoded miR binding site series comprises a miR122 binding site, a miR142 binding site, a miR183 binding site, a miR182 binding site, a miR 96 binding site, or a combination thereof. In some embodiments, the encoded miR binding site series comprises at least 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of SEQ ID NO: 1848, or a nucleotide sequence having at least one, two, or three modifications, but no more than four modifications of SEQ ID NO: 1848.

In some embodiments, an encoded miR binding site series comprises at least 3-5 copies (e.g., 4 copies) of a combination of at least two, three, four, five, or all of a miR122 binding site, a miR142 binding site, a miR183 binding site, a miR182 binding site, a miR96 binding site, wherein each of the miR binding sites within the series are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of SEQ ID NO: 1848, or a nucleotide sequence having at least one, two, or three modifications, but no more than four modifications of SEQ ID NO:

### Viral Genome Component: Polyadenylation Sequence

In some embodiments, the viral genome of the AAV particles of the present disclosure comprises at least one polyadenylation (polyA) sequence. The viral genome of the AAV particle may comprise a polyadenylation sequence between the 3′ end of the payload coding sequence and the 5′ end of the 3′UTR. In some embodiments, the polyA signal region is positioned 3′ relative to the nucleic acid comprising the transgene encoding the payload, e.g., a GBA protein described herein.

In some embodiments, the polyA signal region comprises a length of about 100-600 nucleotides, e.g., about 100-500 nucleotides, about 100-400 nucleotides, about 100-300 nucleotides, about 100-200 nucleotides, about 200-600 nucleotides, about 200-500 nucleotides, about 200-400 nucleotides, about 200-300 nucleotides, about 300-600 nucleotides, about 300-500 nucleotides, about 300-400 nucleotides, about 400-600 nucleotides, about 400-500 nucleotides, or about 500-600 nucleotides. In some embodiments, the polyA signal region comprises a length of about 100 to 150 nucleotides, e.g., about 127 nucleotides. In some embodiments, the polyA signal region comprises a length of about 450 to 500 nucleotides, e.g., about 477 nucleotides. In some embodiments, the polyA signal region comprises a length of about 520 to about 560 nucleotides, e.g., about 552 nucleotides. In some embodiments, the polyA signal region comprises a length of about 127 nucleotides.

In some embodiments, the viral genome comprises a human growth hormone (hGH) polyA sequence. In some embodiments, the viral genome comprises an hGH polyA as described above and a payload region encoding the GCase protein, or the GCase and an enhancement element (e.g., a prosaposin, SapA, or SapC protein, or variant thereof; a cell penetrating peptide (e.g., an ApoEII peptide, a TAT peptide, or an ApoB peptide); or a lysosomal targeting peptide) e.g., encoding a sequence as provided in Tables 3 and 4 or fragment or variant thereof.

### Viral Genome Component: Filler Sequence

In some embodiments, the viral genome comprises one or more filler sequences. The filler sequence may be a wild-type sequence or an engineered sequence. A filler sequence may be a variant of a wild-type sequence. In some embodiments, a filler sequence is a derivative of human albumin.

In some embodiments, the viral genome comprises one or more filler sequences in order to have the length of the viral genome be the optimal size for packaging. In some embodiments, the viral genome comprises at least one filler sequence in order to have the length of the viral genome be about 2.3 kb. In some embodiments, the viral genome comprises at least one filler sequence in order to have the length of the viral genome be about 4.6 kb.

In some embodiments, the viral genome is a single stranded (ss) viral genome and comprises one or more filler sequences that, independently or together, have a length about between 0.1 kb-3.8 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, or 3.8 kb. In some embodiments, the total length filler sequence in the vector genome is 3.1 kb. In some embodiments, the total length filler sequence in the vector genome is 2.7 kb. In some embodiments, the total length filler sequence in the vector genome is 0.8 kb. In some embodiments, the total length filler sequence in the vector genome is 0.4 kb. In some embodiments, the length of each filler sequence in the vector genome is 0.8 kb. In some embodiments, the length of each filler sequence in the vector genome is 0.4 kb.

In some embodiments, the viral genome is a self-complementary (sc) viral genome and comprises one or more filler sequences that, independently or together, have a length about between 0.1 kb-1.5 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, or 1.5 kb. In some embodiments, the total length filler sequence in the vector genome is 0.8 kb. In some embodiments, the total length filler sequence in the vector genome is 0.4 kb. In some embodiments, the length of each filler sequence in the vector genome is 0.8 kb. In some embodiments, the length of each filler sequence in the vector genome is 0.4 kb.

In some embodiments, the viral genome comprises any portion of a filler sequence. The viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of a filler sequence.

In some embodiments, the viral genome is a single stranded (ss) viral genome and comprises one or more filler sequences in order to have the length of the viral genome be about 4.6 kb. In some embodiments, the viral genome comprises at least one filler sequence and the filler sequence is located 3′ to the 5′ ITR sequence. In some embodiments, the viral genome comprises at least one filler sequence and the filler sequence is located 5′ to a promoter sequence. In some embodiments, the viral genome comprises at least one filler sequence and the filler sequence is located 3′ to the polyadenylation signal sequence. In some embodiments, the viral genome comprises at least one filler sequence and the filler sequence is located 5′ to the 3′ ITR sequence. In some embodiments, the viral genome comprises at least one filler sequence, and the filler sequence is located between two intron sequences. In some embodiments, the viral genome comprises at least one filler sequence, and the filler sequence is located within an intron sequence. In some embodiments, the viral genome comprises two filler sequences, and the first filler sequence is located 3′ to the 5′ ITR sequence and the second filler sequence is located 3′ to the polyadenylation signal sequence. In some embodiments, the viral genome comprises two filler sequences, and the first filler sequence is located 5′ to a promoter sequence and the second filler sequence is located 3′ to the polyadenylation signal sequence. In some embodiments, the viral genome comprises two filler sequences, and the first filler sequence is located 3′ to the 5′ ITR sequence and the second filler sequence is located 5′ to the 5′ ITR sequence.

In some embodiments, the viral genome is a self-complementary (sc) viral genome and comprises one or more filler sequences in order to have the length of the viral genome be about 2.3 kb. In some embodiments, the viral genome comprises at least one filler sequence and the filler sequence is located 3′ to the 5′ ITR sequence. In some embodiments, the viral genome comprises at least one filler sequence and the filler sequence is located 5′ to a promoter sequence. In some embodiments, the viral genome comprises at least one filler sequence and the filler sequence is located 3′ to the polyadenylation signal sequence. In some embodiments, the viral genome comprises at least one filler sequence and the filler sequence is located 5′ to the 3′ ITR sequence. In some embodiments, the viral genome comprises at least one filler sequence, and the filler sequence is located between two intron sequences. As a non-limiting example, the viral genome comprises at least one filler sequence, and the filler sequence is located within an intron sequence. In some embodiments, the viral genome comprises two filler sequences, and the first filler sequence is located 3′ to the 5′ ITR sequence and the second filler sequence is located 3′ to the polyadenylation signal sequence. In some embodiments, the viral genome comprises two filler sequences, and the first filler sequence is located 5′ to a promoter sequence and the second filler sequence is located 3′ to the polyadenylation signal sequence. In some embodiments, the viral genome comprises two filler sequences, and the first filler sequence is located 3′ to the 5′ ITR sequence and the second filler sequence is located 5′ to the 5′ ITR sequence.

In some embodiments, the viral genome may comprise one or more filler sequences between one of more regions of the viral genome. In some embodiments, the filler region may be located before a region such as, but not limited to, a payload region, an inverted terminal repeat (ITR), a promoter region, an intron region, an enhancer region, a polyadenylation signal sequence region, and/or an exon region. In some embodiments, the filler region may be located after a region such as, but not limited to, a payload region, an inverted terminal repeat (ITR), a promoter region, an intron region, an enhancer region, a polyadenylation signal sequence region, and/or an exon region. In some embodiments, the filler region may be located before and after a region such as, but not limited to, a payload region, an inverted terminal repeat (ITR), a promoter region, an intron region, an enhancer region, a polyadenylation signal sequence region, and/or an exon region.

In some embodiments, the viral genome may comprise one or more filler sequences that bifurcate(s) at least one region of the viral genome. The bifurcated region of the viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of the of the region to the 5′ of the filler sequence region. In some embodiments, the filler sequence may bifurcate at least one region so that 10% of the region is located 5′ to the filler sequence and 90% of the region is located 3′ to the filler sequence. In some embodiments, the filler sequence may bifurcate at least one region so that 20% of the region is located 5′ to the filler sequence and 80% of the region is located 3′ to the filler sequence. In some embodiments, the filler sequence may bifurcate at least one region so that 30% of the region is located 5′ to the filler sequence and 70% of the region is located 3′ to the filler sequence. In some embodiments, the filler sequence may bifurcate at least one region so that 40% of the region is located 5′ to the filler sequence and 60% of the region is located 3′ to the filler sequence. In some embodiments, the filler sequence may bifurcate at least one region so that 50% of the region is located 5′ to the filler sequence and 50% of the region is located 3′ to the filler sequence. In some embodiments, the filler sequence may bifurcate at least one region so that 60% of the region is located 5′ to the filler sequence and 40% of the region is located 3′ to the filler sequence. In some embodiments, the filler sequence may bifurcate at least one region so that 70% of the region is located 5′ to the filler sequence and 30% of the region is located 3′ to the filler sequence. In some embodiments, the filler sequence may bifurcate at least one region so that 80% of the region is located 5′ to the filler sequence and 20% of the region is located 3′ to the filler sequence. In some embodiments, the filler sequence may bifurcate at least one region so that 90% of the region is located 5′ to the filler sequence and 10% of the region is located 3′ to the filler sequence.

In some embodiments, the viral genome comprises a filler sequence after the 5′ ITR.

In some embodiments, the viral genome comprises a filler sequence after the promoter region. In some embodiments, the viral genome comprises a filler sequence after the payload region. In some embodiments, the viral genome comprises a filler sequence after the intron region. In some embodiments, the viral genome comprises a filler sequence after the enhancer region. In some embodiments, the viral genome comprises a filler sequence after the polyadenylation signal sequence region. In some embodiments, the viral genome comprises a filler sequence after the exon region.

In some embodiments, the viral genome comprises a filler sequence before the promoter region. In some embodiments, the viral genome comprises a filler sequence before the payload region. In some embodiments, the viral genome comprises a filler sequence before the intron region. In some embodiments, the viral genome comprises a filler sequence before the enhancer region. In some embodiments, the viral genome comprises a filler sequence before the polyadenylation signal sequence region. In some embodiments, the viral genome comprises a filler sequence before the exon region.

In some embodiments, the viral genome comprises a filler sequence before the 3′ ITR.

In some embodiments, a filler sequence may be located between two regions, such as, but not limited to, the 5′ ITR and the promoter region. In some embodiments, a filler sequence may be located between two regions, such as, but not limited to, the 5′ ITR and the payload region. In some embodiments, a filler sequence may be located between two regions, such as, but not limited to, the 5′ ITR and the intron region. In some embodiments, a filler sequence may be located between two regions, such as, but not limited to, the 5′ ITR and the enhancer region. In some embodiments, a filler sequence may be located between two regions, such as, but not limited to, the 5′ ITR and the polyadenylation signal sequence region.

In some embodiments, a filler sequence may be located between two regions, such as, but not limited to, the 5′ ITR and the exon region.

In some embodiments, a filler sequence may be located between two regions, such as, but not limited to, the promoter region and the payload region. In some embodiments, a filler sequence may be located between two regions, such as, but not limited to, the promoter region and the intron region. In some embodiments, a filler sequence may be located between two regions, such as, but not limited to, the promoter region and the enhancer region. In some embodiments, a filler sequence may be located between two regions, such as, but not limited to, the promoter region and the polyadenylation signal sequence region. In some embodiments, a filler sequence may be located between two regions, such as, but not limited to, the promoter region and the exon region. In some embodiments, a filler sequence may be located between two regions, such as, but not limited to, the promoter region and the 3′ ITR.

In some embodiments, a filler sequence may be located between two regions, such as, but not limited to, the payload region and the intron region. In some embodiments, a filler sequence may be located between two regions, such as, but not limited to, the payload region and the enhancer region. In some embodiments, a filler sequence may be located between two regions, such as, but not limited to, the payload region and the polyadenylation signal sequence region. In some embodiments, a filler sequence may be located between two regions, such as, but not limited to, the payload region and the exon region.

In some embodiments, a filler sequence may be located between two regions, such as, but not limited to, the payload region and the 3′ ITR.

### Viral Genome Component: Payloads

In some embodiments, an AAV particle, e.g., an AAV particle for the vectorized delivery of a GBA protein, e.g., a GBA protein described herein, comprises a payload. In some embodiments, an AAV particle, e.g., an AAV particle for the vectorized delivery of a GBA protein described herein (e.g., an GBA protein), comprises a viral genome encoding a payload. In some embodiments, the viral genome comprises a promoter operably linked to a nucleic acid comprising a transgene encoding a payload. In some embodiments, the payload comprises an GBA protein.

In some embodiments, the disclosure herein provides constructs that allow for improved expression of GCase protein delivered by gene therapy vectors.

In some embodiments, the disclosure provides constructs that allow for improved biodistribution of GCase protein delivered by gene therapy vectors.

In some embodiments, the disclosure provides constructs that allow for improved sub-cellular distribution or trafficking of GCase protein delivered by gene therapy vectors.

In some embodiments, the disclosure provides constructs that allow for improved trafficking of GCase protein to lysosomal membranes delivered by gene therapy vectors.

In some aspects, the present disclosure relates to a composition containing or comprising a nucleic acid sequence encoding a GCase protein or functional fragment or variants thereof and methods of administering the composition in vitro or in vivo in a subject, e.g., a humans and/or an animal model of disease, e.g., a disease related to expression of GBA.

AAV particles of the present disclosure may comprise a nucleic acid sequence encoding at least one “payload.” As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, e.g., GCase protein or fragment or variant thereof. The payload may comprise any nucleic acid known in the art that is useful for the expression (by supplementation of the protein product or gene replacement using a modulatory nucleic acid) of GCase protein in a target cell transduced or contacted with the AAV particle carrying the payload.

Specific features of a transgene encoding GCase for use in an AAV genome as described herein include the use of a wildtype GBA-encoding sequence and enhanced GBA-encoding constructs. In some instances, the GBA-encoding sequence is a recombinant and/or modified GBA sequence as described in Int'l Pub. No. WO2019040507, the contents of which are herein incorporated by reference in their entirety. In some embodiments, the GBA-encoding sequence is as provided by NCBI Reference Sequence NCBI Reference Sequence NP_000148.2 (SEQ ID NO: 14 of Int'l Pub. No. WO2019070893, incorporated by reference herein). In some embodiments, the GBA-encoding sequence is codon optimized for expression in mammalian cells including human cells, such as the sequence set forth in SEQ ID NO: 15 of WO2019070893. In some embodiments, the viral genome comprises a sequence encoding Prosaposin (PSAP), the precursor of Saposin proteins A, B, C, and D (SapA, SapB, SapC, and SapD, respectively). The sequence encoding Prosaposin can be the sequence as provided by NCBI Reference Sequence NP_002769.1 (SEQ ID NO: 16 of WO2019070893). In some embodiments, the PSAP-encoding sequence is codon optimized for expression in mammalian cells including human cells, such as the sequence set forth in SEQ ID NO: 17 of WO2019070893. In some embodiments, the GBA-encoding sequence is a recombinant and/or modified GBA sequence as described in Int'l Pub. No. WO2019070894.

An enhanced GBA-encoding sequence, as described and exemplified herein, can achieve enhanced catalytic activity of the GCase enzyme by incorporation of prosaposin or saposin C coding sequence in the viral genome. Alternatively, an enhanced GBA-encoding sequence can achieve enhanced cell penetration of secreted GCase product by incorporating, e.g., HIV-derived TAT peptide, Human Apolipoprotein B receptor binding domain, Human Apolipoprotein E II receptor binding domain, or other cell penetration-enhancing sequences. In some embodiments, the enhanced GBA-encoding sequence can achieve enhanced intracellular lysosomal targeting by incorporating one or more of, a) an Rnase A-derived sequence; b) an HSC70-derived sequence; c) a Hemoglobin-derived sequence; d) a combination of Rnase A-, HSC70-, and Hemoglobin-derived lysosomal targeting sequences; or e) other lysosomal targeting enhancer sequences. An enhanced GB A-encoding sequences as described herein can, in some embodiments, incorporate combinatorial enhancements of the enhanced catalytic activity, enhanced cell-penetration activity, and/or enhanced lysosomal targeting features. In some embodiments, the combination(s) of these enhanced features have additive effects on GCase activity or expression in cells infected with AAV particles bearing the AAV genomes described herein. For example, in some embodiments, the AAV genome described herein comprise a GCase-encoding nucleic acid sequence having a lysosomal targeting sequence, GCase-coding sequence, linker, and PSAP/SapC-encoding sequence. In some embodiments, the combination(s) of these enhanced features have synergistic effects on GCase activity or expression in cells infected with AAV particles bearing the AAV genomes described herein.

The payload construct may comprise a combination of coding and non-coding nucleic acid sequences.

Any segment, fragment, or the entirety of the viral genome and therein, the payload region, may be codon optimized.

In some embodiments, the viral genome encodes more than one payload. As a non-limiting example, a viral genome encoding more than one payload may be replicated and packaged into a viral particle. A target cell transduced with a viral particle comprising more than one payload may express each of the payloads in a single cell.

In some embodiments, the viral genome may encode a coding or non-coding RNA. In certain embodiments, the adeno-associated viral vector particle further comprises at least one cis-element selected from the group consisting of a Kozak sequence, a backbone sequence, and an intron sequence.

In some embodiments, the payload is a polypeptide which may be a peptide or protein. A protein encoded by the payload construct may comprise a secreted protein, an intracellular protein, an extracellular protein, and/or a membrane protein. The encoded proteins may be structural or functional. Proteins encoded by the viral genome include, but are not limited to, mammalian proteins. In certain embodiments, the AAV particle contains a viral genome that encodes GCase protein or a fragment or variant thereof. The AAV particles described herein may be useful in the fields of human disease, veterinary applications, and a variety of in vivo and in vitro settings.

In some embodiments, a payload may comprise polypeptides that serve as marker proteins to assess cell transformation and expression, fusion proteins, polypeptides having a desired biological activity, gene products that can complement a genetic defect, RNA molecules, transcription factors, and other gene products that are of interest in regulation and/or expression. In some embodiments, a payload may comprise nucleotide sequences that provide a desired effect or regulatory function (e.g., transposons, transcription factors).

The encoded payload may comprise a gene therapy product. A gene therapy product may include, but is not limited to, a polypeptide, RNA molecule, or other gene product that, when expressed in a target cell, provides a desired therapeutic effect. In some embodiments, a gene therapy product may comprise a substitute for a non-functional gene or a gene that is absent, expressed in insufficient amounts, or mutated. In some embodiments, a gene therapy product may comprise a substitute for a non-functional protein or polypeptide or a protein or polypeptide that is absent, expressed in insufficient amounts, misfolded, degraded too rapidly, or mutated. For example, a gene therapy product may comprise a GCase protein or a polynucleotide encoding GCase protein to treat GCase deficiency or GBA-related disorders.

In some embodiments, the payload encodes a messenger RNA (mRNA). As used herein, the term “messenger RNA” (mRNA) refers to any polynucleotide that encodes a polypeptide of interest and that is capable of being translated to produce the encoded polypeptide of interest in vitro, in vivo, in situ, or ex vivo. Certain embodiments provide the mRNA as encoding GCase or a variant thereof.

The components of an mRNA include, but are not limited to, a coding region, a 5′-UTR (untranslated region), a 3′-UTR, a 5′-cap and a poly-A tail. In some embodiments, the encoded mRNA or any portion of the AAV genome may be codon optimized.

In some embodiments, the protein or polypeptide encoded by the payload construct encoding GCase or a variant thereof is between about 50 and about 4500 amino acid residues in length (hereinafter in this context, “X amino acids in length” refers to X amino acid residues). In some embodiments, the protein or polypeptide encoded is between 50-2000 amino acids in length. In some embodiments, the protein or polypeptide encoded is between 50-1000 amino acids in length. In some embodiments, the protein or polypeptide encoded is between 50-1500 amino acids in length. In some embodiments, the protein or polypeptide encoded is between 50-1000 amino acids in length. In some embodiments, the protein or polypeptide encoded is between 50-800 amino acids in length. In some embodiments, the protein or polypeptide encoded is between 50-600 amino acids in length. In some embodiments, the protein or polypeptide encoded is between 50-400 amino acids in length. In some embodiments, the protein or polypeptide encoded is between 50-200 amino acids in length. In some embodiments, the protein or polypeptide encoded is between 50-100 amino acids in length.

A payload construct encoding a payload may comprise or encode a selectable marker. A selectable marker may comprise a gene sequence or a protein or polypeptide encoded by a gene sequence expressed in a host cell that allows for the identification, selection, and/or purification of the host cell from a population of cells that may or may not express the selectable marker. In some embodiments, the selectable marker provides resistance to survive a selection process that would otherwise kill the host cell, such as treatment with an antibiotic. In some embodiments, an antibiotic selectable marker may comprise one or more antibiotic resistance factors, including but not limited to neomycin resistance (e.g., neo), hygromycin resistance, kanamycin resistance, and/or puromycin resistance.

In some embodiments, a payload construct encoding a payload may comprise a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540); the contents of each of which are herein incorporated by reference in their entirety.

In some embodiments, a payload construct encoding a selectable marker may comprise a fluorescent protein. A fluorescent protein as herein described may comprise any fluorescent marker including but not limited to green, yellow, and/or red fluorescent protein (GFP, YFP, and/or RFP). In some embodiments, a payload construct encoding a selectable marker may comprise a human influenza hemagglutinin (HA) tag.

In certain embodiments, a nucleic acid for expression of a payload in a target cell will be incorporated into the viral genome and located between two ITR sequences.

In some embodiments, a payload construct further comprises a nucleic acid sequence encoding a peptide that binds to the cation-independent mannose 6-phosphate (M6P) receptor (CI-MPR) with high affinity, as described in Int'l Pat. App. Pub. No. WO2019213180A1, the disclosure of which is incorporated herein by reference in its entirety. The peptide that binds CI-MPR can be, e.g., an IGF2 peptide or variant thereof. Binding of CI-MPR can facilitate cellular uptake or delivery and intracellular or sub-cellular targeting of therapeutic proteins provided by gene therapy vectors.

### Payload Component: Linker

In some embodiments, a viral genome described herein may be engineered with one or more spacer or linker regions to separate coding or non-coding regions.

In some embodiments, the nucleic acid comprising a transgene encoding the payload, e.g., a GBA protein described herein, further comprises a nucleic acid sequence encoding a linker. In some embodiments, the nucleic acid encoding the payload encodes two or more linkers. In some embodiments, the encoded linker comprises a linker provided in Table 2 or 5. In some embodiments, the encoded linker comprises an amino acid sequence encoded by any one of the nucleotide sequences provided in Table 2 or 5, or an amino acid sequence with at least 70%, 75%, 80%, 85%, 90%, 95%, or 99% sequence identity thereto. In some embodiments, the nucleic acid sequence encoding the linker comprises any one of the nucleotide sequences provided in Table 2 or 5, or a nucleotide sequence with at least 70%, 75%, 80%, 85%, 90%, 95%, or 99% sequence identity thereto. In some embodiments, the linker comprises any one of the amino acid sequences provided in Table 2, or an amino acid sequence

In some embodiments, the linker may be a peptide linker that may be used to connect the polypeptides encoded by the payload region during expression. In some embodiments, a peptide linkers may be cleaved after expression to separate GCase protein domains, or to separate GCase proteins from an enhancement element described herein, e.g., a prosaposin, SapA and/or SapC protein or functional variant, allowing expression of independent functional GCase protein and enhancement element polypeptide, e.g., a prosaposin, SapA, and/or SapC polypeptides, and other payload polypeptides. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains from a single transcript. In some cases, two or more linkers are encoded by a payload region of the viral genome.

TABLE 2
Linkers

| Linker ID | Description | Length | SEQ ID NO |
|---|---|---|---|
| Linker1 | Furin | 12 | 1724 |
| Linker2 | Furin | 12 | 1725 |
| Linker3 | T2A | 54 | 1726 |
| Linker4 | F2A | 75 | 1727 |
| Linker5 | P2A | 66 | 1728 |
| Linker6 | G4S | 18 | 1729 |
| Linker7 | G4S3 | 45 | 1730 |
| Linker8 | G4S5 | 75 | 1731 |
| Linker9 | IRES | 609 | 1732 |
| Linker10 | IRES-2 | 623 | 1733 |
| Linker11 | hIgG2 hinge | 54 | 1734 |
| Linker12 | hIgG3 hinge | 108 | 1735 |
| Linker13 | hIgG3-2 hinge | 153 | 1736 |
| Linker14 | hIgG3-3 hinge | 198 | 1737 |
| Linker15 | msiGG-1 hinge | 45 | 1738 |
| Linker16 | msiGG1 hinge | 18 | 1739 |
| Linker17 | G4S3 | 45 | 1873 |

In some embodiments, the GBA protein and the enhancement element described herein can be connected directly, e.g., without a linker. In some embodiments, the GBA protein and the enhancement element described herein can be connected via a linker. In some embodiments, the linker is a cleavable linker. In some embodiments, the linker is not cleaved.

In some embodiments, any of the payloads described herein, can have a linker, e.g. a flexible polypeptide linker, of varying lengths, connecting the GBA protein and the enhancement element, e.g., the cell penetrating peptide, e.g., a ApoEII peptide, a TAT peptide, and/or a ApoB peptide. For example, a (Gly4Ser)n linker (SEQ ID NO: 1872), wherein n is 0, 1, 2, 3, 4, 5, 6, 7, or 8 can be used (e.g., any one of SEQ ID NOs: 1729, 1730, 1731, 1843, or 1845). In some embodiments, the linker comprises a (Gly4Ser)3 (SEQ ID NO: 1845). In some embodiments, the nucleotide sequence encoding the linker comprises the nucleotide sequence of SEQ ID NO: 1730, or a nucleotide sequence having at least one, two, or three but no more than four modifications, e.g., substitutions, relative to SEQ ID NO: 1730. In some embodiments, the encoded linker comprises the amino acid sequence of SEQ ID NO: 1845, or an amino acid sequence having at least one, two, or three but no more than four modifications, e.g., substitutions, relative to SEQ ID NO: 1845.

In some embodiments, the encoded linker comprises an enzymatic cleavage site, e.g., for intracellular and/or extracellular cleavage. In some embodiments, the linker is cleaved to separate the GBA protein and the encoded enhancement element, e.g., a prosaposin polypeptide, a SapA polypeptide, a SapC polypeptide, or functional variant thereof. In some embodiments, the encoded linker comprises a furin linker or a functional variant. In some embodiments, the nucleotide sequence encoding the furin linker comprises the nucleotide sequence of SEQ ID NO: 1724, a nucleotide sequence with at least 70%, 75%, 80%, 85%, 90%, 95%, or 99% sequence identity to SEQ ID NO: 1724, or a nucleotide sequence having at least one, two, or three but no more than four modifications, e.g., substitutions, relative to SEQ ID NO: 1724. In some embodiments, the furin linker comprises the amino acid sequence of SEQ ID NO: 1854, or an amino acid sequence having at least one, two, or three but no more than four modifications, e.g., substitutions, relative to SEQ ID NO: 1854. In some embodiments, furin cleaves proteins downstream of a basic amino acid target sequence (e.g., Arg-X-(Arg/Lys)-Arg) (e.g., as described in Thomas, G., 2002. Nature Reviews Molecular Cell Biology 3(10): 753-66; the contents of which are herein incorporated by reference in its entirety). In some embodiments, the encoded linker comprises a 2A self-cleaving peptide (e.g., a 2A peptide derived from foot-and-mouth disease virus (F2A), porcine teschovirus-1 (P2A), Thoseaasigna virus (T2A), or equine rhinitis A virus (E2A)). In some embodiments, the encoded linker comprises a T2A self-cleaving peptide linker. In some embodiments, the nucleotide sequence encoding the T2A linker comprises the nucleotide sequence of SEQ ID NO: 1726, a nucleotide sequence with at least 70%, 75%, 80%, 85%, 90%, 95%, or 99% sequence identity to SEQ ID NO: 1726, or a nucleotide sequence having at least one, two, or three but no more than four modifications, e.g., substitutions, relative to SEQ ID NO: 1726. In some embodiments, the T2A linker comprises the amino acid sequence of SEQ ID NO: 1855, or an amino acid sequence having at least one, two, or three but no more than four modifications, e.g., substitutions, relative to SEQ ID NO: 1855. In some embodiments, the nucleic acid encoding the payload encodes a furin linker and a T2A linker.

In some embodiments, the encoded linker comprises an internal ribosomal entry site (IRES) is a nucleotide sequence (>500 nucleotides) for initiation of translation in the middle of a nucleotide sequence, e.g., an mRNA sequence (Kim, J. H. et al., 2011. PLoS One 6(4): e18556; the contents of which are herein incorporated by reference in its entirety), which can be used, for example, to modulate expression of one or more transgenes. In some embodiments, the encode linker comprises a small and unbranched serine-rich peptide linker, such as those described by Huston et al. in U.S. Pat. No. 5,525,491, the contents of which are herein incorporated in their entirety. In some embodiments, polypeptides comprising a serine-rich linker has increased solubility. In some embodiments, the encoded linker comprises an artificial linker, such as those described by Whitlow and Filpula in U.S. Pat. No. 5,856,456 and Ladner et al. in U.S. Pat. No. 4,946,778, the contents of each of which are herein incorporated by their entirety.

In some embodiments, the encoded linkers comprises a cathepsin, a matrix metalloproteinases or a legumain cleavage sites, such as those described e.g. by Cizeau and Macdonald in International Publication No. WO2008052322, the contents of which are herein incorporated in their entirety.

In some embodiments, the nucleotide sequence encoding the linker comprises about 10 to about 700 nucleotides in length, e.g., about 10 to about 700 nucleotides, e.g. about 10 to about 100, e.g., about 50-200 nucleotides, about 150-300 nucleotides, about 250-400 nucleotides, about 350-500 nucleotides, about 450-600 nucleotides, about 550-700 nucleotides, about 650-700 nucleotides. In some embodiments, the nucleotide sequence encoding the linker comprises about 5 to about 20 nucleotides in length, e.g., about 12 nucleotides in length. In some embodiments, the nucleotide sequence encoding the linker comprises about 40 to about 60 nucleotides in length, e.g., about 54 nucleotides in length.

### Payload Component: Signal Sequence

In some embodiments, the nucleic acid sequence comprising the transgene encoding the payload, e.g., a GBA protein, an enhancement element (e.g., a prosaposin protein, saposin C protein, or variant thereof; a cell penetrating peptide (e.g., a ApoEII peptide, a TAT peptide, and/or an ApoB protein), or a lysosomal targeting signal), or a GBA protein and an enhancement element, comprises a nucleic acid sequence encoding a signal sequence (e.g., a signal sequence region herein). In some embodiments, the nucleic acid sequence comprising the transgene encoding the payload comprises two signal sequence regions. In some embodiments, the nucleic acid sequence comprising the transgene encoding the payload comprises three or more signal sequence regions.

In some embodiments, the nucleotide sequence encoding the signal sequence is located 5′ relative to the nucleotide sequence encoding the GBA protein. In some embodiments, the nucleotide sequence encoding the signal sequence is located 5′ relative to the nucleotide sequence encoding the enhancement element. In some embodiments, the encoded GBA protein and/or the encoded enhancement element comprises a signal sequence at the N-terminus, wherein the signal sequence is optionally cleaved during cellular processing and/or localization of the GBA protein and/or the enhancement element.

In some embodiments, the signal sequence comprises the sequence any one of the signal sequences provided in Table 4 or 14 or a sequence substantially identical (e.g., having at least 70%, 75%, 80%, 85%, 90%, 95%, or 99% identity) thereto. In some embodiments, the encoded signal sequence comprises the amino acid sequence of SEQ ID NO: 1853 or 1857, or an amino acid sequence at least 85% (e.g., at least 90%, 92%, 95%, 97%, 98%, or 99%) identical thereto. In some embodiments, the nucleotide sequence encoding the signal sequence comprises of any of SEQ ID NOs: 1850-1852 or 1856, or a nucleotide sequence at least 85% (e.g., at least 90%, 92%, 95%, 97%, 98%, or 99%) identical thereto.

In some embodiments, the encoded signal sequence comprises the amino acid sequence of SEQ ID NO: 1853 or an amino acid sequence at least 85% (e.g., at least 90%, 92%, 95%, 97%, 98%, or 99%) identical thereto; and the encoded GBA protein comprises the amino acid sequence of SEQ ID NO: 1775, or an amino acid sequence at least 70% (e.g., at least 75%, 80%, 85%, 90%, 92%, 95%, 97%, 98%, or 99%) identical thereto. In some embodiments, the encoded signal sequence is located N-terminal relative to the encoded GBA protein.

In some embodiments, the nucleotide sequence encoding the signal sequence comprises the nucleotide sequence of 1850 or a nucleotide sequence at least 85% (e.g., at least 90%, 92%, 95%, 97%, 98%, or 99%) identical thereto, and the nucleotide sequence encoding the GBA protein comprises the nucleotide sequence of SEQ ID NO: 1773, or a nucleotide sequence at least 70% (e.g., at least 75%, 80%, 85%, 90%, 92%, 95%, 97%, 98%, or 99%) identical thereto. In some embodiments, the nucleotide sequence encoding the signal sequence comprises the nucleotide sequence of 1851 or a nucleotide sequence at least 85% (e.g., at least 90%, 92%, 95%, 97%, 98%, or 99%) identical thereto, and the nucleotide sequence encoding the GBA protein comprises the nucleotide sequence of SEQ ID NO: 1777, or a nucleotide sequence at least 70% (e.g., at least 75%, 80%, 85%, 90%, 92%, 95%, 97%, 98%, or 99%) identical thereto. In some embodiments, the nucleotide sequence encoding the signal sequence comprises the nucleotide sequence of 1852 or a nucleotide sequence at least 85% (e.g., at least 90%, 92%, 95%, 97%, 98%, or 99%) identical thereto, and the nucleotide sequence encoding the GBA protein comprises the nucleotide sequence of SEQ ID NO: 1781, or a nucleotide sequence at least 70% (e.g., at least 75%, 80%, 85%, 90%, 92%, 95%, 97%, 98%, or 99%) identical thereto. In some embodiments, the nucleotide sequence encoding the signal sequence is located 5′ relative to the nucleotide sequence encoding the GBA protein.

### Exemplary GCase (GBA) Protein Payload

In some embodiments, the payload, e.g., of a viral genome described herein, is a GCase protein, e.g., a wild-type GCase protein, or a functional variant thereof. In some embodiments, a functional variant is a variant that retains some or all of the activity of its wild-type counterpart, so as to achieve a desired therapeutic effect. For example, in some embodiments, a functional variant is effective to be used in gene therapy to treat a disorder or condition, for example, a GBA gene product deficiency, PD, or a GBA-related disorders, a neurodegenerative disorder, and/or a neuromuscular disorder. Unless indicated otherwise, a variant of a GCase protein as described herein (e.g., in the context of the constructs, vectors, genomes, methods, kits, compositions, etc. of the disclosure) is a functional variant.

As used herein, “associated with decreased GCase protein levels” or “associated with decreased expression” means that one or more symptoms of a disease are caused by lower-than-normal GCase protein levels in a target tissue or in a biofluid such as blood. A disease or condition associated with decreased GCase protein levels or expression may be a disorder of the central nervous system. Also specifically contemplated herein are Parkinson Disease and related disorders arising from expression of defective GBA gene product, e.g., a PD associated with a GBA mutation. Such a disease or condition may be a neuromuscular or a neurological disorder or condition. For example, a disease associated with decreased GCase protein levels may be Parkinson Disease or related disorder, or may be another neurological or neuromuscular disorder described herein, e.g., a PD associated with a GBA mutation, Gaucher Disease (GD) (e.g., Type 1 GD, Type 2 GD, or Type 3 GD, dementia with Lewy Bodies (DLB), Gaucher disease (GD), Spinal muscular atrophy (SMA), Multiple System Atrophy (MSA), or Multiple sclerosis (MS).

The present disclosure addresses the need for new technologies by providing GCase protein related treatment deliverable by AAV-based compositions and complexes for the treatment of GBA-related disorders.

While delivery is exemplified in the AAV context, other viral vectors, non-viral vectors, nanoparticles, or liposomes may be similarly used to deliver the therapeutic GCase protein(s) and include, but are not limited to, vector genomes of any of the AAV serotypes or other viral delivery vehicles or lentivirus, etc. The observations and teachings extend to any macromolecular structure, including modified cells, introduced into the CNS in the manner as described herein.

Given in Table 3 are the sequence identifiers of exemplary polynucleotide and polypeptide sequences for GCase proteins that may be used in the viral genomes disclosed herein and which may constitute a GCase protein payload. Functional variants, e.g., those retaining at least about 90% or at least 95% sequence identity to a sequence shown in Table 3, may also be used. In some embodiments, a codon-optimized and other variants that encode the same or essentially the same GCase protein amino acid sequence (e.g., those having at least about 90% amino acid sequence identity) may also be used.

In some embodiments, the viral genome comprises a nucleic acid comprising a transgene encoding a GBA protein, or functional variant thereof. In some embodiments, the encoded GBA protein, or functional variant thereof comprises an amino acid sequence from a GBA protein described herein, e.g., as described in Table 3 or 15, or an amino acid sequence substantially identical (e.g., having at least about 70%, 75%, 80%, 85%, 90%, 92%, 95%, 97%, 98%, or 99% sequence identity) to any of the aforesaid sequences. In some embodiments, the encoded GBA protein or functional variant thereof comprises an amino acid sequence from an GBA protein described herein, e.g., as described in Table 3 or 15, or an amino acid sequence having at least one, two or three modifications but not more than 30, 20 or 10 modifications relative to any of the aforesaid amino acid sequences. In some embodiments, the encoded GBA protein or functional variant thereof, comprises an amino acid sequence encoded by a nucleotide sequence encoding a GBA protein described herein, e.g., as described in Table 3 or 15, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the nucleotide sequence encoding the GBA protein or functional variant thereof comprises a nucleotide sequence encoding a GBA protein described herein, e.g., as described in Table 3 or 15, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the GBA protein or functional variant thereof comprises a nucleotide sequence encoding a GBA protein described herein, e.g., as described in Table 3 or 15, or a nucleotide sequence having at least one, two or three modifications but not more than 30, 20 or 10 modifications relative to any of the aforesaid nucleotide sequences. In some embodiments, the nucleotide sequence encoding a GBA protein or functional variant thereof is a codon optimized nucleotide sequence.

TABLE 3
Exemplary GCase Sequences

| SEQ ID NO: | Type | Species | Description |
|---|---|---|---|
| 1740 | Protein | Homo sapiens | GBA protein NP_000148.2 |
| 1741 | DNA | Homo sapiens | GBA mRNA transcript variant 1 |
| | | | NM_000157.4 |
| 1742 | Protein | Homo sapiens | GBA protein NP_001005741.1 |
| 1743 | DNA | Homo sapiens | GBA mRNA transcript variant 2 |
| | | | NM_01005741.3 |
| 1744 | Protein | Homo sapiens | GBA protein NP_001005742.1 |
| 1745 | DNA | Homo sapiens | GBA mRNA transcript variant 3 |
| | | | NM_001005742.3 |
| 1746 | Protein | Homo sapiens | GBA protein NP_001165282.1 |
| 1747 | DNA | Homo sapiens | GBA mRNA transcript variant 4 |
| | | | NM_001171811.2 |
| 1748 | Protein | Homo sapiens | GBA protein NP_001165283.1 |
| 1749 | DNA | Homo sapiens | GBA mRNA transcript variant 5 |
| | | | NM_001171812.2 |

TABLE 15
Exemplary GCase Sequences

| | | SEQ |
|---|---|---|
| | | ID |
| Description | Sequence | NO: |
| GBA Variant 1 | ATGGAATTCTCTAGCCCATCTAGAGAGGAATGTCCTAAGCCTCTGTCAA | 1772 |
| (signal | GAGTGTCCATCATGGCCGGCAGCCTGACAGGCCTGCTGCTGCTGCAGGC | |
| sequence | CGTGTCCTGGGCCAGTGGAGCCCGGCCCTGCATCCCTAAGTCCTTCGGC | |
| underlined)-nt | TATTCTAGCGTGGTCTGCGTGTGTAATGCCACTTACTGCGACAGCTTCG | |
| | ACCCTCCTACCTTCCCCGCCCTTGGAACATTCAGCAGATACGAGAGCAC | |
| | CAGAAGCGGCAGAAGAATGGAACTGAGCATGGGCCCAATCCAGGCCAAC | |
| | CACACCGGCACCGGCCTGCTGCTGACACTGCAACCTGAGCAGAAGTTCC | |
| | AGAAGGTGAAGGGATTTGGAGGCGCCATGACCGACGCTGCTGCTCTGAA | |
| | CATCCTGGCCCTCTCCCCACCTGCTCAGAACCTGCTGCTTAAAAGCTAC | |
| | TTCAGCGAGGAAGGCATCGGCTATAACATCATCAGAGTGCCCATGGCCA | |
| | GCTGCGACTTCAGCATCAGAACATACACCTACGCCGATACACCTGATGA | |
| | CTTCCAACTGCACAACTTCAGCCTGCCTGAAGAGGACACAAAGCTGAAA | |
| | ATCCCCCTGATCCACCGGGCCCTGCAGCTGGCCCAGAGACCTGTGAGCC | |
| | TGCTGGCCTCTCCTTGGACAAGCCCCACCTGGCTGAAGACCAATGGAGC | |
| | TGTGAACGGCAAGGGCAGCCTGAAGGGCCAGCCCGGCGACATCTACCAC | |
| | CAAACCTGGGCTCGCTACTTCGTGAAATTCCTGGACGCCTACGCTGAGC | |
| | ATAAGCTGCAATTTTGGGCCGTTACAGCCGAGAACGAGCCTTCTGCCGG | |
| | CCTGCTGTCTGGATATCCTTTCCAGTGCCTGGGCTTCACCCCTGAGCAC | |
| | CAGAGAGACTTTATCGCCAGAGATCTGGGGCCTACCCTGGCTAACAGCA | |
| | CACACCACAACGTGCGGCTGCTGATGCTGGACGATCAGAGGCTGCTGCT | |
| | CCCCCACTGGGCCAAGGTGGTGCTGACAGATCCGGAGGCCGCCAAATAC | |
| | GTGCACGGCATCGCCGTCCACTGGTACCTGGATTTCCTGGCCCCTGCCA | |
| | AGGCCACCCTGGGCGAGACACATAGACTGTTTCCTAATACCATGCTGTT | |
| | CGCCAGCGAGGCCTGCGTGGGCAGCAAGTTCTGGGAACAGAGCGTGCGG | |
| | CTGGGCAGCTGGGACAGAGGAATGCAGTACAGCCACAGCATCATTACCA | |
| | ACCTGCTGTACCACGTGGTGGGCTGGACCGACTGGAACCTGGCCCTGAA | |
| | CCCCGAAGGCGGCCCCAACTGGGTGCGGAACTTCGTGGACTCTCCTATC | |
| | ATCGTGGATATTACCAAGGATACCTTTTACAAGCAGCCTATGTTCTACC | |
| | ACCTGGGCCACTTCAGCAAGTTCATCCCTGAGGGCTCTCAGCGGGTGGG | |
| | CCTGGTGGCCTCTCAGAAAAACGACCTGGATGCCGTTGCCCTGATGCAC | |
| | CCCGACGGCAGCGCCGTGGTGGTCGTCCTGAATAGAAGCTCCAAGGACG | |
| | TGCCTCTGACCATCAAGGACCCCGCTGTGGGATTTCTGGAAACCATCAG | |
| | CCCTGGCTACAGCATCCACACCTACCTGTGGCGGCGGCAG | |
| GBA Variant 1 | GCCCGGCCCTGCATCCCTAAGTCCTTCGGCTATTCTAGCGTGGTCTGCG | 1773 |
| (no signal | TGTGTAATGCCACTTACTGCGACAGCTTCGACCCTCCTACCTTCCCCGC | |
| sequence)-nt | CCTTGGAACATTCAGCAGATACGAGAGCACCAGAAGCGGCAGAAGAATG | |
| | GAACTGAGCATGGGCCCAATCCAGGCCAACCACACCGGCACCGGCCTGC | |
| | TGCTGACACTGCAACCTGAGCAGAAGTTCCAGAAGGTGAAGGGATTTGG | |
| | AGGCGCCATGACCGACGCTGCTGCTCTGAACATCCTGGCCCTCTCCCCA | |
| | CCTGCTCAGAACCTGCTGCTTAAAAGCTACTTCAGCGAGGAAGGCATCG | |
| | GCTATAACATCATCAGAGTGCCCATGGCCAGCTGCGACTTCAGCATCAG | |
| | AACATACACCTACGCCGATACACCTGATGACTTCCAACTGCACAACTTC | |
| | AGCCTGCCTGAAGAGGACACAAAGCTGAAAATCCCCCTGATCCACCGGG | |
| | CCCTGCAGCTGGCCCAGAGACCTGTGAGCCTGCTGGCCTCTCCTTGGAC | |
| | AAGCCCCACCTGGCTGAAGACCAATGGAGCTGTGAACGGCAAGGGCAGC | |
| | CTGAAGGGCCAGCCCGGCGACATCTACCACCAAACCTGGGCTCGCTACT | |
| | TCGTGAAATTCCTGGACGCCTACGCTGAGCATAAGCTGCAATTTTGGGC | |
| | CGTTACAGCCGAGAACGAGCCTTCTGCCGGCCTGCTGTCTGGATATCCT | |
| | TTCCAGTGCCTGGGCTTCACCCCTGAGCACCAGAGAGACTTTATCGCCA | |
| | GAGATCTGGGGCCTACCCTGGCTAACAGCACACACCACAACGTGCGGCT | |
| | GCTGATGCTGGACGATCAGAGGCTGCTGCTCCCCCACTGGGCCAAGGTG | |
| | GTGCTGACAGATCCGGAGGCCGCCAAATACGTGCACGGCATCGCCGTCC | |
| | ACTGGTACCTGGATTTCCTGGCCCCTGCCAAGGCCACCCTGGGCGAGAC | |
| | ACATAGACTGTTTCCTAATACCATGCTGTTCGCCAGCGAGGCCTGCGTG | |
| | GGCAGCAAGTTCTGGGAACAGAGCGTGCGGCTGGGCAGCTGGGACAGAG | |
| | GAATGCAGTACAGCCACAGCATCATTACCAACCTGCTGTACCACGTGGT | |
| | GGGCTGGACCGACTGGAACCTGGCCCTGAACCCCGAAGGCGGCCCCAAC | |
| | TGGGTGCGGAACTTCGTGGACTCTCCTATCATCGTGGATATTACCAAGG | |
| | ATACCTTTTACAAGCAGCCTATGTTCTACCACCTGGGCCACTTCAGCAA | |
| | GTTCATCCCTGAGGGCTCTCAGCGGGTGGGCCTGGTGGCCTCTCAGAAA | |
| | AACGACCTGGATGCCGTTGCCCTGATGCACCCCGACGGCAGCGCCGTGG | |
| | TGGTCGTCCTGAATAGAAGCTCCAAGGACGTGCCTCTGACCATCAAGGA | |
| | CCCCGCTGTGGGATTTCTGGAAACCATCAGCCCTGGCTACAGCATCCAC | |
| | ACCTACCTGTGGCGGCGGCAG | |
| GBA Variant 1 | MEFSSPSREECPKPLSRVSIMAGSLTGLLLLQAVSWASGARPCIPKSFG | 1774 |
| (signal | YSSVVCVCNATYCDSFDPPTFPALGTFSRYESTRSGRRMELSMGPIQAN | |
| sequence | HTGTGLLLTLQPEQKFQKVKGFGGAMTDAAALNILALSPPAQNLLLKSY | |
| underlined)-aa | FSEEGIGYNIIRVPMASCDFSIRTYTYADTPDDFQLHNFSLPEEDTKLK | |
| | IPLIHRALQLAQRPVSLLASPWTSPTWLKTNGAVNGKGSLKGQPGDIYH | |
| | QTWARYFVKFLDAYAEHKLQFWAVTAENEPSAGLLSGYPFQCLGFTPEH | |
| | QRDFIARDLGPTLANSTHHNVRLLMLDDQRLLLPHWAKVVLTDPEAAKY | |
| | VHGIAVHWYLDFLAPAKATLGETHRLFPNTMLFASEACVGSKFWEQSVR | |
| | LGSWDRGMQYSHSIITNLLYHVVGWTDWNLALNPEGGPNWVRNFVDSPI | |
| | IVDITKDTFYKQPMFYHLGHFSKFIPEGSQRVGLVASQKNDLDAVALMH | |
| | PDGSAVVVVLNRSSKDVPLTIKDPAVGFLETISPGYSIHTYLWRRQ | |
| GBA Variant 1 | ARPCIPKSFGYSSVVCVCNATYCDSFDPPTFPALGTFSRYESTRSGRRM | 1775 |
| (no signal | ELSMGPIQANHTGTGLLLTLQPEQKFQKVKGFGGAMTDAAALNILALSP | |
| sequence)-aa | PAQNLLLKSYFSEEGIGYNIIRVPMASCDFSIRTYTYADTPDDFQLHNF | |
| | SLPEEDTKLKIPLIHRALQLAQRPVSLLASPWTSPTWLKTNGAVNGKGS | |
| | LKGQPGDIYHQTWARYFVKFLDAYAEHKLQFWAVTAENEPSAGLLSGYP | |
| | FQCLGFTPEHQRDFIARDLGPTLANSTHHNVRLLMLDDQRLLLPHWAKV | |
| | VLTDPEAAKYVHGIAVHWYLDFLAPAKATLGETHRLFPNTMLFASEACV | |
| | GSKFWEQSVRLGSWDRGMQYSHSIITNLLYHVVGWTDWNLALNPEGGPN | |
| | WVRNFVDSPIIVDITKDTFYKQPMFYHLGHFSKFIPEGSQRVGLVASQK | |
| | NDLDAVALMHPDGSAVVVVLNRSSKDVPLTIKDPAVGFLETISPGYSIH | |
| | TYLWRRQ | |
| GBA Variant 2 | ATGGAGTTTTCAAGTCCTTCCAGAGAGGAATGTCCCAAGCCTTTGAGTA | 1776 |
| (signal | GGGTAAGCATCATGGCTGGCAGCCTCACAGGATTGCTTCTACTTCAGGC | |
| sequence | AGTGTCGTGGGCATCAGGTGCCCGCCCCTGCATCCCTAAAAGCTTCGGC | |
| underlined)-nt | TACAGCTCGGTGGTGTGTGTCTGCAATGCCACATACTGTGACTCCTTTG | |
| | ACCCCCCGACCTTTCCTGCCCTTGGTACCTTCAGCCGCTATGAGAGTAC | |
| | ACGCAGTGGGCGACGGATGGAGCTGAGTATGGGGCCCATCCAGGCTAAT | |
| | CACACGGGCACAGGCCTGCTACTGACCCTGCAGCCAGAACAGAAGTTCC | |
| | AGAAAGTGAAGGGATTTGGAGGGGCCATGACAGATGCTGCTGCTCTCAA | |
| | CATCCTTGCCCTGTCACCCCCTGCCCAAAATTTGCTACTTAAATCGTAC | |
| | TTCTCTGAAGAAGGAATCGGATATAACATCATCCGGGTACCCATGGCCA | |
| | GCTGTGACTTCTCCATCCGCACCTACACCTATGCAGACACCCCTGATGA | |
| | TTTCCAGTTGCACAACTTCAGCCTCCCAGAGGAAGATACCAAGCTCAAG | |
| | ATACCCCTGATTCACCGAGCCCTGCAGTTGGCCCAGCGTCCCGTTTCAC | |
| | TCCTTGCCAGCCCCTGGACATCACCCACTTGGCTCAAGACCAATGGAGC | |
| | GGTGAATGGGAAGGGGTCACTCAAGGGACAGCCCGGAGACATCTACCAC | |
| | CAGACCTGGGCCAGATACTTTGTGAAGTTCCTGGATGCCTATGCTGAGC | |
| | ACAAGTTACAGTTCTGGGCAGTGACAGCTGAAAATGAGCCTTCTGCTGG | |
| | GCTGTTGAGTGGATACCCCTTCCAGTGCCTGGGCTTCACCCCTGAACAT | |
| | CAGCGAGACTTCATTGCCCGTGACCTAGGTCCTACCCTCGCCAACAGTA | |
| | CTCACCACAATGTCCGCCTACTCATGCTGGATGACCAACGCTTGCTGCT | |
| | GCCCCACTGGGCAAAGGTGGTACTGACAGACCCAGAAGCAGCTAAATAT | |
| | GTTCATGGCATTGCTGTACATTGGTACCTGGACTTTCTGGCTCCAGCCA | |
| | AAGCCACCCTAGGGGAGACACACCGCCTGTTCCCCAACACCATGCTCTT | |
| | TGCCTCAGAGGCCTGTGTGGGCTCCAAGTTCTGGGAGCAGAGTGTGCGG | |
| | CTAGGCTCCTGGGATCGAGGGATGCAGTACAGCCACAGCATCATCACGA | |
| | ACCTCCTGTACCATGTGGTCGGCTGGACCGACTGGAACCTTGCCCTGAA | |
| | CCCCGAAGGAGGACCCAATTGGGTGCGTAACTTTGTCGACAGTCCCATC | |
| | ATTGTAGACATCACCAAGGACACGTTTTACAAACAGCCCATGTTCTACC | |
| | ACCTTGGCCACTTCAGCAAGTTCATTCCTGAGGGCTCCCAGAGAGTGGG | |
| | GCTGGTTGCCAGTCAGAAGAACGACCTGGACGCAGTGGCACTGATGCAT | |
| | CCCGATGGCTCTGCTGTTGTGGTCGTGCTAAACCGCTCCTCTAAGGATG | |
| | TGCCTCTTACCATCAAGGATCCTGCTGTGGGCTTCCTGGAGACAATCTC | |
| | ACCTGGCTACTCCATTCACACCTACCTGTGGCGTCGCCAG | |
| GBA Variant 2 | GCCCGCCCCTGCATCCCTAAAAGCTTCGGCTACAGCTCGGTGGTGTGTG | 1777 |
| (no signal | TCTGCAATGCCACATACTGTGACTCCTTTGACCCCCCGACCTTTCCTGC | |
| sequence)-nt | CCTTGGTACCTTCAGCCGCTATGAGAGTACACGCAGTGGGCGACGGATG | |
| | GAGCTGAGTATGGGGCCCATCCAGGCTAATCACACGGGCACAGGCCTGC | |
| | TACTGACCCTGCAGCCAGAACAGAAGTTCCAGAAAGTGAAGGGATTTGG | |
| | AGGGGCCATGACAGATGCTGCTGCTCTCAACATCCTTGCCCTGTCACCC | |
| | CCTGCCCAAAATTTGCTACTTAAATCGTACTTCTCTGAAGAAGGAATCG | |
| | GATATAACATCATCCGGGTACCCATGGCCAGCTGTGACTTCTCCATCCG | |
| | CACCTACACCTATGCAGACACCCCTGATGATTTCCAGTTGCACAACTTC | |
| | AGCCTCCCAGAGGAAGATACCAAGCTCAAGATACCCCTGATTCACCGAG | |
| | CCCTGCAGTTGGCCCAGCGTCCCGTTTCACTCCTTGCCAGCCCCTGGAC | |
| | ATCACCCACTTGGCTCAAGACCAATGGAGCGGTGAATGGGAAGGGGTCA | |
| | CTCAAGGGACAGCCCGGAGACATCTACCACCAGACCTGGGCCAGATACT | |
| | TTGTGAAGTTCCTGGATGCCTATGCTGAGCACAAGTTACAGTTCTGGGC | |
| | AGTGACAGCTGAAAATGAGCCTTCTGCTGGGCTGTTGAGTGGATACCCC | |
| | TTCCAGTGCCTGGGCTTCACCCCTGAACATCAGCGAGACTTCATTGCCC | |
| | GTGACCTAGGTCCTACCCTCGCCAACAGTACTCACCACAATGTCCGCCT | |
| | ACTCATGCTGGATGACCAACGCTTGCTGCTGCCCCACTGGGCAAAGGTG | |
| | GTACTGACAGACCCAGAAGCAGCTAAATATGTTCATGGCATTGCTGTAC | |
| | ATTGGTACCTGGACTTTCTGGCTCCAGCCAAAGCCACCCTAGGGGAGAC | |
| | ACACCGCCTGTTCCCCAACACCATGCTCTTTGCCTCAGAGGCCTGTGTG | |
| | GGCTCCAAGTTCTGGGAGCAGAGTGTGCGGCTAGGCTCCTGGGATCGAG | |
| | GGATGCAGTACAGCCACAGCATCATCACGAACCTCCTGTACCATGTGGT | |
| | CGGCTGGACCGACTGGAACCTTGCCCTGAACCCCGAAGGAGGACCCAAT | |
| | TGGGTGCGTAACTTTGTCGACAGTCCCATCATTGTAGACATCACCAAGG | |
| | ACACGTTTTACAAACAGCCCATGTTCTACCACCTTGGCCACTTCAGCAA | |
| | GTTCATTCCTGAGGGCTCCCAGAGAGTGGGGCTGGTTGCCAGTCAGAAG | |
| | AACGACCTGGACGCAGTGGCACTGATGCATCCCGATGGCTCTGCTGTTG | |
| | TGGTCGTGCTAAACCGCTCCTCTAAGGATGTGCCTCTTACCATCAAGGA | |
| | TCCTGCTGTGGGCTTCCTGGAGACAATCTCACCTGGCTACTCCATTCAC | |
| | ACCTACCTGTGGCGTCGCCAG | |
| GBA Variant 2 | MEFSSPSREECPKPLSRVSIMAGSLTGLLLLQAVSWASGARPCIPKSFG | 1778 |
| (signal | YSSVVCVCNATYCDSFDPPTFPALGTFSRYESTRSGRRMELSMGPIQAN | |
| sequence | HTGTGLLLTLQPEQKFQKVKGFGGAMTDAAALNILALSPPAQNLLLKSY | |
| underlined)-aa | FSEEGIGYNIIRVPMASCDFSIRTYTYADTPDDFQLHNFSLPEEDTKLK | |
| | IPLIHRALQLAQRPVSLLASPWTSPTWLKTNGAVNGKGSLKGQPGDIYH | |
| | QTWARYFVKFLDAYAEHKLQFWAVTAENEPSAGLLSGYPFQCLGFTPEH | |
| | QRDFIARDLGPTLANSTHHNVRLLMLDDQRLLLPHWAKVVLTDPEAAKY | |
| | VHGIAVHWYLDFLAPAKATLGETHRLFPNTMLFASEACVGSKFWEQSVR | |
| | LGSWDRGMQYSHSIITNLLYHVVGWTDWNLALNPEGGPNWVRNFVDSPI | |
| | IVDITKDTFYKQPMFYHLGHFSKFIPEGSQRVGLVASQKNDLDAVALMH | |
| | PDGSAVVVVLNRSSKDVPLTIKDPAVGFLETISPGYSIHTYLWRRQ | |
| GBA Variant 2 | ARPCIPKSFGYSSVVCVCNATYCDSFDPPTFPALGTFSRYESTRSGRRM | 1779 |
| (no signal | ELSMGPIQANHTGTGLLLTLQPEQKFQKVKGFGGAMTDAAALNILALSP | |
| sequence)-aa | PAQNLLLKSYFSEEGIGYNIIRVPMASCDFSIRTYTYADTPDDFQLHNF | |
| | SLPEEDTKLKIPLIHRALQLAQRPVSLLASPWTSPTWLKTNGAVNGKGS | |
| | LKGQPGDIYHQTWARYFVKFLDAYAEHKLQFWAVTAENEPSAGLLSGYP | |
| | FQCLGFTPEHQRDFIARDLGPTLANSTHHNVRLLMLDDQRLLLPHWAKV | |
| | VLTDPEAAKYVHGIAVHWYLDFLAPAKATLGETHRLFPNTMLFASEACV | |
| | GSKFWEQSVRLGSWDRGMQYSHSIITNLLYHVVGWTDWNLALNPEGGPN | |
| | WVRNFVDSPIIVDITKDTFYKQPMFYHLGHFSKFIPEGSQRVGLVASQK | |
| | NDLDAVALMHPDGSAVVVVLNRSSKDVPLTIKDPAVGFLETISPGYSIH | |
| | TYLWRRQ | |
| GBA Variant 3 | atggaattcagcagccccagcagagaggaatgccccaagcctctgagcc | 1780 |
| (signal | gggtgtcaatcatggccggatctctgacaggactgctgctgcttcaggc | |
| sequence | cgtgtcttgggcttctggcgctagaccttgcatccccaagagcttcggc | |
| underlined)-nt | tacagcagcgtcgtgtgcgtgtgcaatgccacctactgcgacagcttcg | |
| | accctcctacctttcctgctctgggcaccttcagcagatacgagagcac | |
| | cagatccggcagacggatggaactgagcatgggacccatccaggccaat | |
| | cacacaggcactggcctgctgctgacactgcagcctgagcagaaattcc | |
| | agaaagtgaaaggcttcggcggagccatgacagatgccgccgctctgaa | |
| | tatcctggctctgtctccaccagctcagaacctgctgctcaagagctac | |
| | ttcagcgaggaaggcatcggctacaacatcatcagagtgcccatggcca | |
| | gctgcgacttcagcatcaggacctacacctacgccgacacacccgacga | |
| | tttccagctgcacaacttcagcctgcctgaagaggacaccaagctgaag | |
| | atccctctgatccacagagccctgcagctggcacaaagacccgtgtcac | |
| | tgctggcctctccatggacatctcccacctggctgaaaacaaatggcgc | |
| | cgtgaatggcaagggcagcctgaaaggccaacctggcgacatctaccac | |
| | cagacctgggccagatacttcgtgaagttcctggacgcctatgccgagc | |
| | acaagctgcagttttgggccgtgacagccgagaacgaaccttctgctgg | |
| | actgctgagcggctacccctttcagtgcctgggctttacacccgagcac | |
| | cagcgggactttatcgcccgtgatctgggacccacactggccaatagca | |
| | cccaccataatgtgcggctgctgatgctggacgaccagagactgcttct | |
| | gccccactgggctaaagtggtgctgacagatcctgaggccgccaaatac | |
| | gtgcacggaatcgccgtgcactggtatctggactttctggcccctgcca | |
| | aggccacactgggagagacacacagactgttccccaacaccatgctgtt | |
| | cgccagcgaagcctgtgtgggcagcaagttttgggaacagagcgtgcgg | |
| | ctcggcagctgggatagaggcatgcagtacagccacagcatcatcacca | |
| | acctgctgtaccacgtcgtcggctggaccgactggaatctggccctgaa | |
| | tcctgaaggcggccctaactgggtccgaaacttcgtggacagccccatc | |
| | atcgtggacatcaccaaggacaccttctacaagcagcccatgttctacc | |
| | acctgggacacttcagcaagttcatccccgagggctctcagcgcgttgg | |
| | actggtggcttcccagaagaacgatctggacgccgtggctctgatgcac | |
| | cctgatggatctgctgtggtggtggtcctgaaccgcagcagcaaagatg | |
| | tgcccctgaccatcaaggatcccgccgtgggattcctggaaacaatcag | |
| | ccctggctactccatccacacctacctgtggcgtagacag | |
| GBA Variant 3 | gctagaccttgcatccccaagagcttcggctacagcagcgtcgtgtgcg | 1781 |
| (no signal | tgtgcaatgccacctactgcgacagcttcgaccctcctacctttcctgc | |
| sequence)-nt | tctgggcaccttcagcagatacgagagcaccagatccggcagacggatg | |
| | gaactgagcatgggacccatccaggccaatcacacaggcactggcctgc | |
| | tgctgacactgcagcctgagcagaaattccagaaagtgaaaggcttcgg | |
| | cggagccatgacagatgccgccgctctgaatatcctggctctgtctcca | |
| | ccagctcagaacctgctgctcaagagctacttcagcgaggaaggcatcg | |
| | gctacaacatcatcagagtgcccatggccagctgcgacttcagcatcag | |
| | gacctacacctacgccgacacacccgacgatttccagctgcacaacttc | |
| | agcctgcctgaagaggacaccaagctgaagatccctctgatccacagag | |
| | ccctgcagctggcacaaagacccgtgtcactgctggcctctccatggac | |
| | atctcccacctggctgaaaacaaatggcgccgtgaatggcaagggcagc | |
| | ctgaaaggccaacctggcgacatctaccaccagacctgggccagatact | |
| | tcgtgaagttcctggacgcctatgccgagcacaagctgcagttttgggc | |
| | cgtgacagccgagaacgaaccttctgctggactgctgagcggctacccc | |
| | tttcagtgcctgggctttacacccgagcaccagcgggactttatcgccc | |
| | gtgatctgggacccacactggccaatagcacccaccataatgtgcggct | |
| | gctgatgctggacgaccagagactgcttctgccccactgggctaaagtg | |
| | gtgctgacagatcctgaggccgccaaatacgtgcacggaatcgccgtgc | |
| | actggtatctggactttctggcccctgccaaggccacactgggagagac | |
| | acacagactgttccccaacaccatgctgttcgccagcgaagcctgtgtg | |
| | ggcagcaagttttgggaacagagcgtgcggctcggcagctgggatagag | |
| | gcatgcagtacagccacagcatcatcaccaacctgctgtaccacgtcgt | |
| | cggctggaccgactggaatctggccctgaatcctgaaggcggccctaac | |
| | tgggtccgaaacttcgtggacagccccatcatcgtggacatcaccaagg | |
| | acaccttctacaagcagcccatgttctaccacctgggacacttcagcaa | |
| | gttcatccccgagggctctcagcgcgttggactggtggcttcccagaag | |
| | aacgatctggacgccgtggctctgatgcaccctgatggatctgctgtgg | |
| | tggtggtcctgaaccgcagcagcaaagatgtgcccctgaccatcaagga | |
| | tcccgccgtgggattcctggaaacaatcagccctggctactccatccac | |
| | acctacctgtggcgtagacag | |
| GBA Variant 3 | MEFSSPSREECPKPLSRVSIMAGSLTGLLLLQAVSWASGARPCIPKSFG | 1782 |
| (signal | YSSVVCVCNATYCDSFDPPTFPALGTFSRYESTRSGRRMELSMGPIQAN | |
| sequence | HTGTGLLLTLQPEQKFQKVKGFGGAMTDAAALNILALSPPAQNLLLKSY | |
| underlined)-aa | FSEEGIGYNIIRVPMASCDFSIRTYTYADTPDDFQLHNFSLPEEDTKLK | |
| | IPLIHRALQLAQRPVSLLASPWTSPTWLKTNGAVNGKGSLKGQPGDIYH | |
| | QTWARYFVKFLDAYAEHKLQFWAVTAENEPSAGLLSGYPFQCLGFTPEH | |
| | QRDFIARDLGPTLANSTHHNVRLLMLDDQRLLLPHWAKVVLTDPEAAKY | |
| | VHGIAVHWYLDFLAPAKATLGETHRLFPNTMLFASEACVGSKFWEQSVR | |
| | LGSWDRGMQYSHSIITNLLYHVVGWTDWNLALNPEGGPNWVRNFVDSPI | |
| | IVDITKDTFYKQPMFYHLGHFSKFIPEGSQRVGLVASQKNDLDAVALMH | |
| | PDGSAVVVVLNRSSKDVPLTIKDPAVGFLETISPGYSIHTYLWRRQ | |
| GBA Variant 3 | ARPCIPKSFGYSSVVCVCNATYCDSFDPPTFPALGTFSRYESTRSGRRM | 1783 |
| (no signal | ELSMGPIQANHTGTGLLLTLQPEQKFQKVKGFGGAMTDAAALNILALSP | |
| sequence)-aa | PAQNLLLKSYFSEEGIGYNIIRVPMASCDFSIRTYTYADTPDDFQLHNF | |
| | SLPEEDTKLKIPLIHRALQLAQRPVSLLASPWTSPTWLKTNGAVNGKGS | |
| | LKGQPGDIYHQTWARYFVKFLDAYAEHKLQFWAVTAENEPSAGLLSGYP | |
| | FQCLGFTPEHQRDFIARDLGPTLANSTHHNVRLLMLDDQRLLLPHWAKV | |
| | VLTDPEAAKYVHGIAVHWYLDFLAPAKATLGETHRLFPNTMLFASEACV | |
| | GSKFWEQSVRLGSWDRGMQYSHSIITNLLYHVVGWTDWNLALNPEGGPN | |
| | WVRNFVDSPIIVDITKDTFYKQPMFYHLGHFSKFIPEGSQRVGLVASQK | |
| | NDLDAVALMHPDGSAVVVVLNRSSKDVPLTIKDPAVGFLETISPGYSIH | |
| | TYLWRRQ | |

In some embodiments, the encoded GBA protein or functional variant thereof comprises the amino acid sequence of any one of SEQ ID NOs: 1740, 1742, 1744, 1746, 1748, 1774, 1775, 1778, 1779, 1782, or 1783, or an amino acid sequence substantially identical (e.g., having at least about 70%, 75%, 80%, 85%, 90%, 92%, 95%, 97%, 98%, or 99% sequence identity) to any of the aforesaid sequences. In some embodiments, the encoded GBA protein or functional variant thereof comprises the amino acid sequence of any one of SEQ ID NOs: 1740, 1742, 1744, 1746, 1748, 1774, 1775, 1778, 1779, 1782, or 1783, or an amino acid having at least one, two or three modifications but not more than 30, 20 or 10 modifications relative to any of the aforesaid amino acid sequences. In some embodiments, the encoded GBA protein or functional variant thereof comprises an amino acid sequence encoded by the nucleotide sequence of any of SEQ ID NOs: 1741, 1743, 1744, 1745, 1747, 1749, 1772, 1773, 1776, 1777, 1780, or 1781, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the nucleotide sequence encoding the GBA protein or functional variant thereof comprises the nucleotide sequence of any one of SEQ ID NOs: 1741, 1743, 1744, 1745, 1747, 1749, 1772, 1773, 1776, 1777, 1780, or 1781, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the nucleic acid sequence encoding the GBA protein or functional variant thereof comprises the nucleotide sequence of any one of SEQ ID NOs: 1741, 1743, 1744, 1745, 1747, 1749, 1772, 1773, 1776, 1777, 1780, or 1781, or a nucleotide sequence having at least one, two or three modifications but not more than 30, 20 or 10 modifications relative to any of the aforesaid nucleotide sequences. In some embodiments, the nucleotide sequence encoding the GBA protein or functional variant thereof comprises the nucleotide sequence of SEQ ID NO: 1773, a nucleotide sequence substantially identical (e.g., having at least about 70%, 75%, 80%, 85%, 90%, 92%, 95%, 97%, 98%, or 99% sequence identity) to SEQ ID NO: 1773, or a nucleotide sequence having at least one, two or three modifications but not more than 30, 20 or 10 modifications relative to SEQ ID NO: 1773. In some embodiments, the nucleotide sequence encoding the GBA protein or functional variant thereof does not comprise a stop codon. In some embodiments, the nucleotide sequence encoding the GBA protein of functional variant thereof is a codon optimized nucleotide sequence.

In some embodiments, a codon optimized nucleotide sequence encoding a GBA protein described herein (e.g., SEQ ID NO: 1773) replaces a donor splice site, e.g., a nucleotide sequence comprising the sequence of AGGGTAAGC or nucleotides 49 of the 117 numbered according to the nucleotide sequence of SEQ ID NO: 1776, with the nucleotide sequence of AGAGTGTCC, e.g., comprising at least one, two, three, or four modifications, e.g., mutations relative to the nucleotide sequence of AGGGTAAGC, or nucleotides 49 of the 117 numbered according to the nucleotide sequence of SEQ ID NO: 1776. In some embodiments, a codon optimized nucleotide sequence encoding a GBA protein described herein (e.g., SEQ ID NO: 1773) contains more than 121, 122, 123, 124, 125, 126, 127, 128, 129, 130, 131, 132, 133, 134, 135, 136, 137, 138, 139, 140 or more unique modifications, e.g., mutations, compared to the nucleotide sequence of SEQ ID NO: 1776. In some embodiments, a codon optimized nucleotide sequence of a GBA protein described herein (e.g., SEQ ID NO: 1773) comprises a unique GC content profile. Without wishing to be bound by theory, it is believed in some embodiments, that altering the GC-content of a nucleotide sequence of a GBA protein described herein enhances the expression of the codon optimized nucleotide sequence in a cell (e.g., a human cell or a neuronal cell).

In some embodiments, the viral genome comprises a payload region encoding a GCase protein. The encoded GCase protein may be derived from any species, such as, but not limited to human, non-human primate, or rodent.

In some embodiments, the viral genome comprises a payload region encoding a human (Homo sapiens) GCase protein, or a variant thereof.

Various embodiments of the disclosure herein provide an adeno-associated viral (AAV) particle comprising a viral genome, the viral genome comprising at least one inverted terminal repeat region and a nucleic acid sequence encoding a polypeptide having at least 90% sequence identity to a human GCase protein sequence, or a fragment thereof, as provided in Table 3. In some embodiments, the AAV viral genome comprises at least one inverted terminal repeat region and a nucleic acid sequence encoding a polypeptide having at least 95% sequence identity to a GCase protein sequence, or a fragment thereof, as provided in Table 3. In some embodiments, the AAV viral genome comprises at least one inverted terminal repeat region and a nucleic acid sequence encoding a polypeptide having at least 98% sequence identity to a GCase protein sequence, or a fragment thereof, as provided in Table 3. In some embodiments, the AAV viral genome comprises at least one inverted terminal repeat region and a nucleic acid sequence encoding a polypeptide having at least 99% sequence identity to a GCase protein sequence, or a fragment thereof, as provided in Table 3. In some embodiments, the AAV viral genome comprises at least one inverted terminal repeat region and a nucleic acid sequence encoding a GCase protein sequence, or a fragment thereof, provided in Table 3.

In some embodiments, the viral genome comprises a nucleic acid sequence encoding a recombinant glucocerebrosidase according to Imiglucerase (Cerezyme)(Genzyme Corp.), a recombinant GCase for use in treating Gaucher disease; Velaglucerase (Vpriv)(Shire Human Genetic Therapies Inc.), a recombinant GCase for use in treating Gaucher disease; or U.S. Pat. Nos. 8,227,230, 8,741,620, or U.S. Pat. No. 8,790,641, each incorporated by reference herein, describing Taliglucerase alfa (Elelyso)(Pfizer Inc.), a recombinant GCase for use in treating Gaucher disease.

In some embodiments, the GCase protein is derived from a GBA protein encoding sequence of a non-human primate, such as the cynomolgus monkey, Macaca fascicularis. Certain embodiments provide the GCase protein as a humanized version of a Macaca fascicularis sequence.

In some embodiments, the viral genome comprises a payload region encoding a cynomolgus or crab-eating (long-tailed) macaque (Macaca fascicularis) GCase protein, or a variant thereof.

In some embodiments, the viral genome comprises a payload region encoding a rhesus macaque (Macaca mulatta) GCase protein, or a variant thereof.

In some embodiments, the GCase protein may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 3.

In some embodiments, the GCase protein may be encoded by a nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 3.

The GCase protein payloads as described herein can encode any GCase protein, or any portion or derivative of a GCase protein, and are not limited to the GCase proteins or protein-encoding sequences provided in Table 3.

### Payload Component: Enhancement Element

In some embodiments, a viral genome described herein encoding a GBA protein comprises an enhancement element or functional variant thereof. In some embodiments, the encoded enhancement comprises a prosaposin (PSAP) protein, a saposin C (SapC) protein, or functional variant thereof; a cell penetrating peptide (e.g., a ApoEII peptide, a TAT peptide, and/or a ApoB peptide) or functional variant thereof; or a lysosomal targeting signal or functional variant thereof.

In some embodiments, the viral genome comprises a payload region further encoding a prosaposin (PSAP) protein or a saposin C (SapC) protein or functional variant thereof, e.g., as described herein, e.g., in Table 4 or 16.

TABLE 4
Exemplary PSAP and Saposin Sequences

| SEQ | | | |
|---|---|---|---|
| ID | | | |
| NO: | Type | Species | Description |
| 1750 | Pro- | Homo | Prosaposin isoform A preprotein, |
| | tein | sapiens | NP_002769.1 |
| 1751 | DNA | Homo | PSAP transcript variant 1, |
| | | sapiens | NM_002778.4 |
| 1752 | Pro- | Homo | Prosaposin isoform B preprotein, |
| | tein | sapiens | NP_001035930.1 |
| 1753 | DNA | Homo | PSAP transcript variant 2, |
| | | sapiens | NM_001042465.3 |
| 1754 | Pro- | Homo | Prosaposin isoform C preprotein, |
| | tein | sapiens | NP_001035931.1 |
| 1755 | DNA | Homo | PSAP transcript variant 3, |
| | | sapiens | NM_001042466.3 |
| 1756 | Pro- | Homo | hSapA, amino acids 60 to 140 of |
| | tein | sapiens | SEQ ID NO: 1750: |
| | | | SLPCDICKDVVTAAGDMLKDNATEEEILVYL |
| | | | EKTCDWLPKPNMSASCKEIVDSYLPVILDII |
| | | | KGEMSRPGEVCSALNLCES |
| 1757 | Pro- | Homo | hSapB, amino acids 195 to 275 |
| | tein | sapiens | of SEQ ID NO: 1750: |
| | | | GDVCQDCIQMVTDIQTAVRTNSTFVQALVEH |
| | | | VKEECDRLGPGMADICKNYISQYSEIAIQMM |
| | | | MHMQPKEICALVGFCDEVK |
| 1758 | Pro- | Homo | hSapC, amino acids 311 to 390 |
| | tein | sapiens | of SEQ ID NO: 1750: |
| | | | SDVYCEVCEFLVKEVTKLIDNNKTEKEILDA |
| | | | FDKMCSKLPKSLSEECQEVVDTYGSSILSIL |
| | | | LEEVSPELVCSMLHLCSG |
| 1784 | Pro- | Homo | hSapD, amino acids 405 to 486 |
| | tein | sapiens | of SEQ ID NO 1750: |
| | | | DGGFCEVCKKLVGYLDRNLEKNSTKQEILAA |
| | | | LEKGCSFLPDPYQKQCDQFVAEYEPVLIEIL |
| | | | VEVMDPSFVCLKIGACPSAH |
| 1856 | DNA | Homo | Signal Sequence |
| | | sapiens | atgtacgccctcttcctcctggccagcctcc |
| | | | tgggcgcggctctagcc |
| 1857 | Pro- | Homo | Signal Sequence |
| | tein | sapiens | MYALFLLASLLGAALA |

TABLE 16
Exemplary Enhancement Elements

| | | SEQ |
|---|---|---|
| | | ID |
| Description | Sequence | NO: |
| PSAP or Saposin | | |
| PSAP | atgtacgccctcttcctcctggccagcctcctgggcgcggctctagc | 1858 |
| (signal | cggcccggtccttggactgaaagaatgcaccaggggctcggcagtgt | |
| sequence | ggtgccagaatgtgaagacggcgtccgactgcggggcagtgaagcac | |
| underlined)-nt | tgcctgcagaccgtttggaacaagccaacagtgaaatcccttccctg | |
| | cgacatatgcaaagacgttgtcaccgcagctggtgatatgctgaagg | |
| | acaatgccactgaggaggagatccttgtttacttggagaagacctgt | |
| | gactggcttccgaaaccgaacatgtctgcttcatgcaaggagatagt | |
| | ggactcctacctccctgtcatcctggacatcattaaaggagaaatga | |
| | gccgtcctggggaggtgtgctctgctctcaacctctgcgagtctctc | |
| | cagaagcacctagcagagctgaatcaccagaagcagctggagtccaa | |
| | taagatcccagagctggacatgactgaggtggtggcccccttcatgg | |
| | ccaacatccctctcctcctctaccctcaggacggcccccgcagcaag | |
| | ccccagccaaaggataatggggacgtttgccaggactgcattcagat | |
| | ggtgactgacatccagactgctgtacggaccaactccacctttgtcc | |
| | aggccttggtggaacatgtcaaggaggagtgtgaccgcctgggccct | |
| | ggcatggccgacatatgcaagaactatatcagccagtattctgaaat | |
| | tgctatccagatgatgatgcacatgcaacccaaggagatctgtgcgc | |
| | tggttgggttctgtgatgaggtgaaagagatgcccatgcagactctg | |
| | gtccccgccaaagtggcctccaagaatgtcatccctgccctggaact | |
| | ggtggagcccattaagaagcacgaggtcccagcaaagtctgatgttt | |
| | actgtgaggtgtgtgaattcctggtgaaggaggtgaccaagctgatt | |
| | gacaacaacaagactgagaaagaaatactcgacgcttttgacaaaat | |
| | gtgctcgaagctgccgaagtccctgtcggaagagtgccaggaggtgg | |
| | tggacacgtacggcagctccatcctgtccatcctgctggaggaggtc | |
| | agccctgagctggtgtgcagcatgctgcacctctgctctggcacgcg | |
| | gctgcctgcactgaccgttcacgtgactcagccaaaggacggtggct | |
| | tctgcgaagtgtgcaagaagctggtgggttatttggatcgcaacctg | |
| | gagaaaaacagcaccaagcaggagatcctggctgctcttgagaaagg | |
| | ctgcagcttcctgccagacccttaccagaagcagtgtgatcagtttg | |
| | tggcagagtacgagcccgtgctgatcgagatcctggtggaggtgatg | |
| | gatccttccttcgtgtgcttgaaaattggagcctgcccctcggccca | |
| | taagcccttgttgggaactgagaagtgtatatggggcccaagctact | |
| | ggtgccagaacacagagacagcagcccagtgcaatgctgtcgagcat | |
| | tgcaaacgccatgtgtggaactag | |
| PSAP | ggcccggtccttggactgaaagaatgcaccaggggctcggcagtgtg | 1859 |
| (no signal | gtgccagaatgtgaagacggcgtccgactgcggggcagtgaagcact | |
| sequence)-nt | gcctgcagaccgtttggaacaagccaacagtgaaatcccttccctgc | |
| | gacatatgcaaagacgttgtcaccgcagctggtgatatgctgaagga | |
| | caatgccactgaggaggagatccttgtttacttggagaagacctgtg | |
| | actggcttccgaaaccgaacatgtctgcttcatgcaaggagatagtg | |
| | gactcctacctccctgtcatcctggacatcattaaaggagaaatgag | |
| | ccgtcctggggaggtgtgctctgctctcaacctctgcgagtctctcc | |
| | agaagcacctagcagagctgaatcaccagaagcagctggagtccaat | |
| | aagatcccagagctggacatgactgaggtggtggcccccttcatggc | |
| | caacatccctctcctcctctaccctcaggacggcccccgcagcaagc | |
| | cccagccaaaggataatggggacgtttgccaggactgcattcagatg | |
| | gtgactgacatccagactgctgtacggaccaactccacctttgtcca | |
| | ggccttggtggaacatgtcaaggaggagtgtgaccgcctgggccctg | |
| | gcatggccgacatatgcaagaactatatcagccagtattctgaaatt | |
| | gctatccagatgatgatgcacatgcaacccaaggagatctgtgcgct | |
| | ggttgggttctgtgatgaggtgaaagagatgcccatgcagactctgg | |
| | tccccgccaaagtggcctccaagaatgtcatccctgccctggaactg | |
| | gtggagcccattaagaagcacgaggtcccagcaaagtctgatgttta | |
| | ctgtgaggtgtgtgaattcctggtgaaggaggtgaccaagctgattg | |
| | acaacaacaagactgagaaagaaatactcgacgcttttgacaaaatg | |
| | tgctcgaagctgccgaagtccctgtcggaagagtgccaggaggtggt | |
| | ggacacgtacggcagctccatcctgtccatcctgctggaggaggtca | |
| | gccctgagctggtgtgcagcatgctgcacctctgctctggcacgcgg | |
| | ctgcctgcactgaccgttcacgtgactcagccaaaggacggtggctt | |
| | ctgcgaagtgtgcaagaagctggtgggttatttggatcgcaacctgg | |
| | agaaaaacagcaccaagcaggagatcctggctgctcttgagaaaggc | |
| | tgcagcttcctgccagacccttaccagaagcagtgtgatcagtttgt | |
| | ggcagagtacgagcccgtgctgatcgagatcctggtggaggtgatgg | |
| | atccttccttcgtgtgcttgaaaattggagcctgcccctcggcccat | |
| | aagcccttgttgggaactgagaagtgtatatggggcccaagctactg | |
| | gtgccagaacacagagacagcagcccagtgcaatgctgtcgagcatt | |
| | gcaaacgccatgtgtggaactag | |
| PSAP | MYALFLLASLLGAALAGPVLGLKECTRGSAVWCQNVKTASDCGAVKH | 1750 |
| (signal | CLQTVWNKPTVKSLPCDICKDVVTAAGDMLKDNATEEEILVYLEKTC | |
| sequence | DWLPKPNMSASCKEIVDSYLPVILDIIKGEMSRPGEVCSALNLCESL | |
| underlined)-aa | QKHLAELNHQKQLESNKIPELDMTEVVAPFMANIPLLLYPQDGPRSK | |
| | PQPKDNGDVCQDCIQMVTDIQTAVRTNSTFVQALVEHVKEECDRLGP | |
| | GMADICKNYISQYSEIAIQMMMHMQPKEICALVGFCDEVKEMPMQTL | |
| | VPAKVASKNVIPALELVEPIKKHEVPAKSDVYCEVCEFLVKEVTKLI | |
| | DNNKTEKEILDAFDKMCSKLPKSLSEECQEVVDTYGSSILSILLEEV | |
| | SPELVCSMLHLCSGTRLPALTVHVTQPKDGGFCEVCKKLVGYLDRNL | |
| | EKNSTKQEILAALEKGCSFLPDPYQKQCDQFVAEYEPVLIEILVEVM | |
| | DPSFVCLKIGACPSAHKPLLGTEKCIWGPSYWCQNTETAAQCNAVEH | |
| | CKRHVWN | |
| PSAP | GPVLGLKECTRGSAVWCQNVKTASDCGAVKHCLQTVWNKPTVKSLPC | 1785 |
| (no signal | DICKDVVTAAGDMLKDNATEEEILVYLEKTCDWLPKPNMSASCKEIV | |
| sequence) | DSYLPVILDIIKGEMSRPGEVCSALNLCESLQKHLAELNHQKQLESN | |
| | KIPELDMTEVVAPFMANIPLLLYPQDGPRSKPQPKDNGDVCQDCIQM | |
| | VTDIQTAVRTNSTFVQALVEHVKEECDRLGPGMADICKNYISQYSEI | |
| | AIQMMMHMQPKEICALVGFCDEVKEMPMQTLVPAKVASKNVIPALEL | |
| | VEPIKKHEVPAKSDVYCEVCEFLVKEVTKLIDNNKTEKEILDAFDKM | |
| | CSKLPKSLSEECQEVVDTYGSSILSILLEEVSPELVCSMLHLCSGTR | |
| | LPALTVHVTQPKDGGFCEVCKKLVGYLDRNLEKNSTKQEILAALEKG | |
| | CSFLPDPYQKQCDQFVAEYEPVLIEILVEVMDPSFVCLKIGACPSAH | |
| | KPLLGTEKCIWGPSYWCQNTETAAQCNAVEHCKRHVWN | |
| SAPC | atgtacgccctcttcctcctggccagcctcctgggcgcggctctagc | 1786 |
| (signal | cgtgaaagagatgcccatgcagactctggtccccgccaaagtggcct | |
| sequence | ccaagaatgtcatccctgccctggaactggtggagcccattaagaag | |
| underlined)-nt | cacgaggtcccagcaaagtctgatgtttactgtgaggtgtgtgaatt | |
| | cctggtgaaggaggtgaccaagctgattgacaacaacaagactgaga | |
| | aagaaatactcgacgcttttgacaaaatgtgctcgaagctgccgaag | |
| | tccctgtcggaagagtgccaggaggtggtggacacgtacggcagctc | |
| | catcctgtccatcctgctggaggaggtcagccctgagctggtgtgca | |
| | gcatgctgcacctctgctctggc | |
| SAPC | gtgaaagagatgcccatgcagactctggtccccgccaaagtggcctc | 1787 |
| (no signal | caagaatgtcatccctgccctggaactggtggagcccattaagaagc | |
| sequence)-nt | acgaggtcccagcaaagtctgatgtttactgtgaggtgtgtgaattc | |
| | ctggtgaaggaggtgaccaagctgattgacaacaacaagactgagaa | |
| | agaaatactcgacgcttttgacaaaatgtgctcgaagctgccgaagt | |
| | ccctgtcggaagagtgccaggaggtggtggacacgtacggcagctcc | |
| | atcctgtccatcctgctggaggaggtcagccctgagctggtgtgcag | |
| | catgctgcacctctgctctggc | |
| SAPC | MYALFLLASLLGAALAVKEMPMQTLVPAKVASKNVIPALELVEPIKK | 1788 |
| (signal | HEVPAKSDVYCEVCEFLVKEVTKLIDNNKTEKEILDAFDKMCSKLPK | |
| sequence | SLSEECQEVVDTYGSSILSILLEEVSPELVCSMLHLCSG | |
| underlined)-aa | | |
| SAPC | VKEMPMQTLVPAKVASKNVIPALELVEPIKKHEVPAKSDVYCEVCEF | 1789 |
| (no signal | LVKEVTKLIDNNKTEKEILDAFDKMCSKLPKSLSEECQEVVDTYGSS | |
| sequence)-aa | ILSILLEEVSPELVCSMLHLCSG | |
| SAPCv2 | atgtacgccctcttcctcctggccagcctcctgggcgcggctctagc | 1790 |
| (signal | ctctgatgtttactgtgaggtgtgtgaattcctggtgaaggaggtga | |
| sequence | ccaagctgattgacaacaacaagactgagaaagaaatactcgacgct | |
| underlined)-nt | tttgacaaaatgtgctcgaagctgccgaagtccctgtcggaagagtg | |
| | ccaggaggtggtggacacgtacggcagctccatcctgtccatcctgc | |
| | tggaggaggtcagccctgagctggtgtgcagcatgctgcacctctgc | |
| | tctggc | |
| SAPCv2 | tctgatgtttactgtgaggtgtgtgaattcctggtgaaggaggtgac | 1791 |
| (no signal | caagctgattgacaacaacaagactgagaaagaaatactcgacgctt | |
| sequence)-nt | ttgacaaaatgtgctcgaagctgccgaagtccctgtcggaagagtgc | |
| | caggaggtggtggacacgtacggcagctccatcctgtccatcctgct | |
| | ggaggaggtcagccctgagctggtgtgcagcatgctgcacctctgct | |
| | ctggc | |
| SAPCv2 | MYALFLLASLLGAALASDVYCEVCEFLVKEVTKLIDNNKTEKEILDA | 1792 |
| (signal | FDKMCSKLPKSLSEECQEVVDTYGSSILSILLEEVSPELVCSMLHLC | |
| sequence | SG | |
| underlined)-aa | | |
| SAPCv2 | SDVYCEVCEFLVKEVTKLIDNNKTEKEILDAFDKMCSKLPKSLSEEC | 1758 |
| (no signal | QEVVDTYGSSILSILLEEVSPELVCSMLHLCSG | |
| sequence)-aa | | |
| Cell | | |
| Penetrating | | |
| Peptides | | |
| TAT - nt | tatggcaggaaaaagcggaggcaaaggcgccgccccccccag | 1793 |
| TAT - aa | YGRKKRRQRRRPPQ | 1794 |
| ApoB - nt | tccgtaatcgacgccttacagtataagctggagggaaccaccagatt | 1795 |
| | gacaaggaaacgagggcttaagcttgctactgcactatccctgagca | |
| | ataaattt | |
| ApoB - aa | SVIDALQYKLEGTTRLTRKRGLKLATALSLSNKF | 1796 |
| ApoEII - nt | ctacggaagctgcggaagcggctactgctgcggaaacttcggaaacg | 1797 |
| | gctactg | |
| ApoEII - aa | LRKLRKRLLLRKLRKRLL | 1798 |
| Lysosomal | | |
| Targeting | | |
| Sequence (LTS) | | |
| LTS1 - nt | aagtttgaaagacag | 1799 |
| LTS1 - aa | KFERQ | 1800 |
| LTS2 - nt | atgaaggagaccgctgctgcaaagttcgagagacagcatatggatag | 1801 |
| | ctccacaagcgccgca | |
| LTS2 - aa | MKETAAAKFERQHMDSSTSAA | 1802 |
| LTS3 - nt | cagaaaatcctggat | 1803 |
| LTS3 - aa | QKILD | 1804 |
| LTS4 - nt | cagagattcttcgag | 1805 |
| LTS4 - aa | QRFFE | 1806 |
| LTS5 - nt | aagtttgaaagacagcagaaaatcctggatcagagattcttcgag | 1807 |
| LTS5 - aa | KFERQQKILDQRFFE | 1808 |

In some embodiments, the viral genome comprises a payload region encoding a SapC protein. The encoded SapC may be derived from any species, such as, but not limited to human, non-human primate, or rodent. SapC protein is thought to coordinate GCase activity of GBA by locally altering lipid membranes, exposing glucosylceramide molecules for hydrolysis (see Alattia, Jean-Rene, et al. “Molecular imaging of membrane interfaces reveals mode of β-glucosidase activation by saposin C.” Proceedings of the National Academy of Sciences 104.44 (2007): 17394-17399, the contents of which are incorporated by reference herein in their entirety).

In some embodiments, the viral genome comprises a payload region encoding a human (Homo sapiens) SapC, or a variant thereof.

Various embodiments of the disclosure herein provide an adeno-associated viral (AAV) particle comprising a viral genome, the viral genome comprising at least one inverted terminal repeat region and a nucleic acid sequence encoding a polypeptide having at least 90% sequence identity to a human SapC (hSapC) sequence, or a fragment thereof, as provided in Table 4. In some embodiments, the AAV viral genome comprises at least one inverted terminal repeat region and a nucleic acid sequence encoding a polypeptide having at least 95% sequence identity to a Saposin sequence, or a fragment thereof, as provided in Table 4. In some embodiments, the AAV viral genome comprises at least one inverted terminal repeat region and a nucleic acid sequence encoding a polypeptide having at least 98% sequence identity to a Saposin sequence, or a fragment thereof, as provided in Table 4. In some embodiments, the AAV viral genome comprises at least one inverted terminal repeat region and a nucleic acid sequence encoding a polypeptide having at least 99% sequence identity to a Saposin sequence, or a fragment thereof, as provided in Table 4. In some embodiments, the AAV viral genome comprises at least one inverted terminal repeat region and a nucleic acid sequence encoding a Saposin sequence, or a fragment thereof, as provided in Table 4.

In some embodiments, the Saposin polypeptide is derived from a Saposin or PSAP sequence of a non-human primate, such as the cynomolgus monkey, Macaca fascicularis (cynoPSAP or cPSAP). Certain embodiments provide the Saposin polypeptide as a humanized version of a Macaca fascicularis (HcynoSap) sequence.

In some embodiments, the viral genome comprises a payload region encoding a cynomolgus or crab-eating (long-tailed) macaque (Macaca fascicularis) PSAP or Saposin, or a variant thereof.

In some embodiments, the viral genome comprises a payload region encoding a rhesus macaque (Macaca mulatta) PSAP or Saposin, or a variant thereof.

In some embodiments, the viral genome comprises a payload region encoding a murine (Mus musculus) PSAP or Saposin, or variant thereof.

In some embodiments, the PSAP or Saposin polypeptide may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 4.

In some embodiments, the PSAP or Saposin polypeptide may be encoded by a nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 4.

In some embodiments, the viral genome comprises a payload region further encoding a PD-associated gene the lack of expression of which causes or leads to or promotes the development of PD. Such PD-associated gene incudes GCase/GBA1, GBA2, prosapsin, LIMP2/SCARB2 (e.g., the gene product of SCARB2 gene), progranulin, GALC, CTSB, SMPD1, GCH1, RAB7, VPS35, IL-34, TREM2, TMEM106B, a combination of any of the foregoing, or a functional fragment thereof.

Thus in some embodiments, the viral genome comprises a payload region encoding a LIMP2/SCARB2, a membrane protein that regulates lysosomal and endosomal transport within a cell. In some embodiments, the SCARB2 gene encodes a peptide that is represented by NCBI Reference Sequence NP_005497.1 (incorporated herein by reference). In some embodiments the isolated nucleic acid comprises a SCARB2-encoding sequence that has been codon optimized.

In some embodiments, the viral genome comprises a payload region encoding a GBA2 protein (e.g., the gene product of GBA2 gene). In some embodiments, the GBA2-encoding sequence has been codon optimized (e.g., codon optimized for expression in mammalian cells, for example human cells). In some embodiments, the GBA2-encoding sequence encodes a protein comprising an amino acid sequence as set forth in NCBI Reference Sequence NP_065995.1 (incorporated herein by reference).

In some embodiments, the viral genome comprises a payload region encoding a GALC protein (e.g., the gene product of GALC gene). In some embodiments, the GALC-encoding sequence has been codon optimized (e.g., codon optimized for expression in mammalian cells, for example human cells). In some embodiments, the GALC-encoding sequence encodes a protein comprising an amino acid sequence as set forth in NCBI Reference Sequence NP_000144.2 (incorporated herein by reference).

In some embodiments, the viral genome comprises a payload region encoding a CTSB protein (e.g., the gene product of CTSB gene). In some embodiments, the CTSB-encoding sequence has been codon optimized (e.g., codon optimized for expression in mammalian cells, for example human cells). In some embodiments, the CTSB-encoding sequence encodes a protein comprising an amino acid sequence as set forth in NCBI Reference Sequence NP_001899.1 (incorporated by reference).

In some embodiments, the viral genome comprises a payload region encoding a SMPD1 protein (e.g., the gene product of SMPD1 gene). In some embodiments, the SMPD1-encoding sequence has been codon optimized (e.g., codon optimized for expression in mammalian cells, for example human cells). In some embodiments, the SMPD1-encoding sequence encodes a protein comprising an amino acid sequence as set forth in NCBI Reference Sequence NP_000534.3 (incorporated herein by reference).

In some embodiments, the viral genome comprises a payload region encoding a GCH1 protein (e.g., the gene product of GCH1 gene). In some embodiments, the GCH1-encoding sequence has been codon optimized (e.g., codon optimized for expression in mammalian cells, for example human cells). In some embodiments, the GCH1-encoding sequence encodes a protein comprising an amino acid sequence as set forth in NCBI Reference Sequence NP_000534.3 (incorporated by reference).

In some embodiments, the viral genome comprises a payload region encoding a RAB7L protein (e.g., the gene product of RAB7L gene). In some embodiments, the RAB7L-encoding sequence has been codon optimized (e.g., codon optimized for expression in mammalian cells, for example human cells). In some embodiments, the RAB7L encodes a protein comprising an amino acid sequence as set forth in NCBI Reference Sequence NP_003920.1 (incorporated by reference).

In some embodiments, the viral genome comprises a payload region encoding a VPS35 protein (e.g., the gene product of VPS35 gene). In some embodiments, the VPS35-encoding sequence has been codon optimized (e.g., codon optimized for expression in mammalian cells, for example human cells). In some embodiments, the VPS35 encodes a protein comprising an amino acid sequence as set forth in NCBI Reference Sequence NP_060676.2 (incorporated by reference).

In some embodiments, the viral genome comprises a payload region encoding an IL-34 protein (e.g., the gene product of IL34 gene). In some embodiments, the IL-34-encoding sequence has been codon optimized (e.g., codon optimized for expression in mammalian cells, for example human cells). In some embodiments, the IL-34-encoding sequence encodes a protein comprising an amino acid sequence as set forth in NCBI Reference Sequence NP_689669.2 (incorporated by reference).

In some embodiments, the viral genome comprises a payload region encoding a TREM2 protein (e.g., the gene product of TREM gene). In some embodiments, the TREM2-encoding sequence has been codon optimized (e.g., codon optimized for expression in mammalian cells, for example human cells). In some embodiments, the TREM2-encoding sequence encodes a protein comprising an amino acid sequence as set forth in NCBI Reference Sequence NP_061838.1 (incorporated by reference).

In some embodiments, the viral genome comprises a payload region encoding a TMEM106B protein (e.g., the gene product of TMEM106B gene). In some embodiments, the TMEM106B-encoding sequence has been codon optimized (e.g., codon optimized for expression in mammalian cells, for example human cells). In some embodiments, the TMEM106B-encoding sequence encodes a protein comprising an amino acid sequence as set forth in NCBI Reference Sequence NP_060844.2 (incorporated by reference).

In some embodiments, the viral genome comprises a payload region encoding a progranulin (e.g., the gene product of PGRN gene). In some embodiments, the progranulin-encoding sequence has been codon optimized (e.g., codon optimized for expression in mammalian cells, for example human cells). In some embodiments, the nucleic acid sequence encoding the progranulin (PRGN) encodes a protein comprising an amino acid sequence as set forth in NCBI Reference Sequence NP_002078.1 (incorporated by reference).

In certain embodiments, a functional fragment of any of the above protein such as GCase/GBA, GBA2, LIMP2/SCARB2, progranulin, GALC, CTSB, SMPD1, GCH1, RAB7, VPS35, IL-34, TREM2, TMEM106B, and prosapsin (such as SapA-SapD) may comprise about 50%, about 60%, about 70%, about 80% about 90% or about 99% of a protein encoded by the respective wt genes or gene segments (such as coding sequence for SapA-SapD). In some embodiments, a functional fragment of a wt sequence comprises between 50% and 99.9% (e.g., any value between 50% and 99.9%) of a protein encoded by a wt sequence.

### Exemplary GCase/SapC Payloads

In some embodiments, the viral genome comprises a payload region encoding a GCase protein and a SapC protein (a GCase/SapC polypeptide). The encoded GCase/SapC polypeptide may be derived from GCase and SapC protein sequences of any species, such as, but not limited to human, non-human primate, or rodent.

Various embodiments of the disclosure herein provide an adeno-associated viral (AAV) particle comprising a viral genome, the viral genome comprising at least one inverted terminal repeat region and a nucleic acid sequence encoding a GCase/SapC polypeptide having a region of at least 90% sequence identity to a human GCase sequence provided in Table 3 or a fragment or variant thereof and a region of at least 90% sequence identity to a human SapC sequence provided in Table 4 or 16, or a fragment or variant thereof.

In some embodiments, the GCase/SapC polypeptide may comprise a GCase region having 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the those in Table 3 or 15.

In some embodiments, the GCase/SapC polypeptide may comprise a SapC region having 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the those in Table 4 or 16.

In some embodiments, the GCase/SapC polypeptide may be encoded by a nucleic acid sequence having a GCase region with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the those described in Table 3 or 15.

In some embodiments, the GCase/SapC polypeptide may be encoded by a nucleic acid sequence having a SapC region with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the those described in Table 4 or 16.

Viral genomes may be engineered with one or more spacer or linker regions to separate coding or non-coding regions. In some embodiments, the payload region of the AAV particle may optionally encode one or more linker sequences. In some cases, the linker may be a peptide linker that may be used to connect the polypeptides encoded by the payload region (i.e., GCase polypeptides and SapC polypeptides). Some peptide linkers may be cleaved after expression to separate GCase and SapC polypeptides, allowing expression of separate functional polypeptides. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., GCase and SapC domains) from a single transcript. In some cases, two or more linkers are encoded by a payload region of the viral genome. Non-limiting examples of linkers that may be encoded by the payload region of an AAV particle viral genome are given in Table 2.

In some embodiments, GCase and SapC polypeptides are delivered separately in independent AAV vectors.

In certain embodiments, viral genomes for expressing Gcase and/or Saposin may comprise a sequence as described in Table 5.

In some embodiments, the AAV viral genomes described herein comprise an enhancement elements such as a lysosomal targeting peptide sequence (LTS), a cell penetrating peptide (CPP), or both. For example, in some embodiments, a payload may have a sequence encoding a lysosomal targeting peptide. The sequence encoding the lysosomal targeting peptide can be a sequence derived from GCase. In some cases, it is a LIMP-2 binding domain, or a variant thereof, which aides in the intracellular trafficking of a molecule to lysosomes, which is responsible for the intracellular trafficking of GCase to lysosomes via LIMP-2 (Liou, Benjamin, et al. Journal of Biological Chemistry 289.43 (2014): 30063-30074, the contents of which are incorporated herein by reference in their entirety).

### Exemplary GBA AAV Viral Genome Sequence Regions and ITR to ITR Sequences

In some embodiments, a viral genome, e.g., an AAV viral genome or vector genome, described herein, comprises a promoter operably linked to a transgene encoding a GBA protein. In some embodiments, the viral genome further comprises an inverted terminal repeat region, an enhancer, an intron, a miR binding site, a polyA region, or a combination thereof. Exemplary sequence regions within ITR to ITR sequences for viral genomes according to the description are provided in Table 5.

TABLE 5
Exemplary Viral Genome sequence regions in ITR to ITR constructs

| | | SEQ |
|---|---|---|
| | | ID |
| Description | Sequence | NO: |
| ITR (130nt) | ctgcgcgctcgctcgctcactgaggccgcccgggcaaagcccgggcgtcgggcg | 1829 |
| | acctttggtcgcccggcctcagtgagcgagcgagcgcgcagagagggagtggcc | |
| | aactccatcactaggggttcct | |
| ITR(130nt) | aggaacccctagtgatggagttggccactccctctctgcgcgctcgctcgctca | 1830 |
| | ctgaggccgggcgaccaaaggtcgcccgacgcccgggctttgcccgggcggcct | |
| | cagtgagcgagcgagcgcgcag | |
| ITR variant A | TATTAGATCTGATGGCCGC | 1860 |
| ITR variant B | CTCCATCACTAGGGGTTCCT | 1861 |
| ITR variant B | AGGAACCCCTAGTGATGGAGTTGGCCACTCCCTCTCTGCGCGCTCGCTCGCTCA | 1862 |
| | CTGAGGCCGGGCGACCAAAGGTCGCCCGACGCCCGGGCTTTGCCCGGGCGGCCT | |
| | CAGTGAGCGAGCGAGCGCGCAGAGAGGGAGTGGCCAA | |
| ITR variant C | TATTAGATCTGATGGCCGCG | 1863 |
| ITR variant D | TCCATCACTAGGGGTTCCTG | 1864 |
| CMVie | GACATTGATTATTGACTAGTTATTAATAGTAATCAATTACGGGGTCATTAGTTC | 1831 |
| enhancer | ATAGCCCATATATGGAGTTCCGCGTTACATAACTTACGGTAAATGGCCCGCCTG | |
| | GCTGACCGCCCAACGACCCCCGCCCATTGACGTCAATAATGACGTATGTTCCCA | |
| | TAGTAACGCCAATAGGGACTTTCCATTGACGTCAATGGGTGGAGTATTTACGGT | |
| | AAACTGCCCACTTGGCAGTACATCAAGTGTATCATATGCCAAGTACGCCCCCTA | |
| | TTGACGTCAATGACGGTAAATGGCCCGCCTGGCATTATGCCCAGTACATGACCT | |
| | TATGGGACTTTCCTACTTGGCAGTACATCTACGTATTAGTCATCGCTATTACCA | |
| | TG | |
| CMV | gtgatgcggttttggcagtacatcaatgggcgtggatagcggtttgactcacgg | 1832 |
| promoter | ggatttccaagtctccaccccattgacgtcaatgggagtttgttttggcaccaa | |
| | aatcaacgggactttccaaaatgtcgtaacaactccgccccattgacgcaaatg | |
| | ggcggtaggcgtgtacggtgggaggtctatataagcagagctc | |
| CMV | gacattgattattgactagttattaatagtaatcaattacggggtcattagttc | 1833 |
| promoter | atagcccatatatggagttccgcgttacataacttacggtaaatggcccgcctg | |
| region (CMV | gctgaccgcccaacgacccccgcccattgacgtcaataatgacgtatgttccca | |
| enhancer and | tagtaacgccaatagggactttccattgacgtcaatgggtggagtatttacggt | |
| promoter) | aaactgcccacttggcagtacatcaagtgtatcatatgccaagtacgcccccta | |
| | ttgacgtcaatgacggtaaatggcccgcctggcattatgcccagtacatgacct | |
| | tatgggactttcctacttggcagtacatctacgtattagtcatcgctattacca | |
| | tggtgatgcggttttggcagtacatcaatgggcgtggatagcggtttgactcac | |
| | ggggatttccaagtctccaccccattgacgtcaatgggagtttgttttggcacc | |
| | aaaatcaacgggactttccaaaatgtcgtaacaactccgccccattgacgcaaa | |
| | tgggcggtaggcgtgtacggtgggaggtctatataagcagagctc | |
| CB promoter | ccacgttctgcttcactctccccatctcccccccctccccacccccaattttgt | 1834 |
| | atttatttattttttaattattttgtgcagcgatgggggcggggggggggggcg | |
| | cgcgccaggcggggcggggcggggcgaggggcggggcggggcgaggcggagagg | |
| | tgcggcggcagccaatcagagcggcgcgctccgaaagtttccttttatggcgag | |
| | gcggcggcggcggcggccctataaaaagcgaagcgcgcggcggg | |
| CAG promoter | ctagttattaatagtaatcaattacggggtcattagttcatagcccatatatgg | 1835 |
| | agttccgcgttacataacttacggtaaatggcccgcctggctgaccgcccaacg | |
| | acccccgcccattgacgtcaataatgacgtatgttcccatagtaacgccaatag | |
| | ggactttccattgacgtcaatgggtggagtatttacggtaaactgcccacttgg | |
| | cagtacatcaagtgtatcatatgccaagtacgccccctattgacgtcaatgacg | |
| | gtaaatggcccgcctggcattatgcccagtacatgaccttatgggactttccta | |
| | cttggcagtacatctacgtattagtcatcgctattaccatggtcgaggtgagcc | |
| | ccacgttctgcttcactctccccatctcccccccctccccacccccaattttgt | |
| | atttatttattttttaattattttgtgcagcgatgggggcgggggggggggggg | |
| | ggcgcgcgccaggcggggcggggcggggcgaggggcggggcggggcgaggcgga | |
| | gaggtgcggcggcagccaatcagagcggcgcgctccgaaagtttccttttatgg | |
| | cgaggcggcggcggcggcggccctataaaaagcgaagcgcgcggcgggcgGgag | |
| | tcgctgcgcgctgccttcgccccgtgccccgctccgccgccgcctcgcgccgcc | |
| | cgccccggctctgactgaccgcgttactcccacaggtgagcgggcgggacggcc | |
| | cttctcctccgggctgtaattagcgcttggtttaatgacggcttgtttcttttc | |
| | tgtggctgcgtgaaagccttgaggggctccgggagggccctttgtgcgggggga | |
| | gcggctcggggggtgcgtgcgtgtgtgtgtgcgtggggagcgccgcgtgcggct | |
| | ccgcgctgcccggcggctgtgagcgctgcgggcgcggcgcggggctttgtgcgc | |
| | tccgcagtgtgcgcgaggggagcgcggccgggggcggtgccccgcggtgcgggg | |
| | ggggctgcgaggggaacaaaggctgcgtgcggggtgtgtgcgtgggggggtgag | |
| | cagggggtgtgggcgcgtcggtcgggctgcaaccccccctgcacccccctcccc | |
| | gagttgctgagcacggcccggcttcgggtgcggggctccgtacggggcgtggcg | |
| | cggggctcgccgtgccgggcggggggtggcggcaggtgggggtgccgggcgggg | |
| | cggggccgcctcgggccggggagggctcgggggaggggcgcggcggcccccgga | |
| | gcgccggcggctgtcgaggcgcggcgagccgcagccattgccttttatggtaat | |
| | cgtgcgagagggcgcagggacttcctttgtcccaaatctgtgcggagccgaaat | |
| | ctgggaggcgccgccgcaccccctctagcgggcgcggggcgaagcggtgcggcg | |
| | ccggcaggaaggaaatgggcggggagggccttcgtgcgtcgccgcgccgccgtc | |
| | cccttctccctctccagcctcggggctgtccgcggggggacggctgccttcggg | |
| | ggggacggggcagggcggggttcggcttctggcgtgtgaccggcggctctagag | |
| | cctctgctaaccatgttcatgccttcttctttttcctacagctcctgggcaacg | |
| | tgctggttattgtgctgtctcatcattttggcaaagaattc | |
| CBA minimal | catggtcgaggtgagccccacgttctgcttcactctccccatctcccccccctc | 1836 |
| promoter | cccacccccaattttgtatttatttattttttaattattttgtgcagcgatggg | |
| | ggcggggggggggggggggcgcgcgccaggcggggcggggcggggcgaggggcg | |
| | gggcggggcgaggcggagaggtgcggcggcagccaatcagagcggcgcgctccg | |
| | aaagtttccttttatggcgaggcggcggcggcggcggccctataaaaagcgaag | |
| | cgcgcggcgggcg | |
| Intron 1 | Ggagtcgctgcgcgctgccttcgccccgtgccccgctccgccgccgcctcgcgc | 1837 |
| | cgcccgccccggctctgactgaccgcgttactcccacaggtgagcgggcgggac | |
| | ggcccttctcctccgggctgtaattagcgcttggtttaatgacggcttgtttct | |
| | tttctgtggctgcgtgaaagccttgaggggctccgggagggccctttgtgcggg | |
| | gggagcggctcggggggtgcgtgcgtgtgtgtgtgcgtggggagcgccgcgtgc | |
| | ggctccgcgctgcccggcggctgtgagcgctgcgggcgcggcgcggggctttgt | |
| | gcgctccgcagtgtgcgcgaggggagcgcggccgggggcggtgccccgcggtgc | |
| | ggggggggctgcgaggggaacaaaggctgcgtgcggggtgtgtgcgtggggggg | |
| | tgagcagggggtgtgggcgcgtcggtcgggctgcaaccccccctgcacccccct | |
| | ccccgagttgctgagcacggcccggcttcgggtgcggggctccgtacggggcgt | |
| | ggcgcggggctcgccgtgccgggcggggggtggcggcaggtgggggtgccgggc | |
| | ggggcggggccgcctcgggccggggagggctcgggggaggggcgcggcggcccc | |
| | cggagcgccggcggctgtcgaggcgcggcgagccgcagccattgccttttatgg | |
| | taatcgtgcgagagggcgcagggacttcctttgtcccaaatctgtgcggagccg | |
| | aaatctgggaggcgccgccgcaccccctctagcgggcgcggggcgaagcggtgc | |
| | ggcgccggcaggaaggaaatgggcggggagggccttcgtgcgtcgccgcgccgc | |
| | cgtccccttctccctctccagcctcggggctgtccgcggggggacggctgcctt | |
| | cgggggggacggggcagggcggggttcggcttctggcgtgtgaccggcggctct | |
| | agagcctctgctaaccatgttcatgccttcttctttttcctaca | |
| EF-1α | gcatgcgtga | 1838 |
| promoter | | |
| variant 1 | | |
| EF-1α | gcatgcgtgaggctccggtgcccgtcagtgggcagagcgcacatcgcccacagt | 1839 |
| promoter | ccccgagaagttggggggaggggtcggcaattgaaccggtgcctagagaaggtg | |
| variant 2 | gcgcggggtaaactgggaaagtgatgtcgtgtactggctccgcctttttcccga | |
| (intron | gggtgggggagaaccgtatataagtgcagtagtcgccgtgaacgttctttttcg | |
| underlined) | caacgggtttgccgccagaacacaggtaagtgccgtgtgtggttcccgcgggcc | |
| | tggcctctttacgggttatggcccttgcgtgccttgaattacttccacctggct | |
| | gcagtacgtgattcttgatcccgagcttcgggttggaagtgggtgggagagttc | |
| | gaggccttgcgcttaaggagccccttcgcctcgtgcttgagttgaggcctggcc | |
| | tgggcgctggggccgccgcgtgcgaatctggtggcaccttcgcgcctgtctcgc | |
| | tgctttcgataagtctctagccatttaaaatttttgatgacctgctgcgacgct | |
| | ttttttctggcaagatagtcttgtaaatgcgggccaagatctgcacactggtat | |
| | ttcggtttttggggccgcgggcggcgacggggcccgtgcgtcccagcgcacatg | |
| | ttcggcgaggcggggcctgcgagcgcggccaccgagaatcggacgggggtagtc | |
| | tcaagctggccggcctgctctggtgcctggcctcgcgccgccgtgtatcgcccc | |
| | gccctgggcggcaaggctggcccggtcggcaccagttgcgtgagcggaaagatg | |
| | gccgcttcccggccctgctgcagggagctcaaaatggaggacgcggcgctcggg | |
| | agagcgggcgggtgagtcacccacacaaaggaaaagggcctttccgtcctcagc | |
| | cgtcgcttcatgtgactccacggagtaccgggcgccgtccaggcacctcgatta | |
| | gttctcgagcttttggagtacgtcgtctttaggttggggggaggggttttatgc | |
| | gatggagtttccccacactgagtgggtggagactgaagttaggccagcttggca | |
| | cttgatgtaattctccttggaatttgccctttttgagtttggatcttggttcat | |
| | tctcaagcctcagacagtggttcaaagtttttttcttccatttcaggtgtcgtg | |
| | a | |
| EF-1α | ggctccggtgcccgtcagtgggcagagcgcacatcgcccacagtccccgagaag | 1840 |
| promoter | ttggggggaggggtcggcaattgaaccggtgcctagagaaggtggcgcggggta | |
| variant 3 | aactgggaaagtgatgtcgtgtactggctccgcctttttcccgagggtggggga | |
| (intron | gaaccgtatataagtgcagtagtcgccgtgaacgttctttttcgcaacgggttt | |
| underlined) | gccgccagaacacaggtaagtgccgtgtgtggttcccgcgggcctggcctcttt | |
| | acgggttatggcccttgcgtgccttgaattacttccacctggctgcagtacgtg | |
| | attcttgatcccgagcttcgggttggaagtgggtgggagagttcgaggccttgc | |
| | gcttaaggagccccttcgcctcgtgcttgagttgaggcctggcctgggcgctgg | |
| | ggccgccgcgtgcgaatctggtggcaccttcgcgcctgtctcgctgctttcgat | |
| | aagtctctagccatttaaaatttttgatgacctgctgcgacgctttttttctgg | |
| | caagatagtcttgtaaatgcgggccaagatctgcacactggtatttcggttttt | |
| | ggggccgcgggcggcgacggggcccgtgcgtcccagcgcacatgttcggcgagg | |
| | cggggcctgcgagcgcggccaccgagaatcggacgggggtagtctcaagctggc | |
| | cggcctgctctggtgcctggcctcgcgccgccgtgtatcgccccgccctgggcg | |
| | gcaaggctggcccggtcggcaccagttgcgtgagcggaaagatggccgcttccc | |
| | ggccctgctgcagggagctcaaaatggaggacgcggcgctcgggagagcgggcg | |
| | ggtgagtcacccacacaaaggaaaagggcctttccgtcctcagccgtcgcttca | |
| | tgtgactccacggagtaccgggcgccgtccaggcacctcgattagttctcgagc | |
| | ttttggagtacgtcgtctttaggttggggggaggggttttatgcgatggagttt | |
| | ccccacactgagtgggtggagactgaagttaggccagcttggcacttgatgtaa | |
| | ttctccttggaatttgccctttttgagtttggatcttggttcattctcaagcct | |
| | cagacagtggttcaaagtttttttcttccatttcaggtgtcgtga | |
| EF-1α | gtaagtgccgtgtgtggttcccgcgggcctggcctctttacgggttatggccct | 1841 |
| intron A | tgcgtgccttgaattacttccacctggctgcagtacgtgattcttgatcccgag | |
| | cttcgggttggaagtgggtgggagagttcgaggccttgcgcttaaggagcccct | |
| | tcgcctcgtgcttgagttgaggcctggcctgggcgctggggccgccgcgtgcga | |
| | atctggtggcaccttcgcgcctgtctcgctgctttcgataagtctctagccatt | |
| | taaaatttttgatgacctgctgcgacgctttttttctggcaagatagtcttgta | |
| | aatgcgggccaagatctgcacactggtatttcggtttttggggccgcgggcggc | |
| | gacggggcccgtgcgtcccagcgcacatgttcggcgaggcggggcctgcgagcg | |
| | cggccaccgagaatcggacgggggtagtctcaagctggccggcctgctctggtg | |
| | cctggcctcgcgccgccgtgtatcgccccgccctgggcggcaaggctggcccgg | |
| | tcggcaccagttgcgtgagcggaaagatggccgcttcccggccctgctgcaggg | |
| | agctcaaaatggaggacgcggcgctcgggagagcgggcgggtgagtcacccaca | |
| | caaaggaaaagggcctttccgtcctcagccgtcgcttcatgtgactccacggag | |
| | taccgggcgccgtccaggcacctcgattagttctcgagcttttggagtacgtcg | |
| | tctttaggttggggggaggggttttatgcgatggagtttccccacactgagtgg | |
| | gtggagactgaagttaggccagcttggcacttgatgtaattctccttggaattt | |
| | gccctttttgagtttggatcttggttcattctcaagcctcagacagtggttcaa | |
| | agtttttttcttccatttcag | |
| Human beta | tcagatcgcctggagacgccatccacgctgttttgacctccatagaagacaccg | 1842 |
| globin | ggaccgatccagcctccgcggattcgaatcccggccgggaacggtgcattggaa | |
| intron | cgcggattccccgtgccaagagtgacgtaagtaccgcctatagagtctataggc | |
| (hGBint) | ccacaaaaaatgctttcttcttttaatatacttttttgtttatcttatttctaa | |
| | tactttccctaatctctttctttcagggcaataatgatacaatgtatcatgcct | |
| | ctttgcaccattctaaagaataacagtgataatttctgggttaaggcaatagca | |
| | atatttctgcatataaatatttctgcatataaattgtaactgatgtaagaggtt | |
| | tcatattgctaatagcagctacaatccagctaccattctgcttttattttatgg | |
| | ttgggataaggctggattattctgagtccaagctaggcccttttgctaatcatg | |
| | ttcatacctcttatcttcctcccacagctcctgggcaacgtgctggtctgtgtg | |
| | ctggcccatcactttggcaaagaatt | |
| Furin - nt | agaaagaggcga | 1724 |
| Furin - aa | RKRR | 1854 |
| T2A - nt | gagggcagaggaagtcttctaacatgcggtgacgtggaggagaatcccggccct | 1726 |
| T2A - nt | EGRGSLLTCGDVEENPGP | 1855 |
| (G4S) | tccggaggcggcggcagc | 1729 |
| Linker - nt | | |
| (G4S) | GGGGS | 1843 |
| Linker - aa | | |
| (G4S)3 | GGAGGGGGGGGTTCGGGTGGCGGCGGAAGTGGGGGCGGTGGTTCT | 1730 |
| Linker - nt | | |
| (G4S)3-aa | GGGGSGGGGSGGGGS | 1845 |
| polyA signal | gatctttttccctctgccaaaaattatggggacatcatgaagccccttgagcat | 1846 |
| sequence | ctgacttctggctaataaaggaaatttattttcattgcaatagtgtgttggaat | |
| | tttttgtgtctctcactcg | |
| miR183 | AGTGAATTCTACCAGTGCCATA | 1847 |
| binding site | | |
| Spacer | GATAGTTA | 1848 |
| miR183 | AGTGAATTCTACCAGTGCCATAGATAGTTAAGTGAATTCTACCAGTGCCATAGA | 1849 |
| binding site | TAGTTAAGTGAATTCTACCAGTGCCATAGATAGTTAAGTGAATTCTACCAGTGC | |
| series | CATA | |
| Signal | ATGGAATTCTCTAGCCCATCTAGAGAGGAATGTCCTAAGCCTCTGTCAAGAGTG | 1850 |
| Sequence - | TCCATCATGGCCGGCAGCCTGACAGGCCTGCTGCTGCTGCAGGCCGTGTCCTGG | |
| nt | GCCAGTGGA | |
| Signal | ATGGAGTTTTCAAGTCCTTCCAGAGAGGAATGTCCCAAGCCTTTGAGTAGGGTA | 1851 |
| Sequence - | AGCATCATGGCTGGCAGCCTCACAGGATTGCTTCTACTTCAGGCAGTGTCGTGG | |
| nt | GCATCAGGT | |
| Signal | atggaattcagcagccccagcagagaggaatgccccaagcctctgagccgggtg | 1852 |
| Sequence - | tcaatcatggccggatctctgacaggactgctgctgcttcaggccgtgtcttgg | |
| nt | gcttctggc | |
| Signal | MEFSSPSREECPKPLSRVSIMAGSLTGLLLLQAVSWASG | 1853 |
| Sequence - | | |
| aa | | |

In some embodiments, the viral genome comprises an inverted terminal repeat sequence region (ITR) provided in Table 5, or a nucleotide sequence with at least 70%, 75%, 80%, 85%, 90%, 95% or 99% sequence identity to any of the ITR sequences in Table 5.

This disclosure also provides in some embodiments, a GBA protein encoded by any one of SEQ ID NOs: 1759-1771 or 1809-1828, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the viral genome comprises a promoter provided in Table 5 or a nucleotide sequence with at least 70%, 75%, 80%, 85%, 90%, 95% or 99% sequence identity to any of the promoter sequences in Table 5.

In some embodiments, the viral genome of an AAV particle described herein comprises the nucleotide sequence, e.g., the nucleotide sequence from the 5′ ITR to the 3′ ITR, of the nucleotide sequences of GBA_VG1 to GBA_VG34, e.g., as described in Tables 18-21 or 29-32, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the viral genome of an AAV particle described herein comprises the nucleotide sequence, e.g., the nucleic acid sequence from the 5′ ITR to the 3′ ITR, of any of the nucleotide sequences in Table 18-21 or 29-32, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the viral genome of an AAV particle described herein comprises the nucleotide sequence, e.g., the nucleic acid sequence from the 5′ ITR to the 3′ ITR, of any of the nucleotide sequences of SEQ ID NOs: 1759-1771, 1809-1828, or 1870, or a nucleotide sequence substantially identical (e.g., having at least about 70%, 75%, 80%, 85%, 90%, 92%, 95%, 97%, 98%, or 99% sequence identity) to any of the aforesaid sequences.

This disclosure also provides in some embodiments, a GBA protein (e.g., a GCase protein) encoded by any one of SEQ ID NOs: 1759-1771, 1809-1828, or 1870, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, a viral genome encoding a GBA protein is a wtGBA viral genome, wherein the viral genome comprises a transgene encoding a GBA protein (optionally wherein the nucleotide sequence encoding the GBA protein is a codon optimized nucleotide sequence), but does not encode an enhancement element, e.g., an enhancement element described herein. In some embodiments, a viral genome encoding a GBA protein is an enGBA viral genome, wherein the viral genome comprises a transgene encoding a GBA protein (optionally wherein the nucleotide sequence encoding the GBA protein is a codon optimized nucleotide sequence), and further encodes an enhancement element, e.g., an enhancement element described herein.

TABLE 18
Exemplary Viral Genome (ITR to ITR) sequences

| Construct ID | Description (5′ to 3′) | SEQ ID NO: | Length |
|---|---|---|---|
| GBA_VG1 | ITR (SEQ ID NO: 1829); CMVie (SEQ ID NO: 1831); CB | 1759 | 3413 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); polyA signal | | |
| | region (SEQ ID NO: 1846); ITR (SEQ ID NO: 1830) | | |
| GBA_VG2 | ITR (SEQ ID NO: 1829); CMVie (SEQ ID NO: 1831); CB | 1760 | 3428 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); Lysosomal | | |
| | targeting sequence 1 (LTS1) (SEQ ID NO: 1799); polyA signal | | |
| | region (SEQ ID NO: 1846); ITR (SEQ ID NO: 1830) | | |
| GBA_VG3 | ITR (SEQ ID NO: 1829); CMVie (SEQ ID NO: 1831); CB | 1761 | 3476 |
| | promoter (SEQ ID NO: 1834); human beta globin intron (hGBint) | | |
| | (SEQ ID NO: 1842); signal sequence (SEQ ID NO: 1852); | | |
| | Lysosomal targeting sequence 2 (LTS2) (SEQ ID NO: 1801); | | |
| | GBA Variant 3 coding sequence (SEQ ID NO: 1781); polyA | | |
| | signal region (SEQ ID NO: 1846); ITR (SEQ ID NO: 1830) | | |
| GBA_VG4 | ITR (SEQ ID NO: 1829); CMVie (SEQ ID NO: 1831); CB | 1762 | 3428 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); Lysosomal | | |
| | targeting sequence 3 (LTS3) (SEQ ID NO: 1803); polyA signal | | |
| | region (SEQ ID NO: 1846); ITR (SEQ ID NO: 1830) | | |
| GBA_VG5 | ITR (SEQ ID NO: 1829); CMVie (SEQ ID NO: 1831); CB | 1763 | 3428 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); Lysosomal | | |
| | targeting sequence 4 (LTS4) (SEQ ID NO: 1805); polyA signal | | |
| | region (SEQ ID NO: 1846); ITR (SEQ ID NO: 1830) | | |
| GBA_VG6 | ITR (SEQ ID NO: 1829); CMVie (SEQ ID NO: 1831); CB | 1764 | 3512 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); G4S3 linker | | |
| | coding sequence (SEQ ID NO: 1730); ApoEII coding sequence | | |
| | (SEQ ID NO: 1797); polyA signal region (SEQ ID NO: 1846); | | |
| | ITR (SEQ ID NO: 1830) | | |
| GBA_VG7 | ITR (SEQ ID NO: 1829); CMVie (SEQ ID NO: 1831); CB | 1765 | 3500 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); G4S3 linker | | |
| | coding sequence (SEQ ID NO: 1730); TAT coding sequence (SEQ | | |
| | ID NO: 1793); polyA signal region (SEQ ID NO: 1846); ITR | | |
| | (SEQ ID NO: 1830) | | |
| GBA_VG8 | ITR (SEQ ID NO: 1829); CMVie (SEQ ID NO: 1831); CB | 1766 | 4463 |
| | promoter (SEQ ID NO: 1834); signal sequence (SEQ ID NO: | | |
| | 1852); GBA Variant 3 coding sequence (SEQ ID NO: 1781); Furin | | |
| | cleavage site coding sequence (SEQ ID NO: 1724); T2A coding | | |
| | sequence (SEQ ID NO: 1726); signal sequence (SEQ ID NO: | | |
| | 1856); Prosaposin (PSAP) coding sequence (SEQ ID NO: 1859); | | |
| | poly A signal region (SEQ ID NO: 1846); ITR (SEQ ID NO: 1830) | | |
| GBA_VG9 | ITR (SEQ ID NO: 1829); CMVie (SEQ ID NO: 1831); CB | 1767 | 3878 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); Furin cleavage | | |
| | site coding sequence (SEQ ID NO: 1724); T2A coding sequence | | |
| | (SEQ ID NO: 1726); signal sequence (SEQ ID NO: 1856); SAPC | | |
| | coding sequence (SEQ ID NO: 1787); polyA signal region (SEQ | | |
| | ID NO: 1846); ITR (SEQ ID NO: 1830) | | |
| GBA_VG10 | ITR (SEQ ID NO: 1829); CMVie (SEQ ID NO: 1831); CB | 1768 | 3767 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); Furin cleavage | | |
| | site coding sequence (SEQ ID NO: 1724); T2A coding sequence | | |
| | (SEQ ID NO: 1726); signal sequence (SEQ ID NO: 1856); | | |
| | SAPCv2 coding sequence (SEQ ID NO: 1791); polyA signal | | |
| | region (SEQ ID NO: 1846); ITR (SEQ ID NO: 1830) | | |
| GBA_VG11 | ITR (SEQ ID NO: 1829); CMVie (SEQ ID NO: 1831); CB | 1769 | 3560 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); G4S3 linker | | |
| | coding sequence (SEQ ID NO: 1730); ApoB coding sequence | | |
| | (SEQ ID NO: 1795); polyA signal region (SEQ ID NO: 1846); | | |
| | ITR (SEQ ID NO: 1830) | | |
| GBA_VG12 | ITR (SEQ ID NO: 1829); CMVie (SEQ ID NO: 1831); CB | 1770 | 3500 |
| | promoter (SEQ ID NO: 1834); human beta globin intron (hGBint) | | |
| | (SEQ ID NO: 1842); signal sequence (SEQ ID NO: 1852); TAT | | |
| | coding sequence (SEQ ID NO: 1793); G4S3 linker coding | | |
| | sequence (SEQ ID NO: 1730); GBA Variant 3 coding sequence | | |
| | (SEQ ID NO: 1781); polyA signal region (SEQ ID NO: 1846); | | |
| | ITR (SEQ ID NO: 1830) | | |
| GBA_VG13 | ITR (SEQ ID NO: 1829); CMVie (SEQ ID NO: 1831); CB | 1771 | 3458 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); Lysosomal | | |
| | targeting sequence 5 (LTS5) (SEQ ID NO: 1807); polyA signal | | |
| | region (SEQ ID NO: 1846); ITR (SEQ ID NO: 1830) | | |
| GBA_VG14 | ITR (SEQ ID NO: 1829); CMVie (SEQ ID NO: 1831); CB | 1809 | 3941 |
| | promoter (SEQ ID NO: 1834); human beta globin intron (hGBint) | | |
| | (SEQ ID NO: 1842); signal sequence (SEQ ID NO: 1852); | | |
| | Lysosomal targeting sequence 2 (LTS2) (SEQ ID NO: 1801); | | |
| | GBA Variant 3 coding sequence (SEQ ID NO: 1781); Furin | | |
| | cleavage site coding sequence (SEQ ID NO: 1724); T2A coding | | |
| | sequence (SEQ ID NO: 1726); signal sequence (SEQ ID NO: | | |
| | 1856); SAPC coding sequence (SEQ ID NO: 1787); polyA signal | | |
| | region (SEQ ID NO: 1846); ITR (SEQ ID NO: 1830) | | |
| GBA_VG15 | ITR (SEQ ID NO: 1829); CMVie (SEQ ID NO: 1831); CB | 1810 | 3977 |
| | promoter (SEQ ID NO: 1834); human beta globin intron (hGBint) | | |
| | (SEQ ID NO: 1842); signal sequence (SEQ ID NO: 1852); GBA | | |
| | Variant 3 coding sequence (SEQ ID NO: 1781); G4S3 linker | | |
| | coding sequence (SEQ ID NO: 1730); ApoEII coding sequence | | |
| | (SEQ ID NO: 1797); Furin cleavage site coding sequence (SEQ ID | | |
| | NO: 1724); T2A coding sequence (SEQ ID NO: 1726); signal | | |
| | sequence (SEQ ID NO: 1856); SAPC coding sequence (SEQ ID | | |
| | NO: 1787); polyA signal region (SEQ ID NO: 1846); ITR (SEQ | | |
| | ID NO: 1830) | | |
| GBA_VG16 | ITR (SEQ ID NO: 1829); CMVie (SEQ ID NO: 1831); CB | 1811 | 4040 |
| | promoter (SEQ ID NO: 1834); human beta globin intron (hGBint) | | |
| | (SEQ ID NO: 1842); signal sequence (SEQ ID NO: 1852); | | |
| | Lysosomal targeting sequence 2 (LTS2) (SEQ ID NO: 1801); | | |
| | GBA Variant 3 coding sequence (SEQ ID NO: 1781); G4S3 linker | | |
| | coding sequence (SEQ ID NO: 1730); ApoEII coding sequence | | |
| | (SEQ ID NO: 1797); Furin cleavage site coding sequence (SEQ ID | | |
| | NO: 1724); T2A coding sequence (SEQ ID NO: 1726); signal | | |
| | sequence (SEQ ID NO: 1856); SAPC coding sequence (SEQ ID | | |
| | NO: 1787); polyA signal region (SEQ ID NO: 1846); ITR (SEQ | | |
| | ID NO: 1830) | | |
| GBA_VG17 | ITR (SEQ ID NO: 1829); CMVie (SEQ ID NO: 1831); CB | 1812 | 3413 |
| | promoter (SEQ ID NO: 1834); human beta globin intron (hGBint) | | |
| | (SEQ ID NO: 1842); signal sequence (SEQ ID NO: 1850); GBA | | |
| | Variant 1 coding sequence (SEQ ID NO: 1773); polyA signal | | |
| | region (SEQ ID NO: 1846); ITR (SEQ ID NO: 1830) | | |
| GBA_VG18 | ITR (SEQ ID NO: 1829); EF-1α promoter variant 2 (SEQ ID NO: | 1813 | 3375 |
| | 1839); signal sequence (SEQ ID NO: 1850); GBA Variant 1 | | |
| | coding sequence (SEQ ID NO: 1773); polyA signal region (SEQ | | |
| | ID NO: 1846); ITR (SEQ ID NO: 1830) | | |
| GBA_VG19 | ITR (SEQ ID NO: 1829); CMVie (SEQ ID NO: 1831); CMV | 1814 | 3360 |
| | promoter (SEQ ID NO: 1832); human beta globin intron (hGBint) | | |
| | (SEQ ID NO: 1842); signal sequence (SEQ ID NO: 1850); GBA | | |
| | Variant 1 coding sequence (SEQ ID NO: 1773); polyA signal | | |
| | region (SEQ ID NO: 1846); ITR (SEQ ID NO: 1830) | | |
| GBA_VG20 | ITR (SEQ ID NO: 1829); CAG promoter (SEQ ID NO: 1835); | 1815 | 3901 |
| | signal sequence (SEQ ID NO: 1850); GBA Variant 1 coding | | |
| | sequence (SEQ ID NO: 1773); polyA signal region (SEQ ID NO: | | |
| | 1846); ITR (SEQ ID NO: 1830) | | |
| GBA_VG21 | ITR (SEQ ID NO: 1829); CMVie (SEQ ID NO: 1831); CB | 1816 | 3413 |
| | promoter (SEQ ID NO: 1834); human beta globin intron (hGBint) | | |
| | (SEQ ID NO: 1842); signal sequence (SEQ ID NO: 1851); GBA | | |
| | Variant 2 coding sequence (SEQ ID NO: 1777); polyA signal | | |
| | region (SEQ ID NO: 1846); ITR (SEQ ID NO: 1830) | | |
| GBA_VG22 | ITR (SEQ ID NO: 1829); CMVie (SEQ ID NO: 1831); CB | 1817 | 3878 |
| | promoter (SEQ ID NO: 1834); human beta globin intron (hGBint) | | |
| | (SEQ ID NO: 1842); signal sequence (SEQ ID NO: 1851); GBA | | |
| | Variant 2 coding sequence (SEQ ID NO: 1777); Furin cleavage | | |
| | site coding sequence (SEQ ID NO: 1724); T2A coding sequence | | |
| | (SEQ ID NO: 1726); signal sequence (SEQ ID NO: 1856); SAPC | | |
| | coding sequence (SEQ ID NO: 1787); polyA signal region (SEQ | | |
| | ID NO: 1846); ITR (SEQ ID NO: 1830) | | |
| GBA_VG23 | ITR (SEQ ID NO: 1829); CMVie (SEQ ID NO: 1831); CB | 1818 | 3512 |
| | promoter (SEQ ID NO: 1834); human beta globin intron (hGBint) | | |
| | (SEQ ID NO: 1842); signal sequence (SEQ ID NO: 1851); GBA | | |
| | Variant 2 coding sequence (SEQ ID NO: 1777); G4S3 linker | | |
| | coding sequence (SEQ ID NO: 1730); ApoEII coding sequence | | |
| | (SEQ ID NO: 1797); polyA signal region (SEQ ID NO: 1846); | | |
| | ITR (SEQ ID NO: 1830) | | |
| GBA_VG24 | ITR (SEQ ID NO: 1829); CMVie (SEQ ID NO: 1831); CB | 1819 | 3476 |
| | promoter (SEQ ID NO: 1834); human beta globin intron (hGBint) | | |
| | (SEQ ID NO: 1842); signal sequence (SEQ ID NO: 1851); | | |
| | Lysosomal targeting sequence 2 (LTS2) (SEQ ID NO: 1801); | | |
| | GBA Variant 2 coding sequence (SEQ ID NO: 1777); polyA | | |
| | signal region (SEQ ID NO: 1846); ITR (SEQ ID NO: 1830) | | |
| GBA_VG25 | ITR (SEQ ID NO: 1829); CMVie (SEQ ID NO: 1831); CB | 1820 | 3428 |
| | promoter (SEQ ID NO: 1834); human beta globin intron (hGBint) | | |
| | (SEQ ID NO: 1842); signal sequence (SEQ ID NO: 1851); GBA | | |
| | Variant 2 coding sequence (SEQ ID NO: 1777); Lysosomal | | |
| | targeting sequence 4 (LTS4) (SEQ ID NO: 1805); polyA signal | | |
| | region (SEQ ID NO: 1846); ITR (SEQ ID NO: 1830) | | |
| GBA_VG26 | ITR (SEQ ID NO: 1829); EF -1α promoter variant 3 (SEQ ID NO: | 1821 | 3375 |
| | 1840); signal sequence (SEQ ID NO: 1851); GBA Variant 2 | | |
| | coding sequence (SEQ ID NO: 1777); polyA signal region (SEQ | | |
| | ID NO: 1846); ITR (SEQ ID NO: 1830) | | |
| GBA_VG27 | ITR (SEQ ID NO: 1829); CMVie (SEQ ID NO: 1831); CB | 1822 | 3878 |
| | promoter (SEQ ID NO: 1834); human beta globin intron (hGBint) | | |
| | (SEQ ID NO: 1842); signal sequence (SEQ ID NO: 1850); GBA | | |
| | Variant 1 coding sequence (SEQ ID NO: 1773); Furin cleavage | | |
| | site coding sequence (SEQ ID NO: 1724); T2A coding sequence | | |
| | (SEQ ID NO: 1726); signal sequence (SEQ ID NO: 1856); SAPC | | |
| | coding sequence (SEQ ID NO: 1787); polyA signal region (SEQ | | |
| | ID NO: 1846); ITR (SEQ ID NO: 1830) | | |
| GBA_VG28 | ITR (SEQ ID NO: 1829); CMVie (SEQ ID NO: 1831); CB | 1823 | 3512 |
| | promoter (SEQ ID NO: 1834); human beta globin intron (hGBint) | | |
| | (SEQ ID NO: 1842); signal sequence (SEQ ID NO: 1850); GBA | | |
| | Variant 1 coding sequence (SEQ ID NO: 1773); G4S3 linker | | |
| | coding sequence (SEQ ID NO: 1730); ApoEII coding sequence | | |
| | (SEQ ID NO: 1797); polyA signal region (SEQ ID NO: 1846); | | |
| | ITR (SEQ ID NO: 1830) | | |
| GBA_VG29 | ITR (SEQ ID NO: 1829); CMVie (SEQ ID NO: 1831); CB | 1824 | 3476 |
| | promoter (SEQ ID NO: 1834); human beta globin intron (hGBint) | | |
| | (SEQ ID NO: 1842); signal sequence (SEQ ID NO: 1850); | | |
| | Lysosomal targeting sequence 2 (LTS2) (SEQ ID NO: 1801); | | |
| | GBA Variant 1 coding sequence (SEQ ID NO: 1773); polyA | | |
| | signal region (SEQ ID NO: 1846); ITR (SEQ ID NO: 1830) | | |
| GBA_VG30 | ITR (SEQ ID NO: 1829); CMVie (SEQ ID NO: 1831); CB | 1825 | 3428 |
| | promoter (SEQ ID NO: 1834); human beta globin intron (hGBint) | | |
| | (SEQ ID NO: 1842); signal sequence (SEQ ID NO: 1850); GBA | | |
| | Variant 1 coding sequence (SEQ ID NO: 1773); Lysosomal | | |
| | targeting sequence 4 (LTS4) (SEQ ID NO: 1805); polyA signal | | |
| | region (SEQ ID NO: 1846); ITR (SEQ ID NO: 1830) | | |
| GBA_VG31 | ITR (SEQ ID NO: 1829); CMVie (SEQ ID NO: 1831); CB | 1826 | 3500 |
| | promoter (SEQ ID NO: 1834); human beta globin intron (hGBint) | | |
| | (SEQ ID NO: 1842); signal sequence (SEQ ID NO: 1851); GBA | | |
| | Variant 2 coding sequence (SEQ ID NO: 1777); G4S3 linker | | |
| | coding sequence (SEQ ID NO: 1730); TAT coding sequence (SEQ | | |
| | ID NO: 1793); polyA signal region (SEQ ID NO: 1846); ITR | | |
| | (SEQ ID NO: 1830) | | |
| GBA_VG32 | ITR (SEQ ID NO: 1829); CMVie (SEQ ID NO: 1831); CB | 1827 | 3500 |
| | promoter (SEQ ID NO: 1834); human beta globin intron (hGBint) | | |
| | (SEQ ID NO: 1842); signal sequence (SEQ ID NO: 1850); GBA | | |
| | Variant 1 coding sequence (SEQ ID NO: 1773); G4S3 linker | | |
| | coding sequence (SEQ ID NO: 1730); TAT coding sequence (SEQ | | |
| | ID NO: 1793); polyA signal region (SEQ ID NO: 1846); ITR | | |
| | (SEQ ID NO: 1830) | | |
| GBA_VG33 | ITR (SEQ ID NO: 1829); CMVie (SEQ ID NO: 1831); CB | 1828 | 3571 |
| | promoter (SEQ ID NO: 1834); human beta globin intron (hGBint) | | |
| | (SEQ ID NO: 1842); signal sequence (SEQ ID NO: 1850); GBA | | |
| | Variant 1 coding sequence (SEQ ID NO: 1773); miR183 binding | | |
| | site (SEQ ID NO: 1847); Spacer (SEQ ID NO: 1848); miR183 | | |
| | binding site (SEQ ID NO: 1847); Spacer (SEQ ID NO: 1848); | | |
| | miR183 binding site (SEQ ID NO: 1847); Spacer (SEQ ID NO: | | |
| | 1848); mirl83 binding site (SEQ ID NO: 1847); polyA signal | | |
| | region (SEQ ID NO: 1846); ITR (SEQ ID NO: 1830) | | |
| GBA_VG34 | ITR (SEQ ID NO: 1829); CMVie (SEQ ID NO: 1831); CB | 1870 | 3571 |
| | promoter (SEQ ID NO: 1834); human beta globin intron (hGBint) | | |
| | (SEQ ID NO: 1842); signal sequence (SEQ ID NO: 1851); GBA | | |
| | Variant 2 coding sequence (SEQ ID NO: 1777); miR183 binding | | |
| | site (SEQ ID NO: 1847); Spacer (SEQ ID NO: 1848); miR183 | | |
| | binding site (SEQ ID NO: 1847); Spacer (SEQ ID NO: 1848); | | |
| | miR183 binding site (SEQ ID NO: 1847); Spacer (SEQ ID NO: | | |
| | 1848); mir183 binding site (SEQ ID NO: 1847); polyA signal | | |
| | region (SEQ ID NO: 1846); ITR (SEQ ID NO: 1830) | | |

TABLE 19
Exemplary ITR to ITR sequences encoding a GBA protein

| | | SEQ |
|---|---|---|
| Construct | | ID |
| ID | Sequence | NO: |
| GBA_VG17 | ctgcgcgctcgctcgctcactgaggccgcccgggcaaagcccgggcgtcgggcgac | 1812 |
| | ctttggtcgcccggcctcagtgagcgagcgagcgcgcagagagggagtggccaact | |
| | ccatcactaggggttccttgtagttaatgattaacccgccatgctacttatctacc | |
| | agggtaatggggatcctctagaactatagctagtcGACATTGATTATTGACTAGTT | |
| | ATTAATAGTAATCAATTACGGGGTCATTAGTTCATAGCCCATATATGGAGTTCCGC | |
| | GTTACATAACTTACGGTAAATGGCCCGCCTGGCTGACCGCCCAACGACCCCCGCCC | |
| | ATTGACGTCAATAATGACGTATGTTCCCATAGTAACGCCAATAGGGACTTTCCATT | |
| | GACGTCAATGGGTGGAGTATTTACGGTAAACTGCCCACTTGGCAGTACATCAAGTG | |
| | TATCATATGCCAAGTACGCCCCCTATTGACGTCAATGACGGTAAATGGCCCGCCTG | |
| | GCATTATGCCCAGTACATGACCTTATGGGACTTTCCTACTTGGCAGTACATCTACG | |
| | TATTAGTCATCGCTATTACCATGtcgaggccacgttctgcttcactctccccatct | |
| | cccccccctccccacccccaattttgtatttatttattttttaattattttgtgca | |
| | gcgatgggggcggggggggggggcgcgcgccaggcggggcggggcggggcgagggg | |
| | cggggcggggcgaggcggagaggtgcggcggcagccaatcagagcggcgcgctccg | |
| | aaagtttccttttatggcgaggcggcggcggcggcggccctataaaaagcgaagcg | |
| | cgcggcgggcgggagcaagcttcgtttagtgaaccgtcagatcgcctggagacgcc | |
| | atccacgctgttttgacctccatagaagacaccgggaccgatccagcctccgcgga | |
| | ttcgaatcccggccgggaacggtgcattggaacgcggattccccgtgccaagagtg | |
| | acgtaagtaccgcctatagagtctataggcccacaaaaaatgctttcttcttttaa | |
| | tatacttttttgtttatcttatttctaatactttccctaatctctttctttcaggg | |
| | caataatgatacaatgtatcatgcctctttgcaccattctaaagaataacagtgat | |
| | aatttctgggttaaggcaatagcaatatttctgcatataaatatttctgcatataa | |
| | attgtaactgatgtaagaggtttcatattgctaatagcagctacaatccagctacc | |
| | attctgcttttattttatggttgggataaggctggattattctgagtccaagctag | |
| | gcccttttgctaatcatgttcatacctcttatcttcctcccacagctcctgggcaa | |
| | cgtgctggtctgtgtgctggcccatcactttggcaaagaattgggattcgaaccgg | |
| | tgccgccaccATGGAATTCTCTAGCCCATCTAGAGAGGAATGTCCTAAGCCTCTGT | |
| | CAAGAGTGTCCATCATGGCCGGCAGCCTGACAGGCCTGCTGCTGCTGCAGGCCGTG | |
| | TCCTGGGCCAGTGGAGCCCGGCCCTGCATCCCTAAGTCCTTCGGCTATTCTAGCGT | |
| | GGTCTGCGTGTGTAATGCCACTTACTGCGACAGCTTCGACCCTCCTACCTTCCCCG | |
| | CCCTTGGAACATTCAGCAGATACGAGAGCACCAGAAGCGGCAGAAGAATGGAACTG | |
| | AGCATGGGCCCAATCCAGGCCAACCACACCGGCACCGGCCTGCTGCTGACACTGCA | |
| | ACCTGAGCAGAAGTTCCAGAAGGTGAAGGGATTTGGAGGCGCCATGACCGACGCTG | |
| | CTGCTCTGAACATCCTGGCCCTCTCCCCACCTGCTCAGAACCTGCTGCTTAAAAGC | |
| | TACTTCAGCGAGGAAGGCATCGGCTATAACATCATCAGAGTGCCCATGGCCAGCTG | |
| | CGACTTCAGCATCAGAACATACACCTACGCCGATACACCTGATGACTTCCAACTGC | |
| | ACAACTTCAGCCTGCCTGAAGAGGACACAAAGCTGAAAATCCCCCTGATCCACCGG | |
| | GCCCTGCAGCTGGCCCAGAGACCTGTGAGCCTGCTGGCCTCTCCTTGGACAAGCCC | |
| | CACCTGGCTGAAGACCAATGGAGCTGTGAACGGCAAGGGCAGCCTGAAGGGCCAGC | |
| | CCGGCGACATCTACCACCAAACCTGGGCTCGCTACTTCGTGAAATTCCTGGACGCC | |
| | TACGCTGAGCATAAGCTGCAATTTTGGGCCGTTACAGCCGAGAACGAGCCTTCTGC | |
| | CGGCCTGCTGTCTGGATATCCTTTCCAGTGCCTGGGCTTCACCCCTGAGCACCAGA | |
| | GAGACTTTATCGCCAGAGATCTGGGGCCTACCCTGGCTAACAGCACACACCACAAC | |
| | GTGCGGCTGCTGATGCTGGACGATCAGAGGCTGCTGCTCCCCCACTGGGCCAAGGT | |
| | GGTGCTGACAGATCCGGAGGCCGCCAAATACGTGCACGGCATCGCCGTCCACTGGT | |
| | ACCTGGATTTCCTGGCCCCTGCCAAGGCCACCCTGGGCGAGACACATAGACTGTTT | |
| | CCTAATACCATGCTGTTCGCCAGCGAGGCCTGCGTGGGCAGCAAGTTCTGGGAACA | |
| | GAGCGTGCGGCTGGGCAGCTGGGACAGAGGAATGCAGTACAGCCACAGCATCATTA | |
| | CCAACCTGCTGTACCACGTGGTGGGCTGGACCGACTGGAACCTGGCCCTGAACCCC | |
| | GAAGGCGGCCCCAACTGGGTGCGGAACTTCGTGGACTCTCCTATCATCGTGGATAT | |
| | TACCAAGGATACCTTTTACAAGCAGCCTATGTTCTACCACCTGGGCCACTTCAGCA | |
| | AGTTCATCCCTGAGGGCTCTCAGCGGGTGGGCCTGGTGGCCTCTCAGAAAAACGAC | |
| | CTGGATGCCGTTGCCCTGATGCACCCCGACGGCAGCGCCGTGGTGGTCGTCCTGAA | |
| | TAGAAGCTCCAAGGACGTGCCTCTGACCATCAAGGACCCCGCTGTGGGATTTCTGG | |
| | AAACCATCAGCCCTGGCTACAGCATCCACACCTACCTGTGGCGGCGGCAGtagtaa | |
| | ctcgaggacggggtgaactacgcctgaggatccgatctttttccctctgccaaaaa | |
| | ttatggggacatcatgaagccccttgagcatctgacttctggctaataaaggaaat | |
| | ttattttcattgcaatagtgtgttggaattttttgtgtctctcactcggcctaggt | |
| | agataagtagcatggcgggttaatcattaactacaaggaacccctagtgatggagt | |
| | tggccactccctctctgcgcgctcgctcgctcactgaggccgggcgaccaaaggtc | |
| | gcccgacgcccgggctttgcccgggcggcctcagtgagcgagcgagcgcgcag | |
| GBA_VG18 | ctgcgcgctcgctcgctcactgaggccgcccgggcaaagcccgggcgtcgggcgac | 1813 |
| | ctttggtcgcccggcctcagtgagcgagcgagcgcgcagagagggagtggccaact | |
| | ccatcactaggggttccttgtagttaatgattaacccgccatgctacttatctacc | |
| | agggtaatggggatcctctagaactatagctagtcgacataacgcgtgcatgcgtg | |
| | aggctccggtgcccgtcagtgggcagagcgcacatcgcccacagtccccgagaagt | |
| | tggggggaggggtcggcaattgaaccggtgcctagagaaggtggcgcggggtaaac | |
| | tgggaaagtgatgtcgtgtactggctccgcctttttcccgagggtgggggagaacc | |
| | gtatataagtgcagtagtcgccgtgaacgttctttttcgcaacgggtttgccgcca | |
| | gaacacaggtaagtgccgtgtgtggttcccgcgggcctggcctctttacgggttat | |
| | ggcccttgcgtgccttgaattacttccacctggctgcagtacgtgattcttgatcc | |
| | cgagcttcgggttggaagtgggtgggagagttcgaggccttgcgcttaaggagccc | |
| | cttcgcctcgtgcttgagttgaggcctggcctgggcgctggggccgccgcgtgcga | |
| | atctggtggcaccttcgcgcctgtctcgctgctttcgataagtctctagccattta | |
| | aaatttttgatgacctgctgcgacgctttttttctggcaagatagtcttgtaaatg | |
| | cgggccaagatctgcacactggtatttcggtttttggggccgcgggcggcgacggg | |
| | gcccgtgcgtcccagcgcacatgttcggcgaggcggggcctgcgagcgcggccacc | |
| | gagaatcggacgggggtagtctcaagctggccggcctgctctggtgcctggcctcg | |
| | cgccgccgtgtatcgccccgccctgggcggcaaggctggcccggtcggcaccagtt | |
| | gcgtgagcggaaagatggccgcttcccggccctgctgcagggagctcaaaatggag | |
| | gacgcggcgctcgggagagcgggcgggtgagtcacccacacaaaggaaaagggcct | |
| | ttccgtcctcagccgtcgcttcatgtgactccacggagtaccgggcgccgtccagg | |
| | cacctcgattagttctcgagcttttggagtacgtcgtctttaggttggggggaggg | |
| | gttttatgcgatggagtttccccacactgagtgggtggagactgaagttaggccag | |
| | cttggcacttgatgtaattctccttggaatttgccctttttgagtttggatcttgg | |
| | ttcattctcaagcctcagacagtggttcaaagtttttttcttccatttcaggtgtc | |
| | gtgagggattcgaaccggtgccgccaccATGGAATTCTCTAGCCCATCTAGAGAGG | |
| | AATGTCCTAAGCCTCTGTCAAGAGTGTCCATCATGGCCGGCAGCCTGACAGGCCTG | |
| | CTGCTGCTGCAGGCCGTGTCCTGGGCCAGTGGAGCCCGGCCCTGCATCCCTAAGTC | |
| | CTTCGGCTATTCTAGCGTGGTCTGCGTGTGTAATGCCACTTACTGCGACAGCTTCG | |
| | ACCCTCCTACCTTCCCCGCCCTTGGAACATTCAGCAGATACGAGAGCACCAGAAGC | |
| | GGCAGAAGAATGGAACTGAGCATGGGCCCAATCCAGGCCAACCACACCGGCACCGG | |
| | CCTGCTGCTGACACTGCAACCTGAGCAGAAGTTCCAGAAGGTGAAGGGATTTGGAG | |
| | GCGCCATGACCGACGCTGCTGCTCTGAACATCCTGGCCCTCTCCCCACCTGCTCAG | |
| | AACCTGCTGCTTAAAAGCTACTTCAGCGAGGAAGGCATCGGCTATAACATCATCAG | |
| | AGTGCCCATGGCCAGCTGCGACTTCAGCATCAGAACATACACCTACGCCGATACAC | |
| | CTGATGACTTCCAACTGCACAACTTCAGCCTGCCTGAAGAGGACACAAAGCTGAAA | |
| | ATCCCCCTGATCCACCGGGCCCTGCAGCTGGCCCAGAGACCTGTGAGCCTGCTGGC | |
| | CTCTCCTTGGACAAGCCCCACCTGGCTGAAGACCAATGGAGCTGTGAACGGCAAGG | |
| | GCAGCCTGAAGGGCCAGCCCGGCGACATCTACCACCAAACCTGGGCTCGCTACTTC | |
| | GTGAAATTCCTGGACGCCTACGCTGAGCATAAGCTGCAATTTTGGGCCGTTACAGC | |
| | CGAGAACGAGCCTTCTGCCGGCCTGCTGTCTGGATATCCTTTCCAGTGCCTGGGCT | |
| | TCACCCCTGAGCACCAGAGAGACTTTATCGCCAGAGATCTGGGGCCTACCCTGGCT | |
| | AACAGCACACACCACAACGTGCGGCTGCTGATGCTGGACGATCAGAGGCTGCTGCT | |
| | CCCCCACTGGGCCAAGGTGGTGCTGACAGATCCGGAGGCCGCCAAATACGTGCACG | |
| | GCATCGCCGTCCACTGGTACCTGGATTTCCTGGCCCCTGCCAAGGCCACCCTGGGC | |
| | GAGACACATAGACTGTTTCCTAATACCATGCTGTTCGCCAGCGAGGCCTGCGTGGG | |
| | CAGCAAGTTCTGGGAACAGAGCGTGCGGCTGGGCAGCTGGGACAGAGGAATGCAGT | |
| | ACAGCCACAGCATCATTACCAACCTGCTGTACCACGTGGTGGGCTGGACCGACTGG | |
| | AACCTGGCCCTGAACCCCGAAGGCGGCCCCAACTGGGTGCGGAACTTCGTGGACTC | |
| | TCCTATCATCGTGGATATTACCAAGGATACCTTTTACAAGCAGCCTATGTTCTACC | |
| | ACCTGGGCCACTTCAGCAAGTTCATCCCTGAGGGCTCTCAGCGGGTGGGCCTGGTG | |
| | GCCTCTCAGAAAAACGACCTGGATGCCGTTGCCCTGATGCACCCCGACGGCAGCGC | |
| | CGTGGTGGTCGTCCTGAATAGAAGCTCCAAGGACGTGCCTCTGACCATCAAGGACC | |
| | CCGCTGTGGGATTTCTGGAAACCATCAGCCCTGGCTACAGCATCCACACCTACCTG | |
| | TGGCGGCGGCAGtagtaactcgaggacggggtgaactacgcctgaggatccgatct | |
| | ttttccctctgccaaaaattatggggacatcatgaagccccttgagcatctgactt | |
| | ctggctaataaaggaaatttattttcattgcaatagtgtgttggaattttttgtgt | |
| | ctctcactcggcctaggtagataagtagcatggcgggttaatcattaactacaagg | |
| | aacccctagtgatggagttggccactccctctctgcgcgctcgctcgctcactgag | |
| | gccgggcgaccaaaggtcgcccgacgcccgggctttgcccgggcggcctcagtgag | |
| | cgagcgagcgcgcag | |
| GBA_VG27 | ctgcgcgctcgctcgctcactgaggccgcccgggcaaagcccgggcgtcgggcgac | 1822 |
| | ctttggtcgcccggcctcagtgagcgagcgagcgcgcagagagggagtggccaact | |
| | ccatcactaggggttcctTGTAGTTAATGATTAACCCGCCATGCTACTTATCTACC | |
| | AGGGTAATGGGGATCCTCTAGAACTATAGCTAGTCGACATTGATTATTGACTAGTT | |
| | ATTAATAGTAATCAATTACGGGGTCATTAGTTCATAGCCCATATATGGAGTTCCGC | |
| | GTTACATAACTTACGGTAAATGGCCCGCCTGGCTGACCGCCCAACGACCCCCGCCC | |
| | ATTGACGTCAATAATGACGTATGTTCCCATAGTAACGCCAATAGGGACTTTCCATT | |
| | GACGTCAATGGGTGGAGTATTTACGGTAAACTGCCCACTTGGCAGTACATCAAGTG | |
| | TATCATATGCCAAGTACGCCCCCTATTGACGTCAATGACGGTAAATGGCCCGCCTG | |
| | GCATTATGCCCAGTACATGACCTTATGGGACTTTCCTACTTGGCAGTACATCTACG | |
| | TATTAGTCATCGCTATTACCATGTCGAGGccacgttctgcttcactctccccatct | |
| | cccccccctccccacccccaattttgtatttatttattttttaattattttgtgca | |
| | gcgatgggggcggggggggggggcgcgcgccaggcggggcggggcggggcgagggg | |
| | cggggcggggcgaggcggagaggtgcggcggcagccaatcagagcggcgcgctccg | |
| | aaagtttccttttatggcgaggcggcggcggcggcggccctataaaaagcgaagcg | |
| | cgcggcgggCGGGAGCAAGCTTCGTTTAGTGAACCGtcagatcgcctggagacgcc | |
| | atccacgctgttttgacctccatagaagacaccgggaccgatccagcctccgcgga | |
| | ttcgaatcccggccgggaacggtgcattggaacgcggattccccgtgccaagagtg | |
| | acgtaagtaccgcctatagagtctataggcccacaaaaaatgctttcttcttttaa | |
| | tatacttttttgtttatcttatttctaatactttccctaatctctttctttcaggg | |
| | caataatgatacaatgtatcatgcctctttgcaccattctaaagaataacagtgat | |
| | aatttctgggttaaggcaatagcaatatttctgcatataaatatttctgcatataa | |
| | attgtaactgatgtaagaggtttcatattgctaatagcagctacaatccagctacc | |
| | attctgcttttattttatggttgggataaggctggattattctgagtccaagctag | |
| | gcccttttgctaatcatgttcatacctcttatcttcctcccacagctcctgggcaa | |
| | cgtgctggtctgtgtgctggcccatcactttggcaaagaattGGGATTCGAACCGG | |
| | TGCCGCCACCATGGAATTCTCTAGCCCATCTAGAGAGGAATGTCCTAAGCCTCTGT | |
| | CAAGAGTGTCCATCATGGCCGGCAGCCTGACAGGCCTGCTGCTGCTGCAGGCCGTG | |
| | TCCTGGGCCAGTGGAGCCCGGCCCTGCATCCCTAAGTCCTTCGGCTATTCTAGCGT | |
| | GGTCTGCGTGTGTAATGCCACTTACTGCGACAGCTTCGACCCTCCTACCTTCCCCG | |
| | CCCTTGGAACATTCAGCAGATACGAGAGCACCAGAAGCGGCAGAAGAATGGAACTG | |
| | AGCATGGGCCCAATCCAGGCCAACCACACCGGCACCGGCCTGCTGCTGACACTGCA | |
| | ACCTGAGCAGAAGTTCCAGAAGGTGAAGGGATTTGGAGGCGCCATGACCGACGCTG | |
| | CTGCTCTGAACATCCTGGCCCTCTCCCCACCTGCTCAGAACCTGCTGCTTAAAAGC | |
| | TACTTCAGCGAGGAAGGCATCGGCTATAACATCATCAGAGTGCCCATGGCCAGCTG | |
| | CGACTTCAGCATCAGAACATACACCTACGCCGATACACCTGATGACTTCCAACTGC | |
| | ACAACTTCAGCCTGCCTGAAGAGGACACAAAGCTGAAAATCCCCCTGATCCACCGG | |
| | GCCCTGCAGCTGGCCCAGAGACCTGTGAGCCTGCTGGCCTCTCCTTGGACAAGCCC | |
| | CACCTGGCTGAAGACCAATGGAGCTGTGAACGGCAAGGGCAGCCTGAAGGGCCAGC | |
| | CCGGCGACATCTACCACCAAACCTGGGCTCGCTACTTCGTGAAATTCCTGGACGCC | |
| | TACGCTGAGCATAAGCTGCAATTTTGGGCCGTTACAGCCGAGAACGAGCCTTCTGC | |
| | CGGCCTGCTGTCTGGATATCCTTTCCAGTGCCTGGGCTTCACCCCTGAGCACCAGA | |
| | GAGACTTTATCGCCAGAGATCTGGGGCCTACCCTGGCTAACAGCACACACCACAAC | |
| | GTGCGGCTGCTGATGCTGGACGATCAGAGGCTGCTGCTCCCCCACTGGGCCAAGGT | |
| | GGTGCTGACAGATCCGGAGGCCGCCAAATACGTGCACGGCATCGCCGTCCACTGGT | |
| | ACCTGGATTTCCTGGCCCCTGCCAAGGCCACCCTGGGCGAGACACATAGACTGTTT | |
| | CCTAATACCATGCTGTTCGCCAGCGAGGCCTGCGTGGGCAGCAAGTTCTGGGAACA | |
| | GAGCGTGCGGCTGGGCAGCTGGGACAGAGGAATGCAGTACAGCCACAGCATCATTA | |
| | CCAACCTGCTGTACCACGTGGTGGGCTGGACCGACTGGAACCTGGCCCTGAACCCC | |
| | GAAGGCGGCCCCAACTGGGTGCGGAACTTCGTGGACTCTCCTATCATCGTGGATAT | |
| | TACCAAGGATACCTTTTACAAGCAGCCTATGTTCTACCACCTGGGCCACTTCAGCA | |
| | AGTTCATCCCTGAGGGCTCTCAGCGGGTGGGCCTGGTGGCCTCTCAGAAAAACGAC | |
| | CTGGATGCCGTTGCCCTGATGCACCCCGACGGCAGCGCCGTGGTGGTCGTCCTGAA | |
| | TAGAAGCTCCAAGGACGTGCCTCTGACCATCAAGGACCCCGCTGTGGGATTTCTGG | |
| | AAACCATCAGCCCTGGCTACAGCATCCACACCTACCTGTGGCGGCGGCAGAgaaag | |
| | aggcgagagggcagaggaagtcttctaacatgcggtgacgtggaggagaatcccgg | |
| | ccctATGTACGCCCTCTTCCTCCTGGCCAGCCTCCTGGGCGCGGCTCTAGCCgtga | |
| | aagagatgcccatgcagactctggtccccgccaaagtggcctccaagaatgtcatc | |
| | cctgccctggaactggtggagcccattaagaagcacgaggtcccagcaaagtctga | |
| | tgtttactgtgaggtgtgtgaattcctggtgaaggaggtgaccaagctgattgaca | |
| | acaacaagactgagaaagaaatactcgacgcttttgacaaaatgtgctcgaagctg | |
| | ccgaagtccctgtcggaagagtgccaggaggtggtggacacgtacggcagctccat | |
| | cctgtccatcctgctggaggaggtcagccctgagctggtgtgcagcatgctgcacc | |
| | tctgctctggcTAGTAACTCGAGGACGGGGTGAACTACGCCTGAGGATCCgatctt | |
| | tttccctctgccaaaaattatggggacatcatgaagccccttgagcatctgacttc | |
| | tggctaataaaggaaatttattttcattgcaatagtgtgttggaattttttgtgtc | |
| | tctcactcgGCCTAGGTAGATAAGTAGCATGGCGGGTTAATCATTAACTACAagga | |
| | acccctagtgatggagttggccactccctctctgcgcgctcgctcgctcactgagg | |
| | ccgggcgaccaaaggtcgcccgacgcccgggctttgcccgggcggcctcagtgagc | |
| | gagcgagcgcgcag | |
| GBA_VG29 | ctgcgcgctcgctcgctcactgaggccgcccgggcaaagcccgggcgtcgggcgac | 1824 |
| | ctttggtcgcccggcctcagtgagcgagcgagcgcgcagagagggagtggccaact | |
| | ccatcactaggggttcctTGTAGTTAATGATTAACCCGCCATGCTACTTATCTACC | |
| | AGGGTAATGGGGATCCTCTAGAACTATAGCTAGTCGACATTGATTATTGACTAGTT | |
| | ATTAATAGTAATCAATTACGGGGTCATTAGTTCATAGCCCATATATGGAGTTCCGC | |
| | GTTACATAACTTACGGTAAATGGCCCGCCTGGCTGACCGCCCAACGACCCCCGCCC | |
| | ATTGACGTCAATAATGACGTATGTTCCCATAGTAACGCCAATAGGGACTTTCCATT | |
| | GACGTCAATGGGTGGAGTATTTACGGTAAACTGCCCACTTGGCAGTACATCAAGTG | |
| | TATCATATGCCAAGTACGCCCCCTATTGACGTCAATGACGGTAAATGGCCCGCCTG | |
| | GCATTATGCCCAGTACATGACCTTATGGGACTTTCCTACTTGGCAGTACATCTACG | |
| | TATTAGTCATCGCTATTACCATGTCGAGGccacgttctgcttcactctccccatct | |
| | cccccccctccccacccccaattttgtatttatttattttttaattattttgtgca | |
| | gcgatgggggcggggggggggggcgcgcgccaggcggggcggggcggggcgagggg | |
| | cggggcggggcgaggcggagaggtgcggcggcagccaatcagagcggcgcgctccg | |
| | aaagtttccttttatggcgaggcggcggcggcggcggccctataaaaagcgaagcg | |
| | cgcggcgggCGGGAGCAAGCTTCGTTTAGTGAACCGtcagatcgcctggagacgcc | |
| | atccacgctgttttgacctccatagaagacaccgggaccgatccagcctccgcgga | |
| | ttcgaatcccggccgggaacggtgcattggaacgcggattccccgtgccaagagtg | |
| | acgtaagtaccgcctatagagtctataggcccacaaaaaatgctttcttcttttaa | |
| | tatacttttttgtttatcttatttctaatactttccctaatctctttctttcaggg | |
| | caataatgatacaatgtatcatgcctctttgcaccattctaaagaataacagtgat | |
| | aatttctgggttaaggcaatagcaatatttctgcatataaatatttctgcatataa | |
| | attgtaactgatgtaagaggtttcatattgctaatagcagctacaatccagctacc | |
| | attctgcttttattttatggttgggataaggctggattattctgagtccaagctag | |
| | gcccttttgctaatcatgttcatacctcttatcttcctcccacagctcctgggcaa | |
| | cgtgctggtctgtgtgctggcccatcactttggcaaagaattGGGATTCGAACCGG | |
| | TGCCGCCACCATGGAATTCTCTAGCCCATCTAGAGAGGAATGTCCTAAGCCTCTGT | |
| | CAAGAGTGTCCATCATGGCCGGCAGCCTGACAGGCCTGCTGCTGCTGCAGGCCGTG | |
| | TCCTGGGCCAGTGGAatgaaggagaccgctgctgcaaagttcgagagacagcatat | |
| | ggatagctccacaagcgccgcaGCCCGGCCCTGCATCCCTAAGTCCTTCGGCTATT | |
| | CTAGCGTGGTCTGCGTGTGTAATGCCACTTACTGCGACAGCTTCGACCCTCCTACC | |
| | TTCCCCGCCCTTGGAACATTCAGCAGATACGAGAGCACCAGAAGCGGCAGAAGAAT | |
| | GGAACTGAGCATGGGCCCAATCCAGGCCAACCACACCGGCACCGGCCTGCTGCTGA | |
| | CACTGCAACCTGAGCAGAAGTTCCAGAAGGTGAAGGGATTTGGAGGCGCCATGACC | |
| | GACGCTGCTGCTCTGAACATCCTGGCCCTCTCCCCACCTGCTCAGAACCTGCTGCT | |
| | TAAAAGCTACTTCAGCGAGGAAGGCATCGGCTATAACATCATCAGAGTGCCCATGG | |
| | CCAGCTGCGACTTCAGCATCAGAACATACACCTACGCCGATACACCTGATGACTTC | |
| | CAACTGCACAACTTCAGCCTGCCTGAAGAGGACACAAAGCTGAAAATCCCCCTGAT | |
| | CCACCGGGCCCTGCAGCTGGCCCAGAGACCTGTGAGCCTGCTGGCCTCTCCTTGGA | |
| | CAAGCCCCACCTGGCTGAAGACCAATGGAGCTGTGAACGGCAAGGGCAGCCTGAAG | |
| | GGCCAGCCCGGCGACATCTACCACCAAACCTGGGCTCGCTACTTCGTGAAATTCCT | |
| | GGACGCCTACGCTGAGCATAAGCTGCAATTTTGGGCCGTTACAGCCGAGAACGAGC | |
| | CTTCTGCCGGCCTGCTGTCTGGATATCCTTTCCAGTGCCTGGGCTTCACCCCTGAG | |
| | CACCAGAGAGACTTTATCGCCAGAGATCTGGGGCCTACCCTGGCTAACAGCACACA | |
| | CCACAACGTGCGGCTGCTGATGCTGGACGATCAGAGGCTGCTGCTCCCCCACTGGG | |
| | CCAAGGTGGTGCTGACAGATCCGGAGGCCGCCAAATACGTGCACGGCATCGCCGTC | |
| | CACTGGTACCTGGATTTCCTGGCCCCTGCCAAGGCCACCCTGGGCGAGACACATAG | |
| | ACTGTTTCCTAATACCATGCTGTTCGCCAGCGAGGCCTGCGTGGGCAGCAAGTTCT | |
| | GGGAACAGAGCGTGCGGCTGGGCAGCTGGGACAGAGGAATGCAGTACAGCCACAGC | |
| | ATCATTACCAACCTGCTGTACCACGTGGTGGGCTGGACCGACTGGAACCTGGCCCT | |
| | GAACCCCGAAGGCGGCCCCAACTGGGTGCGGAACTTCGTGGACTCTCCTATCATCG | |
| | TGGATATTACCAAGGATACCTTTTACAAGCAGCCTATGTTCTACCACCTGGGCCAC | |
| | TTCAGCAAGTTCATCCCTGAGGGCTCTCAGCGGGTGGGCCTGGTGGCCTCTCAGAA | |
| | AAACGACCTGGATGCCGTTGCCCTGATGCACCCCGACGGCAGCGCCGTGGTGGTCG | |
| | TCCTGAATAGAAGCTCCAAGGACGTGCCTCTGACCATCAAGGACCCCGCTGTGGGA | |
| | TTTCTGGAAACCATCAGCCCTGGCTACAGCATCCACACCTACCTGTGGCGGCGGCA | |
| | GTAGTAACTCGAGGACGGGGTGAACTACGCCTGAGGATCCgatctttttccctctg | |
| | ccaaaaattatggggacatcatgaagccccttgagcatctgacttctggctaataa | |
| | aggaaatttattttcattgcaatagtgtgttggaattttttgtgtctctcactcgG | |
| | CCTAGGTAGATAAGTAGCATGGCGGGTTAATCATTAACTACAaggaacccctagtg | |
| | atggagttggccactccctctctgcgcgctcgctcgctcactgaggccgggcgacc | |
| | aaaggtcgcccgacgcccgggctttgcccgggcggcctcagtgagcgagcgagcgc | |
| | gcag | |
| GBA_VG32 | ctgcgcgctcgctcgctcactgaggccgcccgggcaaagcccgggcgtcgggcgac | 1827 |
| | ctttggtcgcccggcctcagtgagcgagcgagcgcgcagagagggagtggccaact | |
| | ccatcactaggggttcctTGTAGTTAATGATTAACCCGCCATGCTACTTATCTACC | |
| | AGGGTAATGGGGATCCTCTAGAACTATAGCTAGTCGACATTGATTATTGACTAGTT | |
| | ATTAATAGTAATCAATTACGGGGTCATTAGTTCATAGCCCATATATGGAGTTCCGC | |
| | GTTACATAACTTACGGTAAATGGCCCGCCTGGCTGACCGCCCAACGACCCCCGCCC | |
| | ATTGACGTCAATAATGACGTATGTTCCCATAGTAACGCCAATAGGGACTTTCCATT | |
| | GACGTCAATGGGTGGAGTATTTACGGTAAACTGCCCACTTGGCAGTACATCAAGTG | |
| | TATCATATGCCAAGTACGCCCCCTATTGACGTCAATGACGGTAAATGGCCCGCCTG | |
| | GCATTATGCCCAGTACATGACCTTATGGGACTTTCCTACTTGGCAGTACATCTACG | |
| | TATTAGTCATCGCTATTACCATGTCGAGGccacgttctgcttcactctccccatct | |
| | cccccccctccccacccccaattttgtatttatttattttttaattattttgtgca | |
| | gcgatgggggcggggggggggggcgcgcgccaggcggggcggggcggggcgagggg | |
| | cggggcggggcgaggcggagaggtgcggcggcagccaatcagagcggcgcgctccg | |
| | aaagtttccttttatggcgaggcggcggcggcggcggccctataaaaagcgaagcg | |
| | cgcggcgggCGGGAGCAAGCTTCGTTTAGTGAACCGtcagatcgcctggagacgcc | |
| | atccacgctgttttgacctccatagaagacaccgggaccgatccagcctccgcgga | |
| | ttcgaatcccggccgggaacggtgcattggaacgcggattccccgtgccaagagtg | |
| | acgtaagtaccgcctatagagtctataggcccacaaaaaatgctttcttcttttaa | |
| | tatacttttttgtttatcttatttctaatactttccctaatctctttctttcaggg | |
| | caataatgatacaatgtatcatgcctctttgcaccattctaaagaataacagtgat | |
| | aatttctgggttaaggcaatagcaatatttctgcatataaatatttctgcatataa | |
| | attgtaactgatgtaagaggtttcatattgctaatagcagctacaatccagctacc | |
| | attctgcttttattttatggttgggataaggctggattattctgagtccaagctag | |
| | gcccttttgctaatcatgttcatacctcttatcttcctcccacagctcctgggcaa | |
| | cgtgctggtctgtgtgctggcccatcactttggcaaagaattGGGATTCGAACCGG | |
| | TGCCGCCACCATGGAATTCTCTAGCCCATCTAGAGAGGAATGTCCTAAGCCTCTGT | |
| | CAAGAGTGTCCATCATGGCCGGCAGCCTGACAGGCCTGCTGCTGCTGCAGGCCGTG | |
| | TCCTGGGCCAGTGGAGCCCGGCCCTGCATCCCTAAGTCCTTCGGCTATTCTAGCGT | |
| | GGTCTGCGTGTGTAATGCCACTTACTGCGACAGCTTCGACCCTCCTACCTTCCCCG | |
| | CCCTTGGAACATTCAGCAGATACGAGAGCACCAGAAGCGGCAGAAGAATGGAACTG | |
| | AGCATGGGCCCAATCCAGGCCAACCACACCGGCACCGGCCTGCTGCTGACACTGCA | |
| | ACCTGAGCAGAAGTTCCAGAAGGTGAAGGGATTTGGAGGCGCCATGACCGACGCTG | |
| | CTGCTCTGAACATCCTGGCCCTCTCCCCACCTGCTCAGAACCTGCTGCTTAAAAGC | |
| | TACTTCAGCGAGGAAGGCATCGGCTATAACATCATCAGAGTGCCCATGGCCAGCTG | |
| | CGACTTCAGCATCAGAACATACACCTACGCCGATACACCTGATGACTTCCAACTGC | |
| | ACAACTTCAGCCTGCCTGAAGAGGACACAAAGCTGAAAATCCCCCTGATCCACCGG | |
| | GCCCTGCAGCTGGCCCAGAGACCTGTGAGCCTGCTGGCCTCTCCTTGGACAAGCCC | |
| | CACCTGGCTGAAGACCAATGGAGCTGTGAACGGCAAGGGCAGCCTGAAGGGCCAGC | |
| | CCGGCGACATCTACCACCAAACCTGGGCTCGCTACTTCGTGAAATTCCTGGACGCC | |
| | TACGCTGAGCATAAGCTGCAATTTTGGGCCGTTACAGCCGAGAACGAGCCTTCTGC | |
| | CGGCCTGCTGTCTGGATATCCTTTCCAGTGCCTGGGCTTCACCCCTGAGCACCAGA | |
| | GAGACTTTATCGCCAGAGATCTGGGGCCTACCCTGGCTAACAGCACACACCACAAC | |
| | GTGCGGCTGCTGATGCTGGACGATCAGAGGCTGCTGCTCCCCCACTGGGCCAAGGT | |
| | GGTGCTGACAGATCCGGAGGCCGCCAAATACGTGCACGGCATCGCCGTCCACTGGT | |
| | ACCTGGATTTCCTGGCCCCTGCCAAGGCCACCCTGGGCGAGACACATAGACTGTTT | |
| | CCTAATACCATGCTGTTCGCCAGCGAGGCCTGCGTGGGCAGCAAGTTCTGGGAACA | |
| | GAGCGTGCGGCTGGGCAGCTGGGACAGAGGAATGCAGTACAGCCACAGCATCATTA | |
| | CCAACCTGCTGTACCACGTGGTGGGCTGGACCGACTGGAACCTGGCCCTGAACCCC | |
| | GAAGGCGGCCCCAACTGGGTGCGGAACTTCGTGGACTCTCCTATCATCGTGGATAT | |
| | TACCAAGGATACCTTTTACAAGCAGCCTATGTTCTACCACCTGGGCCACTTCAGCA | |
| | AGTTCATCCCTGAGGGCTCTCAGCGGGTGGGCCTGGTGGCCTCTCAGAAAAACGAC | |
| | CTGGATGCCGTTGCCCTGATGCACCCCGACGGCAGCGCCGTGGTGGTCGTCCTGAA | |
| | TAGAAGCTCCAAGGACGTGCCTCTGACCATCAAGGACCCCGCTGTGGGATTTCTGG | |
| | AAACCATCAGCCCTGGCTACAGCATCCACACCTACCTGTGGCGGCGGCAGGGAGGG | |
| | GGGGGTTCGGGTGGCGGCGGAAGTGGGGGCGGTGGTTCTtatggcaggaaaaagcg | |
| | gaggcaaaggcgccgccccccccagTAGTAACTCGAGGACGGGGTGAACTACGCCT | |
| | GAGGATCCgatctttttccctctgccaaaaattatggggacatcatgaagcccctt | |
| | gagcatctgacttctggctaataaaggaaatttattttcattgcaatagtgtgttg | |
| | gaattttttgtgtctctcactcgGCCTAGGTAGATAAGTAGCATGGCGGGTTAATC | |
| | ATTAACTACAaggaacccctagtgatggagttggccactccctctctgcgcgctcg | |
| | ctcgctcactgaggccgggcgaccaaaggtcgcccgacgcccgggctttgcccggg | |
| | cggcctcagtgagcgagcgagcgcgcag | |
| GBA_VG33 | ctgcgcgctcgctcgctcactgaggccgcccgggcaaagcccgggcgtcgggcgac | 1828 |
| | ctttggtcgcccggcctcagtgagcgagcgagcgcgcagagagggagtggccaact | |
| | ccatcactaggggttcctTGTAGTTAATGATTAACCCGCCATGCTACTTATCTACC | |
| | AGGGTAATGGGGATCCTCTAGAACTATAGCTAGTCGACATTGATTATTGACTAGTT | |
| | ATTAATAGTAATCAATTACGGGGTCATTAGTTCATAGCCCATATATGGAGTTCCGC | |
| | GTTACATAACTTACGGTAAATGGCCCGCCTGGCTGACCGCCCAACGACCCCCGCCC | |
| | ATTGACGTCAATAATGACGTATGTTCCCATAGTAACGCCAATAGGGACTTTCCATT | |
| | GACGTCAATGGGTGGAGTATTTACGGTAAACTGCCCACTTGGCAGTACATCAAGTG | |
| | TATCATATGCCAAGTACGCCCCCTATTGACGTCAATGACGGTAAATGGCCCGCCTG | |
| | GCATTATGCCCAGTACATGACCTTATGGGACTTTCCTACTTGGCAGTACATCTACG | |
| | TATTAGTCATCGCTATTACCATGTCGAGGccacgttctgcttcactctccccatct | |
| | cccccccctccccacccccaattttgtatttatttattttttaattattttgtgca | |
| | gcgatgggggcggggggggggggcgcgcgccaggcggggcggggcggggcgagggg | |
| | cggggcggggcgaggcggagaggtgcggcggcagccaatcagagcggcgcgctccg | |
| | aaagtttccttttatggcgaggcggcggcggcggcggccctataaaaagcgaagcg | |
| | cgcggcgggCGGGAGCAAGCTTCGTTTAGTGAACCGtcagatcgcctggagacgcc | |
| | atccacgctgttttgacctccatagaagacaccgggaccgatccagcctccgcgga | |
| | ttcgaatcccggccgggaacggtgcattggaacgcggattccccgtgccaagagtg | |
| | acgtaagtaccgcctatagagtctataggcccacaaaaaatgctttcttcttttaa | |
| | tatacttttttgtttatcttatttctaatactttccctaatctctttctttcaggg | |
| | caataatgatacaatgtatcatgcctctttgcaccattctaaagaataacagtgat | |
| | aatttctgggttaaggcaatagcaatatttctgcatataaatatttctgcatataa | |
| | attgtaactgatgtaagaggtttcatattgctaatagcagctacaatccagctacc | |
| | attctgcttttattttatggttgggataaggctggattattctgagtccaagctag | |
| | gcccttttgctaatcatgttcatacctcttatcttcctcccacagctcctgggcaa | |
| | cgtgctggtctgtgtgctggcccatcactttggcaaagaattGGGATTCGAACCGG | |
| | TGCCGCCACCATGGAATTCTCTAGCCCATCTAGAGAGGAATGTCCTAAGCCTCTGT | |
| | CAAGAGTGTCCATCATGGCCGGCAGCCTGACAGGCCTGCTGCTGCTGCAGGCCGTG | |
| | TCCTGGGCCAGTGGAGCCCGGCCCTGCATCCCTAAGTCCTTCGGCTATTCTAGCGT | |
| | GGTCTGCGTGTGTAATGCCACTTACTGCGACAGCTTCGACCCTCCTACCTTCCCCG | |
| | CCCTTGGAACATTCAGCAGATACGAGAGCACCAGAAGCGGCAGAAGAATGGAACTG | |
| | AGCATGGGCCCAATCCAGGCCAACCACACCGGCACCGGCCTGCTGCTGACACTGCA | |
| | ACCTGAGCAGAAGTTCCAGAAGGTGAAGGGATTTGGAGGCGCCATGACCGACGCTG | |
| | CTGCTCTGAACATCCTGGCCCTCTCCCCACCTGCTCAGAACCTGCTGCTTAAAAGC | |
| | TACTTCAGCGAGGAAGGCATCGGCTATAACATCATCAGAGTGCCCATGGCCAGCTG | |
| | CGACTTCAGCATCAGAACATACACCTACGCCGATACACCTGATGACTTCCAACTGC | |
| | ACAACTTCAGCCTGCCTGAAGAGGACACAAAGCTGAAAATCCCCCTGATCCACCGG | |
| | GCCCTGCAGCTGGCCCAGAGACCTGTGAGCCTGCTGGCCTCTCCTTGGACAAGCCC | |
| | CACCTGGCTGAAGACCAATGGAGCTGTGAACGGCAAGGGCAGCCTGAAGGGCCAGC | |
| | CCGGCGACATCTACCACCAAACCTGGGCTCGCTACTTCGTGAAATTCCTGGACGCC | |
| | TACGCTGAGCATAAGCTGCAATTTTGGGCCGTTACAGCCGAGAACGAGCCTTCTGC | |
| | CGGCCTGCTGTCTGGATATCCTTTCCAGTGCCTGGGCTTCACCCCTGAGCACCAGA | |
| | GAGACTTTATCGCCAGAGATCTGGGGCCTACCCTGGCTAACAGCACACACCACAAC | |
| | GTGCGGCTGCTGATGCTGGACGATCAGAGGCTGCTGCTCCCCCACTGGGCCAAGGT | |
| | GGTGCTGACAGATCCGGAGGCCGCCAAATACGTGCACGGCATCGCCGTCCACTGGT | |
| | ACCTGGATTTCCTGGCCCCTGCCAAGGCCACCCTGGGCGAGACACATAGACTGTTT | |
| | CCTAATACCATGCTGTTCGCCAGCGAGGCCTGCGTGGGCAGCAAGTTCTGGGAACA | |
| | GAGCGTGCGGCTGGGCAGCTGGGACAGAGGAATGCAGTACAGCCACAGCATCATTA | |
| | CCAACCTGCTGTACCACGTGGTGGGCTGGACCGACTGGAACCTGGCCCTGAACCCC | |
| | GAAGGCGGCCCCAACTGGGTGCGGAACTTCGTGGACTCTCCTATCATCGTGGATAT | |
| | TACCAAGGATACCTTTTACAAGCAGCCTATGTTCTACCACCTGGGCCACTTCAGCA | |
| | AGTTCATCCCTGAGGGCTCTCAGCGGGTGGGCCTGGTGGCCTCTCAGAAAAACGAC | |
| | CTGGATGCCGTTGCCCTGATGCACCCCGACGGCAGCGCCGTGGTGGTCGTCCTGAA | |
| | TAGAAGCTCCAAGGACGTGCCTCTGACCATCAAGGACCCCGCTGTGGGATTTCTGG | |
| | AAACCATCAGCCCTGGCTACAGCATCCACACCTACCTGTGGCGGCGGCAGTAGTAA | |
| | CCTCGAGGTACCAGGAGCTCTTCTCCTAGTGAATTCTACCAGTGCCATAGATAGTT | |
| | AAGTGAATTCTACCAGTGCCATAGATAGTTAAGTGAATTCTACCAGTGCCATAGAT | |
| | AGTTAAGTGAATTCTACCAGTGCCATACTGCAGTCAGGTCTATACCATCGAGGACG | |
| | GGGTGAACTACGCCTGAGGATCCgatctttttccctctgccaaaaattatggggac | |
| | atcatgaagccccttgagcatctgacttctggctaataaaggaaatttattttcat | |
| | tgcaatagtgtgttggaattttttgtgtctctcactcgGCCTAGGTAGATAAGTAG | |
| | CATGGCGGGTTAATCATTAACTACAaggaacccctagtgatggagttggccactcc | |
| | ctctctgcgcgctcgctcgctcactgaggccgggcgaccaaaggtcgcccgacgcc | |
| | cgggctttgcccgggcggcctcagtgagcgagcgagcgcgcag | |

In some embodiments, the viral genome of an AAV particle described herein comprises a nucleotide sequence comprising the all of the components or a combination of the components as described, e.g., in Tables 20, 21, or 29-32, or a sequence having at least 70%, 75%, 80%, 85%, 90%, 95%, or 99% sequence identity to any of the aforesaid sequences.

TABLE 20
Sequence Regions in ITR to ITR Sequences

GBA_VG17 (SEQ ID NO: 1812)

| | Region SEQ | Region | Position in SEQ |
|---|---|---|---|
| Sequence Regions | ID NO | length | ID NO: 1812 |
| 5′ ITR | 1829 | 130 | 1-130 |
| CMVie | 1831 | 380 | 204-583 |
| CB promoter | 1834 | 260 | 590-849 |
| Intron | 1842 | 566 | 877-1442 |
| Signal sequence | 1850 | 117 | 1467-1583 |
| GBA Variant 1 | 1773 | 1,491 | 1584-3074 |
| coding sequence | | | |
| PolyA | 1846 | 127 | 3114-3240 |
| 3′ ITR | 1830 | 130 | 3284-3413 |

In some embodiments the AAV particle comprises a viral genome comprising the nucleotide sequence of SEQ ID NO: 1812 (GBA_VG17), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1812, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1812, comprises in 5′ to 3′ order: a 5′ ITR sequence region comprising the nucleotide sequence of SEQ ID NO: 1829, or a nucleotide sequence at least 95% identical thereto; a CMVie enhancer comprising the nucleotide sequence of SEQ ID NO: 1831, or a nucleotide sequence at least 95% identical thereto; a CB promoter comprising the nucleotide sequence of SEQ ID NO: 1834, or a nucleotide sequence at least 95% identical thereto; an intron comprising the nucleotide sequence of SEQ ID NO: 1842, or a nucleotide sequence at least 95% identical thereto; a nucleotide sequence encoding a signal sequence comprising the nucleotide sequence of SEQ ID NO: 1850, or a nucleotide sequence at least 95% identical thereto; a nucleotide sequence encoding a GBA protein comprising the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773; a polyadenylation sequence comprising the nucleotide sequence of SEQ ID NO: 1846, or a nucleotide sequence at least 95% identical thereto; and a 3′ ITR sequence region comprising the nucleotide sequence of SEQ ID NO: 1830, or a nucleotide sequence at least 95% identical thereto.

In some embodiments, the viral genome comprising the nucleotide sequence of SEQ ID NO: 1812, or a nucleotide sequence substantially identical (e.g., having at least 70%, 75%, 80%, 85%, 90%, 95%, or 99% sequence identity) thereto, encodes a GBA protein comprising the amino acid sequence of SEQ ID NO: 1775, or an amino acid sequence substantially identical (e.g., having at least 70%, 75%, 80%, 85%, 90%, 95%, or 99% sequence identity) thereto.

TABLE 29
Sequence Regions in ITR to ITR Sequences

| | GBA_VG18 (SEQ ID NO: 1813) | | |
|---|---|---|---|
| | Region SEQ | Region | Position in SEQ |
| Sequence Regions | ID NO | length | ID NO: 1813 |
| 5′ ITR | 1829 | 130 | 1-130 |
| EF-1α promoter variant 2 | 1839 | 1189 | 216-1404 |
| Signal sequence | 1850 | 117 | 1429-1545 |
| GBA Variant 1 coding | 1773 | 1,491 | 1546-3063 |
| sequence | | | |
| PolyA | 1846 | 127 | 3076-3202 |
| 3′ ITR | 1830 | 130 | 3246-3375 |

In some embodiments the AAV particle comprises a viral genome comprising the nucleotide sequence of SEQ ID NO: 1813 (GBA_VG18), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1813, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1813, comprises in 5′ to 3′ order: a 5′ ITR sequence region comprising the nucleotide sequence of SEQ ID NO: 1829, or a nucleotide sequence at least 95% identical thereto; an EF-1α promoter variant comprising the nucleotide sequence of SEQ ID NO: 1839, or a nucleotide sequence at least 95% identical thereto; a nucleotide sequence encoding a signal sequence comprising the nucleotide sequence of SEQ ID NO: 1850, or a nucleotide sequence at least 95% identical thereto; a nucleotide sequence encoding a GBA protein comprising the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773; a polyadenylation sequence comprising the nucleotide sequence of SEQ ID NO: 1846, or a nucleotide sequence at least 95% identical thereto; and a 3′ ITR sequence region comprising the nucleotide sequence of SEQ ID NO: 1830, or a nucleotide sequence at least 95% identical thereto.

In some embodiments, the viral genome comprising the nucleotide sequence of SEQ ID NO: 1813, or a nucleotide sequence substantially identical (e.g., having at least 70%, 75%, 80%, 85%, 90%, 95%, or 99% sequence identity) thereto, encodes a GBA protein comprising the amino acid sequence of SEQ ID NO: 1775, or an amino acid sequence substantially identical (e.g., having at least 70%, 75%, 80%, 85%, 90%, 95%, or 99% sequence identity) thereto.

TABLE 30
Sequence Regions in ITR to ITR Sequences

| | GBA_VG27 (SEQ ID NO: 1822) | | |
|---|---|---|---|
| | Region SEQ | Region | Position in SEQ |
| Sequence Regions | ID NO | length | ID NO: 1822 |
| 5′ ITR | 1829 | 130 | 1-130 |
| CMVie | 1831 | 380 | 204-583 |
| CB promoter | 1834 | 260 | 590-849 |
| Intron | 1842 | 566 | 877-1442 |
| Signal sequence | 1850 | 117 | 1467-1583 |
| GBA Variant 1 coding | 1773 | 1,491 | 1584-3074 |
| sequence | | | |
| Furin cleavage site | 1724 | 12 | 3075-3086 |
| T2A | 1726 | 54 | 3087-3140 |
| Signal sequence | 1856 | 48 | 3141-3188 |
| SAPC coding sequence | 1787 | 351 | 3189-3539 |
| PolyA | 1846 | 127 | 3579-3705 |
| 3′ ITR | 1830 | 130 | 3749-3878 |

In some embodiments the AAV particle comprises a viral genome comprising the nucleotide sequence of SEQ ID NO: 1822 (GBA_VG27), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1822, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1822, comprises in 5′ to 3′ order: a 5′ ITR sequence region comprising the nucleotide sequence of SEQ ID NO: 1829, or a nucleotide sequence at least 95% identical thereto; a CMVie enhancer comprising the nucleotide sequence of SEQ ID NO: 1831, or a nucleotide sequence at least 95% identical thereto; a CB promoter comprising the nucleotide sequence of SEQ ID NO: 1834, or a nucleotide sequence at least 95% identical thereto; an intron comprising the nucleotide sequence of SEQ ID NO: 1842, or a nucleotide sequence at least 95% identical thereto; a nucleotide sequence encoding a first signal sequence comprising the nucleotide sequence of SEQ ID NO: 1850, or a nucleotide sequence at least 95% identical thereto; a nucleotide sequence encoding a GBA protein comprising the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773; a nucleotide sequence encoding a furin cleavage site comprising the nucleotide sequence of SEQ ID NO: 1724, or a nucleotide sequence having at least one, two, or three but no more than four modifications, e.g., substitutions, relative to SEQ ID NO: 1724; a nucleotide sequence encoding a T2A polypeptide comprising the nucleotide sequence of SEQ ID NO: 1726, or a nucleotide sequence having at least one, two, or three but no more than four modifications, e.g., substitutions, relative to SEQ ID NO: 1726; a nucleotide sequence encoding a second signal sequence comprising the nucleotide sequence of SEQ ID NO: 1856, or a nucleotide sequence at least 85% (e.g., at least 90%, 92%, 95%, 97%, 98%, or 99%) identical thereto; a nucleotide sequence encoding a SAPC polypeptide comprising the nucleotide sequence of SEQ ID NO: 1787, or a nucleotide sequence at least 85% (e.g., at least 90%, 92%, 95%, 97%, 98%, or 99%) identical thereto; a polyadenylation sequence comprising the nucleotide sequence of SEQ ID NO: 1846, or a nucleotide sequence at least 95% identical thereto; and a 3′ ITR sequence region comprising the nucleotide sequence of SEQ ID NO: 1830, or a nucleotide sequence at least 95% identical thereto.

In some embodiments, the viral genome comprising the nucleotide sequence of SEQ ID NO: 1822, or a nucleotide sequence substantially identical (e.g., having at least 70%, 75%, 80%, 85%, 90%, 95%, or 99% sequence identity) thereto, encodes a GBA protein comprising the amino acid sequence of SEQ ID NO: 1775, or an amino acid sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1822, or a nucleotide sequence substantially identical (e.g., having at least 70%, 75%, 80%, 85%, 90%, 95%, or 99% sequence identity) thereto, encodes a SAPC protein comprising the amino acid sequence of SEQ ID NO: 1789, or an amino acid sequence at least 85% (e.g., at least 90%, 92%, 95%, 96%, 97%, 98%, or 99%) identical thereto.

TABLE 31
Sequence Regions in ITR to ITR Sequences

| | GBA_VG29 (SEQ ID NO: 1824) | | |
|---|---|---|---|
| | Region SEQ | Region | Position in SEQ |
| Sequence Regions | ID NO | length | ID NO: 1824 |
| 5′ ITR | 1829 | 130 | 1-130 |
| CMVie | 1831 | 380 | 204-583 |
| CB promoter | 1834 | 260 | 590-849 |
| Intron | 1842 | 566 | 877-1442 |
| Signal sequence | 1850 | 117 | 1467-1583 |
| Lysosomal targeting | 1801 | 63 | 1584-1646 |
| sequence 2 (LTS2) | | | |
| GBA Variant 1 coding | 1773 | 1,491 | 1647-3137 |
| sequence | | | |
| PolyA | 1846 | 127 | 3177-3303 |
| 3′ ITR | 1830 | 130 | 3347-3476 |

In some embodiments the AAV particle comprises a viral genome comprising the nucleotide sequence of SEQ ID NO: 1824 (GBA_VG29), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1824, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1824, comprises in 5′ to 3′ order: a 5′ ITR sequence region comprising the nucleotide sequence of SEQ ID NO: 1829, or a nucleotide sequence at least 95% identical thereto; a CMVie enhancer comprising the nucleotide sequence of SEQ ID NO: 1831, or a nucleotide sequence at least 95% identical thereto; a CB promoter comprising the nucleotide sequence of SEQ ID NO: 1834, or a nucleotide sequence at least 95% identical thereto; an intron comprising the nucleotide sequence of SEQ ID NO: 1842, or a nucleotide sequence at least 95% identical thereto; a nucleotide sequence encoding a signal sequence comprising the nucleotide sequence of SEQ ID NO: 1850, or a nucleotide sequence at least 95% identical thereto; a lysosomal targeting sequence 2 (LTS2) comprising the nucleotide sequence of SEQ ID NO: 1801, or a nucleotide sequence at least 85% (e.g., at least 90%, 92%, 95%, 96%, 97%, 98%, or 99%) identical thereto; a nucleotide sequence encoding a GBA protein comprising the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773; a polyadenylation sequence comprising the nucleotide sequence of SEQ ID NO: 1846, or a nucleotide sequence at least 95% identical thereto; and a 3′ ITR sequence region comprising the nucleotide sequence of SEQ ID NO: 1830, or a nucleotide sequence at least 95% identical thereto.

In some embodiments, the viral genome comprising the nucleotide sequence of SEQ ID NO: 1824, or a nucleotide sequence substantially identical (e.g., having at least 70%, 75%, 80%, 85%, 90%, 95%, or 99% sequence identity) thereto, encodes a GBA protein comprising the amino acid sequence of SEQ ID NO: 1775, or an amino acid sequence substantially identical (e.g., having at least 70%, 75%, 80%, 85%, 90%, 95%, or 99% sequence identity) thereto.

TABLE 32
Sequence Regions in ITR to ITR Sequences

| | GBA_VG32 (SEQ ID NO: 1827) | | |
|---|---|---|---|
| | Region SEQ | Region | Position in SEQ |
| Sequence Regions | ID NO | length | ID NO: 1827 |
| 5′ ITR | 1829 | 130 | 1-130 |
| CMVie | 1831 | 380 | 204-583 |
| CB promoter | 1834 | 260 | 590-849 |
| Intron | 1842 | 566 | 877-1442 |
| Signal sequence | 1850 | 117 | 1467-1583 |
| GBA Variant 1 coding | 1773 | 1,491 | 1584-3074 |
| sequence | | | |
| G4S3 linker | 1730 | 45 | 3075-3119 |
| TAT coding sequence | 1793 | 42 | 3120-3161 |
| PolyA | 1846 | 127 | 3201-3327 |
| 3′ ITR | 1830 | 130 | 3371-3500 |

In some embodiments the AAV particle comprises a viral genome comprising the nucleotide sequence of SEQ ID NO: 1827 (GBA_VG32), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1827, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1827, comprises in 5′ to 3′ order: a 5′ ITR sequence region comprising the nucleotide sequence of SEQ ID NO: 1829, or a nucleotide sequence at least 95% identical thereto; a CMVie enhancer comprising the nucleotide sequence of SEQ ID NO: 1831, or a nucleotide sequence at least 95% identical thereto; a CB promoter comprising the nucleotide sequence of SEQ ID NO: 1834, or a nucleotide sequence at least 95% identical thereto; an intron comprising the nucleotide sequence of SEQ ID NO: 1842, or a nucleotide sequence at least 95% identical thereto; a nucleotide sequence encoding a signal sequence comprising the nucleotide sequence of SEQ ID NO: 1850, or a nucleotide sequence at least 95% identical thereto; a nucleotide sequence encoding a GBA protein comprising the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773; a nucleotide sequence encoding a G4S3 linker comprising the nucleotide sequence of SEQ ID NO: 1730, or a nucleotide sequence at least 85% (e.g., at least 90%, 92%, 95%, 96%, 97%, 98%, or 99%) identical thereto; a nucleotide sequence encoding a TAT peptide comprising the nucleotide sequence of SEQ ID NO: 1793, or a nucleotide sequence at least 85% (e.g., at least 90%, 92%, 95%, 96%, 97%, 98%, or 99%) identical thereto; a polyadenylation sequence comprising the nucleotide sequence of SEQ ID NO: 1846, or a nucleotide sequence at least 95% identical thereto; and a 3′ ITR sequence region comprising the nucleotide sequence of SEQ ID NO: 1830, or a nucleotide sequence at least 95% identical thereto.

In some embodiments, the viral genome comprising the nucleotide sequence of SEQ ID NO: 1827, or a nucleotide sequence substantially identical (e.g., having at least 70%, 75%, 80%, 85%, 90%, 95%, or 99% sequence identity) thereto, encodes a GBA protein comprising the amino acid sequence of SEQ ID NO: 1775, or an amino acid sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1827, or a nucleotide sequence substantially identical (e.g., having at least 70%, 75%, 80%, 85%, 90%, 95%, or 99% sequence identity) thereto, encodes a TAT peptide comprising the amino acid sequence of SEQ ID NO: 1794, or an amino acid sequence having at least one, two, or three but no more than four modifications, e.g., substitutions, relative to SEQ ID NO: 1794.

TABLE 21
Sequence Regions in ITR to ITR Sequences

| | GBA_VG33 (SEQ ID NO: 1828) | | |
|---|---|---|---|
| | Region SEQ | Region | Position in SEQ |
| Sequence Regions | ID NO | length | ID NO: 1828 |
| 5′ ITR | 1829 | 130 | 1-130 |
| CMVie | 1831 | 380 | 204-583 |
| CB promoter | 1834 | 260 | 590-849 |
| Intron | 1842 | 566 | 877-1442 |
| Signal sequence | 1850 | 117 | 1467-1583 |
| GBA Variant 1 coding | 1773 | 1,491 | 1584-3074 |
| sequence | | | |
| miR183 binding site | 1847 | 22 | 3108-3129 |
| Spacer | 1848 | 8 | 3130-3137 |
| miR183 binding site | 1847 | 22 | 3138-3159 |
| Spacer | 1848 | 8 | 3160-3167 |
| miR183 binding site | 1847 | 22 | 3168-3189 |
| Spacer | 1848 | 8 | 3190-3197 |
| miR183 binding site | 1847 | 22 | 3198-3219 |
| PolyA | 1846 | 127 | 3272-3398 |
| 3′ ITR | 1830 | 130 | 3442-3751 |

In some embodiments, the AAV particle comprises a viral genome comprising the nucleotide sequence of SEQ ID NO: 1828 (GBA_VG33), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1828, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1828, comprises in 5′ to 3′ order: a 5′ ITR sequence region comprising the nucleotide sequence of SEQ ID NO: 1829, or a nucleotide sequence at least 95% identical thereto; a CMVie enhancer comprising the nucleotide sequence of SEQ ID NO: 1831, or a nucleotide sequence at least 95% identical thereto; a CB promoter comprising the nucleotide sequence of SEQ ID NO: 1834, or a nucleotide sequence at least 95% identical thereto; an intron comprising the nucleotide sequence of SEQ ID NO: 1842, or a nucleotide sequence at least 95% identical thereto; a nucleotide sequence encoding a signal sequence comprising the nucleotide sequence of SEQ ID NO: 1850, or a nucleotide sequence at least 95% identical thereto; a nucleotide sequence encoding a GBA protein comprising the nucleotide sequence of SEQ ID NO: 1773 or a nucleotide sequence at least 88% (e.g., at least 89, 90, 92, 95, 96, 97, 98, or 99%) identical to the nucleotide sequence of SEQ ID NO: 1773; a nucleotide sequence encoding a miR183 binding site comprising the nucleotide sequence of SEQ ID NO: 1847, or a nucleotide sequence having at least one, two, three, four, five, six, or seven modifications, but no more than ten modifications of SEQ ID NO: 1847; a spacer comprising the nucleotide sequence of SEQ ID NO: 1848, or a nucleotide sequence having at least one, two, or three modifications, but no more than four modifications of SEQ ID NO: 1848; a nucleotide sequence encoding a miR183 binding site comprising the nucleotide sequence of SEQ ID NO: 1847, or a nucleotide sequence having at least one, two, three, four, five, six, or seven modifications, but no more than ten modifications of SEQ ID NO: 1847; a spacer comprising the nucleotide sequence of SEQ ID NO: 1848, or a nucleotide sequence having at least one, two, or three modifications, but no more than four modifications of SEQ ID NO: 1848; a nucleotide sequence encoding a miR183 binding site comprising the nucleotide sequence of SEQ ID NO: 1847, or a nucleotide sequence having at least one, two, three, four, five, six, or seven modifications, but no more than ten modifications of SEQ ID NO: 1847; a spacer comprising the nucleotide sequence of SEQ ID NO: 1848, or a nucleotide sequence having at least one, two, or three modifications, but no more than four modifications of SEQ ID NO: 1848; a nucleotide sequence encoding a miR183 binding site comprising the nucleotide sequence of SEQ ID NO: 1847, or a nucleotide sequence having at least one, two, three, four, five, six, or seven modifications, but no more than ten modifications of SEQ ID NO: 1847; a polyadenylation sequence comprising the nucleotide sequence of SEQ ID NO: 1846, or a nucleotide sequence at least 95% identical thereto; and a 3′ ITR sequence region comprising the nucleotide sequence of SEQ ID NO: 1830, or a nucleotide sequence at least 95% identical thereto.

In some embodiments, the viral genome comprising the nucleotide sequence of SEQ ID NO: 1828, or a nucleotide sequence substantially identical (e.g., having at least 70%, 75%, 80%, 85%, 90%, 95%, or 99% sequence identity) thereto, encodes a GBA protein comprising the amino acid sequence of SEQ ID NO: 1775, or an amino acid sequence substantially identical (e.g., having at least 70%, 75%, 80%, 85%, 90%, 95%, or 99% sequence identity) thereto.

In some embodiments, the AAV particle comprises an AAV viral genome comprising the nucleotide sequence of any of the viral genomes described herein, e.g., as described in Tables 18-21 or 29-32, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the AAV viral genome further comprises a nucleic acid encoding a capsid protein, e.g., a structural protein. In some embodiments, the capsid protein comprises a VP1 polypeptide, a VP2 polypeptide, and/or a VP3 polypeptide. In some embodiments, the VP1 polypeptide, the VP2 polypeptide, and/or the VP3 polypeptide are encoded by at least one Cap gene. In some embodiments, the AAV viral genome further comprises a nucleic acid encoding a Rep protein, e.g., a non-structural protein. In some embodiments, the Rep protein comprises a Rep78 protein, a Rep68, Rep52 protein, and/or a Rep40 protein. In some embodiments, the Rep78 protein, the Rep68 protein, the Rep52 protein, and/or the Rep40 protein are encoded by at least one Rep gene.

In some embodiment, the AAV particle comprising a viral comprising the nucleotide sequence of any of the viral genomes described herein, e.g., as described in Tables 18-21 or 29-32, or a nucleotide sequence substantially identical (e.g., having at least about 70%, 75%, 80%, 85%, 90%, 92%, 95%, 97%, 98%, or 99% sequence identity) to any of the aforesaid sequences comprises, e.g., is packaged in, a capsid protein having a serotype or a functional variant thereof selected from Table 1. In some embodiments, the capsid protein comprise a VOY101, VOY201, AAVPHP.N (PHP.N), AAVPHP.B (PHP.B), AAVPHP.A (PHP.A), PHP.B2, PHP.B3, G2B4, G2B5, AAV9, AAVrh10, or a functional variant thereof. In some embodiments, the capsid protein comprises a VOY101 capsid protein, or functional variant thereof.

In some embodiments, the AAV particle comprising a viral genome comprising the nucleotide sequence of any of the viral genomes described herein, e.g., as described in Tables 18-21 or 29-32, or a nucleotide sequence substantially identical (e.g., having at least about 70%, 75%, 80%, 85%, 90%, 92%, 95%, 97%, 98%, or 99% sequence identity) to any of the aforesaid sequences comprises a capsid protein comprising the amino acid sequence of SEQ ID NO: 138, or a sequence substantially identical (e.g., having at least about 70%, 75%, 80%, 85%, 90%, 92%, 95%, 97%, 98%, or 99% sequence identity) thereto. In some embodiments, the capsid protein comprises an amino acid sequence having at least one, two or three modifications, but not more than 30, 20 or 10 modifications of the amino acid sequence of SEQ ID NO: 138. In some embodiments, the capsid protein is encoded by the nucleotide sequence of SEQ ID NO: 137, or a nucleotide sequence substantially identical (e.g., having at least 70%, 75%, 80%, 85%, 90%, 95% or 99%) thereto. In some embodiments, the capsid protein comprises an amino acid substitution at position K449, e.g., a K449R substitution, numbered according to SEQ ID NO:138. In some embodiments, the capsid protein comprises an insert comprising the amino acid sequence of TLAVPFK (SEQ ID NO: 1262), wherein the insert is present immediately subsequent to position 588, relative to a reference sequence numbered according to SEQ ID NO:138. In some embodiments, the capsid protein comprises an amino acid other than “A” at position 587 and/or an amino acid other than “Q” at position 588, numbered according to SEQ ID NO:138. In some embodiments, the capsid protein comprises the amino acid substitution of A587D and/or Q588G, numbered according to SEQ ID NO:138.

In some embodiments, the AAV particle comprising a viral genome comprising the nucleotide sequence of any of the viral genomes described herein, e.g., as described in Tables 18-21 or 29-32, or a nucleotide sequence substantially identical (e.g., having at least about 70%, 75%, 80%, 85%, 90%, 92%, 95%, 97%, 98%, or 99% sequence identity) to any of the aforesaid sequences comprises a capsid protein comprising the amino acid sequence of SEQ ID NO: 1, or a sequence substantially identical (e.g., having at least about 70%, 75%, 80%, 85%, 90%, 92%, 95%, 97%, 98%, or 99% sequence identity) thereto. In some embodiments, the capsid protein comprises an amino acid sequence having at least one, two or three modifications, but not more than 30, 20 or 10 modifications of the amino acid sequence of SEQ ID NO: 1. In some embodiments, the capsid protein is encoded by the nucleotide sequence of SEQ ID NO: 2, or a nucleotide sequence substantially identical (e.g., having at least 70%, 75%, 80%, 85%, 90%, 95% or 99%) thereto.

The present disclosure provides in some embodiments, vectors, cells, and/or AAV particles comprising the above identified viral genomes.

### Self-Complementary and Single Strand Vectors

In some embodiments, the AAV vector used in the present disclosure is a single strand vector (ssAAV).

In some embodiments, the AAV vectors may be self-complementary AAV vectors (scAAVs). See, e.g., U.S. Pat. No. 7,465,583. scAAV vectors contain both DNA strands that anneal together to form double stranded DNA. By skipping second strand synthesis, scAAVs allow for rapid expression in the cell.

In some embodiments, the AAV vector used in the present disclosure is a scAAV.

Methods for producing and/or modifying AAV vectors are disclosed in the art such as pseudotyped AAV vectors (International Patent Publication Nos. WO200028004; WO200123001; WO2004112727; WO 2005005610 and WO 2005072364, the content of each of which are incorporated herein by reference in their entirety).

### Viral Genome Size

In some embodiments, the viral genome of the AAV particles of the present disclosure may be single or double stranded. The size of the vector genome may be small, medium, large or the maximum size.

In some embodiments, the vector genome, which comprises a nucleic acid sequence encoding GCase protein described herein, may be a small single stranded vector genome. A small single stranded vector genome may be about 2.7 kb to about 3.5 kb in size such as about 2.7, about 2.8, about 2.9, about 3.0, about 3.1, about 3.2, about 3.3, about 3.4, or about 3.5 kb in size. In some embodiments, the small single stranded vector genome may be 3.2 kb in size.

In some embodiments, the vector genome, which comprises a nucleic acid sequence encoding GCase protein described herein, may be a small double stranded vector genome. A small double stranded vector genome may be about 1.3 to about 1.7 kb in size such as about 1.3, about 1.4, about 1.5, about 1.6, or about 1.7 kb in size. In some embodiments, the small double stranded vector genome may be 1.6 kb in size.

In some embodiments, the vector genome, which comprises a nucleic acid sequence encoding GCase protein described herein, may be a medium single stranded vector genome. A medium single stranded vector genome may be about 3.6 to about 4.3 kb in size such as about 3.6, about 3.7, about 3.8, about 3.9, about 4.0, about 4.1, about 4.2, or about 4.3 kb in size. In some embodiments, the medium single stranded vector genome may be 4.0 kb in size.

In some embodiments, the vector genome, which comprises a nucleic acid sequence encoding GCase protein described herein, may be a medium double stranded vector genome. A medium double stranded vector genome may be about 1.8 to about 2.1 kb in size such as about 1.8, about 1.9, about 2.0, or about 2.1 kb in size. In some embodiments, the medium double stranded vector genome may be 2.0 kb in size. Additionally, the vector genome may comprise a promoter and a polyA tail.

In some embodiments, the vector genome which comprises a nucleic acid sequence encoding GCase protein described herein may be a large single stranded vector genome. A large single stranded vector genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded vector genome may be 4.7 kb in size. As another non-limiting example, the large single stranded vector genome may be 4.8 kb in size. As yet another non-limiting example, the large single stranded vector genome may be 6.0 kb in size.

In some embodiments, the vector genome which comprises a nucleic acid sequence encoding GCase protein described herein may be a large double stranded vector genome. A large double stranded vector genome may be 2.2 to 3.0 kb in size such as about 2.2, 2.3, 2.4, 2.5, 2.6, 2.7, 2.8, 2.9 and 3.0 kb in size. As a non-limiting example, the large double stranded vector genome may be 2.4 kb in size.

### Backbone

In certain embodiments, a cis-element such as a vector backbone is incorporated into the viral particle encoding, e.g., a GBA protein or a GBA protein and an enhancement element described herein. Without wishing to be bound by theory, it is believed, in some embodiments, the backbone sequence may contribute to the stability of GBA protein expression, and/or the level of expression of the GBA protein.

The present disclosure also provides in some embodiments, a nucleic acid encoding a viral genome, e.g., a viral genome comprising the nucleotide sequence of any of the viral genomes in Tables 18-21 or 29-32, or a nucleotide sequence substantially identical (e.g., having at least 70%, 75%, 80%, 85%, 90%, 95%, 99% or 100%, sequence identity) thereto, an a backbone region suitable for replication of the viral genome in a cell, e.g., a bacterial cell (e.g., wherein the backbone region comprises one or both of a bacterial origin of replication and a selectable marker).

II. Viral production

### General Viral Production Process

Cells for the production of AAV, e.g., rAAV, particles may comprise, in some embodiments, mammalian cells (such as HEK293 cells) and/or insect cells (such as Sf9 cells).

In various embodiments, AAV production includes processes and methods for producing AAV particles and vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, disclosed herein is a vector comprising a viral genome of the present disclosure. In some embodiments, disclosed herein is a cell comprising a viral genome of the present disclosure. In some embodiments, the cell is a bacterial cell, a mammalian cell (e.g., a HEK293 cell), or an insect cell (e.g., an Sf9 cell).

In some embodiments, disclosed herein is a method of making a viral genome. The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral genome from the backbone region, e.g., by cleaving the nucleic acid molecule at upstream and downstream of the viral genome. In some embodiments, the viral genome comprising a promoter operably linked to nucleic acid comprising a transgene encoding a GBA protein (e.g., a GBA protein described herein), will be incorporated into an AAV particle produced in the cell. In some embodiments, the cell is a bacterial cell, a mammalian cell (e.g., a HEK293 cell), or an insect cell (e.g., an Sf9 cell).

In some embodiments, disclosed herein is a method of making a recombinant AAV particle of the present disclosure, the method comprising (i) providing a host cell comprising a viral genome described herein and incubating the host cell under conditions suitable to enclose the viral genome in a capsid protein, e.g., a capsid protein described herein (e.g., a capsid protein listed in Table 1, e.g., a VOY101 capsid protein or functional variant thereof), thereby making the recombinant AAV particle. In some embodiments, the method comprises prior to step (i), introducing a first nucleic acid comprising the viral genome into a cell. In some embodiments, the host cell comprises a second nucleic acid encoding the capsid protein. In some embodiments, the second nucleic acid is introduced into the host cell prior to, concurrently with, or after the first nucleic acid molecule. In some embodiments, the host cell is a bacterial cell, a mammalian cell (e.g., a HEK293 cell), or an insect cell (e.g., an Sf9 cell).

In various embodiments, methods are provided herein of producing AAV particles or vectors by (a) contacting a viral production cell with one or more viral expression constructs encoding at least one AAV capsid protein, and one or more payload constructs encoding a payload molecule, which can be selected from: a transgene, a polynucleotide encoding protein, and a modulatory nucleic acid; (b) culturing the viral production cell under conditions such that at least one AAV particle or vector is produced, and (c) isolating the AAV particle or vector from the production stream.

In these methods, a viral expression construct may encode at least one structural protein and/or at least one non-structural protein. The structural protein may include any of the native or wild type capsid proteins VP1, VP2, and/or VP3, or a chimeric protein thereof. The non-structural protein may include any of the native or wild type Rep78, Rep68, Rep52, and/or Rep40 proteins or a chimeric protein thereof.

In certain embodiments, contacting occurs via transient transfection, viral transduction, and/or electroporation.

In certain embodiments, the viral production cell is selected from a mammalian cell and an insect cell. In certain embodiments, the insect cell includes a Spodoptera frugiperda insect cell. In certain embodiments, the insect cell includes a Sf9 insect cell. In certain embodiments, the insect cell includes a Sf21 insect cell.

The payload construct vector of the present disclosure may include, in various embodiments, at least one inverted terminal repeat (ITR) and may include mammalian DNA.

Also provided are AAV particles and viral vectors produced according to the methods described herein.

In various embodiments, the AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell or a mammalian cell) with at least one viral expression construct encoding at least one capsid protein and at least one payload construct vector. The viral production cell may be contacted by transient transfection, viral transduction, and/or electroporation. The payload construct vector may include a payload construct encoding a payload molecule such as, but not limited to, a transgene, a polynucleotide encoding protein, and a modulatory nucleic acid. The viral production cell can be cultured under conditions such that at least one AAV particle or vector is produced, isolated (e.g., using temperature-induced lysis, mechanical lysis and/or chemical lysis) and/or purified (e.g., using filtration, chromatography, and/or immunoaffinity purification). As a non-limiting example, the payload construct vector may include mammalian DNA.

In certain embodiments, the AAV particles are produced in an insect cell (e.g., Spodoptera frugiperda (Sf9) cell) using a method described herein. As a non-limiting example, the insect cell is contacted using viral transduction which may include baculoviral transduction.

In certain embodiments, the AAV particles are produced in an mammalian cell (e.g., HEK293 cell) using a method described herein. As a non-limiting example, the mammalian cell is contacted using viral transduction which may include multiplasmid transient transfection (such as triple plasmid transient transfection).

In certain embodiments, the AAV particle production method described herein produces greater than 10¹, greater than 10², greater than 10³, greater than 10⁴, or greater than 10⁵ AAV particles in a viral production cell.

In certain embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

In various embodiments, once administered, an AAV particle disclosed herein may, without being bound by theory, contact a target cell and enter the cell, e.g., in an endosome. The AAV particles, e.g., those released from the endosome, may subsequently contact the nucleus of the target cell to deliver the payload construct. The payload construct, e.g. recombinant viral construct, may be delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

In some embodiments, large scale production of AAV particles utilizes a bioreactor. Without being bound by theory, the use of a bioreactor may allow for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In some embodiments, the bioreactor is used for continuous production in which a portion of the culture is harvested at an experimentally determined time point for purification of AAV particles, and the remaining culture in the bioreactor is refreshed with additional growth media components.

In various embodiments, AAV viral particles can be extracted from viral production cells in a process which includes cell lysis, clarification, sterilization and purification. Cell lysis includes any process that disrupts the structure of the viral production cell, thereby releasing AAV particles. In certain embodiments, cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

In various embodiments, the end result of viral production is a purified collection of AAV particles which include two components: (1) a payload construct (e.g. a recombinant AAV vector genome construct) and (2) a viral capsid.

In certain embodiments, a viral production system or process of the present disclosure includes steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure includes steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BIICs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

### Viral Expression Constructs

In various embodiments, the viral production system of the present disclosure includes one or more viral expression constructs that can be transfected/transduced into a viral production cell. In certain embodiments, a viral expression construct or a payload construct of the present disclosure can be a bacmid, also known as a baculovirus plasmid or recombinant baculovirus genome. In certain embodiments, the viral expression includes a protein-coding nucleotide sequence and at least one expression control sequence for expression in a viral production cell. In certain embodiments, the viral expression includes a protein-coding nucleotide sequence operably linked to least one expression control sequence for expression in a viral production cell. In certain embodiments, the viral expression construct contains parvoviral genes under control of one or more promoters. Parvoviral genes can include nucleotide sequences encoding non-structural AAV replication proteins, such as Rep genes which encode Rep52, Rep40, Rep68, or Rep78 proteins. Parvoviral genes can include nucleotide sequences encoding structural AAV proteins, such as Cap genes which encode VP1, VP2, and VP3 proteins.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of each of which are herein incorporated by reference in their entirety as related to viral expression constructs and uses thereof.

In certain embodiments, the viral expression construct is an AAV expression construct which includes one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may contain a nucleotide sequence which includes start codon region, such as a sequence encoding AAV capsid proteins which include one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG).

In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in their entirety as related to AAV capsid proteins and the production thereof.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG, and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may promote high vector yields.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art.

In certain embodiments, the polynucleotide incorporated into the bacmid (i.e. polynucleotide insert) can include an expression control sequence operably linked to a protein-coding nucleotide sequence. In certain embodiments, the polynucleotide incorporated into the bacmid can include an expression control sequence which includes a promoter, such as p10 or polh, and which is operably linked to a nucleotide sequence which encodes a structural AAV capsid protein (e.g. VP1, VP2, VP3 or a combination thereof). In certain embodiments, the polynucleotide incorporated into the bacmid can include an expression control sequence which includes a promoter, such as p10 or polh, and which is operably linked to a nucleotide sequence which encodes a non-structural AAV capsid protein (e.g. Rep78, Rep52, or a combination thereof).

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is: 1:1:10 (VP1:VP2:VP3); 2:2:10 (VP1:VP2:VP3); 2:0:10 (VP1:VP2:VP3); 1-2:0-2:10 (VP1:VP2:VP3); 1-2:1-2:10 (VP1:VP2:VP3); 2-3:0-3:10 (VP1:VP2:VP3); 2-3:2-3:10 (VP1:VP2:VP3); 3:3:10 (VP1:VP2:VP3); 3-5:0-5:10 (VP1:VP2:VP3); or 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the ie-1 promoter, the Δie-1 promoter, has about 20% of the transcriptional activity of that ie-1 promoter. A promoter substantially homologous to the Δie-1 promoter may be used. In respect to promoters, a homology of at least 50%, 60%, 70%, 80%, 90% or more, is considered to be a substantially homologous promoter.

### Mammalian Cells

Viral production of the present disclosure disclosed herein describes processes and methods for producing AAV particles or viral vector that contacts a target cell to deliver a payload construct, e.g. a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO, W138, HeLa, HEK293, HEK293T (293T), Saos, C2C12, L cells, HT1080, Huh7, HepG2, C127, 3T3, CHO, HeLa cells, KB cells, BHK and primary fibroblast, hepatocyte, and myoblast cells derived from mammals. Viral production cells can include cells derived from any mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of each of which are herein incorporated by reference in their entireties insofar as they do no conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the contents of which are herein incorporated by reference in their entirety as related to the 293-10-3 packaging cell line and uses thereof.

In certain embodiments, of the present disclosure a cell line, such as a HeLA cell line, for trans-complementing E1 deleted adenoviral vectors, which encoding adenovirus Ela and adenovirus E1b under the control of a phosphoglycerate kinase (PGK) promoter can be used for AAV particle production as described in U.S. Pat. No. 6,365,394, the contents of which are incorporated herein by reference in their entirety as related to the HeLa cell line and uses thereof.

In certain embodiments, AAV particles are produced in mammalian cells using a multiplasmid transient transfection method (such as triple plasmid transient transfection). In certain embodiments, the multiplasmid transient transfection method includes transfection of the following three different constructs: (i) a payload construct, (ii) a Rep/Cap construct (parvoviral Rep and parvoviral Cap), and (iii) a helper construct. In certain embodiments, the triple transfection method of the three components of AAV particle production may be utilized to produce small lots of virus for assays including transduction efficiency, target tissue (tropism) evaluation, and stability. In certain embodiments, the triple transfection method of the three components of AAV particle production may be utilized to produce large lots of materials for clinical or commercial applications.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other Ela trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference in their entirety as related to the production and processing of recombinant AAV virus particles. Production may also involve methods known in the art including those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g. medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80C to 37C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated by reference in their entireties as related to the measurement of particle concentrations).

### Insect Cells

Viral production of the present disclosure includes processes and methods for producing AAV particles or viral vectors that contact a target cell to deliver a payload construct, e.g., a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety as related to the growth and use of insect cells in viral production.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir.219:37-44 (1996); Zhao et al., Vir.272:382-93 (2000); and Samulski et al., U.S. Pat. No. 6,204,059, the contents of each of which are herein incorporated by reference in their entirety as related to the use of insect cells in viral production.

In some embodiments, the AAV particles are made using the methods described in WO2015/191508, the contents of which are herein incorporated by reference in their entirety insofar as they do not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the contents of which are herein incorporated by reference in their entirety as related to the production of viral particles.

Expansion, culturing, transfection, infection and storage of insect cells can be carried out in any cell culture media, cell transfection media or storage media known in the art, including Hyclone™ SFX-Insect™ Cell Culture Media, Expression System ESF AF™ Insect Cell Culture Medium, ThermoFisher Sf-900II™ media, ThermoFisher Sf-900III™ media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production Systems

In certain embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can include the viral expression construct (Expression BEV), and one or more group which can include the payload construct (Payload BEV). The baculoviruses may be used to infect a viral production cell for production of AAV particles or viral vector.

In certain embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In certain embodiments, BEVs are produced using a Bacmid Transfection agent, such as Promega FuGENE® HD, WFI water, or ThermoFisher Cellfectin® II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN2 vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety as related to the production and use of BEVs and viral particles.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture. Wasilko D J et al. Protein Expr Purif. 2009 June; 65(2):122-32, the contents of which are herein incorporated by reference in their entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such embodiments, the corresponding bacmid vector is engineered with replication control elements, including but not limited to promoters, enhancers, and/or cell-cycle regulated replication elements.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, the AAV particle of the present disclosure may be produced in insect cells (e.g., Sf9 cells).

In some embodiments, the AAV particle of the present disclosure may be produced using triple transfection.

In some embodiments, the AAV particle of the present disclosure may be produced in mammalian cells.

In some embodiments, the AAV particle of the present disclosure may be produced by triple transfection in mammalian cells.

In some embodiments, the AAV particle of the present disclosure may be produced by triple transfection in HEK293 cells.

The AAV viral genomes encoding GCase protein described herein may be useful in the fields of human disease, veterinary applications and a variety of in vivo and in vitro settings. The AAV particles of the present disclosure may be useful in the field of medicine for the treatment, prophylaxis, palliation, or amelioration of neurological or neuromuscular diseases and/or disorders. In some embodiments, the AAV particles of the disclosure are used for the prevention and/or treatment of GBA-related disorders.

Various embodiments of the disclosure herein provide a pharmaceutical composition comprising the AAV particle described herein and a pharmaceutically acceptable excipient.

Various embodiments of the disclosure herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of: intravenous, intracerebroventricular, intraparenchymal, intrathecal, subpial, and intramuscular, or a combination thereof. Certain embodiments of the method provide that the subject is treated for GBA-related disorders and/or other neurological disorder arising from a deficiency in the quantity or function of GBA gene products. In one aspect of the method, a pathological feature of the GBA-related disorders or the other neurological disorder is alleviated and/or the progression of the GBA-related disorders or the other neurological disorder is halted, slowed, ameliorated, or reversed.

Various embodiments of the disclosure herein describe a method of increasing the level of GCase protein in the central nervous system of a subject in need thereof comprising administering to said subject via infusion, an effective amount of the pharmaceutical composition described herein.

Also described herein are compositions, methods, processes, kits and devices for the design, preparation, manufacture and/or formulation of AAV particles. In some embodiments, payloads, such as but not limited to payloads comprising GCase protein, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of GBA-related disorders. Such methods may involve gene replacement or gene activation. Such outcomes are achieved by utilizing the methods and compositions taught herein.

### III. Pharmaceutical Compositions

The present disclosure additionally provides a method for treating GBA-related disorders and disorders related to deficiencies in the function or expression of GCase protein(s) in a mammalian subject, including a human subject, comprising administering to the subject any of the AAV polynucleotides or AAV genomes described herein (i.e., “vector genomes,” “viral genomes,” or “VGs”) or administering to the subject a particle comprising said AAV polynucleotide or AAV genome, or administering to the subject any of the described compositions, including pharmaceutical compositions.

As used herein the term “composition” comprises an AAV polynucleotide or AAV genome or AAV particle and at least one excipient.

As used herein the term “pharmaceutical composition” comprises an AAV polynucleotide or AAV genome or AAV particle and one or more pharmaceutically acceptable excipients.

Although the descriptions of pharmaceutical compositions, e.g., AAV comprising a payload encoding a GCase protein to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients, or subjects.

In some embodiments, the AAV particle formulations described herein may contain a nucleic acid encoding at least one payload. In some embodiments, the formulations may contain a nucleic acid encoding 1, 2, 3, 4, or 5 payloads. In some embodiments, the formulation may contain a nucleic acid encoding a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic proteins, cytoskeletal proteins, intracellular membrane bound proteins, nuclear proteins, proteins associated with human disease, and/or proteins associated with non-human diseases. In some embodiments, the formulation contains at least three payload constructs encoding proteins. Certain embodiments provide that at least one of the payloads is GCase protein or a variant thereof.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### IV. Formulations

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

For example, the composition may comprise between 0.1% and 99% (w/w) of the active ingredient. By way of example, the composition may comprise between 0.1% and 100%, e.g., between 0.5% and 50%, between 1-30%, between 5-80%, or at least 80% (w/w) active ingredient.

The AAV particles of the disclosure can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

In some embodiments, the viral vectors encoding GCase protein may be formulated to optimize baricity and/or osmolality. In some embodiments, the baricity and/or osmolality of the formulation may be optimized to ensure optimal drug distribution in the central nervous system or a region or component of the central nervous system.

In some embodiments, the AAV particles of the disclosure may be formulated in PBS with 0.001% of pluronic acid (F-68) at a pH of about 7.0.

In some embodiments, the AAV particles of the disclosure may be formulated in PBS, in combination with an ethylene oxide/propylene oxide copolymer (also known as pluronic or poloxamer).

In some embodiments, the AAV particles of the disclosure may be formulated in PBS with 0.001% pluronic acid (F-68) (poloxamer 188) at a pH of about 7.0.

In some embodiments, the AAV particles of the disclosure may be formulated in PBS with 0.001% pluronic acid (F-68) (poloxamer 188) at a pH of about 7.3.

In some embodiments, the AAV particles of the disclosure may be formulated in PBS with 0.001% pluronic acid (F-68) (poloxamer 188) at a pH of about 7.4.

In some embodiments, the AAV particles of the disclosure may be formulated in a solution comprising sodium chloride, sodium phosphate and an ethylene oxide/propylene oxide copolymer.

In some embodiments, the AAV particles of the disclosure may be formulated in a solution comprising sodium chloride, sodium phosphate dibasic, potassium chloride, potassium phosphate monobasic, and poloxamer 188/pluronic acid (F-68).

In some embodiments, the AAV particles of the disclosure may be formulated in a solution comprising 192 mM sodium chloride, 10 mM sodium phosphate (dibasic), 2.7 mM potassium chloride, 2 mM potassium phosphate (monobasic) and 0.001% pluronic F-68 (v/v), at pH 7.4. This formulation is referred to as Formulation 1 in the present disclosure.

In some embodiments, the AAV particles of the disclosure may be formulated in a solution comprising about 192 mM sodium chloride, about 10 mM sodium phosphate dibasic and about 0.001% poloxamer 188, at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate dibasic in the final solution may be 1 mM-50 mM. As non-limiting examples, the concentration of sodium phosphate dibasic in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the disclosure may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

### Excipients

The formulations of the disclosure can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; the contents of which are herein incorporated by reference in their entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none, or some of the inactive ingredients which may be used in the formulations of the present disclosure may be approved by the US Food and Drug Administration (FDA).

Formulations of AAV particles disclosed herein may include cations or anions. In some embodiments, the formulations include metal cations such as, but not limited to, Zn²⁺, Ca²⁺, Cu²⁺, Mg⁺, or combinations thereof. In some embodiments, formulations may include polymers or polynucleotides complexed with a metal cation (see, e.g., U.S. Pat. Nos. 6,265,389 and 6,555,525, the contents of each of which are herein incorporated by reference in their entirety).

### V. Uses and Applications

The compositions of the disclosure may be administered to a subject or used in the manufacture of a medicament for administration to a subject having a deficiency in the quantity or function of GCase protein or having a disease or condition associated with decreased GCase protein expression. In some embodiments, the disease is Parkinson Disease (PD), e.g., a PD with a mutation in a GBA gene. In certain embodiments, the AAV particles including GCase protein may be administered to a subject to treat Parkinson Disease, e.g., as PD associated with a mutation in a GBA gene. In some embodiments, administration of the AAV particles comprising viral genomes that encode GCase protein may protect central nervous system pathways from degeneration. The compositions and methods described herein are also useful for treating Gaucher disease (such as Type 1 or 2 GD) and Dementia with Lewy Bodies, and other GBA-related disorders.

In some embodiments, the delivery of the AAV particles may halt or slow progression of GBA-related disorders as measured by cholesterol accumulation in CNS cells (as determined, for example, by filipin staining and quantification). In certain embodiments, the delivery of the AAV particles improves symptoms of GBA-related disorders, including, for example, cognitive, muscular, physical, and sensory symptoms of GBA-related disorders.

In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

In certain embodiments, the pharmaceutical compositions described herein are used as research tools, particularly in in vitro investigations using human cell lines such as HEK293T and in vivo testing in nonhuman primates which will occur prior to human clinical trials.

### CNS Diseases

The present disclosure provides a method for treating a disease, disorder and/or condition in a mammalian subject, including a human subject, comprising administering to the subject any of the viral particles e.g., AAV, AAV particle, or AAV genome that produces GCase protein described herein (i.e., viral genomes or “VG”) or administering to the subject a particle comprising said AAV particle or AAV genome, or administering to the subject any of the described compositions, including pharmaceutical compositions.

In some embodiments, AAV particles of the present disclosure, through delivery of a functional payload that is a therapeutic product comprising a GCase protein or variant thereof that can modulate the level or function of a gene product in the CNS.

A functional payload may alleviate or reduce symptoms that result from abnormal level and/or function of a gene product (e.g., an absence or defect in a protein) in a subject in need thereof or that otherwise confers a benefit to a CNS disorder in a subject in need thereof.

As non-limiting examples, companion or combination therapeutic products delivered by AAV particles of the present disclosure may include, but are not limited to, growth and trophic factors, cytokines, hormones, neurotransmitters, enzymes, anti-apoptotic factors, angiogenic factors, GCase proteins, and any protein known to be mutated in pathological disorders such as GBA-related disorders.

In some embodiments, AAV particles of the present disclosure may be used to treat diseases that are associated with impairments of the growth and development of the CNS, i.e., neurodevelopmental disorders. In some aspects, such neurodevelopmental disorders may be caused by genetic mutations.

In some embodiments, the neurological disorders may be functional neurological disorders with motor and/or sensory symptoms which have neurological origin in the CNS. As non-limiting examples, functional neurological disorders may be chronic pain, seizures, speech problems, involuntary movements, or sleep disturbances.

In some embodiments, the neurological or neuromuscular disease, disorder, and/or condition is GBA-related disorders. In some embodiments, the delivery of the AAV particles may halt or slow the disease progression of GBA-related disorders by 10%, 20%, 30%, 40%, 50%, 60%, 70%, 80%, 90%, 95%, or more than 95% using a known analysis method and comparator group for GBA-related disorders. As a non-limiting example, the delivery of the AAV particles may halt or slow progression of GBA-related disorders as measured by cholesterol accumulation in CNS cells (as determined, for example, by filipin staining and quantification).

In some embodiments, the AAV particles described herein increase the amount of GCase protein in a tissue by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or more than 100%. In some embodiments, the AAV particle encoding a payload may increase the amount of GCase protein in a tissue to be comparable to (e.g., approximately the same as) the amount of GCase protein in the corresponding tissue of a healthy subject. In some embodiments, the AAV particle encoding a payload may increase the amount of GCase protein in a tissue effective to reduce one or more symptoms of a disease associated with decreased GCase protein expression or a deficiency in the quantity and/or function of GCase protein.

In some embodiments, the AAV particles and AAV vector genomes described herein, upon administration to subject or introduction to a target cell, increase GBA activity 2-3 fold over baseline GBA activity. In the case of subjects or target cells with deficient GBA activity, as in the case of subjects having a GBA-related disorder or cells or tissues harboring a mutation in a GBA gene, the AAV particles and AAV vector genomes described herein restore GBA activity to normal levels, as defined by GBA activity levels in subjects, tissues, and cells not afflicted with a GBA-related disorder or not harboring a GBA gene mutation. In some embodiments, the AAV particles and AAV vector genomes described herein effectively reduce α-synuclein levels in subjects having a GBA-related disorder or cells or tissues harboring a mutation in a GBA gene. In some embodiments, the AAV particles and AAV vector genomes described herein effectively prevent α-synuclein mediated pathology.

### Therapeutic Applications

The present disclosure additionally provides methods for treating non-infectious diseases and/or disorders in a mammalian subject, including a human subject, comprising administering to the subject any of the AAV particles or pharmaceutical compositions described herein. In some embodiments, non-infectious diseases and/or disorders treated according to the methods described herein include, but are not limited to, Parkinson's Disease (PD) (e.g., PD associated with a mutation in a GBA gene), Dementia with Lewy Bodies (DLB), Multiple System Atrophy (MSA), Decreased muscle mass, Spinal muscular atrophy (SMA), Alzheimer's disease (AD), Amyotrophic lateral sclerosis (ALS), Huntington's Disease (HD), Multiple sclerosis (MS), Stroke, Migraine, Pain, Neuropathies, Psychiatric disorders including schizophrenia, bipolar disorder, and autism, Cancer, ocular diseases, systemic diseases of the blood, heart and bone, Immune system and Autoimmune diseases and Inflammatory diseases.

The present disclosure provides a method for administering to a subject in need thereof, including a human subject, a therapeutically effective amount of the AAV particles of the invention to slow, stop or reverse disease progression. As a non-limiting example, disease progression may be measured by tests or diagnostic tool(s) known to those skilled in the art. As another non-limiting example, disease progression may be measured by change in the pathological features of the brain, CSF, or other tissues of the subject.

### Gaucher Disease

Homozygous or compound heterozygous GBA mutations lead to Gaucher disease (“GD”). See Sardi, S. Pablo, Jesse M. Cedarbaum, and Patrik Brundin. Movement Disorders 33.5 (2018): 684-696, the contents of which are incorporated by reference in their entirety. Gaucher disease is one of the most prevalent lysosomal storage disorders, with an estimated standardized birth incidence in the general population of between 0.4 to 5.8 individuals per 100,000. Heterozygous GBA mutations can lead to PD. Indeed, GBA mutations occur in 7-10% of total PD patients, making GBA mutations the most important genetic risk factor of PD. PD-GBA patients have reduced levels of lysosomal enzyme beta-glucocerebrosidase (GCase), which results in increased accumulations of glycosphingolipid glucosylceramide (GluCer), which in turn is correlated with exacerbated α-Synuclein aggregation and concomitant neurological symptoms. Gaucher disease and PD, as well as other lysosomal storage disorders including Lewy body dieseases such as Dementia with Lewy Bodies, and related diseases, in some cases, share common etiology in the GBA gene. See Sidransky, E. and Lopez, G. Lancet Neurol. 2012 November; 11(11): 986-998, the contents of which are incorporated by reference in their entirety.

Gaucher disease can present as GD1 (Type 1 GD), which is the most common type of Gaucher disease among Asheknazi Jewish populations. In some embodiments, a Type I GD is a non-neuronopathic GD (e.g., does not affect the CNS, e.g., impacts cells and tissues outside of the CNS, e.g., a peripheral cell or tissue, e.g., a heart tissue, a liver tissue, a spleen tissue, or a combination thereof). The carrier frequency among Ashkenazi Jewish populations is approximately 1 in 12 individuals. GD2 (Type 2 GD) is characterized by acute neuronopathic GD (e.g., affects the CNS, e.g., cells and tissues of the brain, spinal cord, or both), and has an estimated incidence of 1 in 150,000 live births. GD2 is an early onset disease, typically presenting at about 1 year of age. Visceral involvement is extensive and severe, with numerous attributes of CNS disease, including oculomotor dysfunction, and bulbar palsy and generalized weakness, and progressive development delay. GD2 progresses to severe hypertonia, rigidity, opisthotonos, dysphagia, and seizures, typically resulting in death before age 2. GD3 (type 3 GD) is characterized by sub-acute neuropathic GD and as an estimated incidence of 1 in 200,000 live births. GD3 typically presents with pronounced neurologic signs, including a characteristic mask-like face, strabismus, supranuclear gaze palsy, and poor upward gaze initiation. GD2 and GD3 are each further characterized as associated with progressive encephalopathy, with developmental delay, cognitive impairment, progressive dementia, ataxia, myoclonus, and various gaze palsies. GD1, on the other hand, can have variable etiology, with visceromegaly, marrow and skeletal and pulmonary pathology, bleeding diatheses, and developmental delay. GD is further associated with increased rates of hematologic malignancies.

Deficiency of Glucocerebrosidase (GCase) is the underlying mechanism of GD. Low GCase activity leads to accumulation of glucocerebroside and other glycolipids within the lysosomes of macrophages. Accumulation can amount to about 20-fold to about 100-fold higher than in control cells or subjects without GCase deficiency. Pathologic lipid accumulation in macrophages accounts for <2% of additional tissue mass observed in the liver and spleen of GD patients. Additional increase in organ weight and volume is attributed to an inflammatory and hyperplastic cellular response.

Current treatments of GD include administration of recombinant enzymes, imiglucerase, taliglucerase alfa, and velaglucerase alfa. However, these intravenous enzyme therapies do not cross the blood brain barrier (BBB), and are not suitable for treatment of GD with Parkinson's disease or other neuronopathic forms of GD.

### Parkinson's Disease

Parkinson's Disease (PD) is a progressive disorder of the nervous system affecting especially the substantia nigra of the brain. PD develops as a result of the loss of dopamine producing brain cells. Typical early symptoms of PD include shaking or trembling of a limb, e.g. hands, arms, legs, feet and face. Additional characteristic symptoms are stiffness of the limbs and torso, slow movement or an inability to move, impaired balance and coordination, cognitional changes, and psychiatric conditions e.g. depression and visual hallucinations. PD has both familial and idiopathic forms and it is suggestion to be involved with genetic and environmental causes. PD affects more than 4 million people worldwide. In the US, approximately 60,000 cases are identified annually. Generally PD begins at the age of 50 or older. An early-onset form of the condition begins at age younger than 50, and juvenile-onset PD begins before age of 20.

Death of dopamine producing brain cells related to PD has been associated with aggregation, deposition and dysfunction of alpha-synuclein protein (see, e.g. Marques and Outeiro, 2012, Cell Death Dis. 3:e350, Jenner, 1989, J Neurol Neurosurg Psychiatry. Special Supplement, 22-28, and references therein). Studies have suggested that alpha-synuclein has a role in presynaptic signaling, membrane trafficking and regulation of dopamine release and transport. Alpha-synuclein aggregates, e.g. in forms of oligomers, have been suggested to be species responsible for neuronal dysfunction and death. Mutations of the alpha-synuclein gene (SNCA) have been identified in the familial forms of PD, but also environmental factors, e.g. neurotoxin affect alpha-synuclein aggregation. Other suggested causes of brain cell death in PD are dysfunction of proteasomal and lysosomal systems, reduced mitochondrial activity.

PD is related to other diseases related to alpha-synuclein aggregation, referred to as “synucleinopathies.” Such diseases include, but are not limited to, Parkinson's Disease Dementia (PDD), multiple system atrophy (MSA), dementia with Lewy bodies, juvenile-onset generalized neuroaxonal dystrophy (Hallervorden-Spatz disease), pure autonomic failure (PAF), neurodegeneration with brain iron accumulation type-1 (NBIA-1) and combined Alzheimer's and Parkinson's disease.

As of today, no cure or prevention therapy for PD has been identified. A variety of drug therapies available provide relief to the symptoms. Non-limiting examples of symptomatic medical treatments include carbidopa and levodoba combination reducing stiffness and slow movement, and anticholinergics to reduce trembling and stiffness. Other optional therapies include e.g. deep brain stimulation and surgery. There remains a need for therapy affecting the underlying pathophysiology. For example, antibodies targeting alpha-synuclein protein, or other proteins relevant for brain cell death in PD, may be used to prevent and/or treat PD.

In some embodiment, methods of the present invention may be used to treat subjects suffering from PD (e.g., PD associated with a mutation in a GBA gene) and other synucleinopathies. In some cases, methods of the present invention may be used to treat subjects suspected of developing PD (e.g., a PD associated with a mutation in a GBA gene) and other synucleinopathies.

AAV Particles and methods of using the AAV particles described herein may be used to prevent, manage and/or treat PD, e.g., a PD associated with a mutation in a GBA gene.

Approximately 5% of PD patients carry a GBA mutation: 10% of patients with type 1 GD develop PD before the age of 80 years, compared to about 3-4% in the normal population. Additionally, heterozygous or homozygous GBA mutations have been shown to increase the risk of PD 20-30 fold.

Dementia with Lewy Bodies

Dementia with Lewy Bodies (DLB), also known as diffuse Lewy body disease, is a form of progressive dementia, characterized by cognitive decline, fluctuating alertness and attention, visual hallucinations and parkinsonian motor symptoms. DLB may be inherited by an autosomal dominant pattern. DLB affects more than 1 million individuals in the US. The condition typically shows symptoms at the age of 50 or older.

DLB is caused by the abnormal build-up of Lewy bodies, aggregates of the alpha-synuclein protein, in the cytoplasm of neurons in the brain areas controlling memory and motor control. The pathophysiology of these aggregates is very similar to aggregates observed in Parkinson's disease and DLB also has similarities to Alzheimer's disease. Inherited DLB has been associated with gene mutations in GBAs.

As of today, there is no cure or prevention therapy for DLB. A variety of drug therapies available are aimed at managing the cognitive, psychiatric and motor control symptoms of the condition. Non-limiting examples of symptomatic medical treatments include e.g. acetylcholinesterase inhibitors to reduce cognitive symptoms, and levodopa to reduce stiffness and loss of movement. There remains a need for therapy affecting the underlying pathophysiology.

In some embodiments, methods of the present disclosure may be used to treat subjects suffering from DLB (e.g., a DLB associated with a mutation in a GBA gene). In some cases, the methods may be used to treat subjects suspected of developing DLB (e.g., a DLB associated with a mutation in a GBA gene).

AAV Particles and methods of using the AAV particles described in the present invention may be used to prevent, manage and/or treat DLB (e.g., a DLB associated with a mutation in a GBA gene).

### VI. Dosing and Administration

### Administration

In some aspects, the present disclosure provides administration and/or delivery methods for vectors and viral particles, e.g., AAV particles, encoding GCase protein or a variant thereof, for the prevention, treatment, or amelioration of diseases or disorders of the CNS. For example, administration of the AAV particles prevents, treats, or ameliorates GBA-related disorders. Thus, robust widespread GCase protein distribution throughout the CNS and periphery is desired for maximal efficacy. Particular target tissues for administration or delivery include CNS tissues, brain tissue, and, more specifically, caudate-putamen, thalamus, superior colliculus, cortex, and corpus collosum. Particular embodiments provide administration and/or delivery of the AAV particles and AAV vector genomes described herein to caudate-putamen and/or substantia nigra. Other particular embodiments provide administration and/or delivery of the AAV particles and AAV vector genomes described herein to thalamus.

The AAV particles of the present disclosure may be administered by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), intracranial (into the skull), picutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intraparenchymal (into the substance of), intrathecal (into the spinal canal), intraperitoneal, (infusion or injection into the peritoneum), intravesicular infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, subpial, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In some embodiments, AAV particles of the present disclosure are administered so as to be delivered to a target cell or tissue. Delivery to a target cell results in GCase protein expression. A target cell may be any cell in which it is considered desirable to increase GCase protein expression levels. A target cell may be a CNS cell. Non-limiting examples of such cells and/or tissues include, dorsal root ganglia and dorsal columns, proprioceptive sensory neurons, Clark's column, gracile and cuneate nuclei, cerebellar dentate nucleus, corticospinal tracts and the cells comprising the same, Betz cells, and cells of the heart.

In some embodiments, compositions may be administered in a way that allows them to cross the blood-brain barrier, vascular barrier, or other epithelial barrier.

In some embodiments, delivery of GCase protein by adeno-associated virus (AAV) particles to cells of the central nervous system (e.g., parenchyma) comprises infusion into cerebrospinal fluid (CSF). CSF is produced by specialized ependymal cells that comprise the choroid plexus located in the ventricles of the brain. CSF produced within the brain then circulates and surrounds the central nervous system including the brain and spinal cord. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced approximately four to six times per day or approximately once every four hours, though values for individuals may vary.

In some embodiments, the AAV particles may be delivered by systemic delivery. In some embodiments, the systemic delivery may be by intravascular administration. In some embodiments, the systemic delivery may be by intravenous (IV) administration.

In some embodiments, the AAV particles may be delivered by intravenous delivery.

In some embodiments, the AAV particle is administered to the subject via focused ultrasound (FUS), e.g., coupled with the intravenous administration of microbubbles (FUS-MB), or MRI-guided FUS coupled with intravenous administration, e.g., as described in Terstappen et al. (Nat Rev Drug Discovery, https://doi.org/10.1038/s41573-021-00139-y (2021)), Burgess et al. (Expert Rev Neurother. 15(5): 477-491 (2015)), and/or Hsu et al. (PLOS One 8(2): 1-8), the contents of which are incorporated herein by reference in its entirety.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by thalamic delivery.

In some embodiments, the AAV particles may be delivered by intracerebral delivery.

In some embodiments, the AAV particles may be delivered by intracardiac delivery.

In some embodiments, the AAV particles may be delivered by intracranial delivery.

In some embodiments, the AAV particles may be delivered by intra cisterna magna (ICM) delivery.

In some embodiments, the AAV particles may be delivered by direct (intraparenchymal) injection into an organ (e.g., CNS (brain or spinal cord)). In some embodiments, the intraparenchymal delivery may be to any region of the brain or CNS.

In some embodiments, the AAV particles may be delivered by intrastriatal injection.

In some embodiments, the AAV particles may be delivered into the putamen.

In some embodiments, the AAV particles may be delivered into the spinal cord.

In some embodiments, the AAV particles of the present disclosure may be administered to the ventricles of the brain.

In some embodiments, the AAV particles of the present disclosure may be administered to the ventricles of the brain by intracerebroventricular delivery.

In some embodiments, the AAV particles of the present disclosure may be administered by intramuscular delivery.

In some embodiments, the AAV particles of the present disclosure are administered by more than one route described above. As a non-limiting example, the AAV particles may be administered by intravenous delivery and thalamic delivery.

In some embodiments, the AAV particles of the present disclosure are administered by more than one route described above. As a non-limiting example, the AAV particles may be administered by intravenous delivery and intracerebral delivery.

In some embodiments, the AAV particles of the present disclosure are administered by more than one route described above. As a non-limiting example, the AAV particles may be administered by intravenous delivery and intracranial delivery.

In some embodiments, the AAV particles of the present disclosure are administered by more than one route described above. In some embodiments, the AAV particles of the present disclosure may be delivered by intrathecal and intracerebroventricular administration.

In some embodiments, the AAV particles may be delivered to a subject to improve and/or correct mitochondrial dysfunction.

In some embodiments, the AAV particles may be delivered to a subject to preserve neurons. The neurons may be primary and/or secondary sensory neurons. In some embodiments, AAV particles are delivered to dorsal root ganglia and/or neurons thereof.

In some embodiments, administration of the AAV particles may preserve and/or correct function in the sensory pathways.

In some embodiments, the AAV particles may be delivered via intravenous (IV), intracerebroventricular (ICV), intraparenchymal, and/or intrathecal (IT) infusion and the therapeutic agent may also be delivered to a subject via intramuscular (IM) limb infusion in order to deliver the therapeutic agent to the skeletal muscle. Delivery of AAVs by intravascular limb infusion is described by Gruntman and Flotte, Human Gene Therapy Clinical Development, 2015, 26(3), 159-164, the contents of which are herein incorporated by reference in their entirety.

In some embodiments, delivery of viral vector pharmaceutical compositions in accordance with the present disclosure to cells of the central nervous system (e.g., parenchyma) comprises a rate of delivery defined by VG/hour=mL/hour*VG/mL, wherein VG is viral genomes, VG/mL is composition concentration, and mL/hour is rate of infusion.

In some embodiments, delivery of AAV particle pharmaceutical compositions in accordance with the present disclosure to cells of the central nervous system (e.g., parenchyma) comprises infusion of up to 1 mL. In some embodiments, delivery of viral vector pharmaceutical compositions in accordance with the present disclosure to cells of the central nervous system (e.g., parenchyma) may comprise infusion of 0.0001, 0.0002, 0.001, 0.002, 0.003, 0.004, 0.005, 0.008, 0.010, 0.015, 0.020, 0.025, 0.030, 0.040, 0.050, 0.060, 0.070, 0.080, 0.090, 0.1, 0.2, 0.3, 0.4, 0.5, 0.6, 0.7, 0.8, or 0.9 mL.

In some embodiments, delivery of AAV particle pharmaceutical compositions in accordance with the present disclosure to cells of the central nervous system (e.g., parenchyma) comprises infusion of between about 1 mL to about 120 mL. In some embodiments, delivery of viral vector pharmaceutical compositions in accordance with the present disclosure to cells of the central nervous system (e.g., parenchyma) may comprise an infusion of 0.1, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In some embodiments delivery of AAV particles to cells of the central nervous system (e.g., parenchyma) comprises infusion of at least 3 mL. In some embodiments, delivery of AAV particles to cells of the central nervous system (e.g., parenchyma) consists of infusion of 3 mL. In some embodiments, delivery of AAV particles to cells of the central nervous system (e.g., parenchyma) comprises infusion of at least 10 mL. In some embodiments, delivery of AAV particles to cells of the central nervous system (e.g., parenchyma) consists of infusion of 10 mL.

In some embodiments, the volume of the AAV particle pharmaceutical composition delivered to the cells of the central nervous system (e.g., parenchyma) of a subject is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 ill, 1200 μl, 1300 μl, 1400 μl, 1500 μl, 1600 μl, 1700 μl, 1800 μl, 1900 μl, 2000 μl, or more than 2000 μl.

In some embodiments, the volume of the AAV particle pharmaceutical composition delivered to a region in both hemispheres of a subject brain is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 In some embodiments, the volume delivered to a region in both hemispheres is 200 As another non-limiting example, the volume delivered to a region in both hemispheres is 900 As yet another non-limiting example, the volume delivered to a region in both hemispheres is 1800 μl.

In certain embodiments, AAV particle or viral vector pharmaceutical compositions in accordance with the present disclosure may be administered at about 10 to about 600 μl/site, about 50 to about 500 μl/site, about 100 to about 400 μl/site, about 120 to about 300 μl/site, about 140 to about 200 μl/site, or about 160 μl/site.

In some embodiments, the total volume delivered to a subject may be split between one or more administration sites e.g., 1, 2, 3, 4, 5, or more than 5 sites. In some embodiments, the total volume is split between administration to the left and right hemisphere.

### Delivery of AAV Particles

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particles of the present disclosure may be administered or delivered using the methods for the delivery of AAV virions described in European Patent Application No. EP1857552, the contents of which are herein incorporated by reference in their entirety.

In some embodiments, the AAV particle or pharmaceutical compositions of the present disclosure may be administered or delivered using the methods for delivering proteins using AAV vectors described in European Patent Application No. EP2678433, the contents of which are herein incorporated by reference in their entirety.

In some embodiments, the viral vector encoding GCase protein may be administered or delivered using the methods for delivering DNA molecules using AAV vectors described in U.S. Pat. No. 5,858,351, the contents of which are herein incorporated by reference in their entirety.

In some embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the viral vector encoding GCase protein may be administered or delivered using the methods for delivering AAV virions described in U.S. Pat. No. 6,325,998, the contents of which are herein incorporated by reference in their entirety.

In some embodiments, the viral vector encoding GCase protein may be administered or delivered using the methods for delivering DNA to muscle cells described in U.S. Pat. No. 6,335,011, the contents of which are herein incorporated by reference in their entirety.

In some embodiments, the viral vector encoding GCase protein may be administered or delivered using the methods for delivering DNA to muscle cells and tissues described in U.S. Pat. No. 6,610,290, the contents of which are herein incorporated by reference in their entirety.

In some embodiments, the viral vector encoding GCase protein may be administered or delivered using the methods for delivering DNA to muscle cells described in U.S. Pat. No. 7,704,492, the contents of which are herein incorporated by reference in their entirety.

In some embodiments, the viral vector encoding GCase protein may be administered or delivered using the methods for delivering a payload to skeletal muscles described in U.S. Pat. No. 7,112,321, the contents of which are herein incorporated by reference in their entirety.

In some embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In some embodiments, the AAV particle or pharmaceutical compositions of the present disclosure may be administered or delivered using the methods for delivering a payload for the treatment of Alzheimer disease described in U.S. Pat. No. 8,318,687, the contents of which are herein incorporated by reference in their entirety.

In some embodiments, the AAV particle or pharmaceutical compositions of the present disclosure may be administered or delivered using the methods for delivering a payload described in International Patent Publication No. WO2012144446, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

In some embodiments, the AAV particle or pharmaceutical compositions of the present disclosure may be administered or delivered using the methods for delivering a payload described in International Patent Publication No. WO2001096587, the contents of which are herein incorporated by reference in their entirety.

In some embodiments, the AAV particle or pharmaceutical compositions of the present disclosure may be administered or delivered using the methods for delivering a payload to muscle tissue described in International Patent Publication No. WO2002014487, the contents of which are herein incorporated by reference in their entirety.

In some embodiments, a catheter may be used to administer the AAV particles. In certain embodiments, the catheter or cannula may be located at more than one site in the spine for multi-site delivery. The viral particles encoding may be delivered in a continuous and/or bolus infusion. Each site of delivery may be a different dosing regimen or the same dosing regimen may be used for each site of delivery. In some embodiments, the sites of delivery may be in the cervical and the lumbar region. In some embodiments, the sites of delivery may be in the cervical region. In some embodiments, the sites of delivery may be in the lumbar region.

In some embodiments, a subject may be analyzed for spinal anatomy and pathology prior to delivery of the AAV particles described herein. As a non-limiting example, a subject with scoliosis may have a different dosing regimen and/or catheter location compared to a subject without scoliosis.

In some embodiments, the delivery method and duration is chosen to provide broad transduction in the spinal cord. In some embodiments, intrathecal delivery is used to provide broad transduction along the rostral-caudal length of the spinal cord. In some embodiments, multi-site infusions provide a more uniform transduction along the rostral-caudal length of the spinal cord.

### Delivery to Cells

In some aspects, the present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

In some aspects, the present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

In some embodiments, the AAV particles may be delivered to bypass anatomical blockages such as, but not limited to the blood brain barrier.

In some embodiments, the AAV particles may be formulated and delivered to a subject by a route which increases the speed of drug effect as compared to oral delivery.

In some embodiments, the AAV particles may be delivered by a method to provide uniform transduction of the spinal cord and dorsal root ganglion (DRG). In some embodiments, the AAV particles may be delivered using intrathecal infusion.

In some embodiments, a subject may be administered the AAV particles described herein using a bolus infusion. As used herein, a “bolus infusion” means a single and rapid infusion of a substance or composition.

In some embodiments, the AAV particles encoding GCase protein may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, the AAV particles may be delivered to a subject via a single route administration.

In some embodiments, the AAV particles may be delivered to a subject via a multi-site route of administration. For example, a subject may be administered the AAV particles at 2, 3, 4, 5, or more than 5 sites.

In some embodiments, a subject may be administered the AAV particles described herein using sustained delivery over a period of minutes, hours or days. The infusion rate may be changed depending on the subject, distribution, formulation or another delivery parameter known to those in the art.

In some embodiments, if continuous delivery (continuous infusion) of the AAV particles is used, the continuous infusion may be for 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more than 24 hours.

In some embodiments, the intracranial pressure may be evaluated prior to administration. The route, volume, AAV particle concentration, infusion duration and/or vector titer may be optimized based on the intracranial pressure of a subject.

In some embodiments, the AAV particles may be delivered by systemic delivery. In some embodiments, the systemic delivery may be by intravascular administration.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by direct (intraparenchymal) injection into the substance of an organ, e.g., one or more regions of the brain.

In some embodiments, the AAV particles may be delivered by subpial injection into the spinal cord. For example, subjects may be placed into a spinal immobilization apparatus. A dorsal laminectomy may be performed to expose the spinal cord. Guiding tubes and XYZ manipulators may be used to assist catheter placement. Subpial catheters may be placed into the subpial space by advancing the catheter from the guiding tube and AAV particles may be injected through the catheter (Miyanohara et al., Mol Ther Methods Clin Dev. 2016; 3: 16046). In some cases, the AAV particles may be injected into the cervical subpial space. In some cases, the AAV particles may be injected into the thoracic subpial space.

In some embodiments, the AAV particles may be delivered by direct injection to the CNS of a subject. In some embodiments, direct injection is intracerebral injection, intraparenchymal injection, intrathecal injection, intra-cisterna magna injection, or any combination thereof. In some embodiments, direct injection to the CNS of a subject comprises convection enhanced delivery (CED). In some embodiments, administration comprises peripheral injection. In some embodiments, peripheral injection is intravenous injection.

In some embodiments, the AAV particles may be delivered to a subject in order to increase the GCase protein levels in the caudate-putamen, thalamus, superior colliculus, cortex, and/or corpus callosum as compared to endogenous levels. The increase may be 0.1× to 5×, 0.5× to 5×, 1× to 5×, 2× to 5×, 3× to 5×, 4× to 5×, 0.1× to 4×, 0.5× to 4×, 1× to 4×, 2× to 4×, 3× to 4×, 0.1× to 3×, 0.5× to 3×, 1× to 3×, 2× to 3×, 0.1× to 2×, 0.5× to 2×, 0.1× to 1×, 0.5× to 1×, 0.1× to 0.5×, 1× to 2×, 0.1×, 0.2×, 0.3×, 0.4×, 0.5×, 0.6×, 0.7×, 0.8×, 0.9×, 1.0×, 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9×, 4.0×, 4.1×, 4.2×, 4.3×, 4.4×, 4.5×, 4.6×, 4.7×, 4.8×, 4.9× or more than 5× as compared to endogenous levels.

In some embodiments, the AAV particles may be delivered to a subject in order to increase the GCase protein levels in the caudate, putamen, thalamus, superior colliculus, cortex, and/or corpus callosum by transducing cells in these CNS regions. Transduction may also be referred to as the amount of cells that are positive for GCase protein. The transduction may be greater than or equal to 1%, 5%, 10%, 15%, 20%, 25%, 30%, 35%, 40%, 45%, 50%, 55%, 60%, 65%, 70%, 75%, 80%, 85%, 90%, 95%, or 99% of cells in these CNS regions.

In some embodiments, delivery of AAV particles comprising a viral genome encoding GCase protein described herein to neurons in the caudate-putamen, thalamus, superior colliculus, cortex, and/or corpus callosum will lead to an increased expression of GCase protein. The increased expression may lead to improved survival and function of various cell types in these CNS regions and subsequent improvement of GBA-related disorder symptoms.

In particular embodiments, the AAV particles may be delivered to a subject in order to establish widespread distribution of the GCase throughout the nervous system by administering the AAV particles to the thalamus of the subject.

Specifically, in some embodiments, the increased expression of GCase protein may lead to improved gait, sensory capability, coordination of movement and strength, functional capacity, cognition, and/or quality of life.

### Dosing

In some aspects, the present disclosure provides methods comprising administering viral vectors and their payloads in accordance with the disclosure to a subject in need thereof. Viral vector pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition associated with decreased GCase protein expression or a deficiency in the quantity and/or function of GCase protein). In some embodiments, the disease, disorder, and/or condition is GBA-related disorders. The exact amount required will vary from subject to subject, depending on the species, age, and general condition of the subject, the severity of the disease, the particular composition, its mode of administration, its mode of activity, and the like. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific peptide(s) employed; the duration of the treatment; drugs used in combination or coincidental with the specific compound employed; and like factors well known in the medical arts.

In certain embodiments, AAV particle pharmaceutical compositions in accordance with the present disclosure may be administered at dosage levels sufficient to deliver GCase protein from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to VG or viral genomes per kg or into total viral genomes administered by one of skill in the art.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic composition administered in one dose/at one time/single route/single point of contact, i.e., single administration event. In some embodiments, a single unit dose is provided as a discrete dosage form (e.g., a tablet, capsule, patch, loaded syringe, vial, etc.). As used herein, a “total daily dose” is an amount given or prescribed in 24-hour period. It may be administered as a single unit dose. The viral particles may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

In some embodiments, delivery of the AAV particles described herein results in minimal serious adverse events (SAEs) as a result of the delivery of the AAV particles.

In some embodiments, delivery of AAV particle pharmaceutical compositions in accordance with the present disclosure to cells of the central nervous system (e.g., parenchyma) may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 1.8×10¹¹, 2×10¹¹, 3×10¹¹, 4×10¹¹, 5×10¹¹, 5.5×10¹¹, 6×10¹¹, 7×10¹¹, 8×10¹¹, 9×10¹¹, 0.8×10¹², 0.83×10¹², 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.3×10¹³, 3×10¹³, 4×10¹³, 5×10¹³, 6×10¹³, 7×10¹³, 8×10¹³, 9×10¹³, 1×10¹⁴, 1.9×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In some embodiments, the concentration of the viral vector in the composition is 1×10¹³ VG/mL. In some embodiments, the concentration of the viral vector in the composition is 1.1×10¹² VG/mL. In some embodiments, the concentration of the viral vector in the composition is 3.7×10¹² VG/mL. In some embodiments, the concentration of the viral vector in the composition is 8×10¹¹ VG/mL. In some embodiments, the concentration of the viral vector in the composition is 2.6×10¹² VG/mL. In some embodiments, the concentration of the viral vector in the composition is 4.9×10¹² VG/mL. In some embodiments, the concentration of the viral vector in the composition is 0.8×10¹² VG/mL. In some embodiments, the concentration of the viral vector in the composition is 0.83×10¹² VG/mL. In some embodiments, the concentration of the viral vector in the composition is the maximum final dose which can be contained in a vial. In some embodiments, the concentration of the viral vector in the composition is 1.6×10¹¹ VG/mL. In some embodiments, the concentration of the viral vector in the composition is 5×10¹¹ VG/mL. In some embodiments, the concentration of the viral vector in the composition is 2.3×10¹³ VG/mL. In some embodiments, the concentration of the viral vector in the composition is 1.9×10¹⁴ VG/mL.

In some embodiments, delivery of AAV particle pharmaceutical compositions in accordance with the present disclosure to cells of the central nervous system (e.g., parenchyma) may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 4.6×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.3×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject. In some embodiments, the concentration of the viral vector in the composition is 2.3×10¹¹ VG/subject. In some embodiments, the concentration of the viral vector in the composition is 7.2×10¹¹ VG/subject. In some embodiments, the concentration of the viral vector in the composition is 7.5×10¹¹ VG/subject. In some embodiments, the concentration of the viral vector in the composition is 1.4×10¹² VG/subject. In some embodiments, the concentration of the viral vector in the composition is 4.8×10¹² VG/subject. In some embodiments, the concentration of the viral vector in the composition is 8.8×10¹² VG/subject. In some embodiments, the concentration of the viral vector in the composition is 2.3×10¹² VG/subject. In some embodiments, the concentration of the viral vector in the composition is 2×10¹⁰ VG/subject. In some embodiments, the concentration of the viral vector in the composition is 1.6×10¹¹ VG/subject. In some embodiments, the concentration of the viral vector in the composition is 4.6×10¹¹ VG/subject.

In some embodiments, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. In some embodiments, the total dose is 1×10¹³ VG. In some embodiments, the total dose is 3×10¹³ VG. In some embodiments, the total dose is 3.73×10¹⁰ VG. In some embodiments, the total dose is 1.9×10¹⁰ VG. In some embodiments, the total dose is 2.5×10¹¹ VG. In some embodiments, the total dose is 5×10¹¹ VG. In some embodiments, the total dose is 1×10¹² VG. In some embodiments, the total dose is 5×10¹² VG.

### Combinations

The AAV particles may be used in combination with one or more other therapeutic, prophylactic, diagnostic, or imaging agents. The phrase “in combination with,” is not intended to require that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

The therapeutic agents may be approved by the US Food and Drug Administration or may be in clinical trial or at the preclinical research stage. The therapeutic agents may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Measurement of Expression

Expression of GCase protein from viral genomes may be determined using various methods known in the art such as, but not limited to immunochemistry (e.g., IHC), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, PCR, and/or in situ hybridization (ISH). In some embodiments, transgenes encoding GCase protein delivered in different AAV capsids may have different expression levels in different CNS tissues.

In certain embodiments, the GCase protein is detectable by Western blot.

Alternatively methods of detecting GBA expression are known, including, for example, use of the methods and compounds as described in Int'l Pub. No. WO2019136484, incorporated herein by reference in its entirety.

### VII. Kits and Devices

### Kits

In some aspects, the present disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the vectors, constructs, or GCase proteins of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, compounds and/or compositions of the present disclosure may be combined with, coated onto or embedded in a device. Devices may include, but are not limited to, dental implants, stents, bone replacements, artificial joints, valves, pacemakers and/or other implantable therapeutic device.

The present disclosure provides for devices which may incorporate viral vectors that encode one or more GCase protein molecules. These devices contain in a stable formulation the viral vectors which may be immediately delivered to a subject in need thereof, such as a human patient.

Devices for administration may be employed to deliver the viral vectors encoding GCase protein of the present disclosure according to single, multi- or split-dosing regimens taught herein.

Method and devices known in the art for multi-administration to cells, organs and tissues are contemplated for use in conjunction with the methods and compositions disclosed herein as embodiments of the present disclosure.

### VIII. Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV capsid polypeptide, e.g., a parent capsid sequence with at least one peptide, e.g., targeting peptide, insert. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. In some embodiments, the AAV particle may comprises a peptide, e.g., targeting peptide, present, e.g., inserted into, the capsid to enhance tropism for a desired target tissue. It is to be understood that reference to the AAV particle of the disclosure also includes pharmaceutical compositions thereof, even if not explicitly recited.

Active Ingredient: As used herein, the term “active ingredient” refers to a molecule or complex thereof that is biologically active and responsible for a generating a biological effect. The active ingredient in a pharmaceutical composition may be referred to as an active pharmaceutical ingredient. For the purposes of the present disclosure, the phrase “active ingredient” generally refers either to the viral particle carrying the payload or to the payload (or its gene product) delivered by the viral particle as described herein. In contrast, an “inactive ingredient” refers to a substance which is biologically inert. An excipient is an example of an inactive ingredient.

Administered in combination: As used herein, the term “administered in combination” or “delivered in combination” or “combined administration” refers to exposure of two or more agents (e.g., AAV) administered at the same time or within an interval such that the subject is at some point in time exposed to both agents and/or such that there is an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatuses in administration. In some embodiments, the administration of individual doses of one or more compounds and/or compositions of the present disclosure, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of a neurodegenerative disorder, amelioration includes the reduction or stabilization of neuron loss.

Animal: As used herein, the term “animal” refers to any member of the animal kingdom. In some embodiments, the terms subject or animal refers to humans at any stage of development. In some embodiments, animal refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.25%, 0.1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any substance (e.g., an AAV) that has activity in or on a biological system and/or organism. For instance, a substance that, when administered to an organism, has a biological effect on that organism, is considered to be biologically active. In particular embodiments, a compound, and/or a composition of the present disclosure may be considered biologically active if even a portion of it is biologically active or mimics an activity considered to be biologically relevant. In some embodiments, biological activity refers to inducing expression of GCase protein or a variant thereof. In some embodiments, biological activity refers to preventing and/or treating a disease associated with decreased GCase protein expression or a deficiency in the quantity and/or function of GCase protein. In some embodiments, biological activity refers to preventing and/or treating disorders associated with reduced GBA gene expression, including, for example, Parkinson Disease (PD) and related disorders, including Gaucher Disease, and Dementia with Lewy Bodies (collectively, “GBA-related disorders”).

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Central Nervous System or CNS: As used herein, “central nervous system” or “CNS” refers to one of the two major subdivisions of the nervous system, which in vertebrates includes the brain and spinal cord. The central nervous system coordinates the activity of the entire nervous system.

Cervical Region: As used herein, “cervical region” refers to the region of the spinal cord comprising the cervical vertebrae C1, C2, C3, C4, C5, C6, C7, and C8.

Cis-Elements: As used herein, cis-elements or the synonymous term “cis-regulatory elements” refer to regions of non-coding DNA which regulate the transcription of nearby genes. The Latin prefix “cis” translates to “on this side.” Cis-elements are found in the vicinity of the gene, or genes, they regulate. Examples of cis-elements include a Kozak sequence, SV40 introns, or a portion of the backbone.

CNS tissue: As used herein, “CNS tissue” or “CNS tissues” refers to the tissues of the central nervous system, which in vertebrates, include the brain and spinal cord and sub-structures thereof.

CNS structures: As used herein, “CNS structures” refers to structures of the central nervous system and sub-structures thereof. Non-limiting examples of structures in the spinal cord may include, ventral horn, dorsal horn, white matter, and nervous system pathways or nuclei within. Non-limiting examples of structures in the brain include, forebrain, midbrain, hindbrain, diencephalon, telencephalon, myelencephalon, metencephalon, mesencephalon, prosencephalon, rhombencephalon, cortices, frontal lobe, parietal lobe, temporal lobe, occipital lobe, cerebrum, thalamus, hypothalamus, tectum, tegmentum, cerebellum, pons, medulla, amygdala, hippocampus, basal ganglia, corpus callosum, pituitary gland, putamen, striatum, ventricles and sub-structures thereof.

CNS Cells: As used herein, “CNS cells” refers to cells of the central nervous system and sub-structures thereof. Non-limiting examples of CNS cells include, neurons and sub-types thereof, glia, microglia, oligodendrocytes, ependymal cells and astrocytes. Non-limiting examples of neurons include sensory neurons, motor neurons, interneurons, unipolar cells, bipolar cells, multipolar cells, pseudounipolar cells, pyramidal cells, basket cells, stellate cells, Purkinje cells, Betz cells, amacrine cells, granule cell, ovoid cell, medium aspiny neurons and large aspiny neurons.

Codon optimization: As used herein, the term “codon optimization” refers to a process of changing codons of a given gene in such a manner that the polypeptide sequence encoded by the gene remains the same while the changed codons improve the process of expression of the polypeptide sequence. For example, if the polypeptide is of a human protein sequence and expressed in E. coli, expression will often be improved if codon optimization is performed on the DNA sequence to change the human codons to codons that are more effective for expression in E. coli.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In some embodiments, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering a parvovirus e.g., AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present disclosure, e.g., viral particles or AAV vectors) to targeted cells.

Delivery route: As used herein, the term “delivery route” and the synonymous term “administration route” refers to any of the different methods for providing a therapeutic agent to a subject. Routes of administration are generally classified by the location at which the substance is applied and may also be classified based on where the target of action is. Examples include, but are not limited to: intravenous administration, subcutaneous administration, oral administration, parenteral administration, enteral administration, topical administration, sublingual administration, inhalation administration, and injection administration, or other routes of administration described herein.

Derivative: As used herein, the term “derivative” refers to a composition (e.g., sequence, compound, formulation, etc.) that is derived from, or finds its basis in, a parent composition. Non-limiting examples of a parent composition include a wild-type or original amino acid or nucleic acid sequence, or an undiluted formulation. In some embodiments, a derivative is a variant of a parent composition. A derivative may differ from the parent composition by less than about 1%, less than about 5%, less than about 10%, less than about 15%, less than about 20%, less than about 25%, less than about 30%, less than about 35%, less than about 40%, less than about 45%, or less than about 50%. In certain embodiments, a derivative may differ from a parent composition by more than about 50%. In certain embodiments, a derivative may differ from a parent composition by more than about 75%. In some embodiments, a derivative may be a fragment or truncation of a parent amino acid or nucleotide sequence. As a non-limiting example, a derivative may be a sequence with a nucleotide or peptide insert as compared to a parent nucleic acid or amino acid sequence (e.g., AAVPHP.B as compared to AAV9).

Effective amount: As used herein, the term “effective amount” of an agent is that amount sufficient to effect beneficial or desired results, for example, upon single or multiple dose administration to a subject or a cell, in curing, alleviating, relieving or improving one or more symptoms of a disorder and, as such, an “effective amount” depends upon the context in which it is being applied. For example, in the context of administering an agent that treats Parkinson Disease (PD) and related disorders, including Gaucher Disease, and Dementia with Lewy Bodies (collectively, “GBA-related disorders”), an effective amount of an agent is, for example, an amount sufficient to achieve treatment, as defined herein, of a GBA-related disorder as compared to the response obtained without administration of the agent.

Engineered: As used herein, embodiments of the disclosure are “engineered” when they are designed to have a feature or property, whether structural or chemical, that varies from a starting point, wild-type or native molecule. Thus, engineered agents or entities are those whose design and/or production include an act of the hand of man.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and/or (5) post-translational modification of a polypeptide or protein.

Excipient: As used herein, the term “excipient” refers to an inactive substance that serves as the vehicle or medium for an active pharmaceutical agent or other active substance.

Formulation: As used herein, a “formulation” includes at least a compound and/or composition of the present disclosure (e.g., a vector, AAV particle, etc.) and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. A fragment may also refer to a truncation (e.g., an N-terminal and/or C-terminal truncation) of a protein or a truncation (e.g., at the 5′ and/or 3′ end) of a nucleic acid. A protein fragment may be obtained by expression of a truncated nucleic acid, such that the nucleic acid encodes a portion of the full-length protein.

GBA-related disorder: The terms “GBA-related disorder,” “GBA-related disease,” “GBA patient,” and the like refer to diseases or disorders having a deficiency in the GBA gene, such as a heritable, e.g., autosomal recessive, mutation in GBA resulting in deficient or defective GCase protein expression in patient cells. GBA-related disorders expressly include, but are not limited to Parkinson disease (PD), Gaucher disease, and Dementia with Lewy Bodies; and may include additional Lewy body disorders, lysosomal storage disorders, and related disorders. GBA patients are individuals harboring one or more mutation in the GBA gene, including, e.g., biallelic mutations, making them more susceptible to GBA-related disorders.

GCase protein: As used herein, the terms “GCase”, “GCase protein,” “GCase proteins,” and the like refer to protein products or portions of protein products including peptides of the GBA gene (Ensemble gene ID: ENSG00000177628), homologs or variants thereof, and orthologs thereof, including non-human proteins and homologs thereof. GCase proteins include fragments, derivatives, and modifications of GBA gene products.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides, and peptides are well known in the art.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% identical for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50, or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Humanized: As used herein, the term “humanized” refers to a non-human sequence of a polynucleotide or a polypeptide which has been altered to increase its similarity to a corresponding human sequence.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molecular Biol., 215, 403 (1990)).

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Lumbar Region: As used herein, the term “lumbar region” refers to the region of the spinal cord comprising the lumbar vertebrae L1, L2, L3, L4, and L5.

miR binding site series: As used herein, the “miR binding site series” or the “miR binding site” includes an RNA sequence on the RNA transcript produced by transcribing the AAV vector genome. The “miR binding site series” or the “miR binding site” also includes the DNA sequence corresponding to the RNA sequence, in that they differ only by the T in DNA and the U in RNA. The reverse complement of such DNA is the coding sequence for the RNA sequence. That is, in some embodiments, in an expression cassette containing a DNA positive strand, the miR binding site sequence is the reverse complement of the miRNA to which it binds.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides. One or more mutations may result in a “mutant,” “derivative,” or “variant,” e.g., of a nucleic acid sequence or polypeptide or protein sequence.

Naturally occurring: As used herein, “naturally occurring” or “wild-type” means existing in nature without artificial aid, or involvement of the hand of man. “Naturally occurring” or “wild-type” may refer to a native form of a biomolecule, sequence, or entity.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid: As used herein, the terms “nucleic acid,” “polynucleotide,” and “oligonucleotide” refer to any nucleic acid polymers composed of either polydeoxyribonucleotides (containing 2-deoxy-D-ribose), or polyribonucleotides (containing D-ribose), or any other type of polynucleotide that is an N glycoside of a purine or pyrimidine base, or modified purine or pyrimidine bases. There is no intended distinction in length between the term “nucleic acid,” “polynucleotide,” and “oligonucleotide,” and these terms will be used interchangeably. These terms refer only to the primary structure of the molecule. Thus, these terms include double- and single-stranded DNA, as well as double- and single-stranded RNA.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells. The payload construct vector may also comprise a component for viral expression in a viral replication cell.

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and/or xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), the contents of each of which are incorporated herein by reference in their entirety.

Pharmaceutical Composition: As used herein, the term “pharmaceutical composition” or pharmaceutically acceptable composition” comprises AAV polynucleotides, AAV genomes, or AAV particle and one or more pharmaceutically acceptable excipients, solvents, adjuvants, and/or the like.

Polypeptide: As used herein, the term “polypeptide” refers to an organic polymer consisting of a large number of amino-acid residues bonded together in a chain. A monomeric protein molecule is a polypeptide.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Promoter: As used herein, the term “promoter” refers to a nucleic acid site to which a polymerase enzyme will bind to initiate transcription (DNA to RNA) or reverse transcription (RNA to DNA).

Protein of interest: As used herein, the terms “proteins of interest” or “desired proteins” include those provided herein and fragments, mutants, variants, or alterations thereof.

Purified: As used herein, the term “purify” means to make substantially pure or clear from one or more unwanted components, material defilement, admixture or imperfection. “Purified” refers to the state of being pure. “Purification” refers to the process of making pure. As used herein, a substance is “pure” if it is substantially free of (substantially isolated from) one or more components, e.g., one or more components found in a native context.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may comprise the N- and/or C-termini as well as surrounding amino acids. In some embodiments, N- and/or C-terminal regions comprise from about 3 amino acids to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecules. In some embodiments, a “primary” sample is an aliquot of the source. In some embodiments, a primary sample is subjected to one or more processing (e.g., separation, purification, etc.) steps to prepare a sample for analysis or other use.

Serotype: As used herein, the term “serotype” refers to distinct variations in a capsid of an AAV based on surface antigens which allow epidemiologic classifications of the AAVs at the sub-species level.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Spacer: As used herein, a “spacer” is generally any selected nucleic acid sequence of, e.g., 1, 2, 3, 4, 5, 6, 7, 8, 9, or 10 nucleotides in length, which is located between two or more consecutive miR binding site sequences.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the disclosure may be administered, e.g., for experimental, diagnostic, prophylactic, and/or therapeutic purposes. Similarly, “subject” or “patient” refers to an organism who may seek, who may require, who is receiving, or who will receive treatment or who is under care by a trained professional for a particular disease or condition. Typical subjects include animals (e.g., mammals such as mice, rats, rabbits, non-human primates, and humans). In certain embodiments, a subject or patient may be susceptible to or suspected of having a GBA-related disorder. In certain embodiments, a subject or patient may be diagnosed with PD, Gaucher Disease, or Dementia with Lewy Bodies disease.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “target cells” or “targeted cells” refers to any one or more cells of interest. The cells may be found in vitro, in vivo, in situ or in the tissue or organ of an organism. The organism may be an animal, a mammal, a human and/or a patient. The target cells may be CNS cells or cells in CNS tissue.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Thoracic Region: As used herein, a “thoracic region” refers to a region of the spinal cord comprising the thoracic vertebrae T1, T2, T3, T4, T5, T6, T7, T8, T9, T10, T11, and T12.

Treating: As used herein, the term “treating” refers to partially or completely alleviating, ameliorating, improving, relieving, reversing, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence(s). Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, having a sequence that may be wild-type or modified from wild-type and which sequence may encode full-length or partial sequence of a protein, protein domain, or one or more subunits of GCase protein and variants thereof; a polynucleotide encoding GCase protein and variants thereof, having a sequence that may be wild-type or modified from wild-type; and a transgene encoding GCase protein and variants thereof that may or may not be modified from wild-type sequence.

Viral construct vector. As used herein, a “viral construct vector” is a vector which comprises one or more polynucleotide regions encoding or comprising Rep and or Cap protein. A viral construct vector may also comprise one or more polynucleotide region encoding or comprising components for viral expression in a viral replication cell.

Viral genome: As used herein, a “viral genome” or “vector genome” is a polynucleotide comprising at least one inverted terminal repeat (ITR) and at least one encoded payload. A viral genome encodes at least one copy of the payload.

Wild-type: As used herein, “wild-type” is a native form of a biomolecule, sequence, or entity.

### Examples

The present disclosure is further illustrated by the following non-limiting examples. Experiments described in the Examples establish that enhanced AAV-based GCase gene therapy treatments are superior to and/or additive with wild-type GCase-based treatment in ameliorating GBA-related disorders.

### Cell Lines, Tissues, and Animal Models

In vitro experiments: Human fibroblasts from GBA patients (all 3 types) were obtained from Corielle. The following Gaucher patient fibroblasts were chosen based on significantly depleted GCase activity (4-6%) and availability of age- and race-matched healthy control fibroblasts: GM04394-fibroblast, GM00852-fibroblast, GM00877-fibroblast, GM05758-fibroblast from skin/inguinal area, and GM02937-fibroblast from skin/unspecified (all available from Corielle).

GBA-4L/PS-NA primary neurons can be generated from pregnant GBA-4L/PS-NA females from QPS. GBA-knockout (GBA-KO) neuroblastoma cell line (IMR-32 background, available from ATCC) was obtained from Synthego.

Animal models: GBA-4L/PS-NA mouse models (available at QPS): 4L/PS-NA mice express low level of prosaposin and saposin C, as well as GCase with a point mutation at position V394L/V394L. Strong enlargement of leukocytes and macrophages in visceral organs like spleen, thymus, lung and liver develop as early as 5 week of age. Most deficits and reduced muscle strength accompanied by neuroinflammation in the cortex, and hippocampus increase as animals age. There is significant increase in glucosylceramide and glucosylsphingosine. GBA-4L/PS-NA can survival up to 22 weeks. Homozygous Prnp-SNCA-A53T (M83) mice, by 8-months of age, develop α-syn aggregates and progressively severe motor phenotype.

### Example 1. Vector Design and Synthesis

An AAV viral genome expressing a payload region comprising a polynucleotide encoding a human GBA polypeptide is generated. The viral genome comprises polynucleotides encoding an AAV capsid of a serotype provided in Table 1. A promoter region regulates expression of the payload region. Widespread GBA distribution is achieved by use of a ubiquitous promoter, such as CBA, to achieve transduction within different CNS cell types.

Single-stranded codon optimized GBA cDNA sequence under ubiquitous CBA promoter packaged within AAV2 ITRs is generated (wtGBA). Enhanced GBA (enGBA) constructs (see Examples 2-5) are generated and compared against wtGBA. wtGBA and GFP reporter vectors are compared side-by-side to test multiplicity of infection for in vitro experiments. The final AAV transgene design nominations are made based on vectorized in vitro experiments and tested in the proposed in vivo models, including those used for GLP and tolerability studies.

PD-GBA patients demonstrate a global reduction in GCase levels in the CNS. Consequently, high GCase levels in CSF, caudate, substantia nigra, cortex and cerebellum is targeted. Although the disease pathology is largely neuronal, the therapeutic strategy is expected to benefit by transduction of other CNS cell-types, e.g. astrocytes, via cross-correction benefit.

In addition to PD-GBA, efficacy in secondary disease indications in patients with GBA mutations is tested, including Gaucher disease (including Neuronopathic Gaucher disease) and Dementia with Lewy bodies.

Transgenes designed as described above are tested for plasmid-level expression: all cassettes are engineered in single stranded AAV transgene configuration driven by ubiquitous CBA promoter flanked by AAV2 ITRs. The following transgene constructs are engineered and synthesized: 1) codon optimized GBA cDNA construct; 2) enhanced GBA construct comprising GBA cDNA and further encoding prosaposin/saposin C in the same transgene (optimal co-activator gene and linker sequences are selected for vectorization based on plasmid-level expression analysis); 3) enhanced GBA constructs comprising a cell-penetration peptide; and 4) enhanced GBA constructs comprising lysosomal targeting peptides (LTP); and 5) combinatorial enhanced GBA constructs comprising a combination of GBA cDNA, saposin sequence(s), lysosomal targeting sequence(s) and/or cell penetrating peptide sequence(s).

These constructs are tested for expression/GCase activity in cell culture with ITR plasmid transfections as a first pass. Specifically, plasmids are tested in CHO/HEK-293 cells at 48 hours post transfections. Both lysates and media are assessed for expression. Based on the results, GBA transgene ITR cassettes (wt, enGBA and enGBAcombo constructs) are selected for vectorization and evaluation within in vitro disease model setting.

Upon plasmid-level expression/GCase activity confirmation, select AAV ITR cassettes (wtGBA, enGBA and enGBAcombo) are packaged into HEK 293 small-scale AAV6 or AAV2 preps for initial in vitro evaluations.

### Example 2. Co-Administration of SapC Enhances GBA Gene Therapy

Viral genomes encoding a GBA protein can also be designed to further encode an enhancement element, e.g., a prosaposin protein, a Saposin C protein, or functional variant thereof. GCase coactivator Saposin C (SapC) is one of the cleavage products of saposin precursor protein Prosaposin. Saposin C is the essential activator of GCase lysosomal enzyme. In mouse models of PD-GBA and Gaucher disease, the combination of loss of function in GBA and Saposin C results in significantly exacerbated disease phenotype. Thus, AAV mediated co-delivery of GBA (e.g., a viral genome encoding a GBA protein, e.g., comprising the nucleotide sequence of SEQ ID NO: 1772, 1773, 1776, 1777, 1780, or 1781, or a functional variant thereof) and cDNA encoding a prosaposin protein (e.g., a prosaposin protein comprising the amino acid sequence of SEQ ID NO: 1750 or 1758, or a functional variant thereof; or encoded by a nucleotide sequence comprising SEQ ID NO: 1858 or 1859, or a functional variant thereof) or a Saposin C (SapC) protein or functional variant thereof (e.g., a SapC protein or functional variant thereof comprising the amino acid sequence of SEQ ID NO: 1788, 1789, 1791, or 1792; or encoded by the nucleotide sequence of SEQ ID NO: 1786, 1787, 1790, or 1791) is tested to increase potency of GBA gene therapy by enhancing catalytic activity of GCase enzyme.

### Example 3. Cell-Penetration Peptides Enhance Cellular Penetration/Uptake

Viral genomes encoding a GBA protein can also be designed to further encode an enhancement element, e.g., a cell penetrating peptide or functional variant thereof. Without wishing to be bound by theory, it is believed that a cell-penetration peptide (CPP) signal added to the GCase sequence of the transgenes of the disclosure results in increased cellular uptake of secreted GCase product in circulation, in cerebrospinal fluid, and in interstitial fluid from AAV-transduced cells; and this enhanced cell penetration thus increases cross-correction potential of the secreted GCase enzyme. Exemplary CPPs used herein include: HIV-derived TAT peptide (e.g., comprising the amino acid sequence of 1794 and/or encoded by the nucleotide sequence of SEQ ID NO: 1794), human apoliprotein B receptor binding domain (e.g., comprising the amino acid sequence of 1796 and/or encoded by the nucleotide sequence of SEQ ID NO: 1795), and/or human apolipoprotein E-II receptor binding domain (e.g., comprising the amino acid sequence of 1798 and/or encoded by the nucleotide sequence of SEQ ID NO: 1797).

### Example 4. CMA Recognition Sequences Enhance Intracellular Lysosomal Targeting

Viral genomes encoding a GBA protein can also be designed to further encode an enhancement element, e.g., a lysosomal targeting sequence or functional variant thereof.

Chaperone sequences including glycosylation-independent lysosomal targeting peptides (which is an M-6-P independent lysosomal targeting mechanism) have demonstrated ability to enable enhanced delivery of lysosomal enzyme product. GBA utilizes LIMP-2 (encoded by SCARB2 gene) as the lysosomal surface receptor (important for lysosomal localization). Co-delivery of SCARB2 with GBA provides an alternative strategy to enhance lysosomal targeting of GCase. Chaperone-mediated autophagy signals are incorporated into transgenes of the disclosure to increase lysosomal targeting of the GCase enzyme. Highly conserved recognition sequences of chaperone-mediated-autophagy (CMA) pathway is analyzed for improved lysosomal targeting of GCase enzyme. Such sequences include, for example, RNase A-derived CMA recognition sequence, HSC70-derived CMA recognition sequence, or hemoglobin-derived CMA recognition sequence.

Lysosomal targeting sequences (LTS) are also included in viral genomes encoding a GBA protein described herein. Exemplary LTS peptides used herein include LTS1 (e.g., comprising the amino acid sequence of SEQ ID NO 1800 and/or encoded by the nucleotide sequence of SEQ ID NO: 1799), LTS2 (e.g., comprising the amino acid sequence of SEQ ID NO 1802 and/or encoded by the nucleotide sequence of SEQ ID NO: 1801), LTS3 (e.g., comprising the amino acid sequence of SEQ ID NO 1804 and/or encoded by the nucleotide sequence of SEQ ID NO: 1803), LTS4 (e.g., comprising the amino acid sequence of SEQ ID NO 1806 and/or encoded by the nucleotide sequence of SEQ ID NO: 1805), and/or LTS5 (e.g., comprising the amino acid sequence of SEQ ID NO 1808 and/or encoded by the nucleotide sequence of SEQ ID NO: 1807).

### Example 5. Combinatorial Enhancements

Combinations of the aforementioned enhancement elements (Examples 2-4) are tested for ability of different combinations to additively or synergistically increase potency of AAV mediated delivery of GCase enzyme in vivo (by various possible combinations of enhanced cross-correction, enhanced lysosomal targeting, enhanced catalytic activity). These combinatorial approaches are also compared against a reference transgene (SEQ ID NO: 1759) for various aforementioned outcomes. Without wishing to be bound by theory, it is believed that transgene-level enhancements can increase the potency of AAV gene therapy and reduce the minimal efficacious dose for in vivo evaluations and clinic applications.

### Example 6. In Vitro Screen

Early in vitro experiments are designed to enable validation of the functional enhancements made in the GBA transgene by conducting side-by-side comparison against a reference GBA construct (e.g., SEQ ID NO: 1759). Experiments are run as AAV vectorized transduction studies on in vitro models of GBA LOF (patient fibroblasts or GBA knockout mouse primary neurons). Dose response is determined. Post-translational modifications and activity of the final GCase product can also be determined.

In vitro evaluations with AAV vectors packaging wtGBA, enGBA and enGBAcombo are carried out on patient fibroblasts with GBA mutations and primary neurons derived from WT and/or 4L/PS-NA GBA mouse model. In preparation for generating 3-point dose-response curves of all AAV.GBA vectors generated, AAV6.CBA.Luciferase reporter-gene transduction assay is performed on the 2 cell lines to verify optimal experimental conditions, e.g. Multiplicity Of Infection (MOI) for AAV6 vectors to be applied across in vitro screening. Once the in vitro experiments are optimized, head-to-head comparisons to identify optimal enGBA transgene configurations for further in vivo evaluations can be conducted.

In vitro dose-response comparisons of AAV-enGBA or AAV-wtGBA constructs (e.g., any one of SEQ ID NOs: 1759-1771 or 1809-1828, e.g., as described in Tables 18-21 or 29-32) for GCase activity: To determine if GBA transgene enhancement strategies confer increased GCase activity in disease-relevant in vitro models, human fibroblasts with and without GBA mutations, and WT/GBA mutant mouse primary neurons are treated with AAV6 vectors packaging enhanced GBA viral genome variants at 3 different MOIs. At terminal time point, secreted and intracellular GCase expression and activity are measured in both media and cell lysate. Additionally, ddPCR based vector genome analysis is conducted to ensure successful in vitro gene transfer across different conditions.

In vitro comparison of AAV-enGBA constructs for subcellular localization: Whether the GBA transgene enhancement strategies confer increased lysosomal localization properties in healthy and disease relevant in vitro models is determined. Human fibroblasts with/without GBA mutations; and WT/GBA mutant mouse primary neurons are treated with AAV6 vectors packaging enhanced GBA viral genome variants. At terminal time point, cells are fixed and co-immunostained for HA (AAV transduction) and lysosomal markers (e.g. Lamp1). Transduction efficiency and % colocalization in the lysosomes are assessed for all AAV vectors using the Bio-Tek Cytation 5 for image analysis and quantification.

In vitro comparison of AAV-enGBA constructs for enzyme cross-correction: In addition to intracellular and secreted GCase activity, AAV-GBA transgene enhancement strategies are assessed for cross-correction properties in disease relevant in vitro conditions. Non-GBA/GBA human fibroblasts and GBA mutant/WT mouse primary neurons are treated with AAV6 vectors packaging enhanced GBA viral genomes. Conditioned media from transduced cells is collected at 24-, 48- and 72-hours post AAV treatments. In order to recapitulate in vivo cross-correction, untreated human fibroblasts and mouse primary neurons are then treated with different conditioned media for 24 hours. At this point, a subset of wells is co-immunostained for HA (visualization of cross-corrected GCase protein product) and lysosomal markers (e.g. Lamp1). Another subset of wells is lysed and evaluated for GCase activity. Cross-correction efficiency and % colocalization in the lysosomes is visualized and quantified for all AAV vector treatments.

In vitro GBA MOI dose-response study with AAVwtGBA and AAVenGBA vectors: Small-scale preps of optimal AAV capsid packaging wtGBA and enGBA constructs. A 3-point dose-response infection study is conducted with wtGBA and enGBA packaging AAVs. GBA-KO neuroblastoma cells (wtGBA neuroblastoma control) and Gaucher disease patient fibroblasts (healthy controls) are used for evaluations.

Select wtGBA or enGBA constructs are identified for further analysis in vivo.

### Example 7. Assay Development

One method of detecting GCase activity involves measuring turnover of an artificial substrate, 4-Methylumbelliferyl β-D-galactopyranoside (4-MUG) as described, for example in Rogers et al., “Discovery, SAR, and biological evaluation of non-inhibitory chaperones of glucocerebrosidase.” (2010), incorporated herein by reference in its entirety. The 4-MUG assay is used to determine GCase activity and GCase concentration in cell lysates.

Another method of detecting GCase activity involves use of a SensoLyte Blue Glucocerebrosidase assay (AnaSpec, Fremont, Calif.), a fluorometric assay, according to the manufacturer's instructions. Sensolyte Blue Glucocerebrosidase assay detects GCase activity using a fluorogenic analog-substrate, wherein the output is fluorescent excitation/emission at 365 nm/445 nm on a standard plate reader.

Fluorescence-based detection of hGBA in mouse tissue, and assessment of hGCase activity in mouse can be determined using the methods as described in Morabito, Giuseppe et al., “AAV-PHP. B-mediated global-scale expression in the mouse nervous system enables GBA gene therapy for wide protection from synucleinopathy.” Molecular Therapy 25.12 (2017): 2727-2742, the contents of which are incorporated by reference herein in their entirety. Alternative methods of visualizing GCase activity are described, for example, in Chao, Daniela Herrera Moro et al., “Visualization of active glucocerebrosidase in rodent brain with high spatial resolution following in situ labeling with fluorescent activity based probes.” PLoS One 10.9 (2015), the contents of which are incorporated herein by reference in their entirety. See also Witte, Martin D., et al. Nature Chemical Biology 6, 907-13 (2010), incorporated herein by reference in its entirety, describing “ultra-sensitive” cyclophellitol β-oxide (CBE) based probes for highly specific GBA labeling in vitro and in vivo. CBE, a GCase inhibitor, irreversibly binds GBA and inhibits its GCase activity, has been shown to cross the blood-brain-barrier, and induces biochemical, clinical and histological manifestations of Gaucher disease (Kuo, Chi-Lin, et al. “In vivo inactivation of glycosidases by conduritol B epoxide and cyclophellitol as revealed by activity-based protein profiling.” The FEBS journal 286.3 (2019): 584-600, incorporated herein by reference in its entirety).

For these assays, GCase/GBA protein concentration and activity are determined and normalized to total protein/activity levels in lysate. Negative control lysates are prepared from, for example, hippocampus and brainstem of vehicle (PBS) treated 6-8 week C57/B16 female mice (n=4). Positive control lysates are from human recombinant GBA-infected/expressing cells. Inhibitor control lysates are from human recombinant GBA+GBA-inhibitor infected/expressing cells to test specificity of the enzyme. An example GCase inhibitor for use in such studies is CBE. Vehicle/Lysis buffer/matrix controls consist of lysis buffer (Sigma) and substrate only. Background controls consist of substrate only. Minimal protein concentrations needed to observe GCase activity are identified by analysis of additional dilutions of lysate.

An assay is validated for evaluating increase in glucosylceramidase activity and glucosylceramidase protein concentration within in vitro GBA disease models (Gaucher patient fibroblasts and GBA-KO neuroblastoma cells) post AAV administration.

### Example 8. In Vivo Screen

In vivo target engagement in GBA disease model: After in vitro screening, target engagement in a GBA mouse model (GBA-4L/PS-NA) is demonstrated. A side-by-side comparison with AAV-wtGBA can be used to further bolster findings and demonstrate efficacy in vivo. In vivo evaluations determine whether AAV9-enGBA and AAV9-enGBAcombo candidate treatments selected during in vitro evaluations result in comparable/significantly higher GCase activity and reduction in GluCer and Glucosylsphingosine substrate-level reduction benefit as compared to AAV9-GBA reference construct in GBA a mouse model.

Up to 10 top enGBA constructs with significantly favorable attributes as compared to GBA reference construct using the GBA 4L/PS-NA mouse model (available at QPS) are tested. AAV-untreated non transgenic (NT) mice are used as controls for biochemical analyses. GBA-4L/PS-NA mice show relevant features of human GBA mutations including significantly reduced GCase activity and increased Glucosylceramide and Glucosylsphingosine as early as 5 weeks post birth. Neuroinflammation in the CTx and hippocampus is also seen in these mice.

In order to assess target engagement in the GBA disease model, intrastriatal administration of AAV9 vectors packaging GBA reference construct and up to top 10 of the enhanced GBA variant viral genomes at three doses 5×10⁹, 1×10¹⁰, 5×10¹⁰ vg/inj via bilateral injections is performed. Animals are euthanized 4 weeks post injections and CNS, peripheral tissues, and fluid compartments (serum and CSF) are collected for AAV biodistribution and transduction (GCase activities and GluCer substrate levels) analyses. Successful/lead candidates cause modest increase (˜30% over baseline) of GCase activity in GBA animal models. Untreated strain- and age-matched WT mice are included to compare physiological levels of GCase and GluCer in healthy animals. Thus, intrastriatal enGBA/enGBAcombo treatments result in equivalent/superior physiological restoration of GCase enzyme levels in the CNS tissues and CSF of GBA mutant mice as compared to GBA reference construct. Concomitantly similar comparison is also made for Glucosylceramide or Glycosylsphingosine levels for different treatments for substrate reduction. Up to 3 top AAV9-enGBA treatments are advanced for efficacy studies.

### Example 9. In Vivo Efficacy Evaluations

Dose selection: In vivo target engagement hits identified in Examples 2-5 are evaluated for GCase expression, target engagement, and efficacy based on readouts in murine disease models of GBA-PD. For efficacy determining in vivo studies, both GBA-4L/PS-NA and SNCA-A53T (M83) mice are used; WT animals are compared as controls. Both mouse models (GBA-4L/PS-NA and M83), n=6-10 mice per group, receive bilateral intrastriatal injections of 5×10⁹, 1×10¹⁰, 5×10¹⁰ vg/inj (or other appropriate concentration based on study results) of the top hits. Mice are euthanized 4 or 8 weeks post-dose. CNS and peripheral tissues and fluid samples including cortex, striatum, thalamus, brain stem, cerebellum, CSF, serum and liver are collected. GCase expression and activity and GluCer substrate levels are measured. Early immunohistochemical readouts using Iba1, GFAP, and H&E stains of mouse brain, spinal cord and liver are performed in order to confirm tolerability at various AAV doses.

Efficacy evaluations will determine whether AAV-enGBA candidate treatments result in efficacious and sustained increase in GCase activity in the brain resulting in reduction in GCase substrate within GBA-4L/PS-NA mouse model. Based on dose selection studies, AAV vectors that are well-tolerated and showing >30% increase in GBA protein expression in relevant CNS tissues are further tested in a time-response study. Briefly, GBA-4L/PS-NA mice (n=6-10) are injected with an intrastriatal injection of lead constructs (dose determined based on dose-selection study), multiple CNS and peripheral tissues and fluid compartments (serum and CSF) are collected at various time-points (e.g. 4, 8 and 12 weeks) and GCase expression and activities, substrate reduction in the CNS and periphery are quantified. Lysosomal localization of the transduced GCase enzyme product are confirmed with immune-colocalization of AAV transduction with lysosomal marker.

Further evaluations will assess whether AAV-enGBA candidate treatments result in efficacious and sustained increase in GCase activity in the brain resulting in reduction of α-Syn pathology within GBA1/α-Synuclein A53T mouse model. Based on dose selection study, AAV vectors that are well-tolerated and showing >30% increase in GBA protein expression in relevant CNS tissues are further tested in SNCA-A53T (M83) mouse model. M83 mice are known to start developing α-syn pathology at 6-7 months of age with progressive motor deficits. M83 mice (n=8-12) are injected with most efficacious constructs (Intrastriatal; dose according to study results) at ˜6 months of age; and evaluated for GCase expression and activity, and α-syn pathology 3 months post administration. Previous studies have shown therapeutic benefit of AAV-GBA in reducing α-syn aggregates in SNCA transgenic mouse models. Here, in addition to GCase expression and activities, AAV-enGBA candidate treatments which result in physiological restoration of GCase enzyme levels (>30%) in the CNS tissue and CSF are evaluated for α-syn pathology reduction in A53T (M83) mice using immunohistochemical analyses.

### Example 10. Natural History Study

In parallel with Stage 1 screening efforts, phenotypic, biochemical and immunohistochemical analysis were performed on 4L/PS-NA, 4L control, and wild-type mice to establish disease-relevant efficacy readouts and timelines.

GBA and Saposin C expression levels were determined in forebrain, midbrain, and hindbrain sections of the mice by LC-MS/MS, and were normalized to actin levels. Consistently across 5, 12, and 18 weeks of age, in all regions of the brain, 4L/PS-NA mice had lower GBA expression levels compared to that of wild-type mice and similar levels of GBA expression as 4L mice (Table 23). The brain of wild-type mice generally showed a trend of increased GBA expression in the hindbrain relative to the midbrain the forebrain (Table 23). Additionally, a decrease in GBA levels in the forebrain and midbrain was observed in the wild-type mice between 5 weeks and 12-18 weeks of age.

TABLE 23
Avg. GBA level (GBA/Actin) in GBA-related disease mouse models

| | 5 Weeks | | | 12 Weeks | | | 18 Weeks | | |
|---|---|---|---|---|---|---|---|---|---|
| | Region of Brain | | | Region of Brain | | | Region of Brain | | |
| Animal | Fore- | Mid- | Hind- | Fore- | Mid- | Hind- | Fore- | Mid- | Hind- |
| Model | brain | brain | brain | brain | brain | brain | brain | brain | brain |
| 4L/PS- | 473.5 | 715.2 | 996.7 | 591.0 | 760.5 | 779.0 | 801.8 | 726.6 | 907.6 |
| NA | | | | | | | | | |
| 4L | 687.5 | 676.8 | 939.8 | 614.5 | 764.9 | 702.8 | 512.3 | 691.8 | 1094.2 |
| Control | | | | | | | | | |
| Wild- | 3463.2 | 3644.1 | 5377.4 | 2616.6 | 2902.2 | 4468.3 | 2551.3 | 3273.6 | 5129.8 |
| type | | | | | | | | | |

Saposin C (SapC)/Actin levels in 4L/PS-NA mice were lower than those observed in the 4L or wild-type mice in the forebrain, midbrain, and hindbrain (Table 24). SapC levels increased in the brains of wild-type mice, with the highest level quantified at 18 weeks of age (Table 24).

TABLE 24
Avg. SapC level (SapC/Actin) in GBA-related disease mouse models

| | 5 Weeks | | | 12 Weeks | | | 18 Weeks | | |
|---|---|---|---|---|---|---|---|---|---|
| | Region of Brain | | | Region of Brain | | | Region of Brain | | |
| Animal | Fore- | Mid- | Hind- | Fore- | Mid- | Hind- | Fore- | Mid- | Hind- |
| Model | brain | brain | brain | brain | brain | brain | brain | brain | brain |
| 4L/PS- | 232.1 | 289.6 | 430.6 | 315.8 | 432.1 | 523.3 | 227.5 | 552.8 | 591.4 |
| NA | | | | | | | | | |
| 4L | 748.6 | 1102.9 | 1769.3 | 844.2 | 1486.5 | 1506.3 | 646.5 | 1123.6 | 1546.4 |
| Control | | | | | | | | | |
| Wild- | 1500.3 | 2283.3 | 3087.9 | 1662.3 | 1971.0 | 2968.9 | 1656.3 | 2143.9 | 3253.1 |
| type | | | | | | | | | |

At five-weeks, 12 weeks, and 18 weeks of age, GCase activity also was measured in forebrain, midbrain and hindbrain tissue sections of 4L/PS-NA (model having decreased GCase and prosaposin), 4L control (model having decreased GCase) and wild-type (normal GCase and prosaposin) mice (Table 7).

At 5 weeks of age, decreased GCase activity was confirmed in both the 4L/PS-NA and 4L control mice, with significant GCase deficits as compared to wild-type mice. GCase activity was not significantly different between the 4L/PS-NA and 4L control mice (Table 7).

TABLE 7
Avg. GCase activity [RFU per mL] in GBA-related disease mouse models

| | 5 Weeks | | | 12 Weeks | | | 18 Weeks | | |
|---|---|---|---|---|---|---|---|---|---|
| | Region of Brain | | | Region of Brain | | | Region of Brain | | |
| Animal | Fore- | Mid- | Hind- | Fore- | Mid- | Hind- | Fore- | Mid- | Hind- |
| Model | brain | brain | brain | brain | brain | brain | brain | brain | brain |
| 4L/PS- | 4318.36 | 6448.92 | 5581.51 | 6343.1 | 6423.7 | 5386.7 | 4109.2 | 6466.4 | 5981.6 |
| NA | | | | | | | | | |
| 4L | 4442.15 | 5614.69 | 5598.62 | 4969.0 | 4640.2 | 6798.4 | 5837.2 | 5915.6 | 8310.4 |
| Control | | | | | | | | | |
| Wild- | 9531.29 | 10448.20 | 10520.70 | 8379.2 | 7596.1 | 9286.1 | 5488.1 | 9730.8 | 8227.2 |
| type | | | | | | | | | |

Similarly, in 12 and 18 weeks old mice, decreased GCase activity was quantified in both the 4L/PS-NA and 4L control mice, with significant GCase deficits as compared to wild-type mice (Table 7). GCase activity was also not significantly different between the 4L/PS-NA and 4L control mice at 12 and 18 weeks of age (Table 7).

Also, at five-weeks, 12 weeks, and 18 weeks of age, GBA substrate levels, specifically glucosylsphingosine (GlcSph) and glucosylceramide (GlcCer), were measured by LC-MS/MS in forebrain, midbrain and hindbrain tissue sections of 4L/PS-NA (model having decreased GCase and prosaposin), 4L control (model having decreased GCase) and wild-type (normal GCase and prosaposin) mice and normalized to actin. As shown in Table 25, the greatest increase in GlcSph levels was observed in the brains of the 4L/PS-NA mice followed by 4L-control mouse brains, relative to the wild-type mouse. Additionally, GlcSph levels in the 4L/PS-NA mouse brains and 4L control mouse brains increased with age and higher levels were observed in the hindbrain, as compared to the forebrain or midbrain. These data demonstrate the effects of reduced GCase activity and decreased GBA levels in these mice, as measured above.

TABLE 25
Avg. glucosylsphingosine level (GlcSph/Actin) in GBA-related disease mouse models

| | 5 Weeks | | | 12 Weeks | | | 18 Weeks | | |
|---|---|---|---|---|---|---|---|---|---|
| | Region of Brain | | | Region of Brain | | | Region of Brain | | |
| Animal | Fore- | Mid- | Hind- | Fore- | Mid- | Hind- | Fore- | Mid- | Hind- |
| Model | brain | brain | brain | brain | brain | brain | brain | brain | brain |
| 4L/PS- | 1086.1 | 1050.9 | 1384.0 | 1792.6 | 1897.0 | 2543.6 | 2571.0 | 3113.1 | 5672.8 |
| NA | | | | | | | | | |
| 4L | 268.0 | 317.0 | 308.7 | 404.5 | 435.2 | 520.1 | 465.8 | 453.4 | 455.1 |
| Control | | | | | | | | | |
| Wild- | 0 | 0 | 0 | 0 | 0 | 0 | 0 | 0 | 0 |
| type | | | | | | | | | |

As shown in Table 26, the levels of GlcCer was increased in the 4L/PS-NA mouse brains, and the levels were higher in the hindbrain as compared to the forebrain and the midbrain. Levels of GlcCer were also higher at 18 weeks of age in the 4L/PS-NA mouse brains. These data also support the effects of reduced GCase activity and decreased GBA levels in these mice, as measured above.

TABLE 26
Avg. glucosylceramide (GlcCer) 18:1/18:0/Actin in GBA-related disease mouse models

| | 5 Weeks | | | 12 Weeks | | | 18 Weeks | | |
|---|---|---|---|---|---|---|---|---|---|
| | Region of Brain | | | Region of Brain | | | Region of Brain | | |
| Animal | Fore- | Mid- | Hind- | Fore- | Mid- | Hind- | Fore- | Mid- | Hind- |
| Model | brain | brain | brain | brain | brain | brain | brain | brain | brain |
| 4L/PS- | 2179.2 | 1875.6 | 2247.5 | 7894.8 | 6425.0 | 12123.0 | 14774.5 | 15368.6 | 25601.5 |
| NA | | | | | | | | | |
| 4L | 695.8 | 528.6 | 454.7 | 703.2 | 547.7 | 488.9 | 738.6 | 627.3 | 545.8 |
| Control | | | | | | | | | |
| Wild- | 510.4 | 361.9 | 332.2 | 375.6 | 359.0 | 355.7 | 517.9 | 439.6 | 374.2 |
| type | | | | | | | | | |

Taken together, these data support use of the 4L/PS-NA mice as model for neuropathic Gaucher disease, and for assessing efficacy of viral constructs encoding a GBA protein e.g., constructs GBA_VG1-GBA_VG34, e.g., as described in Tables 18-21 or 29-32 above.

### Example 11: Exemplary Lead Identification

A. Generation of wild-type and enhanced GBA viral genome variants

Viral genomes were designed for AAV delivery of a GBA protein, e.g., a wild-type GBA protein (wtGBA) that does not further comprise an enhancement element; or an enhanced GBA protein (enGBA) that further comprises an enhancement element described herein, e.g., a prosaposin protein, a SapC protein, or functional variant thereof; a cell penetrating peptide (e.g., an ApoEII peptide, a TAT peptide, and/or an ApoB peptide) or functional variant thereof; a lysosomal targeting signal (LTS) or functional variant thereof; or a combination thereof (enGBAcombo). The nucleotide sequence from 5′ ITR to 3′ ITR of the viral genome constructs that comprise a transgene encoding an GBA protein with or without an enhancement element, are provided as GBA_VG1-GBA_VG33 herein, which are SEQ ID NOs: 1759-1771, 1809-1828, or 1870, respectively. These constructs are also summarized in Table 18, as well as Tables 19-21 and 29-32.

Each of these viral genome constructs comprise a nucleic acid comprising a transgene encoding a GBA protein. The transgene was designed to comprise a wild type nucleotide sequence encoding GBA (SEQ ID NO: 1777), or one of two different codon optimized nucleotide sequence encoding a GBA protein, SEQ ID NO: 1773 or 1781. In designing these viral genome constructs for expression of GBA, several promoters were selected and tested (e.g., promoters as described in Table 5), including a CMV promoter (SEQ ID NO: 1833); a CMVie enhancer and a CMV promoter (SEQ ID NO: 1831 and 1832, respectively); a CMVie enhancer and a CBA promoter (SEQ ID NO: 1831 and 1834 respectively); or an EF-1α promoter variant (SEQ ID NOs: 1839 or 1840).

Some of the viral genome constructs further comprised an intron region, of SEQ ID NO: 1842; a nucleotide sequence encoding a signal sequence (SEQ ID NO: 1850, 1851, or 1852); and/or 4 copies of a miR183 binding site (SEQ ID NO: 1847) separated by a spacer (SEQ ID NO: 1848), or miR183 binding series (SEQ ID NO: 1849). The viral constructs comprised a 5′ ITR of SEQ ID NO: 1829; and a 3′ ITR of SEQ ID NO: 1830. The polyadenylation sequence (SEQ ID NO: 1846) was the same across all viral genome constructs designed.

Wild-type GBA viral genome variants encoding a GBA protein were prepared as described and are outlined in Table 18-21 or 29-32 (e.g., GBA_VG1, GBA_VG17-GBA_VG21, GBA_VG26, and GBA_VG33; SEQ ID NOs: 1759, 1812-1816, 1821, and 1828).

Enhanced GBA viral genome variants encoding a GBA protein and an enhancement element described herein (e.g., an enhancement element of Table 4 or 16) were prepared and are outlined in Table 18 (GBA_VG2-GBA_VG16, GBA_VG22-GBA_VG25, GBA_VG27-GBA_VG32; SEQ ID NO: 1760-1771, 1809-1811, 1817-1820, 1822-1827). The enhanced viral genomes were designed to further encode an enhancement element comprising a prosaposin protein (encoded by SEQ ID NO: 1859); saposin C protein or a functional variant (encoded by SEQ ID NO: 1787 or 1791); a cell penetrating peptide, including an ApoEII peptide (encoded by SEQ ID NO: 1797), a TAT protein (encoded by SEQ ID NO: 1793), or an ApoB peptide (encoded by SEQ ID NO: 1795); a lysosomal targeting signal (LTS) (encoded by any of SEQ ID NOs: 1799, 1801, 1803, 1805, or 1807); or a combination thereof. Some of the enhanced viral genome constructs further comprise a nucleotide sequence encoding a signal sequence (e.g., SEQ ID NO: 1856), and/or a linker (e.g., SEQ ID NO: 1724, 1726, or 1730). Some constructs, e.g., those encoding a prosaposin protein or a saposin C protein, encode a cleavable linker such as a furin and/or T2A cleavage site (encoded by SEQ ID NO: 1724 or 1726, respectively). Some constructs, e.g., those encoding a cell penetrating peptide, encode a flexible, glycine-serine linker (encoded by SEQ ID NO: 1730).

The viral construct GBA_VG1 (SEQ ID NO: 1759), comprising the nucleotide sequence of SEQ ID NO: 1781, with no additional enhancement elements (e.g., a saposin protein, a lysosomal targeting sequence, a cell penetrating sequence, or a combination thereof) was used as a reference or benchmark construct, e.g., in the experiments described herein.

B. In-vitro assessment of payload expression

Prior to vectorization, the wild-type and enhanced GBA viral genome variants were first used to validate the tools necessary for conducting lead identification studies, such as, but not limited to assays and cell-systems.

LC-MS/MS assays were established to quantify GBA (ng/mg of total protein) and SapC (ng/mg of total protein) in lysates collected from HEK293 cells transfected with a wild-type or enhanced GBA viral genome variant plasmid DNA including, GBA_VG1 (SEQ ID NO: 1759, encoding a GBA protein), GBA_VG8 (SEQ ID NO: 1766, encoding a GBA protein and a prosaposin protein of SEQ ID NO: 1785), GBA_VG9 (SEQ ID NO: 1767, encoding a GBA and a Saposin C protein of SEQ ID NO: 1789) and GBA_VG10 (SEQ ID NO: 1768, encoding a GBA protein and a Saponin C protein of SEQ ID NO: 1758). Lysates were also run on Western blot to confirm the presence of expressed GBA.

These validation experiments demonstrated that transfection of cells with the wild-type or enhanced GBA variant construct DNA resulted in increases in measured GBA or SapC in the lysate as determined by LC-MS/MS and Western blot when compared to lysate of untransfected cells.

C. In-vitro cell-system assessment and validation

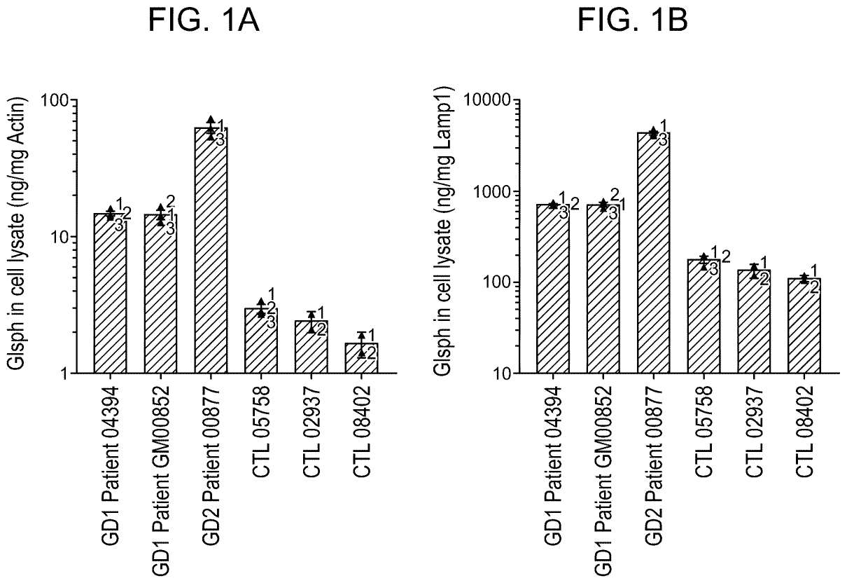

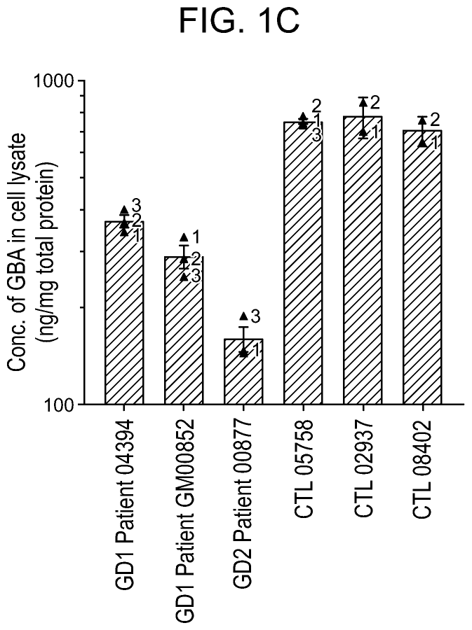

Additional LC-MS/MS assays were used to quantify GCase activity and/or levels of GBA substrates (e.g., glycosphingolipids (GlcSph) quantified as ng/mg Actin in the FIG. 1A, or as ng/mg Lamp 1 in FIG. 1B) in Gaucher disease patient derived (GM04394-fibroblast (GD1 patient), GM00852-fibroblast (GD1 patient), GM00877-fibroblast (GD2 patient) or healthy control (GM05758-fibroblast from skin/inguinal area and GM02937-fibroblast from skin/unspecified) fibroblasts. Again, these quantifications were supplemented with Western blot analyses.

As anticipated, GBA substrate levels, specifically glycosphingolipids, as quantified as ng/mg Actin in FIG. 1A, or as ng/mg Lamp 1 in the in FIG. 1B, were increased across all three Gaucher disease patient derived fibroblast samples, as compared to control fibroblast levels, when measured by LC-MS/MS (FIGS. 1A-1B). Meanwhile, GBA protein detection (measured as the concentration of GBA in the cell lysate relative to total protein (ng/mg total protein) in GD patient fibroblasts was decreased compared to the healthy controls (FIG. 1C).

Quantification of GCase activity in lysate collected from Gaucher disease patient derived fibroblasts transfected with enhanced GBA viral genome variants was measured as Relative Fluorescence units per ng protein (RFU per ng protein) and was shown to be decreased when by 96.5%, 98.4% and 99.2% (GM04394-fibroblast, GM00852-fibroblast, GM00877-fibroblast, respectively) as compared to an average of the normal controls. Data are shown below in Table 8.

TABLE 8
Avg. GCase activity[RFU per ng protein] in
Gaucher disease patient derived fibroblasts

| Fibroblast cell line | GCase activity |
|---|---|
| GM04394 | 465.53 |
| GM00852 | 221.29 |
| GM00877 | 109.44 |
| GM05758 (Control) | 14997.59 |
| GM02937 (Control) | 11750.07 |

Quantification of GBA substrate in lysate collected from Gaucher disease patient derived fibroblasts transfected with enhanced GBA viral genome variants was measured as glucosylsphingosine/Lamp1 (ng/mg Lamp1) and was shown to be increased when compared to control. Data are shown below in Table 9.

TABLE 9
Avg. glucosylsphingosine (ng/mg Lamp1) in
Gaucher disease patient derived fibroblasts

| Fibroblast cell line | Glucosylsphingosine |
|---|---|
| GM04394 | 716.33 |
| GM00852 | 710.67 |
| GM00877 | 4348.00 |
| GM05758 (Control) | 146.00 |
| GM02937 (Control) | 134.5 |

D. In-vitro packaging into AAV particles and capsid selection

The wild-type and enhanced GBA viral genome variants (GBA_VG1 to GBA_VG13; SEQ ID NO: 1759-SEQ ID NO: 1771) were each packaged into AAV2 or AAV6 capsids.

In vitro capsid selection studies were conducted wherein cells were transduced with AAV particles comprising an enhanced GBA viral genome variant packaged in AAV2 or AAV6 at a series of increasing MOIs (1.00E1, 1.00E2, 1.00E3, 1.00E4, 1.00E5 and 1.00E6) and vector genome per cell quantified. Based on transduction efficiency, AAV2 was selected for further studies.

E. In-vitro dose-range finding studies

AAV particles comprising reference viral genome GBA_VG1 (SEQ ID NO: 1759) was screened in a dose-range finding study, wherein GCase activity was quantified subsequent to transduction of cells with an increasing series of MOIs (1.00E1, 1.00E2, 1.00E3, 1.00E4, 1.00E5 and 1.00E6). Based on these findings, a mid-range MOI of 1.00E3 was selected for further studies.

F. AAV2-GBA transduction in Gaucher Disease patient fibroblast cells

AAV2-GBA particles comprising viral genomes GBA_VG1 (SEQ ID NO: 1759), GBA_VG2 (SEQ ID NO: 1760), GBA_VG3 (SEQ ID NO: 1761), GBA_VG4 (SEQ ID NO: 1762), GBA_VG5 (SEQ ID NO: 1763), GBA_VG6 (SEQ ID NO: 1764), GBA_VG7 (SEQ ID NO: 1765) were administered at MOI 1.00E3 to Gaucher disease patient fibroblasts (GM00877-fibroblasts) and GCase activity quantified and normalized to mg protein. The results are shown in Table 10 below.

TABLE 10
GCase activity in Gaucher disease patient fibroblasts

| | | GCase Activity |
|---|---|---|
| Construct ID | SEQ ID NO: | [RFU per mL] |
| GBA_VG1 | 1759 | 9814.48 |
| | | 16616.84 |
| | | 3686.62 |
| | | 3478.15 |
| Avg. | | 8399.02 |
| GBA_VG2 | 1760 | 6375.98 |
| | | 3150.89 |
| | | 7426.56 |
| | | — |
| Avg. | | 5651.14 |
| GBA_VG3 | 1761 | 11580.6 |
| | | 22812.97 |
| | | 14152.31 |
| | | — |
| Avg. | | 16181.96 |
| GBA_VG4 | 1762 | 10963.45 |
| | | 11695.58 |
| | | 7980.39 |
| | | — |
| Avg. | | 10213.14 |
| GBA_VG5 | 1763 | 17076.48 |
| | | 8665.12 |
| | | 16343.61 |
| | | — |
| Avg. | | 14028.40 |
| GBA_VG6 | 1764 | 5117.13 |
| | | 3391.47 |
| | | 4177.62 |
| | | — |
| Avg. | | 4228.74 |
| GBA_VG7 | 1765 | 9842.00 |
| | | 9366.20 |
| | | 13187.36 |
| | | — |
| Avg. | | 10798.52 |

Western blot analysis further confirmed a ˜70 kD mature GBA protein in those samples transduced with AAV2-enhanced GBA particles. Negligible GBA was evident in Gaucher disease patient derived fibroblast samples that had not been transduced with an AAV2-enhanced GBA particle.

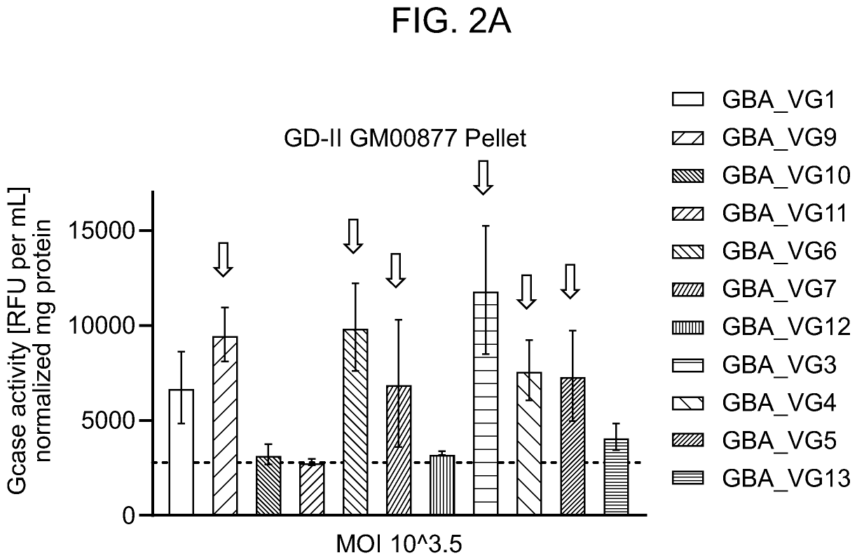

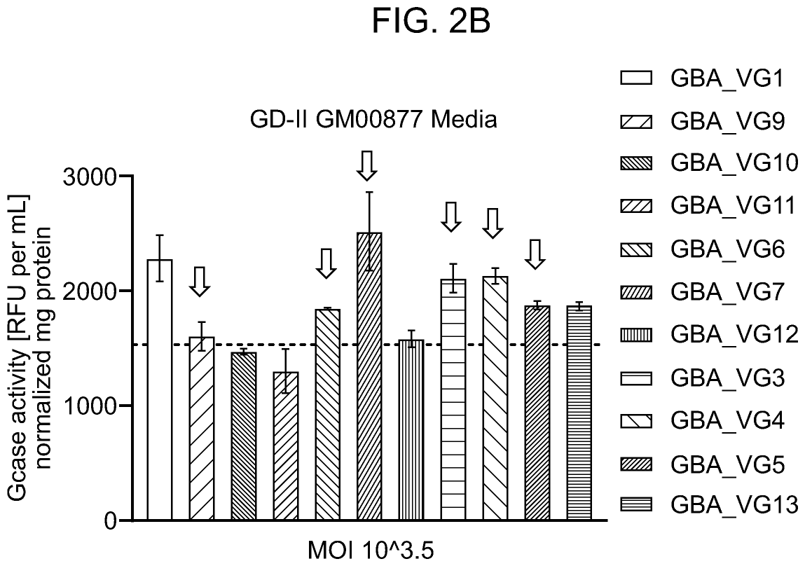

Additional AAV2 particles comprising viral genome constructs encoding a GBA protein and an enhancement element such as saposin C protein, a lysosomal targeting signal (LTS), a cell penetrating peptide (CPP), or a combination thereof (e.g., as outlined in Table 18 above), were vectorized for screening at an MOI of 10³⁵ in Gaucher disease (GD) patient-derived fibroblasts (GD-II GM00877). The vectorized viral genome constructs included GBA_VG1 (SEQ ID NO: 1759), GBA_VG9 (SEQ ID NO: 1767), GBA_VG10 (SEQ ID NO: 1768), GBA_VG11 (SEQ ID NO: 1769), GBA_VG6 (SEQ ID NO: 1764), GBA_VG7 (SEQ ID NO: 1765), GBA_VG12 (SEQ ID NO: 1770), GBA_VG3 (SEQ ID NO: 1761), GBA_VG4 (SEQ ID NO: 1762), GBA_VG5 (SEQ ID NO: 1763), and GBA_VG13 (SEQ ID NO: 1771). GCase activity was quantified in the pelleted patient cells (FIG. 2A) and the corresponding conditioned media (FIG. 2B), as relative fluorescence units per mg protein (RFU per mg protein). Treatment with six of the vectorized viral genome constructs (GBA_VG9, GBA_VG6, GBA_VG7, GBA_VG3, GBA_VG4, and GBA_VG5) resulted in an increase in GCase activity measured in the pelleted GD patient fibroblasts (FIG. 2A) and the corresponding conditioned media (FIG. 2B).

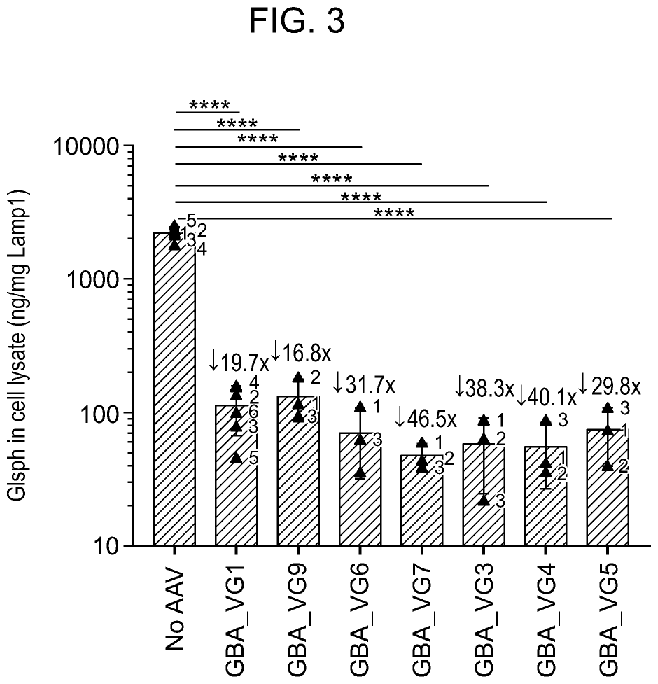

An LC-MS/MS assay was then used to quantify levels of the GBA substrate glycosylsphingosine (GlcSph, ng/mg Lamp1) in the cell lysis from GD II patient-derived fibroblasts transduced with the viral genomes constructs GBA_VG1 (SEQ ID NO: 1759), GBA_VG9 (SEQ ID NO: 1767), GBA_VG6 (SEQ ID NO: 1764), GBA_VG7 (SEQ ID NO: 1765), GBA_VG3 (SEQ ID NO: 1761), GBA_VG4 (SEQ ID NO: 1762), and GBA_VG5 (SEQ ID NO: 1763) vectorized in AAV2 particle. As shown in FIG. 3, the buildup of GBA substrate levels was reduced significantly in GD patient-derived fibroblasts transduced with the AAV2 GBA vectors, compared to the no AAV control. The data demonstrated that the AAV-mediated gene therapy can be effective in increasing GCase activity to treat diseases associated with GBA deficiency.

### Example 12: AAV2 Enhanced GBA Vectors Containing Combinations of Enhancement Elements

Additional viral genome constructs were generated encoding a GBA protein, wherein the GBA protein is encoded by the wild-type nucleotide sequence of SEQ ID NO: 1777, or the codon optimized nucleotide sequence of SEQ ID NO: 1773, and further encoding an enhancement element such as a cell penetration peptide (CPP) (e.g., ApoEII), a lysosomal targeting sequence (e.g., LTS2), a SapC protein, or combination thereof. These constructs also comprised different promoters, including a CBA, a CMV, or a CAG promoter. These exemplary GBA viral genome constructs are included in Table 18-20.

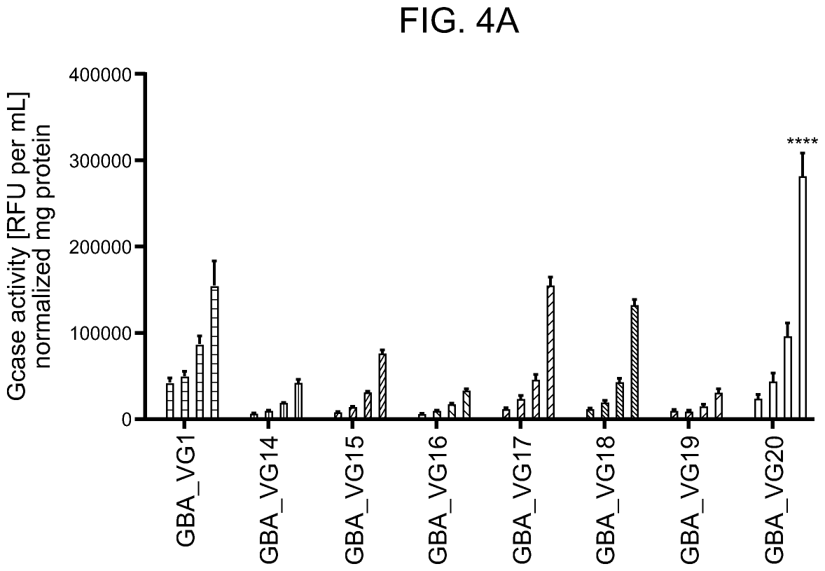

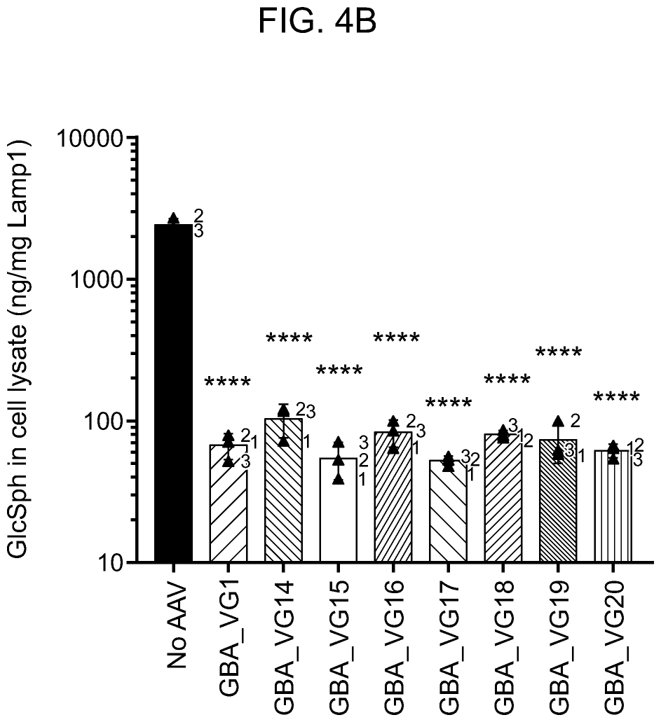

GD-II patient fibroblasts (GM00877) were transduced with an AAV2 vector comprising the viral genome constructs: GBA_VG1 (SEQ ID NO: 1759), GBA_VG14 (SEQ ID NO: 1809), GBA_VG15 (SEQ ID NO: 1810), GBA_VG16 (SEQ ID NO: 1811), GBA_VG17 (SEQ ID NO: 1812), GBA_VG18 (SEQ ID NO: 1813), GBA_VG19 (SEQ ID NO: 1814), and GBA_VG20 (SEQ ID NO: 1815), at an MOI of 10²⁵ (first bar), 10³ (second bar), 10³⁵ and 10⁴. The GCase activity was quantified on day 7 post-transduction after lysing the treated patient cells (FIG. 4A), as relative fluorescence units per mg protein (RFU per mg protein As shown in FIG. 4A, all viral genome constructs tested resulted in a dose-responsive increase in GCase activity in GD II patient-derived fibroblasts. The GBA_VG20 construct comprising the CAG promoter operably linked to a codon-optimized nucleotide sequence of SEQ ID NO: 1773 encoding a GBA protein vectorized in AAV2 vector showed significantly higher GCase activity compared to the GBA_VG1 construct comprising a CMVie enhancer and CBA promoter operably linked to the nucleotide sequence of SEQ ID NO: 1781 encoding the GBA protein at the MOI of 10⁴.

An LC-MS/MS assay was then used to quantify levels of the GBA substrate glycosylsphingosine (GlcSph, ng/mg Lamp1) in the cell lysis from GD II patient-derived fibroblasts transduced with the viral genomes constructs GBA_VG1 (SEQ ID NO: 1759), GBA_VG14 (SEQ ID NO: 1809), GBA_VG15 (SEQ ID NO: 1810), GBA_VG16 (SEQ ID NO: 1811), GBA_VG17 (SEQ ID NO: 1812), GBA_VG18 (SEQ ID NO: 1813), GBA_VG19 (SEQ ID NO: 1814), and GBA_VG20 (SEQ ID NO: 1815) vectorized in an AAV2 vector. As shown in FIG. 4B, all viral genome constructs tested reduced GBA substrate buildup indicating successful target engagement within GD patient cells.

### Example 13: Bioinformatics Analysis of Wild-Type and Codon-Optimized Sequences Encoding a GBA Protein

Bioinformatics analysis on the sequence level was performed to differentiate between viral genome constructs encoding a GBA protein, wherein the GBA protein is encoded by a wild-type nucleotide sequence of SEQ ID NO: 1777 (e.g., the nucleotide sequence encoding the GBA protein of GBA_VG21, as shown in Table 18), a first codon-optimized nucleotide sequence of SEQ ID NO: 1773 (e.g., the nucleotide sequence encoding the GBA protein of GBA_VG17, as shown in Table 18-20), or a second codon-optimized of SEQ ID NO: 1781 (the nucleotide sequence encoding the GBA protein of GBA_VG1, as shown in Table 18). Briefly, sequence-level differentiation criteria, such as GC content, RNA accessibility, miRNA binding, transcriptional motifs and splicing events, were assessed using mRNA-based sequence analysis tools (RegRNA 2.0 by Chang et al., 2013, BMC Bioinformatics, 14, Suppl 2:S4; miRDB by Chen & Wang, 2020, Nucleic Acids Res, 48(D1): D127-D131; the contents of each herein incorporated by reference in its entirety).

Based on the above analysis using miRDB (Chen & Wang, 2020, supra) with respect to miRNA binding, a series of putative recognition sites were found in the construct with first codon-optimized nucleotide sequence encoding a GBA protein of SEQ ID NO: 1773 (GBA_VG17). Specifically, this codon-optimized sequence of SEQ ID NO: 1773 had 42 total miRNA binding sites including 4 high confidence hits. Among those, 21 sites were distinct from the second codon optimized sequence of SEQ ID NO: 1781 of GBA_VG1, and 11 sites were new and not present in the wild type nucleotide sequence of SEQ ID NO: 1777 of GBA_VG21. This is summarized in Table 11. A second miRNA analysis tool (RegRNA 2.0 by Chang et al., 2013 supra), as shown in Table 28, further confirmed that miRNA binding sites tended to be unique to each sequence codon optimized sequence analyzed.

TABLE 11
miRDB Summary of miRNA Binding

| | WT (SEQ ID | WT (SEQ ID | SEQ ID | |
|---|---|---|---|---|
| | NO: 1777) | NO: 1777) | NO: 1773 | “High |
| | vs SEQ ID | vs SEQ ID | vs SEQ ID | Confidence” |
| | NO: 1781 | NO: 1773 | NO: 1781 | miRNA |
| SEQ ID NO: 1773 | N/A | 30 | 21 | 4 |
| (GBA_VG17) Unique | | | | |
| Wild Type (SEQ ID NO: | 42 | 41 | N/A | 1 |
| 1777) (GBA_VG21) Unique | | | | |
| SEQ ID NO: 1781 | 27 | N/A | 17 | 2 |
| (GBA_VG1) Unique | | | | |
| Similar | 11 | 12 | 21 | 0 |

TABLE 28
RegRNA 2.0 Summary of miRNA Binding

| | WT (SEQ ID | WT (SEQ ID | SEQ ID |
|---|---|---|---|
| | NO: 1777) | NO: 1777) | NO: 1773 |
| | vs SEQ ID | vs SEQ ID | vs SEQ ID |
| | NO: 1781 | NO: 1773 | NO: 1781 |
| SEQ ID NO: 1781 | 3 | N/A | 3 |
| (GBA_VG1) | | | |
| Unique | | | |
| SEQ ID NO: 1773 | N/A | 2 | 3 |
| (GBA_VG17) | | | |
| Unique | | | |
| WT (SEQ ID NO: | 6 | 6 | N/A |
| 1777) (GBA_VG21) | | | |
| Unique | | | |
| Similar | 0 | 1 | 0 |

With respect to the analysis of the transcriptional motifs, the first codon-optimized sequence of SEQ ID NO: 1773 in GBA_VG17 had 70 total sites; 32 sites were distinct from the second codon optimized sequence of SEQ ID NO: 1781 in GBA_VG1, and 54 sites were new and not present in the wild type sequence of SEQ ID NO: 1777 in GBA_VG21 (Table 12).

TABLE 12
RegRNA 2.0 summary of regulatory motifs

| | WT (SEQ ID | WT (SEQ ID | SEQ ID |
|---|---|---|---|
| | NO: 1777) | NO: 1777) | NO: 1773 |
| | vs SEQ ID | vs SEQ ID | vs SEQ ID |
| | NO: 1781 | NO: 1773 | NO: 1781 |
| SEQ ID NO: 1781 | 49 | N/A | 24 |
| (GBA_VG1) Unique | | | |
| SEQ ID NO: 1773 | N/A | 54 | 32 |
| (GBA_VG17) Unique | | | |
| Wild Type (SEQ ID | 39 | 34 | N/A |
| NO: 1777) (GBA_VG21) | | | |
| Unique | | | |
| Similar | 11 | 16 | 38 |

With respect to the splicing events analysis, the first codon-optimized sequence of SEQ ID NO: 1773 in GBA_VG17 had 5 total sites; 3 sites were distinct form the second codon-optimized sequence of SEQ ID NO: 1781 GBA sequence in GBA_VG1, and all 3 of these were completely new and not present in the wild-type sequence of SEQ ID NO: 1777 in GBA_VG21 (Table 13).

TABLE 13
Summary of RegRNA 2.0 Splice Events

| | WT (SEQ ID | WT (SEQ ID | SEQ ID |
|---|---|---|---|
| | NO: 1777) | NO: 1777) | NO: 1773 |
| | vs SEQ ID | vs SEQ ID | vs SEQ ID |
| | NO: 1781 | NO: 1773 | NO: 1781 |
| Wild Type (SEQ ID | 6 | 6 | N/A |
| NO: 1777) (GBA_VG21) | | | |
| Unique | | | |
| SEQ ID NO: 1773 | N/A | 5 | 3 |
| (GBA_VG17) Unique | | | |
| SEQ ID NO: 1781 | 3 | N/A | 2 |
| (GBA_VG1) Unique | | | |
| Similar | 1 | 0 | 2 |

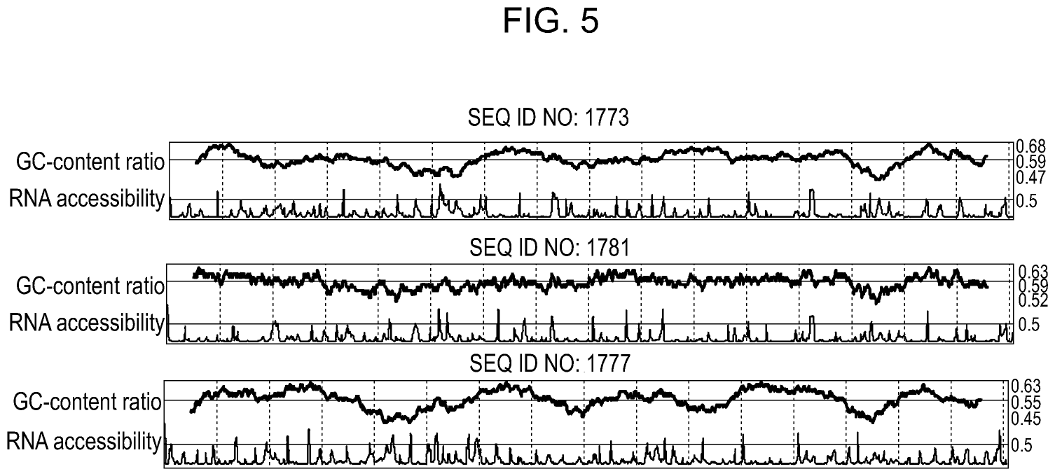

The GC content of the first codon optimized sequence (SEQ ID NO: 1773) was compared to the second codon optimized sequence (SEQ ID NO: 1781) and the wild-type sequence (SEQ ID NO: 1777). For RNA accessibility and GC content, the wild type GC biodistribution pattern was maintained in the first codon optimized sequence of SEQ ID NO: 1773 of GBA_VG17 (FIG. 5). However, the second codon optimized sequence of SEQ ID NO: 1781 (GBA_VG1) had balanced GC content across the entire length of the nucleotide sequence (FIG. 5).

The percentage homology for SEQ ID NO: 1781 (GBA_VG1) and SEQ ID NO: 1773 (GBA_VG17) with respect to the wild type sequence (SEQ ID NO: 1777; GBA_VG21) is shown in Table 14. The first codon-optimized sequence of SEQ ID NO: 1773 in GBA_VG17 shares about 80.6% and about 80.0% sequence homology with respect to the wild type sequence of SEQ ID NO: 1777 in GBA_VG21, without and with the signal sequence, respectively. The second codon-optimized sequence (SEQ ID NO: 1781) in GBA_VG1 shares about 81.3% and about 80.7% sequence homology with respect to the wild type GBA sequence, without and with signal sequence, respectively. The first codon-optimized sequence of SEQ ID NO: 1773 in GBA_VG17 has about 87.0% and about 86.3% sequence homology with respect to the second codon optimized nucleotide sequence of SEQ ID NO: 1781, in GBA_VG1 without and with signal sequence, respectively. There were 131 unique mutations introduced into the first codon-optimized nucleotide sequence of SEQ ID NO: 1773 (GBA_VG17) relative to the wild type nucleotide sequence of SEQ ID NO: 1777 (GBA_VG21). The second codon-optimized nucleotide sequence of SEQ ID NO: 1781 (GBA_VG1) had 120 unique mutations relative to the wild type nucleotide sequence of SEQ ID NO: 1777 (GBA_VG21).

TABLE 14
GC Content and Percentage Homology of Codon
Optimized Sequences (SEQ ID NO: 1773 or 1781) Relative
to the Wild Type Sequence (SEQ ID NO: 1777)
% Homology to WT GBA

| | Without | With | Splice | | G/C |
|---|---|---|---|---|---|
| cDNA | signal | Signal | site | Unique | content |
| Source | sequence | sequence | only | Mutations | (WT 55%) |
| SEQ ID NO: | 80.6% | 80.0% | 4 bp | 131 | 59% |
| 1773 | | | muta- | | |
| (GBA_VG17) | | | tion | | |
| SEQ ID NO: | 81.3% | 80.7% | 5 bp | 120 | 58.5% |
| 1781 | | | muta- | | |
| (GBA_VG1) | | | tion | | |
| % SEQ ID | 87.0% | 86.3% | 3 bp | 190 | |
| NO: 1773 to | | | differ- | (similar) | |
| SEQ ID NO: | | | ence | | |
| 1781 | | | | | |
| (GBA_VG17/ | | | | | |
| GBA_VG1) | | | | | |

### Example 14: Functional Comparison of Wild-Type and Codon-Optimized Sequences Encoding a GBA Protein

GBA expression and GCase activity of a GBA protein was compared for the vectorized viral genome constructs GBA_VG17 (SEQ ID NO: 1812) comprising a first codon optimized sequence (SEQ ID NO: 1773) encoding the GBA protein, GBA_VG1 (SEQ ID NO: 1759) comprising a second codon optimized sequence (SEQ ID NO: 1781) encoding the GBA protein, and GBA_VG21 (SEQ ID NO: 1816) comprising a wild-type GBA sequence (SEQ ID NO: 1777) encoding a GBA protein.

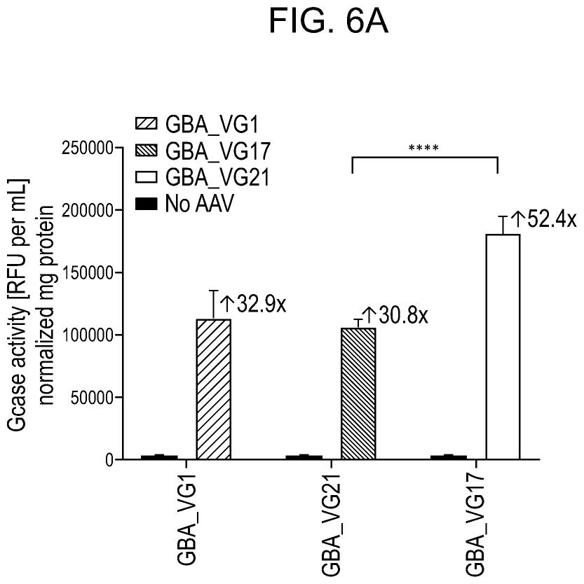

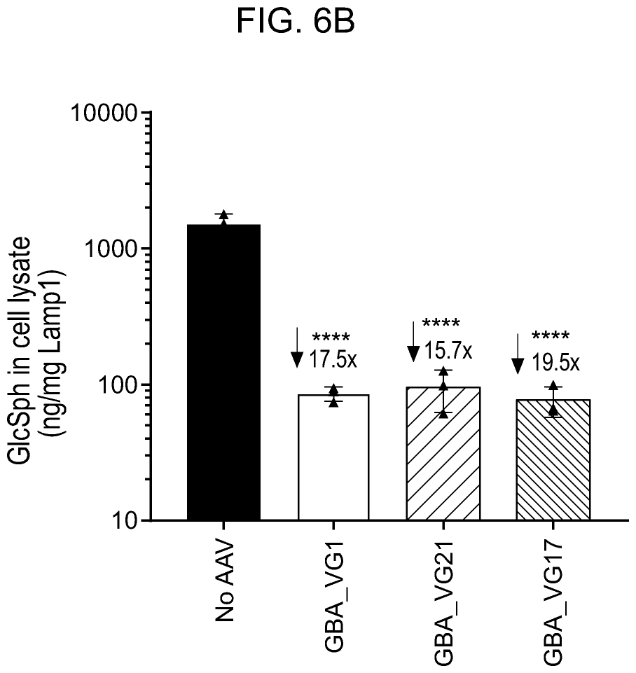

GD-II patient fibroblasts (GD-II GM00877) were treated at 10⁴⁵ MOI of AAV2 vectors comprising the following constructs: GBA_VG17 (AAV2.GBA_VG17), GBA_VG1 (AAV2.GBA_VG1), or GBA_VG21 (AAV2.GBA_VG21), and GCase activity was quantified as RFU per mL and normalized to mg of total protein. As shown in FIG. 6A, AAV2.GBA_VG17 resulted in superior enzymatic GCase activity compared to AAV2.GBA_VG1 and AAV2.GBA_VG21 treated cells. GCase activity was 52.4 fold higher in AAV2.GBA_VG17 treated GD patient cells compared to a no AAV control; but only 30.8 fold and 32.9 fold higher in AAV2.GBA_VG21 and AAV2.GB_VG1 treated GD patient cells, respectively, compared to a no AAV control.

GD-II patient fibroblasts (GD-II GM00877) were then treated at an MOI of 10⁶ of AAV2 vectors comprising the following constructs: GBA_VG17 (AAV2.GBA_VG17), GBA_VG1 (AAV2.GBA_VG1), or GBA_VG21 (AAV2.GBA_VG21), and glucosylsphingosine levels (GlcSph in cell lysate (ng/mg Lamp1) and GBA substrate reduction activity was measured by LC-MS/MS. As shown in FIG. 6B, all constructs tested resulted in similar glucosylsphingosine levels and GBA substrate reduction, which were significantly reduced compared to the no AAV control. Expression of the encoded GBA protein by these GD patient cells treated with AAV2.GBA_VG17, AAV2.GBA_VG1, and AAV2.GBA_17 vectors was confirmed by Western blot.

Taken together, these data demonstrate higher GCase activity, stable GBA protein expression, and significant reduction of glucosylsphingosine levels with AAV2 vectorized GB_VG17 (SEQ ID NO: 1812), comprising the codon-optimized nucleotide sequence of SEQ ID NO: 1773 encoding the GBA protein compared to AAV2 vectorized GBA_VG1 and GBA_VG21.

### Example 15. Route of Administration and Production Platform Comparison Study

In this Example, HEK and Sf9-produced AAV9 vectors, for biodistribution and GBA expression in wild type rat brain were assessed. The vectors were administered to the animals either by a single route of administration by intra-cisterna magna (ICM) or intra-thalamic (ITH) delivery, or a dual route of administration, comprising a combination of ICM and ITH delivery.

AAV9 vectors packaged with GBA_VG1 (SEQ ID NO: 1759) (AAV9.GBA_VG1) produced in HEK and SF9 cells were injected into wild-type rat. For bilateral ITH administration, 7.5×10⁹ AAV9.GBA_VG1 viral genomes were injected into the thalamus of each hemisphere, resulting in a total dose of 1.5 10⁹ vector genomes. For ICM injection, 1.5×10¹⁰ AV9.GBA_VG1 viral genomes were injected. For dual ITH and ICM administration, 1.5×10¹⁰ AV9.GBA_VG1 viral genomes were injected for ICM delivery, and 7.5×10⁹ AAV9.GBA_VG1 viral genomes were injected into the thalamus of each hemisphere for bilateral ITH delivery, for a total dose of 3×10¹⁰ AV9.GBA_VG1 viral genomes. Four weeks post-injection, the brains of the rats were assayed for bio-distribution of the viral genome and GCase activity in the central nervous system and peripheral tissues.

First, all animals throughout all treatment groups continued to gain weight consistently post operatively until time of euthanasia (4 week in life). Daily clinical observations showed normal healthy subjects. Therefore, at both selected doses of 1.5×10¹⁰ for single route of administration, and 3×10¹⁰ for combination route of administration, all animals tolerated the AAV treatments.

Viral genome distribution was also assessed at 28 days post intrathalamic dosing of HEK and Sf9 produced AAV9-GBA vectors in the wild-type rat brain, particularly in the thalamus, hippocampus striatum, and cortex. In the thalamus, hippocampus and striatum, there was a trend of higher average biodistribution with HEK-produced AAV9 vector compared to Sf9 (˜3-6 fold VG/cell increase). In the cortex, a similar average biodistribution profile was observed for HEK and Sf9 produced AAV9 vectors. These data show increased biodistribution with AAV9-GBA vectors produced by an HEK platform as compared to Sf9 following intrathalamic dosing in rats.

GCase activity was also compared at 28 days post intrathalamic dosing of HEK and Sf9 produced AAV9-GBA vectors in the wild-type rat brain. GCase activity was generally increased over baseline. However, the average increase in GCase activity was the greatest in the thalamus (site of injection) (70% over endogenous for Sf9 produced vectors and 20% over endogenous for HEK vectors, and in the thalamus, a moderate increase in average GCase activity was observed for HEK produced vectors relative to Sf9 produced vectors. Low to moderate increase was observed in forebrain and midbrain regions (cortex, striatum and hippocampus, ˜5-40% over endogenous).

Taken together, these data demonstrate that bilateral ITH AAV9.GBA_VG1 dosing resulted in successful distribution of AAV VGs in the CNS tissues and detectable overexpression of GBA (increased GCase activity over endogenous GCase activity) within a wild-type rat brain.

The effects of routes of administration on viral genome distribution and GCase activity of HEK produced AAV9.GBA_VG1 vectors were evaluated at day 28 post ICM, ITH, or dual ICM and ICM delivery of the vectors. A significantly higher viral genome distribution in ITH group was observed in deep-brain structures, including the thalamus and hippocampus, compared to ICM or dual ITH and ICM dosing. Similarly, higher viral genome distribution was observed in ITH group compared to ICM and dual ITH and ICM dosing. For cortex tissue, dual ITH and ICM dosing resulted in a significantly higher viral genome biodistribution compared to ITH and ICM dosing. Taken together, ITH delivery of AAV9-GBA vector displayed a higher viral genome (VG) biodistribution profile in the deep-midbrain structures (especially in the hippocampus and thalamus) as compared to ICM and dual ITH+ICM delivery. Further, ITH delivery appeared to drive the VG biodistribution profile in fore/mid brain observed with dual ITH+ICM injection.

For hindbrain tissues, a significantly higher cerebellar viral genome distribution was observed in ITH group compared to ICM or dual ITH and ICM dosing. Similarly, a trend of higher viral genome biodistribution was observed in brainstem for ITH group compared to ICM and dual ITH and ICM dosing. Therefore, similar to forebrain and midbrain structures, ITH delivery of AAV9-GBA_VG1 vector produced by HEK cells displayed a higher viral genome biodistribution profile in the hindbrain structure as compared to ICM and dual ITH and ICM delivery.

For forebrain and midbrain tissues with respect to GCase activity, the highest increase was observed at site of injection in thalamus (about 250% post ITH deliver followed by about 207% in dual ITH and ICM dosing). Moderate increase was observed post ITH delivery in cortex (about 123% compared to vehicle control, and about 141% after dual ITH and ICM dosing). For ICM dosing, minimal or no increase in GCase activity was observed in thalamus (about 110%) and cortex (about 91%) post ICM delivery. Combinatorial dosing (ICM and ITH) showed the highest GCase activity in cortex (about 141%). Overall, based on the viral genome distribution results and the GCase results, ITH dosing appears to be driving the increase in GCase activity in thalamus and cortex. Additionally, the AAV genome per cell biodistribution shows similar trend of GCase activity in the thalamus and cortex.

For hindbrain tissues, the highest increase was observed post ITH injection in cerebellum (about 178%), and a low increase was seen post both ICM and dual ICM and ITH delivery in cerebellum. Combinatorial dosing group did not show a higher increase in GCase activity readout within cerebellum. Overall, based on the viral genome distribution results and the GCase results, ITH dosing appears to be driving the increase in GCase activity in the cerebellum and the AAV genome per cell biodistribution shows similar trend of GCase activity in the cerebellum.

GCase activity was also evaluated in CSF fluid. Different levels of increase in CSF GCase activity were observed with different routes of administration. Specifically, the highest increase in GCase activity was observed in combinatorial dosing (ITH and ICM), followed by ITH delivery, and only moderate increase was observed by ICM delivery. These data demonstrated that AAV delivered GBA gene transfer in rats resulted in secretion of active GCase product in CSF.

This experiment demonstrated that intrathalamic injection and dual mode injection resulted in a more efficient delivery of AAV-GBA viral particles and a higher GCase expression/activity in the CNS tissues and the CSF.

### Example 16: In Vivo Evaluation of a Vectorized Viral Genome Comprising a Codon Optimized Nucleotide Sequence Encoding GBA

This Example investigates the distribution and efficacy of the viral genome construct GB_VG17 (SEQ ID NO: 1812) comprising a codon-optimized nucleotide sequence (SEQ ID NO: 1773) encoding a GBA protein, vectorized in a VOY101 capsid (VOY101.GBA_VG17) in wild-type C57BL/6 mice. VOY101 is a capsid protein that enables blood brain barrier penetration after IV injection.

Mice were intravenously injected with 2e13 VG/kg of VOY101.GBA_VG17 or a vehicle control, into the lateral tail vein. At 28-days post IV injection, various CNS tissues (e.g., cortex, striatum, hippocampus, thalamus, cerebellum, brainstem, and/or spinal cord) and peripheral tissues (e.g., heart, liver, and/or spleen) were harvested to measure viral genome (VG) biodistribution (VG/cell), GCase activity, and GBA mRNA expression (transgene specific and endogenous). All animals treated remained healthy and there was no significant difference in the body weight between mice treated with VOY101.GBA_VG17 and mice treated with the vehicle control.

With respect to VG biodistribution, high levels (approximately >50 vg/cell) of VOY101.GBA_VG17 distribution was observed across the forebrain and midbrain. In the cortex, 81.31 VG/cell were quantified on average (range: ˜75-85 vg/cell); in the striatum, 150.39 VG/cell were quantified on average (range: ˜90-330 vg/cell); in the hippocampus, 152.91 VG/cell were quantified on average (range: ˜70-195 vg/cell); and in the thalamus, 117.94 VG/cell were quantified on average (range: ˜70-190 vg/cell). Therefore, successful VOY101.GBA_VG17 gene transfer was achieved across the forebrain and midbrain regions.

Similarly, high levels (approximately >50 vg/cell) of VOY101.GBA_VG17 distribution was observed across the hind brain and spinal cord (cervical region). In the cerebellum, 65.77 VG/cell were quantified on average (range: ˜23-105 vg/cell); in the brainstem, 159.22 VG/cell were quantified on average (range: ˜110-305 vg/cell); and in the spinal cord, 176.29 VG/cell were quantified on average (range: ˜95-280 vg/cell). Therefore, successful VOY101.GBA_VG17 gene transfer was also achieved across the hindbrain and spinal cord.

With respect to VG biodistribution in peripheral tissues, detectable levels of VOY101.GBA_VG17 were observed in the heart, spleen, and liver, but this was approximately 4-10 fold lower than the levels observed in the CNS tissues. In the heart, 11.58 VG/cell were quantified on average (range: ˜5-21 vg/cell); in the spleen, 29.99 VG/cell were quantified on average (range: ˜7-82 vg/cell); and in the liver, 18.76 VG/cell were quantified on average (range: ˜5-60 vg/cell).

With respect to GCase activity (measured as RFU/mL normalized to mg of protein), the forebrain and midbrain following IV injection of VOY101.GBA_VG17 demonstrated a significant increase in GCase activity over baseline (vehicle control). The highest increase was observed in the cortex (4.86 fold higher than the vehicle control). A similar increase was observed in the striatum (4.6 fold higher than the vehicle control) and the thalamus (4.74 fold higher than the vehicle control). Therefore, a significant increase in GCase activity was observed in the forebrain and midbrain which had high VOY101.GBA_VG17 biodistribution. Similarly, the hindbrain structures following IV injection of VOY101.GBA_VG17 demonstrated a significant increase in GCase activity over baseline (vehicle control). The cerebellum showed a 4.04 fold higher GCase activity than the vehicle control and the brainstem showed a 5.26 fold higher GCase activity than the vehicle control. Therefore, a significant increase in GCase activity was also observed in the hind brain which had high VOY101.GBA_VG17 biodistribution. Overall, all brain regions tested showed a 4-5 fold increase in GCase activity relative to the vehicle control and IV delivery of VOY101.GBA_VG17 resulted in a successful and uniform increase in GCase activity across pertinent CNS tissues.

GCase activity was also measured in the liver (as RFU/mL normalized to mg of protein) following IV injection of VOY101.GBA_VG17. The liver showed a 4.12 fold increase in GCase activity relative to the vehicle control, demonstrating a successful increase in GBA activity in a non-CNS tissue.

In additional to the cellular and tissue GCase activity levels quantified in both the CNS and the liver, GCase activity was also measured in the fluid of the mice, including in the cerebral spinal fluid (CSF) and the serum, post-IV injection of VOY101.GBA_VG17. GCase activity was higher in the serum as compared to the CSF. A 5.9 fold increase in GCase activity relative to the vehicle treated control was observed in the CSF and a 22.3 fold increase in GCase activity relative to the vehicle treated control was observed in the serum. These data demonstrate active GCase is secreted into extracellular compartments following IV injection of VOY101.GBA_VG17.

The GCase activity levels quantified and the fold increase in activity relative to the vehicle in the CNS and peripheral tissues and fluid measured following IV injection of VOY101.GBA_VG17 are summarized in Table 27.

TABLE 27
Summary of GCase activity levels (RFU/per
mL) normalized to mg of protein

| GCase Activity (RFU/per mL) | Fold Increase in GCase activity |
|---|---|
| normalized to mg of protein | relative to no vehicle control |

| | | |
|---|---|---|
| Cortex | Vehicle | 4.86 |
| 62246 | 12789 | |
| Striatum | Vehicle | 4.64 |
| 32063 | 6898 | |
| Thalamus | Vehicle | 4.74 |
| 40506 | 9061 | |
| Cerebellum | Vehicle | 4.04 |
| 36082 | 8926 | |
| Brainstem | Vehicle | 5.26 |
| 44706 | 8495 | |
| Liver | Vehicle | 4.1 |
| 100123 | 24272 | |
| CSF | Vehicle | 5.9 |
| 3707 | 627 | |
| Serum | Vehicle | 22.3 |
| 6919 | 310 | |

Both endogenous and transgene specific GBA mRNA (payload) expression was quantified post-IV injection of VOY101.GBA_VG17 in the cortex, thalamus, and brainstem. GBA mRNA was quantified as GBA mRNA expression per 1,000 transcripts normalized to geomean (GAPDH, HPRT1, PPIA). With respect to endogenous GBA mRNA, approximately 38-63 copies per 1000 transcripts were measured across the brain. More specifically, in the cortex, thalamus, and brainstem, 63.10 endogenous GBA mRNA/1,000 transcripts, 38.81 endogenous GBA mRNA/1,000 transcripts, and 38.95 endogenous GBA mRNA/1,000 transcripts were quantified, respectively. With respect transgene specific GBA mRNA, approximately 1314-1765 copies per 1000 transcripts were measured across the brain. In the cortex, thalamus, and brainstem, 1314.39 transgene specific GBA mRNA/1,000 transcripts, 1547.21 transgene specific GBA mRNA/1,000 transcripts, and 1764.02 transgene specific GBA mRNA/1,000 transcripts were quantified, respectively. Accordingly, in the cortex, thalamus, and brain stem, there was an 874 fold, 1032 fold, and 1244 fold increase in transcript specific GBA mRNA compared to the vehicle control, respectively. Therefore, successful transcription of the transgene comprising the codon-optimized nucleotide sequence (SEQ ID NO: 1773) encoding the GBA protein was achieved in the brain at 28-days post IV injection of a blood brain barrier penetrant VOY101.GBA_VG17 vector. Endogenous GBA mRNA levels in the brain maintained similar levels in the tissues treated with VOY101. GBA_VG17 and the tissues treated with the vehicle control. These data demonstrate successful transgene transcription and expression of a GBA payload in the CNS following IV injection of VOY101.GBA_VG17.

Both endogenous and transgene specific GBA mRNA expression was also quantified post-IV injection of VOY101.GBA_VG17 in the liver. In the liver, 182.29 endogenous GBA mRNA/1,000 transcripts were quantified (range: ˜188-240 per 1000 transcripts), and there was no significant difference in the endogenous GBA mRNA levels between treated and untreated mice. Approximately, 1372.45 transgene specific GBA mRNA/1,000 transcripts were quantified in the liver, and a 739 fold increase in GBA mRNA was observed in the treated mice compared to the vehicle control. These data demonstrate successful transgene transcription and expression of a GBA payload in the liver following IV injection of VOY101.GBA_VG17.

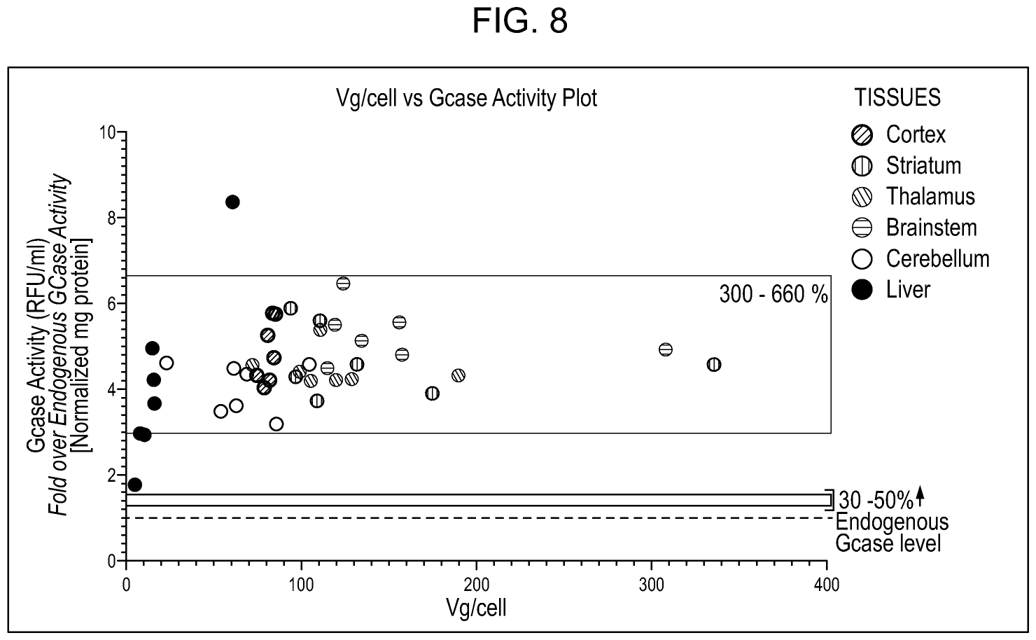

The relationship between biodistribution (VG/cell) and GCase activity (RFU/mL, fold over endogenous GCase activity, normalized to mg of protein) following IV injection of VOY101.GBA_VG17 was also evaluated in the cortex, striatum, thalamus, brainstem, cerebellum, and liver of the mice. In the CNS tissues, approximately a 300-660% fold increase in GCase activity over endogenous GCase activity was observed (FIG. 8) and 595-1825 transgene-specific GBA mRNA copies/1000 transcripts were quantified. In the liver, while there was intra-group variability, a 180-850% fold increase in GCase activity relative to endogenous GCase activity was measured, with approximately 330-2450 transgene specific GBA mRNA copies per 1000 transcripts quantified. The GCase levels quantified in the liver were comparable to the CNS tissues at lower VG/cell levels (FIG. 8). It is predicted that a 30-50% fold increase in GCase activity over endogenous is clinically impactful. Therefore, intravenous injection of VOY101.GBA_VG17 was able to increase the levels of GCase activity in various CNS and peripheral tissues relative to endogenous, well above the predicted fold increase thought to be clinically impactful.

Some of these data discussed above are summarized in Table 22 below. In summary, VOY101.GBA_VG17 which comprises the codon-optimized nucleotide sequence of SEQ ID NO: 1773 encoding the GBA protein demonstrated high biodistribution in the CNS, increased GCase activity in the CNS and peripheral tissues and fluid, and successful transgene transcription and expression. GBA_VG17 could therefore be used in the treatment of disorders associated with a lack of a GBA protein and/or GCase activity, such as neuronopathic (affects the CNS) and non-neuronopathic (affects non-CNS) Gaucher's disease, PD associated with a mutation in the GBA gene, and dementia with Lewy Bodies.

TABLE 22
Summary of VG biodistribution, GCase activity, and GBA mRNA
data 28 day post IV injection of VOY101.GBA_VG17 at
2e13 vg/kg in the cortex, thalamus, brain stem and liver

| | | | | GCase |
|---|---|---|---|---|
| | | GBA mRNA | GBA mRNA | expression |
| | VG/ | (over endogenous | (over vehicle | (over vehicle |
| Region | Cell | GBA) | baseline) | baseline) |
| Cortex | 83.31 | 20.83 | 874 | 4.86 |
| Thalamus | 117.94 | 39.87 | 1032 | 4.74 |
| Brain Stem | 159.22 | 45.29 | 1244 | 5.26 |
| Liver | 18.76 | 7.29 | 739 | 4.12 |

### Example 17. De-targeting GBA Expression in the Dorsal Root Ganglia (DRG)

This Example demonstrates the use of a miR183 binding site to reduce GBA expression in the dorsal root ganglion (DRG) neurons, which express the corresponding endogenous microRNA, miR183.

A viral genome construct, GBA_VG33 (SEQ ID NO: 1828, described in Tables 18-19 and 21), comprising a codon-optimized nucleotide sequence (SEQ ID NO: 1773) encoding a GBA protein and a miR183 binding site series (SEQ ID NO: 1849), comprising four miR183 binding sites (each comprising SEQ ID NO: 1847), each separated by an 8 nucleotide spacer (SEQ ID NO: 1848). The GBA_VG33 were also vectorized into an AAV2 vector (AAV2.GBA_VG33).

HEK293 cells were transfected with the GBA_VG33 construct and GBA protein expression was compared to cells transfected with the GB_VG17 control construct (SEQ ID NO: 1812) which comprises a codon-optimized nucleotide sequence (SEQ ID NO: 1773) encoding a GBA protein but does not comprise a miR183 binding site series. Similar GBA protein expression levels were observed following transfection with the GBA_VG33 construct and the GBA_VG17 control construct. HEK293 cells were also co-transfected with either the GBA_VG33 construct comprising the miR183 binding site series, or the GBA_VG17 control construct, and miR183, and GBA protein expression was measured. A significant reduction of GBA expression was observed in HEK293 cells co-transfected the GBA_VG33 construct and miR183 compared to those cells co-transfected with the GBA_VG17 control and miR183. These data demonstrated that the GBA_VG33 construct comprising the miR183 binding site series was able to reduce GBA expression in the presence of the corresponding microRNA (miR183).

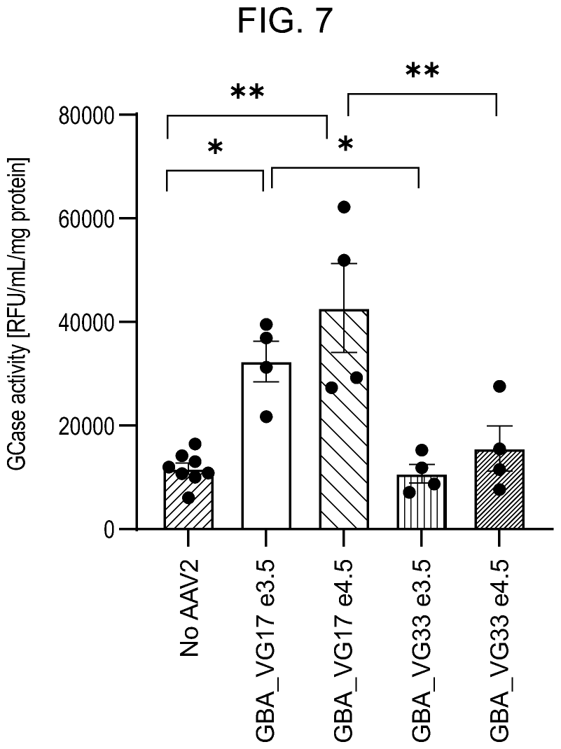

De-targeting of GBA expression in the DRG was also investigated in rat embryonic DRG neurons. The rat embryonic DRG neurons were transduced with AAV2.GBA_VG33 (comprises the miR183 binding site series) or AAV2.GBA_VG17 control at an MOI of 10³⁵ or 10⁴⁵, or a no AAV control. GCase activity was measured as RFU/mL per mg of total protein. As shown in FIG. 7, GCase activity was significantly reduced in the rat embryonic DRG neurons transduced with AAV2.GBA_VG33 compared to those transfected with AAV2.GBA_VG17, at both MOIs tested. Also, the levels of GCase activity measured in the rat embryonic DRG neurons transduced with AAV2.GBA_VG33 at both MOIs was similar to the level of GCase measured in the no AAV control.

Taken together, these data demonstrate that successful GBA expression de-targeting in the DRG was achieved with the GBA_VG33 (SEQ ID NO: 1828), comprising a codon-optimized nucleotide sequence (SEQ ID NO: 1773) encoding the GBA protein and the miR183 binding site series (SEQ ID NO: 1849).

### IX. Equivalents and Scope

Those skilled in the art will recognize, or be able to ascertain using no more than routine experimentation, many equivalents to the specific embodiments in accordance with the Detailed Description provided herein. The scope of the present disclosure is not intended to be limited to the above Detailed Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any, composition, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. An isolated, e.g., recombinant, nucleic acid comprising a transgene encoding a β-glucocerebrosidase (GBA) protein, wherein the nucleotide sequence encoding the GBA protein comprises a nucleotide sequence at least 90% identical to the nucleotide sequence of SEQ ID NO: 1773.

2. The isolated nucleic acid of claim 1, wherein the nucleotide sequence encoding the GBA protein comprises the nucleotide sequence of SEQ ID NO: 1773, or a nucleotide sequence at least 95% identical to SEQ ID NO: 1773.

3. The isolated nucleic acid of claim 1 or 2, which further comprises a nucleotide sequence encoding a miR binding site that reduces expression of the GBA protein encoded by the nucleic acid in a cell or tissue where the corresponding miRNA is expressed, optionally wherein the encoded miRNA binding site is complementary, e.g., fully complementary or partially complementary, to a miRNA expressed in a cell or tissue of the DRG, liver, hematopoietic, or a combination thereof.

4. An isolated, e.g., recombinant viral genome comprising a nucleic acid comprising a transgene encoding a GBA protein, and further comprising a nucleotide sequence encoding a miR binding site that modulates, e.g., reduces, expression of the encoded GBA protein in a cell or tissue of the DRG, liver, hematopoietic lineage, or a combination thereof.

5. The isolated nucleic acid of claim 3, or the viral genome of claim 4, wherein the encoded miR binding site comprises a miR183 binding site, a miR122 binding site, a miR-142-3p, or a combination thereof, optionally wherein:
(i) the encoded miR183 binding site comprises the nucleotide sequence of SEQ ID NO: 1847, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 1847;
(ii) the encoded miR122 binding site comprises the nucleotide sequence of SEQ ID NO: 1865, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 1865; and/or
(iii) the encoded miR-142-3p binding site comprises the nucleotide sequence of SEQ ID NO: 1869, or a nucleotide sequence having at least 70%, 75%, 80%, 85%, 90%, 92%, 95%, 97%, 98%, or 99% sequence identity thereto; or a nucleotide sequence having at least one, two, three, four, five, six, or seven modifications, but no more than ten modifications of SEQ ID NO: 1869.

6. The isolated nucleic acid of any one of claims 1-3, or the viral genome of claim 4 or 5, wherein the nucleic acid further encodes an enhancement element, wherein the encoded enhancement element comprises one, two, or all of:
(a) a prosaposin polypeptide, Saposin C polypeptide, or functional fragment or variant thereof, optionally comprising the amino acid sequence of SEQ ID NOs: 1789, 1758, 1750, 1752, 1754, 1756-1758, 1784, or 1785, an amino acid sequence having at least one, two, or three but no more than four modifications, e.g., substitutions (e.g., conservative substitutions), relative to SEQ ID NOs: 1789, 1758, 1750, 1752, 1754, 1756-1758, 1784, or 1785; or an amino acid sequence at least 85% identical thereto;
(b) a cell penetrating peptide, optionally comprising the amino acid sequence of any of SEQ ID NOs: 1794, 1796, or 1798, or an amino acid sequence having at least one, two, or three but no more than four modifications, e.g., substitutions (e.g., conservative substitutions), relative to SEQ ID NOs: 1794, 1796, or 1798; or
(c) a lysosomal targeting sequence, optionally comprising the amino acid sequence of any of SEQ ID NOs: 1800, 1802, 1804, 1806, or 1808, or an amino acid sequence having at least one, two, or three but no more than four modifications, e.g., substitutions (e.g., conservative substitutions), relative to SEQ ID NOs: 1800, 1802, 1804, 1806, or 1808.

7. An isolated, e.g., recombinant, nucleic acid comprising a transgene encoding a GBA protein and an enhancement element, wherein the encoded enhancement element comprises one, two, or all of:
(a) a Saposin C polypeptide or functional fragment or variant thereof, optionally comprising the amino acid sequence of SEQ ID NO: 1789 or 1758, or an amino acid sequence at least 85% identical thereto;
(b) a cell penetrating peptide, optionally comprising the amino acid sequence of any of SEQ ID NOs: 1794, 1796, or 1798, or an amino acid sequence having at least one, two, or three but no more than four modifications, e.g., substitutions (e.g., conservative substitutions), relative to SEQ ID NOs: 1794, 1796, or 1798; or
(c) a lysosomal targeting sequence, optionally comprising the amino acid sequence of any of SEQ ID NOs: 1800, 1802, 1804, 1806, or 1808, or an amino acid sequence having at least one, two, or three but no more than four modifications, e.g., substitutions (e.g., conservative substitutions), relative to SEQ ID NOs: 1800, 1802, 1804, 1806, or 1808.

8. The isolated nucleic acid of claim 6 or 7, or the viral genome of claim 6, wherein:
(i) the encoded enhancement element comprises the amino acid sequence of SEQ ID NO: 1789, or an amino acid sequence having at least one, two, or three but no more than four modifications, e.g., substitutions (e.g., conservative substitutions), relative to SEQ ID NO: 1789;
(ii) the nucleotide sequence encoding the enhancement element comprises the nucleotide sequence of 1787, or a nucleotide sequence or a nucleotide sequence at least 85% identical thereto;
(ii) the encoded enhancement element comprises the amino acid sequence of 1802, or an amino acid sequence having at least one, two, or three but no more than four modifications, e.g., substitutions (e.g., conservative substitutions), relative to SEQ ID NO: 1802;
(iii) the nucleotide sequence encoding the enhancement element comprises the nucleotide sequence of 1801, or a nucleotide sequence having at least one, two, or three but no more than four modifications, e.g., substitutions (e.g., conservative substitutions), relative to SEQ ID NO: 1801;
(iv) the encoded enhancement element comprises the amino acid sequence of 1794, or an amino acid sequence having at least one, two, or three but no more than four modifications, e.g., substitutions (e.g., conservative substitutions), relative to SEQ ID NO: 1794; or
(v) the nucleotide sequence encoding the enhancement element comprises the nucleotide sequence of 1793, or a nucleotide sequence having at least one, two, or three but no more than four modifications, e.g., substitutions (e.g., conservative substitutions), relative to SEQ ID NO: 1793.

9. The isolated nucleic acid of claim 7 or 8, or the viral genome of any one of claim 4-6 or 8, wherein the nucleotide sequence encoding the GBA protein comprises the nucleotide sequence of any one of SEQ ID NOs: 1773, 1777, or 1781, or a nucleotide sequence at least 90% (e.g., at least 92%, 95%, 97%, 98%, or 99%) identical thereto.

10. The isolated nucleic acid of any one of claims 6-9, or the viral genome of any one of claim 6 or 8-9, wherein the encoded enhancement element and the encoded GBA protein are connected directly (e.g., without a linker) or are connected via an encoded linker.

11. The isolated nucleic acid or the viral genome of claim 10, wherein:
(i) the encoded linker comprises the amino acid sequence of any of SEQ ID NOs: 1854, 1855, 1843, 1845, or an amino acid sequence having at least one, two, or three but no more than four modifications, e.g., substitutions (e.g., conservative substitutions), relative to SEQ ID NOs: 1854, 1855, 1843, 1845;
(ii) the nucleotide sequence encoding the linker comprises any of the nucleotide sequences of Table 2, or a nucleotide sequence having at least one, two, or three but no more than four modifications, e.g., substitutions (e.g., conservative substitutions), relative to the sequences of Table 2;
(iii) the nucleotide sequence encoding the linker comprises the nucleotide sequence of any one of SEQ ID NOs: 1724, 1726, 1729, or 1730, or a nucleotide sequence having at least one, two, or three but no more than four modifications, e.g., substitutions (e.g., conservative substitutions), relative to SEQ ID NOs: 1724, 1726, 1729, or 1730;
(iv) the encoded linker comprises a furin cleavage site;
(v) the encoded linker comprises a T2A polypeptide;
(vi) the encoded linker comprises a (Gly4Ser)n linker, wherein n is 1-10, e.g., n is 3, 4, or 5; and/or
(vii) the encoded linker comprises a (Gly4Ser)3 linker.

12. The isolated nucleic acid of any one of claims 6-11, or the viral genome of any one of claim 6 or 8-11, wherein:
(i) the nucleotide sequence encoding the enhancement element is located 5′ relative to the nucleotide sequence encoding the GBA protein; and/or
(ii) the nucleotide sequence encoding the enhancement element is located 3′ relative to the nucleotide sequence encoding the GBA protein.

13. The isolated nucleic acid of any one of claim 1-3 or 5-12, or the viral genome of any one of claim 4-6 or 8-12, further encoding a signal sequence, optionally wherein:
(i) the encoded signal sequence comprises the amino acid sequence of SEQ ID NO: 1853 or 1857, or an amino acid sequence at least 85% identical thereto; and/or
(ii) the nucleotide sequence encoding the signal sequence is located 5′ relative to the nucleotide sequence encoding the GBA protein; and/or 5′ relative to the encoded enhancement element.

14. The isolated nucleic acid of any one of claim 1-3 or 5-13, or the viral genome of any one of claim 4-6 or 8-13, wherein:
(i) the nucleic acid comprises in 5′ to 3′ order: a nucleotide sequence encoding a signal sequence comprising the nucleotide sequence of SEQ ID NO: 1850, or a nucleotide sequence at least 85% identical thereto; and a nucleotide sequence encoding a GBA protein comprising the nucleotide sequence of SEQ ID NO: 1773, or a nucleotide sequence at least 85% identical thereto; or
(ii) the nucleic acid encodes in 5′ to 3′ order: a signal sequence comprising the amino acid sequence of SEQ ID NO: 1853, or an amino acid sequence at least 85% identical thereto; and a GBA protein comprising the amino acid sequence of SEQ ID NO: 1775, or an amino acid sequence at least 85% identical thereto.

15. The isolated nucleic acid of any one of claims 7-13, or the viral genome of any one of claim 4-6 or 8-13, wherein the nucleic acid encodes in 5′ to 3′ order:
(i) a signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; and an enhancement element comprising the amino acid sequence of SEQ ID NO: 1800, or an amino acid sequence having at least one, two, or three but no more than four modifications, e.g., substitutions, relative to SEQ ID NO: 1800;
(ii) a signal sequence comprising the amino acid sequence of SEQ ID NO: 1853, or an amino acid sequence at least 85% identical thereto; an enhancement element comprising the amino acid sequence of SEQ ID NO: 1802, or an amino acid sequence at least 85% identical thereto; and a GBA protein comprising the amino acid sequence of SEQ ID NO: 1775, or an amino acid sequence at least 85% identical thereto;
(iii) a signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; and an enhancement element comprising the amino acid sequence of SEQ ID NO: 1804, or an amino acid sequence having at least one, two, or three but no more than four modifications, e.g., substitutions, relative to SEQ ID NO: 1804;
(iv) a signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; and an enhancement element comprising the amino acid sequence of SEQ ID NO: 1806, or an amino acid sequence having at least one, two, or three but no more than four modifications, e.g., substitutions, relative to SEQ ID NO: 1806;
(v) a signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; a linker comprising the amino acid sequence of SEQ ID NO: 1845, or an amino acid sequence having at least one, two, or three but no more than four modifications, e.g., substitutions, relative to SEQ ID NO: 1845; and an enhancement element comprising the amino acid sequence of SEQ ID NO: 1798, or an amino acid sequence having at least one, two, or three but no more than four modifications, e.g., substitutions, relative to SEQ ID NO: 1798;
(vi) a signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; a linker comprising the amino acid sequence of SEQ ID NO: 1845, or an amino acid sequence having at least one, two, or three but no more than four modifications, e.g., substitutions, relative to SEQ ID NO: 1845; and an enhancement element comprising the amino acid sequence of SEQ ID NO: 1794, or an amino acid sequence having at least one, two, or three but no more than four modifications, e.g., substitutions, relative to SEQ ID NO: 1794;
(vii) a first signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1785, or an amino acid sequence at least 85% identical thereto;
(viii) a first signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1789, or an amino acid sequence at least 85% identical thereto;
(ix) a first signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1758, or an amino acid sequence at least 85% identical thereto;
(x) a signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; a linker comprising the amino acid sequence of SEQ ID NO: 1845, or an amino acid sequence having at least one, two, or three but no more than four modifications, e.g., substitutions, relative to SEQ ID NO: 1845; and an enhancement element comprising the amino acid sequence of SEQ ID NO: 1796, or an amino acid sequence at least 85% identical thereto;
(xi) a signal sequence comprising the amino acid sequence of SEQ ID NO: 1853, or an amino acid sequence at least 85% identical thereto; an enhancement element comprising the amino acid sequence of SEQ ID NO: 1794, or an amino acid sequence having at least one, two, or three but no more than four modifications, e.g., substitutions, relative to SEQ ID NO: 1794; a linker comprising the amino acid sequence of SEQ ID NO: 1845, or an amino acid sequence having at least one, two, or three but no more than four modifications, e.g., substitutions, relative to SEQ ID NO: 1845; and a GBA protein comprising the amino acid sequence of SEQ ID NO: 1775, or an amino acid sequence at least 85% identical thereto;
(xii) a signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; and an enhancement element comprising the amino acid sequence of SEQ ID NO: 1808, or an amino acid sequence having at least one, two, or three but no more than four modifications, e.g., substitutions, relative to SEQ ID NO: 1808;
(xiii) a first signal sequence comprising the amino acid sequence of SEQ ID NO: 1853, or an amino acid sequence at least 85% identical thereto; a first enhancement element comprising the amino acid sequence of SEQ ID NO: 1802, or an amino acid sequence at least 85% identical thereto; a GBA protein comprising the amino acid sequence of SEQ ID NO: 1775, or an amino acid sequence at least 85% identical thereto; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and a second enhancement element comprising the amino acid sequence of SEQ ID NO: 1789, or an amino acid sequence at least 85% identical thereto;
(xiv) a first signal sequence comprising the amino acid sequence of SEQ ID NO: 1853, or an amino acid sequence at least 85% identical thereto; a GBA protein comprising the amino acid sequence of SEQ ID NO: 1775, or an amino acid sequence at least 85% identical thereto; a linker comprising the amino acid sequence of SEQ ID NO: 1845, or an amino acid sequence having at least one, two, or three but no more than four modifications, e.g., substitutions, relative to SEQ ID NO: 1845; a first enhancement element comprising the amino acid sequence of SEQ ID NO: 1798, or an amino acid sequence having at least one, two, or three but no more than four modifications, e.g., substitutions, relative to SEQ ID NO: 1798; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1789, or an amino sequence at least 85% identical thereto; or
(xv) a first signal sequence comprising the amino acid sequence of SEQ ID NO: 1853, or an amino acid sequence at least 85% identical thereto; a first enhancement element comprising the amino acid sequence of SEQ ID NO: 1802, or an amino acid sequence at least 85% identical thereto; a GBA protein comprising the amino acid sequence of SEQ ID NO: 1775, or an amino acid sequence at least 85% identical thereto; a linker comprising the amino acid sequence of SEQ ID NO: 1845, or an amino acid sequence having at least one, two, or three but no more than four modifications, e.g., substitutions, relative to SEQ ID NO: 1845; a first enhancement element comprising the amino acid sequence of SEQ ID NO: 1798, or an amino acid sequence having at least one, two, or three but no more than four modifications, e.g., substitutions, relative to SEQ ID NO: 1798; a furin cleavage site comprising the amino acid sequence of SEQ ID NO: 1854, or an amino acid sequence having at least one, two, or three but no more than four modifications, e.g., substitutions, relative to SEQ ID NO: 1854; a T2A polypeptide comprising the amino acid sequence of SEQ ID NO: 1855, or an amino acid sequence having at least one, two, or three but no more than four modifications, e.g., substitutions, relative to SEQ ID NO: 1855; a second signal sequence comprising the nucleotide sequence of SEQ ID NO: 1857, or an amino acid sequence having at least one, two, or three but no more than four modifications, e.g., substitutions, relative to SEQ ID NO: 1857; and an enhancement element comprising the amino acid sequence of SEQ ID NO: 1789, or an amino acid sequence at least 85% identical thereto.

16. An isolated, e.g., recombinant, viral genome comprising a promoter operably linked to the nucleic acid of any one of claim 1-3 or 7-15.

17. The viral genome of claim 4-6 or 7-15, further comprising a promoter operably linked to the nucleic acid comprising the transgene encoding the GBA protein, wherein the promoter comprises a tissue specific promoter or a ubiquitous promoter.

18. The viral genome of claim 16 or 17, wherein the promoter comprises:
(i) a chicken β-actin (CBA) promoter and/or its derivative CAG, an EF-1α promoter, a CMV immediate-early enhancer and/or promoter, a (3 glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof; and/or
(ii) the nucleotide sequence of any of SEQ ID NOs: 1832, 1833, 1834, 1835, 1836, 1839, 1840, or a nucleotide sequence at least 95% identical thereto.

19. The viral genome of any one of claims 16-18, wherein the promoter or functional variant thereof comprises:
(i) the nucleotide sequence of SEQ ID NO: 1834, or a nucleotide sequence at least 95% identical thereto; or
(ii) the nucleotide sequence of SEQ ID NO: 1839 or 1840, or a nucleotide sequence at least 95% identical thereto.

20. The viral genome of any one of claim 4-6 or 8-19, which further comprises an enhancer, optionally comprising the nucleotide sequence of SEQ ID NO: 1831, or a nucleotide sequence at least 95% identical thereto.

21. The viral genome of claim 4-6 or 8-20, which comprises an enhancer comprising the nucleotide sequence of SEQ ID NO: 1831, or a nucleotide sequence at least 95% identical thereto, and a promoter comprising the nucleotide sequence of SEQ ID NO: 1834, or a nucleotide sequence at least 95% identical thereto.

22. The viral genome of any one of claim 4-6 or 8-20, which further comprises:
(i) an inverted terminal repeat (ITR) sequence, optionally wherein the ITR sequence is positioned 5′ relative to the transgene encoding the GBA protein and/or the ITR sequence is positioned 3′ relative to the transgene encoding the GBA protein;
(ii) a polyadenylation (polyA) signal region;
(iii) an intron region;
(iv) an exon region, e.g., at least one, two, or three exon regions;
(v) a Kozak sequence

23. The viral genome of claim 22, wherein:
(i) the ITR comprises a nucleotide sequence of SEQ ID NO: 1829 or 1830, or a nucleotide sequence at least 95% identical thereto; or
(ii) the ITR positioned 5′ relative to the nucleic acid comprising the transgene encoding the GBA protein comprises the nucleotide sequence of SEQ ID NO: 1829, or a nucleotide sequence at least 95% identical thereto; and/or the ITR positioned 3′ relative to the nucleic acid comprising the transgene encoding the GBA protein comprises the nucleotide sequence of SEQ ID NO: 1830, or a nucleotide sequence at least 95% identical thereto.

24. The viral genome of claim 22 or 23, wherein:
(i) the polyadenylation (polyA) signal region comprises the nucleotide sequence of SEQ ID NO: 1846, or a nucleotide sequence at least 95% identical thereto;
(ii) the intron comprises a beta-globin intron; and/or
(iii) the intron comprises the nucleotide sequence of SEQ ID NO: 1842, or a nucleotide sequence at least 95% identical thereto.

25. The isolated nucleic acid of any one of claim 3, 5-6, or 8-24, or the viral genome of any one of claim 4-6 or 8-24, which comprises:
(i) at least 1-5 copies of the encoded miR binding site, e.g., at least 1, 2, 3, 4, or 5 copies;
(ii) at least 4 copies of an encoded miR binding sites, optionally wherein all four copies comprise the same miR binding site, or at least one, two, three, or all of the copies comprise a different miR binding site, optionally wherein the 4 copies of the encoded miR binding sites are continuous, e.g., not separated by a spacer; or are separated by a spacer.

26. The isolated nucleic acid of any one of claim 3, 5-6, or 8-25, or the viral genome of any one of claim 4-6 or 8-25, wherein the viral genome comprises:
(i) a first encoded miR183 binding site comprising the nucleotide sequence of SEQ ID NO: 1847, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 1847;
(ii) a first spacer sequence comprising the nucleotide sequence of SEQ ID NO: 1848, or a nucleotide sequence having at least one, two, or three modifications, but no more than four modifications of SEQ ID NO: 1848;
(iii) a second encoded miR183 binding site comprising the nucleotide sequence of SEQ ID NO: 1847, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 1847;
(iv) a second spacer sequence comprising the nucleotide sequence of SEQ ID NO: 1848, or a nucleotide sequence having at least one, two, or three modifications, but no more than four modifications of SEQ ID NO: 1848;
(v) a third encoded miR183 binding site comprising the nucleotide sequence of SEQ ID NO: 1847, or a nucleotide sequence having at least 85%, 90%, 92%, 95%, 97%, 98%, or 99% sequence identity thereto; or a nucleotide sequence having at least one, two, three, four, five, six, or seven modifications, but no more than ten modifications of SEQ ID NO: 1847;
(vi) a third spacer sequence comprising the nucleotide sequence of SEQ ID NO: 1848, or a nucleotide sequence having at least one, two, or three modifications, but no more than four modifications of SEQ ID NO: 1848; and
(vii) a fourth encoded miR183 binding site comprising the nucleotide sequence of SEQ ID NO: 1847, or a nucleotide sequence having at least 70%, 75%, 80%, 85%, 90%, 92%, 95%, 97%, 98%, or 99% sequence identity thereto; or a nucleotide sequence having at least one, two, three, four, five, six, or seven modifications, but no more than ten modifications of SEQ ID NO: 1847.

27. An isolated, e.g., recombinant, viral genome comprising in 5′ to 3′ order:
(i) a 5′ adeno-associated (AAV) ITR, optionally wherein the 5′ AAV ITR comprises the nucleotide sequence of SEQ ID NO: 1829, or a nucleotide sequence at least 95% identical thereto;
(ii) a CMVie enhancer, optionally wherein the CMVie enhancer comprises the nucleotide sequence of SEQ ID NO: 1831, or a nucleotide sequence at least 95% identical thereto;
(iii) a CB promoter or functional variant thereof, optionally wherein the CB promoter or functional variant thereof comprises the nucleotide sequence of SEQ ID NO: 1834, or a nucleotide sequence at least 95% identical thereto;
(iv) an intron, optionally wherein the intron comprises the nucleotide sequence of SEQ ID NO: 1842, or a nucleotide sequence at least 95% identical thereto;
(v) a nucleotide sequence encoding a signal sequence, optionally wherein the nucleotide sequence encoding the signal sequence comprises the nucleotide sequence of SEQ ID NO: 1850, or a nucleotide sequence at least 95% identical thereto;
(vi) a transgene encoding a GBA protein, wherein the nucleotide sequence encoding the GBA protein comprises the nucleotide sequence of SEQ ID NO: 1773 or a nucleotide sequence at least 88% identical to the nucleotide sequence of SEQ ID NO: 1773;
(vii) a polyA signal region, optionally wherein the polyA signal region comprises the nucleotide sequence of SEQ ID NO: 1846, or a nucleotide sequence at least 95% identical thereto; and
(viii) a 3′ AAV ITR, optionally wherein the 3′ AAV ITR comprises the nucleotide sequence of SEQ ID NO: 1830, or a nucleotide sequence at least 95% identical thereto.

28. An isolated, e.g., recombinant, viral genome comprising in 5′ to 3′ order:
(i) a 5′ adeno-associated (AAV) ITR, optionally wherein the 5′ AAV ITR comprises the nucleotide sequence of SEQ ID NO: 1829, or a nucleotide sequence at least 95% identical thereto;
(ii) a CMVie enhancer, optionally wherein the CMVie enhancer comprises the nucleotide sequence of SEQ ID NO: 1831, or a nucleotide sequence at least 95% identical thereto;
(iii) a CB promoter or functional variant thereof, optionally wherein the CB promoter or functional variant thereof comprises the nucleotide sequence of SEQ ID NO: 1834, or a nucleotide sequence at least 95% identical thereto;
(iv) an intron, optionally wherein the intron comprises the nucleotide sequence of SEQ ID NO: 1842, or a nucleotide sequence at least 95% identical thereto;
(v) a nucleotide sequence encoding a signal sequence, optionally wherein the nucleotide sequence encoding the signal sequence comprises the nucleotide sequence of SEQ ID NO: 1850, or a nucleotide sequence at least 95% identical thereto;
(vi) a transgene encoding a GBA protein, optionally wherein the nucleotide sequence encoding the GBA protein comprises the nucleotide sequence of SEQ ID NO: 1773 or a nucleotide sequence at least 88% identical to the nucleotide sequence of SEQ ID NO: 1773;
(vii) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;
(viii) a spacer sequence, optionally wherein the spacer comprises the nucleotide sequence of SEQ ID NO: 1848, or a nucleotide sequence having at least one, two, or three modifications, but no more than four modifications of SEQ ID NO: 1848;
(ix) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;
(x) a spacer sequence, optionally wherein the spacer comprises the nucleotide sequence of SEQ ID NO: 1848, or a nucleotide sequence having at least one, two, or three modifications, but no more than four modifications of SEQ ID NO: 1848;
(xi) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;
(xii) a spacer sequence, optionally wherein the spacer comprises the nucleotide sequence of SEQ ID NO: 1848, or a nucleotide sequence having at least one, two, or three modifications, but no more than four modifications of SEQ ID NO: 1848;
(xiii) an encoded miR183 binding site, optionally wherein the encoded miR183 binding site comprises the nucleotide sequence of SEQ ID NO: 1847, or a nucleotide sequence having at least one, two, three, four, five, six, or seven modifications, but no more than ten modifications of SEQ ID NO: 1847;
(xiv) a polyA signal region, optionally wherein the polyA signal region comprises the nucleotide sequence of SEQ ID NO: 1846, or a nucleotide sequence at least 95% identical thereto; and
(xv) a 3′ AAV ITR, optionally wherein the 3′ AAV ITR comprises the nucleotide sequence of SEQ ID NO: 1830, or a nucleotide sequence at least 95% identical thereto.

29. The viral genome of any one of claim 4-6 or 8-28, which comprises the nucleotide sequence of SEQ ID NO: 1812, 1829, 1759-1771, 1809-1811, 1813-1827, or 1870, ora nucleotide sequence at least 95% identical thereto.

30. The viral genome of any one of claim 4-6 or 8-29, which:
(i) is single stranded;
(ii) further comprises a nucleic acid encoding a capsid protein, e.g., a structural protein, wherein the capsid protein comprises a VP1 polypeptide, a VP2 polypeptide, and/or a VP3 polypeptide, optionally wherein the VP1 polypeptide, the VP2 polypeptide, and/or the VP3 polypeptide are encoded by at least one Cap gene; and/or
(iii) further comprises a nucleic acid encoding a Rep protein, e.g., a non-structural protein, wherein the Rep protein comprises a Rep78 protein, a Rep68, Rep52 protein, and/or a Rep40 protein, optionally wherein the Rep78 protein, the Rep68 protein, the Rep52 protein, and/or the Rep40 protein are encoded by at least one Rep gene.

31. An isolated, e.g., recombinant GBA protein encoded by the isolated nucleic acid of any one of claim 1-3 or 5-15 or the viral genome of any one of claim 4-6 or 8-30.

32. An isolated, e.g., recombinant, AAV particle comprising:
(i) a capsid protein; and
(ii) the viral genome of any one of claim 4-6 or 8-30.

33. The AAV particle of claim 32, wherein:
(i) the capsid protein comprises the amino acid sequence of SEQ ID NO: 138, or an amino acid sequence with at least 90% sequence identity thereto;
(ii) the capsid protein comprises an amino acid sequence having at least one, two or three modifications but not more than 30, 20 or 10 modifications of the amino acid sequence of SEQ ID NO: 138;
(iii) the capsid protein comprises the amino acid sequence of SEQ ID NO: 11, or an amino acid sequence with at least 90% sequence identity thereto;
(iv) the capsid protein comprises an amino acid sequence having at least one, two or three modifications but not more than 30, 20 or 10 modifications of the amino acid sequence of SEQ ID NO: 11;
(v) the capsid protein comprises an amino acid sequence encoded by the nucleotide sequence of SEQ ID NO: 137, or a sequence with at least 90% sequence identity thereto; and/or
(vi) the nucleotide sequence encoding the capsid protein comprises the nucleotide sequence of SEQ ID NO: 137, or a sequence with at least 90% sequence identity thereto.

34. The AAV particle of claim 32 or 33, wherein the capsid protein comprises:
(i) an amino acid substitution at position K449, e.g., a K449R substitution, numbered according to SEQ ID NO:138;
(ii) an insert comprising the amino acid sequence of TLAVPFK (SEQ ID NO: 1262), optionally wherein the insert is present immediately subsequent to position 588, relative to a reference sequence numbered according to SEQ ID NO:138;
(iii) an amino acid other than “A” at position 587 and/or an amino acid other than “Q” at position 588, numbered according to SEQ ID NO: 138;
(iv) the amino acid substitution of A587D and/or Q588G, numbered according to SEQ ID NO:138.

35. The AAV particle of any one of claims 32-34, wherein:
(i) the capsid protein comprises the amino acid sequence of SEQ ID NO: 1, or an amino acid sequence having at least 90%, 92%, 95%, 97%, 98%, or 99% sequence identity thereto;
(ii) the capsid protein comprises an amino acid sequence comprising at least one, two, or three modifications but no more than 30, 20, or 10 modifications, e.g., substitutions, relative to the amino acid sequence of SEQ ID NO: 1;
(iii) the capsid protein comprises an amino acid sequence encoded by the nucleotide sequence of SEQ ID NO: 2 or a nucleotide sequence having at least 90%, 92%, 95%, 97%, 98%, or 99% sequence identity thereto; and/or
(iv) the nucleotide sequence encoding the capsid protein comprises the nucleotide sequence of SEQ ID NO: 2, or a nucleotide sequence having at least 90%, 92%, 95%, 97%, 98%, or 99% sequence identity thereto.

36. A vector comprising the viral genome of any one of claim 4-6 or 8-30 or the nucleic acid of any one of claim 1-3 or 5-15.

37. A cell comprising the viral genome of any one of claim 4-6 or 8-30, the viral particle of any one of claims 32-35, or the vector of claim 36, optionally wherein the cell is a mammalian cell, e.g., an HEK293 cell, an insect cell, e.g., an Sf9 cell, or a bacterial cell.

38. A method of making an isolated, e.g., recombinant, AAV particle, the method comprising
(i) providing a host cell comprising the viral genome of any one of claim 4-6 or 8-30; and
(ii) incubating the host cell under conditions suitable to enclose the viral genome in a capsid protein, e.g., a VOY101 capsid protein;
thereby making the isolated AAV particle.

39. A pharmaceutical composition comprising the AAV particle of any one of claims 32-35, or an AAV particle comprising the viral genome of any one of claim 4-6 or 8-30, and a pharmaceutically acceptable excipient.

40. A method of delivering an exogenous GBA protein to a subject, comprising administering an effective amount of the pharmaceutical composition of claim 39, the AAV particle of any one of claims 32-35, an AAV particle comprising the viral genome of any one of claim 4-6 or 8-30, or an AAV particle comprising a viral genome comprising the nucleic acid of any one of claim 1-3 or 5-15, thereby delivering the exogenous GBA protein to the subject.

41. The method of claim 40, wherein the subject has, has been diagnosed with having, or is at risk of having:
(i) a disease associated with expression of GBA, e.g., aberrant or reduced GBA expression, e.g., expression of an GBA gene, GBA mRNA, and/or GBA protein; or
(ii) a neurodegenerative or neuromuscular disorder.

42. A method of treating a subject having or diagnosed with having a disease associated with GBA expression comprising administering an effective amount of the pharmaceutical composition of claim 39, the AAV particle of any one of claims 32-35, an AAV particle comprising the viral genome of any one of claim 4-6 or 8-30, or an AAV particle comprising a viral genome comprising the nucleic acid of any one of claim 1-3 or 5-15 thereby treating the disease associated with GBA expression in the subject.

43. A method of treating a subject having or diagnosed with having a neurodegenerative or neuromuscular disorder, comprising administering an effective amount of the pharmaceutical composition of claim 39, the AAV particle of any one of claims 32-35, an AAV particle comprising the viral genome of any one of claim 4-6 or 8-30, or an AAV particle comprising a viral genome comprising the nucleic acid of any one of claim 1-3 or 5-15, thereby treating the neurodegenerative or neuromuscular disorder in the subject.

44. The method of any one of claims 41-43, wherein the disease associated with expression of GBA or the neurodegenerative or neuromuscular disorder comprises Parkinson's Disease (PD), dementia with Lewy Bodies (DLB), Gaucher disease (GD), Spinal muscular atrophy (SMA), Multiple System Atrophy (MSA), or Multiple sclerosis (MS).

45. The method of claim 44, wherein the PD is:
(i) associated with a mutation in a GBA gene;
(ii) early onset PD (e.g., before 50 years of age) or juvenile PD (e.g., before 20 years of age);
(iii) a tremor dominant, postural instability gait difficulty PD (PIGD); or
(iv) a sporadic PD (e.g., a PD not associated with a mutation).

46. The method of claim 44, wherein the GD is:
(i) neuronopathic GD (e.g., affect a cell or tissue of the CNS, e.g., a cell or tissue of the brain and/or spinal cord), non-neuronopathic GD (e.g., does not affect a cell or tissue of the CNS), or combination thereof; or
(ii) Type I GD (GD1), Type 2 GD (GD2), or Type 3 GD (GD3), optionally wherein the GD1 is non-neuronopathic GD and the GD2 is a neuronopathic GD.

47. The method of any one of claims 40-46, wherein the subject:
(i) has a mutation in a GBA gene, GBA mRNA, and/or GBA protein; and/or
(ii) is a human, optionally wherein the subject is a juvenile (e.g., between 6 years of age to 20 years of age) or an adult (e.g., above 20 years of age).

48. The method of any one of claims 40-47, wherein the AAV particle is administered to the subject intravenously, intracerebrally, via intrathalamic (ITH) administration, intramuscularly, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, via intra-cisterna magna injection (ICM), or via dual ITH and ICM administration.

49. The method of any one of claims 40-48, wherein the AAV particle is administered via intravenous administration, optionally wherein the intravenous administration is via focused ultrasound (FUS), e.g., coupled with the intravenous administration of microbubbles (FUS-MB), or MRI-guided FUS coupled with intravenous administration.

50. The method of any one of claims 40-49, wherein the administration results in an increase in at least one, two, or all of:
(i) the level of GCase activity in a cell, tissue, (e.g., a cell or tissue of the CNS, e.g., the cortex, striatum, thalamus, cerebellum, and/or brainstem), and/or fluid (e.g., CSF and/or serum), of the subject, optionally wherein the level of GCase activity is increased by at least 3, 4, 4.5, 4.6, 4.7, 4.8, 4.9, 5, 5.1, 5.2, 5.3, 5.4, or 5.5 fold, as compared to a reference level, e.g., a subject that has not received treatment, e.g., has not been administered the AAV particle;
(ii) the level of viral genomes (VG) per cell in a CNS tissue (e.g., the cortex, striatum, thalamus, cerebellum, brainstem, and/or spinal cord) of the subject, optionally wherein the VG level is increased by greater than 50 VGs per cell, as compared to a peripheral tissue, wherein the level of VGs per cell is at least 4-10 fold lower than the levels in the CNS tissue, e.g., as measured by an assay as described herein; and/or
(iii) the level of GBA mRNA expression in a cell or tissue (e.g. a cell or tissue of the CNS, e.g., the cortex, thalamus, and/or brainstem), optionally wherein the level of GBA mRNA is increased by at least 100-1300 fold, e.g., 100 fold, 200 fold, 500 fold, 600 fold, 850 fold, 900 fold, 950 fold, 1000 fold, 1050 fold, 1100 fold, 1150 fold, 1200 fold, 1250 fold, or 1300 fold as compared to a reference level, e.g., a subject that has not received treatment (e.g., has not been administered the AAV particle), or endogenous GBA mRNA levels, e.g., as measured by an assay as described herein.

51. The method of any one of claims 40-50, further comprising administration of an additional therapeutic agent and/or therapy suitable for treatment or prevention of the disease associated GBA expression, the neurodegenerative disorder, and/or the neuromuscular disorder, optionally wherein the additional therapeutic agent comprises enzyme replacement therapy (ERT) (e.g., imiglucerase, velaglucerase alfa, or taliglucerase alfa); substrate reduction therapy (SRT) (e.g., eliglustat or miglustat), blood transfusion, levodopa, carbidopa, Safinamide, dopamine agonists (e.g., pramipexole, rotigotine, or ropinirole), anticholinergics (e.g., benztropine or trihexyphenidyl), cholinesterase inhibitors (e.g., rivastigmine, donepezil, or galantamine), an N-methyl-d-aspartate (NMDA) receptor antagonist (e.g., memantine), or a combination thereof.

52. The isolated nucleic acid of any one of claim 1-3 or 5-15, the viral genome of any one of claim 4-6 or 8-30, the AAV particle of any one of claims 32-35, or the pharmaceutical composition of claim 39, for use in the manufacture of a medicament.

53. The isolated nucleic acid of any one of claim 1-3 or 5-15, the viral genome of any one of claim 4-6 or 8-30, the AAV particle of any one of claims 32-35, or the pharmaceutical composition of claim 39, for use in the treatment of a disease associated with GBA expression, a neuromuscular and/or a neurodegenerative disorder.

54. Use of an effective amount of an AAV particle comprising the genome of any one of claim 4-6 or 8-30, an AAV particle comprising a genome comprising the nucleic acid of any one of claim 1-3 or 5-15, the AAV particle of any one of claims 32-35, or the pharmaceutical composition of claim 39, in the manufacture of a medicament for the treatment of a disease associated with GBA expression, a neuromuscular and/or a neurodegenerative disorder.
